(19) United States
(12) Patent Application Publication
(10) Pub. No.: US 2023/0165812 A1
(43) Pub. Date: Jun. 1, 2023

(54) NOCICEPTOR NEURONS CONTROL CANCER IMMUNOSURVEILLANCE

(71) Applicants: President and Fellows of Harvard College, Cambridge, MA (US); Université de Montréal, Montréal (CA); Children's Medical Center Corporation, Boston, MA (US)

(72) Inventors: Clifford J. Woolf, Newton, MA (US); Sebastien Talbot, Montreal (CA); Bruce P. Bean, Cambridge, MA (US)

(73) Assignees: President and Fellows of Harvard College, Cambridge, MA (US); Université de Montréal, Montréal, QC (CA); Children's Medical Center Corporation, Boston, MA (US)

(21) Appl. No.: 17/802,398

(22) PCT Filed: Feb. 26, 2021
(86) PCT No.: PCT/US21/19796

§ 371 Date: Aug. 25, 2022

Related U.S. Application Data
(60) Provisional application No. 62/982,622, filed on Feb. 27, 2020.

(51) Int. Cl. A61K 31/167; A61K 31/40; A61K 31/4425; A61K 45/06

(52) U.S. Cl. CPC A61K 31/167; A61K 31/40; A61K 31/4425; A61K 45/06

(57) ABSTRACT

The present disclosure provides methods of treating cancer by silencing tumor-innervating sensory neurons. The methods include treating cancer by genetic ablation of ion channels (e.g., TRPV1 or Na_{V}1.8), local pharmacological silencing or blockade of neuropeptide release from tumor-innervating nociceptor (e.g., with QX-314 and BoNT/a), as well as the antagonism of the CGRP receptor RAMP1 (e.g., with BIBN 4096).

### RELATED APPLICATIONS

This application claims priority under 35 U.S.C. § 119(e) to U.S. Provisional Application, U.S. Ser. No. 62/982,622, filed on Feb. 27, 2020, which is incorporated herein by reference.

### GOVERNMENT SUPPORT

This invention was made with government support under NS105076 awarded by the National Institutes of Health. The government has certain rights in the invention.

### BACKGROUND

Cytotoxic T cells express a variety of receptors, including PD-1 (Programmed Death-1), Tim-3 (T cell immunoglobulin and mucin domain-containing protein 3), and Lag-3 (Lymphocyte Activation Gene-3) (Dougan, M. et al. Annu Rev Immunol 27, 83-117 (2009); Chambers, C. A. et al. Annu Rev Immunol 19, 565-594 (2001); Topalian, S. L. et al. N Engl J Med 366, 2443-2454 (2012); Das, M. et al. Immunol Rev 276, 97-111 (2017)), that upon interaction with their cognate ligands, inhibit T cell function. These checkpoints ensure that immune responses to damage or infection are kept in check, preventing overly intense responses that might damage healthy cells (Baumeister, S. H. et al. Annu Rev Immunol 34, 539-573 (2016)). Tumor cells also express ligands for these immune checkpoints, which, once activated, block the cytolytic functions of T cells, thereby favoring survival of cancer cells (Baumeister, S. H., et al. Annu Rev Immunol 34, 539-573 (2016); Vesely, M. D. et al. Annu Rev Immunol 29, 235-271 (2011); Woo, S. R. et al. Annu Rev Immunol 33, 445-474 (2015)). Blocking the activity of immune checkpoint proteins releases this cancer cell-induced “brake” on the immune system, increasing the ability of the system to now eliminate tumor (Dougan, M. et al. Annu Rev Immunol 27, 83-117 (2009); Chambers, C. A. et al. Annu Rev Immunol 19, 565-594 (2001); Topalian, S. L. et al. N Engl J Med 366, 2443-2454 (2012); Baumeister, S. H. et al. Annu Rev Immunol 34, 539-573 (2016)). Several immune checkpoint inhibitors, including those targeting PD-L1, improve clinical outcomes for metastatic melanoma (Topalian, S. L. et al. N Engl J Med 366, 2443-2454 (2012); Wolchok, J. D. et al. N Engl J Med 369, 122-133, (2013); Long, G. V. et al. JAMA Oncol 3, 1511-1519 (2017)), but with varying degrees of effectiveness.

In addition to noxious stimuli detection leading to pain and prompting defensive reflexes, high threshold nociceptor neurons also promote host defenses through direct interaction with immune cells (Talbot, S. et al. Annu Rev Immunol 34, 421-447 (2016)). Neuro-immunity is mediated by the release of cytokines and neuropeptides and activation of their cognate receptors. While this dialogue helps to protect from danger, it may also contribute to disease. The somatosensory nervous system is positioned in epithelial and mucosal surfaces as well as primary and secondary lymphoid tissues to modulate immune responses (Talbot, S. et al. Neuron 87, 341-354, (2015); Rosas-Ballina, M. et al. Science 334, 98-101 (2011); Downing, J. E. et al. Immunol Today 21, 281-289 (2000); Veiga-Fernandes, H. et al. Cell 165, 801-811 (2016); McMahon, S. B. et al. Nat Rev Neurosci 16, 389-402 (2015); Foster, S. L. et al. Immunity 42, 403-405 (2015)). Nociceptors directly detect and respond to foreign antigens (Talbot, S. et al. Journal of neuroinflammation 6, 11 (2009)), immune cell-released cytokines (Talbot, S. et al. Neuron 87, 341-354), as well as toxins and surface receptors expressed by microbes and fungi (Kashem, S. W. et al. Trends Immunol 37, 440-450 (2016); Kashem, S. W. et al. Immunity 43, 515-526 (2015)). Nociceptor activation not only results in pain and itch but also local neuropeptide release from their peripheral terminals (Long, G. V. et al. JAMA Oncol 3, 1511-1519 (2017); Foster, S. L. et al. Front Immunol 8, 1463 (2017)). For example, Calcitonin Gene-Related Peptide (CGRP) increases T cell adhesion, blocks CD8 proliferation, reduces DC migration to lymph nodes, and suppresses FAS-L expression (Ding, W. et al. J Immunol 181, 6020-6026 (2008); Jimeno, R. et al. Immunol Cell Biol 90, 178-186 (2012); Mikami, N. et al. Journal of immunology 186, 6886-6893, (2011)). Overall, the peptides produced by and released from the peripheral terminals of nociceptors block the chemotaxis and polarization of lymphocytes, influencing the localization, duration, and type of inflammation (Talbot, S. et al. Neuron 87, 341-354, (2015); Goetzl, E. J. et al. Proc Natl Acad Sci USA 98, 13854-13859 (2001); Nussbaum, J. C. et al. Nature 502, 245-248 (2013); Cunin, P. et al. Journal of immunology 186, 4175-4182 (2011); Ganea, D. et al. Arch Immunol Ther Exp (Warsz) 49, 101-110 (2001)).

Cancer cells actively secrete growth factors promoting tumor hyper-innervation (neo-axonogenesis (Boilly, B. et al. Cancer Cell 31, 342-354 (2017); Saloman, J. L. et al. Proc Natl Acad Sci USA 113, 3078-3083 (2016); Zhao, C. M. et al. Sci Transl Med 6, 250ra115(2014); Isaacs, J. T. Science 341, 134-135 (2013); Magnon, C. et al. Science 341, 1236361 (2013)), and tumors are often painful or itchy (Walter, F. M. et al. BMC Fam Pract 11, 62 (2010); Yosipovitch, G. et al. Dermatol Ther 23, 590-596 (2010); Yosipovitch, G. et al. JAMA Dermatol 150, 1160-1166 (2014)). In a preclinical prostate cancer model, sensory neurons were found to promote tumor cell proliferation (Magnon, C. et al. Science 341, 1236361 (2013)), while nerve-derived noradrenaline activate an angiogenic switch that fuels exponential tumor growth (Zahalka, A. H. et al. Science 358, 321-326, (2017)). In addition, doublecortin⁺ neural progenitors were found to infiltrate prostate tumours, initiating neurogenesis, while their selective genetic depletion inhibits the early phases of tumour development (Mauffrey, P. et al. Nature 569, 672-678 (2019)). Experimental surgical denervation of a cancer-affected organ, such as gastric tumor, limits cancer growth (Boilly, B. et al. Cancer Cell 31, 342-354 (2017); Saloman, J. L. et al. Proc Natl Acad Sci USA 113, 3078-3083 (2016); Zhao, C. M. et al. Sci Transl Med 6, 250ra115(2014)), while vagotomised subjects have lower intestinal cancer mortality rates than their untreated counterparts (Boilly, B. et al. Cancer Cell 31, 342-354 (2017); Zhao, C. M. et al. Sci Transl Med 6, 250ra115(2014); Isaacs, J. T. Science 341, 134-135 (2013); Magnon, C. et al. Science 341, 1236361 (2013)). Similarly, blockade of synaptic vesicle release from neurons with botulinum toxin reduces growth in prostate cancer (Isaacs, J. T. Science 341, 134-135 (2013); Magnon, C. et al. Science 341, 1236361 (2013)). These findings led us to postulate that local release of neuropeptides from activated nociceptors suppresses immune surveillance and thereby increase metastasis (Isaacs, J. T. Science 341, 134-135 (2013); Magnon, C. et al. Science 341, 1236361 (2013)), Saloman, J. L. Trends Neurosci 39, 880-889 (2016)).

### SUMMARY OF INVENTION

As provided herein, nociceptors play a regulatory role in the immune response to tumor growth, through the regulation of immune checkpoint receptor expression on cytotoxic CD8⁺ T-cells. Silencing tumor-innervating sensory neurons represents an innovative strategy for attenuating the immunomodulatory power of the nervous system and promoting anti-tumor activity. Genetic TRPV1 or Na_{V}1.8 lineage ablation, local pharmacological silencing or blockade of neuropeptide release from tumor-innervating nociceptors (e.g., with QX-314 or BoNT/a), and the antagonism of the CGRP receptor RAMP1 (e.g., with BIBN 4096) enhance tumor-infiltrating leukocyte (TIL) numbers and survival in mice, blunting tumor growth and TIL exhaustion.

In one aspect, provided herein are methods of treating cancer in a subject, the method comprising silencing tumor-innervating sensory neurons.

In another aspect, provided herein are methods of treating cancer in a subject, the method comprising administering to the subject a therapeutically effective amount of a nociceptor modulating agent. In some embodiments, the nociceptor modulating agent is a nociceptor antagonist.

In a further aspect, provided herein are methods of treating cancer in a subject, the method comprising administering to the subject a therapeutically effective amount of a neuropeptide modulating agent.

In one aspect, provided herein are methods of treating cancer in a subject, the method comprising administering to the subject a therapeutically effective amount of an agent that blocks the release or action of a neuropeptide from tumor-innervating neurons.

In another aspect, provided herein are methods of treating cancer in a subject, the method comprising administrating to the subject a therapeutically effective amount of an agent that blocks vesicle release from tumor-innervating nociceptors.

In a further aspect, provided herein are methods of treating cancer in a subject, the method comprising administering to a subject a therapeutically effective amount of a calcitonin gene-related peptide (CGRP) modulating agent. In some embodiments, the CGRP modulating agent is a CGRP receptor antagonist. In some embodiments, the CGRP receptor antagonist is a RAMP1 blocker.

In one aspect, provided herein are methods of treating cancer in a subject, the method comprising administering to a subject a therapeutically effective amount of QX-314, BoNT/a, and/or BIBN 4096.

In another aspect, provided herein are methods of treating cancer in a subject, the method comprising ablating an ion channel in a subject, wherein the ion channel is a sodium ion channel or TRPV ion channel. In some embodiments, the ion channel is Na_{V}1.8 and/or TRPV1.

In a further aspect, provided herein are compositions comprising (i) an anti-cancer agent, (ii) a nociceptor modulating agent, nociceptor antagonist, neuropeptide modulating agent, an agent that blocks vesicle release, or agent that blocks the release or action of a neuropeptide from tumor-innervating nociceptor described herein, and (iii) optionally a pharmaceutically acceptable excipient. In certain embodiments, the pharmaceutical composition described herein comprises (i) an anti-cancer agent, (ii) a nociceptor modulating agent, nociceptor antagonist, neuropeptide modulating agent, an agent that blocks vesicle release, or agent that blocks the release or action of a neuropeptide from tumor-innervating nociceptor described herein, and (iii) a pharmaceutically acceptable carrier or excipient.

In another aspect, provided herein are kits comprising a pharmaceutical composition described herein or a nociceptor modulating agent, nociceptor antagonist, neuropeptide modulating agent, an agent that blocks vesicle release, or agent that blocks the release or action of a neuropeptide from tumor-innervating nociceptor described herein and instructions for use (e.g., to treat cancer).

The details of certain embodiments of the disclosure are set forth in the Detailed Description of Certain Embodiments, as described below. Other features, objects, and advantages of the disclosure will be apparent from the Definitions, Figures, Examples, and Claims. It should be understood that the aspects described herein are not limited to specific embodiments, methods, apparati, or configurations, and as such can, of course, vary. It is also to be understood that the terminology used herein is for the purpose of describing particular aspects only and, unless specifically defined herein, is not intended to be limiting.

### BRIEF DESCRIPTION OF THE DRAWINGS

FIGS. 1A-1L: Melanoma sensitizes nociceptors. In comparison to control keratinocytes-injected skin (FIG. 1A), 14 days after B16F10-inoculation, abundant TRPV1⁺ nociceptors (red; TRPV1^{Cre}::Tdtomato^{fl/wt}) were found to innervate hyper-proliferating (green; BrdU⁺) cells (FIG. 1B). TRPV1⁺ nociceptors co-cultured with B16F10 have longer neurites (FIGS. 1C, 1E, 1F), reduced arborization (FIG. 1G), often form neuro-neoplastic contacts (FIG. 1C), then when cultured alone (FIGS. 1E-1G) or with non-tumorigenic keratinocytes (FIG. 1D). In co-culture, B16F0 or B16F10 cells sensitize the response of nociceptors to channel ligand (Capsaicin (100 nM), AITC (100 μM), ATP (1 μM)) used as control)) measured by calcium influx (FIG. 1H). Low concentration of the ligands induced minimal response in control neurons, while B16F10 show enhanced responses to ATP (FIG. 1H). L3-L5 DRG neurons harvested from mice 2-weeks post implantation with B16F10- or non-tumorigenic keratinocytes in the hindpaw were cultured and calcium flux generated by ligands tested. In comparison to keratinocytes, neurons from tumor-bearing mice were more sensitive to ATP (10 μM), and Capsaicin (1 μM; FIG. 1I). Neurons co-cultured with B16F10 cells release the neuropeptides CGRP (FIG. 1J). B16F10 cells alone do not releases neuropeptides (FIG. 1J). In comparison to DRG neurons(25) (FIG. 1K), B16F10 cells(26) (FIG. 1L) do not express the transcripts for Calca, Na_{V}1.8, Snap25, or TRPV1 as revealed by RNA sequencing. TRPV1⁺ nociceptors are labelled in red (FIGS. 1A-1D), BrdU proliferating cells are labelled in green (FIGS. 1A-1B), and nuclei are labelled in blue (FIGS. 1A, 1B, ID). B16F10 are labelled in green (FIG. 1C). Scale bar=100 μm. Mean±S.E.M; One-way ANOVA post-hoc Bonferroni (FIGS. 1E-1G) or unpaired Student's t-test (FIGS. 1H-1J); p values are showed in figure.

FIGS. 2A-2O: Nociceptor-released neuropeptides drive cytotoxic T-cell exhaustion. Cytotoxic CD8⁺ T-cells challenged with capsaicin-activated neurons conditioned media (CM) have increased proportion of PD1⁺Lag3⁺Tim3⁺ cells (FIG. 2A) and reduced INFγ⁺ (FIG. 2B) expressing cells. For cytotoxic CD8⁺ T-cells co-cultured with DRG neurons (FIGS. 2C-2D), capsaicin-stimulation increased the proportion of PD1⁺Lag3⁺Tim3⁺ cells (FIG. 2C), while it reduced proportion of INFγ⁺ (FIG. 2D) cells. The driving effects of neurons on CD8 T cells was null in absence of peptidergic neurons (TRPV1^{cre}DTA^{fl/wt} neurons, FIGS. 2E-2F). The active crosstalk between DRG neurons and cytotoxic CD8⁺ T-cells when co-cultured for 48 h, leads to T-cells overexpression of the CGRP receptor Ramp1 (FIG. 2G). Cytotoxic CD8⁺ T-cells directly stimulated with CGRP (0.1 μM) increased the proportion of PD1⁺Lag3⁺Tim3⁺ cells (FIG. 2H), while it reduced proportion of INFγ⁺ (FIG. 21) cells. When co-cultured, T_{c1}-stimulated OT1-CD8⁺ T-cells leads to B16F10-OVA cells apoptosis (AnnexinV⁺7AAD⁺; FIGS. 2J-2L). When co-cultured, neurons and cytotoxic CD8⁺ T-cells actively crosstalk. Such interplay prompts nociceptors to release neuropeptides which, in turn, exhaust CD8⁺ T-cells preventing their elimination of B16F10-OVA (FIG. 2J). B16F10-OVA apoptosis decrease when CD8⁺ T-cells are challenged with capsaicin-stimulated nociceptor conditioned media (FIG. 2K) or CGRP (FIG. 2L). Representative image of DRG nociceptors (TRPV1^{Cre}::QuASR2-eGFP^{fl/wt}; green) cultured with B16F10-OVA-mCherry (red) cells (FIG. 2M). Representative image of cytotoxic CD8 T cells cultured with B16F10-OVA-mCherry cells without (FIG. 2N) or with nociceptors (FIG. 2O). B16F10-OVA-mCherry2 are labelled in red (FIGS. 2M-2O), TRPV1^{Cre}::QuASR2-eGFP^{fl/wt} nociceptors are labelled in green (FIGS. 2M, 2O), and OT1-CD8⁺ T-cells are labelled in blue (FIGS. 2N-2O). Scale bar 100 μm. Mean±S.E.M; One-way ANOVA post-hoc Bonferroni (FIGS. 2A-2L); p values are showed in figure.

FIGS. 3A-3I: Genetic elimination of nociceptors reduces TIL exhaustion. Orthotopic B16F10 tumor growth (FIG. 3A) was reduced in mice whose nociceptors are genetically ablated (TRPV1^{Cre}::DTA^{fl/wt}) while their median length of survival was increased by ˜250% (Mantel-Haenszel Hazard Ratio; FIG. 3B). Sensory neuron ablation also increased tumor infiltration of total (FIG. 3C) and INFγ⁺ CD8⁺ T-cells (FIG. 3D); while the proportion of PD1⁺Lag3⁺Tim3⁺ CD8⁺ T-cells was decreased (FIG. 3E). The reduction in B16F10 tumor growth observed in nociceptors ablated mice was absent following systemic CD3 depletion (FIG. 3F; αCD3, 200 μg/mice; i.p.; every 3 days). Nociceptor ablation also potentiated αPDL1 (6 mg/kg, i.p.; d7, d10, d13) mediated reduction in B16F10-OVA tumor growth (FIG. 3G). The daily optogenetic activation (3.5 ms, 10 Hz, 478 nm, 100 mW, giving approx. 2-6 mW/mm² with a 0.39 NA fiber placed 5-10 mm from the skin, for 20 min) of B16F10-inoculated skin in light sensitive Na_{V}1.8^{Cre}::ChR2^{fl/wt} mice enhanced tumor growth (FIG. 3H); while the genetic ablation of Na_{V}1.8⁺ lineage neurons (Na_{V}1.8^{Cre}::DTA^{fl/wt}) had the opposite effects (FIG. 3H). Finally, it was also found that sensory neuron ablation (TRPV1^{Cre}::DTA^{fl/wt}) decreases the growth of YUMMER1.7 cells; an immunogenic version of a Braf^{V600E}Cdkn2a^{-/-}Pten^{-/-} melanoma cell line (FIG. 3I). Mean±S.E.M; Two-way ANOVA post-hoc Bonferroni (FIG. 3A, FIGS. 3F-3I); Mantel-Cox regression (FIG. 3B) or unpaired Student's t-test (FIG. 3C-3E); p values are showed in figure.

FIGS. 4A-4L: Silencing tumor-innervating nociceptors rescues anti-tumor immunity. B16F10 tumor growth (FIGS. 4A, 4C) and number of PD1⁺Lag3⁺Tim3⁺ CD8⁺ T-cells (FIGS. 4B, 4D) were reduced in mice whose tumor-innervating neuron are silenced by BoNT/a (FIGS. 4A-4B; 25 pg/μl; twice, i.d.) or QX-314 (FIGS. 4C-4D, 0.3%, i.d., q.d.). Tumor-innervating nociceptor silencing heighten αPDL1 (6 mg/kg, i.p.) decreased in B16F10-OVA tumor growth (FIG. 4E) and prolong the animals' median length of survival (FIG. 4F) by ˜270% (QX-314) and ˜800% (BoNT/a). CGRP levels are increased in B16F10 tumor surrounding skin explant in comparison to control skin; an effect further enhanced by capsaicin (1 μM; 3 h) but absent in skin pre-treated with BoNT/a (25 pg/μl) or QX-314 (0.3%; FIG. 4G). The RAMP1 antagonist BIBN4096 (5 mg/kg, i.p., day 6, 8, 10, 12, 14) decreased B16F10 tumor growth (FIG. 4H), and the proportion of intra-tumor PD1⁺Lag3⁺Tim3⁺ CD8⁺ T-cells (FIG. 4I), while it increased the numbers of INFγ⁺ CD8⁺ T-cells (FIG. 4J). In-silico analysis of single-cell RNA-sequencing of human melanoma (45) revealed that Ramp1⁺ CD8⁺ T-cells strongly overexpressed several immune checkpoint receptors (PD1, Tim3, Lag3, CTLA-4, CD28, ICOS, BTLA, CD27) in comparison to Ramp1⁻ CD8⁺ T-cells (FIG. 4K). Amongst other nociceptor markers, RNA sequencing data also showed that Calca, the gene encoding for CGRP, is overexpressed in melanoma skin biopsies in comparison to healthy skin (41) (FIG. 4L). Mean±S.E.M; Two-way ANOVA post-hoc Bonferroni (FIGS. 4A, 4C, 4E, 4H), one-way ANOVA post-hoc Bonferroni (FIG. 4G), Mantel-Cox regression (FIG. 4F), or unpaired Student's t-test (FIGS. 4B, 4D, 4I, 4J, 4L); p values are showed in figure.

FIGS. 5A-5C: Patient melanomas are innervated. Tubb3⁺ nerve fibers (IHC, brown; FIGS. 5A-5C) are present in three aggressive melanoma patient biopsies (FIGS. 5A-5C). Scale=100 μm.

FIG. 6: Immunocytes profiling. RNA sequencing of leukocyte subpopulations (47) revealed their basal expression of Cd45 and Ramp1. In contrast, immune cells do not express Snap25, Trpv1 and Na_{V}1.8.

FIGS. 7A-7C: Neurons-conditioned media drive CD8⁺ T-cells exhaustion. Cytotoxic CD8⁺ T-cells challenged with KCl-stimulated neuron conditioned media have increased proportion of PD1⁺Lag3⁺Tim3⁺ (FIG. 7A) and decreased level of INFγ⁺ (FIG. 7B) and TNFα⁺ (FIG. 7C) cells. Mean±S.E.M; unpaired Student's t-test; p values are showed in figure.

FIGS. 8A-8D: Nociceptors-conditioned media drive CD8⁺ T-cells exhaustion. Cytotoxic CD8⁺ T-cells challenged with KCl-stimulated neuron conditioned media have increased proportion of PD1⁺Lag3⁺Tim3⁺ cells (FIG. 8A) and decreased level of INFγ⁺ (FIG. 8B), TNFα⁺ (FIG. 8C) and IL2+(FIG. 8D) cells. These effects were absent when cytotoxic CD8⁺ T-cells were challenged with KCl-stimulated TRPV1^{cre}DTA^{fl/wt} neuron conditioned media. Mean±S.E.M; One-way ANOVA post-hoc Bonferroni; p values are showed in figure.

FIGS. 9A-9G: Nociceptors drive CD8⁺ T-cells exhaustion. Capsaicin-challenge of DRG neurons co-cultured with cytotoxic CD8⁺ T-cells increases the proportion of PD1⁺ T cells (FIGS. 9A, 9D), while it decreased the level of TNFα⁺ (FIGS. 9B, 9E), IL2⁺ (FIGS. 9C, 9F) and INFγ⁺ (FIG. 9G) cells. Mean±S.E.M; unpaired Student's t-test; p values are showed in figure.

FIGS. 10A-10E: CGRP drives CD8⁺ T-cells exhaustion. CGRP increases cytotoxic CD8⁺ T-cells expression of PD1⁺ (FIG. 10A), Lag3⁺ (FIG. 10B), and Tim3⁺ (FIG. 10C) while it decreased level of INFγ⁺ (FIG. 10D) and TNFα⁺ (FIG. 10E). Mean±S.E.M; unpaired Student's t-test; p values are showed in figure.

FIGS. 11A-11G: Sensory neuron-released neuropeptides blunt cytotoxic T-cell anti-tumor immunity. Capsaicin-stimulated nociceptor conditioned media (FIGS. 11A-11C), neuron co-culture (FIGS. 11D-11F) or CGRP (FIG. 11G) reduces B16F10-OVA (Annexin V⁺) elimination by OVA-specific cytotoxic CD8⁺ T-cells (FIGS. 11A, 11D, 11G), while it increased mean fluorescence intensity of PD1⁺ (FIGS. 11B, 11E) and Lag3⁺ (FIGS. 11C, 11F) on the OT1 CD8⁺ T-cells. Mean±S.E.M; one-way ANOVA post-hoc Bonferroni (FIGS. 11A, 11D); or unpaired Student's t-test (FIGS. 11B, 11C, 11E-11G); p values are showed in figure.

FIGS. 12A-12I: Ablation of nociceptors decreases tumor growth. Orthotopic B16F10 tumor weight (FIG. 12A), size (FIG. 12B) and growth (FIGS. 12D-12I) were reduced in mice whose nociceptors are genetically ablated (TRPV1^{Cre}::DTA^{fl/wt}). The median length of survival of B16F10-inoculated sensory neuron ablated mice increased by 23%, while the one procured by αPDL1 blockade average 15.3% (FIG. 12C; meta-analysis of 16 published studies). Mean±S.E.M; unpaired Student's t-test (FIG. 12A); two-way ANOVA post-hoc Bonferroni (FIGS. 12D-12I); p values are showed in figure.

FIGS. 13A-13B: Nociceptors ablation prevent intra-tumoral CD8⁺ T-cells exhaustion. Sensory neuron ablation (TRPV1^{Cre}::DTA^{fl/wt}) increased B16F10 tumor infiltration of TNFα⁺ (FIG. 13A) and granzyme B⁺ (FIG. 13B) CD8⁺ T-cells. Mean±S.E.M; Unpaired Student's t-test; p values are showed in figure.

FIGS. 14A-14D: Nociceptors ablation prevent intra-tumoral CD4⁺ T-cells exhaustion. Sensory neuron ablation (TRPV1^{Cre}::DTA^{fl/wt}) increased B16F10 tumor infiltration of total (FIG. 14A), TNFα⁺ (FIG. 14C) and INFγ⁺ (FIG. 14D) CD4⁺ T-cells while the relative proportion of PD1⁺Lag3⁺Tim3⁺ CD4⁺ T-cells was decreased (FIG. 14B). Mean±S.E.M; Unpaired Student's t-test; p values are showed in figure.

FIGS. 15A-15F: Sensory neuron ablation decreases CD4⁺ and CD8⁺ T-cells exhaustion in tumor-draining lymph node. In comparison to B16F10-inoculated wildtype mice, nociceptor ablated animals have increased total (FIGS. 15A, 15B, 15D, 15E), INFγ⁺ (FIGS. 15C, 15F), CD8⁺ (FIGS. 15A-15C) and CD4⁺ (FIGS. 15D-15F) T-cells in tumor-draining lymph node. Mean±S.E.M; Unpaired Student's t-test; p values are showed in figure.

FIGS. 16A-16B: Nociceptors control CD3 anti-tumor immunity. Systemic CD3 depletion (FIG. 16A; αCD3, 200 μg/mice; i.p.; every 3 days) rescue tumor growth in nociceptor ablated mice (FIG. 16B). Mean±S.E.M.

FIGS. 17A-17D: Nociceptor neurons ablation potentiated αPDL1 reduction in tumor growth. Nociceptor ablation potentiated αPDL1 (6 mg/kg, i.p.; d7, d10, d13) mediated reduction in B16F10-OVA tumor growth (FIGS. 17A, 17B). The ablation also increased the infiltration of tumor specific CD8⁺ T-cells (FIG. 17C) which are less exhausted (FIG. 17D). These effects were further enhanced by αPDL1 treatment (FIGS. 17C-17D). Mean±S.E.M; One-way ANOVA post-hoc Bonferroni; p values are showed in figure.

FIGS. 18A-18C: Optogenetic activation of Na_{V}1.8⁺ nociceptors promotes TIL exhaustion. Daily optogenetic activation (3.5 ms, 10 Hz, 478 nm, 100 mW, giving approx. 2-6 mW/mm² with a 0.39 NA fiber placed 5-10 mm from the skin, for 20 min) of B16F10-inoculated skin in light sensitive Na_{V}1.8^{Cre}::ChR2^{fl/wt} mice decreased intra-tumor number of CD8 T cells (FIG. 18A), but increased the relative proportion of PD1⁺Tim3⁺ CD8⁺ T-cells (FIG. 18B) as well as secretion of CGRP in tumor surrounding skin explants (FIG. 18C). Genetic ablation of Na_{V}1.8⁺ lineage neurons (Na_{V}1.8^{Cre}::DTA^{fl/wt}) had the opposite effects (FIGS. 18A-18B). Mean±S.E.M; One-way ANOVA post-hoc Bonferroni; p values are showed in figure.

FIGS. 19A-19H: Sensory neuron ablation decreases CD8⁺ T-cells exhaustion in YUMMER1.7-inoculated mice. In comparison to YUMMER1.7-inoculated wildtype mice, nociceptor ablated animals have decreased tumor volume (FIG. 19A) and weight (FIG. 19B), as well as proportion of PD1⁺Lag3⁺Tim3⁺ CD8⁺ (FIG. 19E) and CD4⁺ (FIG. 19H) T-cells. Sensory neuron ablation also increased intra-tumor number of total (FIGS. 19C, 19D), INFγ⁺ (FIG. 19F), and TNFα⁺ (FIG. 19G) CD8⁺ (FIGS. 19C, 19E-19G) and CD4⁺ (FIGS. 19D, 19H) T-cells. Mean±S.E.M; Unpaired Student's t-test; p values are showed in figure.

FIG. 20A-20H: Botox silencing of B16F10-innervating neurons decrease tumor growth. When given prior to B16F10 inoculation, tumor volume (FIG. 20A), and weight (FIG. 20B) were reduced in mice treated with BoNT/a (i.d.; 25 pg/μl, day −3 and −1). In addition, silencing tumor-innervating neurons increased intra-tumoral number of total (FIG. 20C), INFγ⁺ (FIG. 20D) and Granzyme B⁺ (FIG. 20E) CD8⁺ T-cells. While t intra-tumoral CD4⁺ T-cells counts were not impacted (FIG. 20F), their exhaustion (FIG. 20G) was decreased in mice pre-treated with BoNT/A. Tumor-innervating nociceptor silencing heightens αPDL1 (6 mg/kg, i.p.) decreased B16F10-OVA tumor growth (FIG. 20H). Mean±S.E.M; Unpaired Student's t-test (FIGS. 20A-20G); p values are showed in figure.

FIGS. 21A-21E: Botox do not impact CD8⁺ T-cells function. When compared to vehicle exposed cells, BoNT/a (10-50 pg/ul; 24 h) do not impact the survival (FIG. 21A) of cultured cytotoxic CD8⁺ T-cells, nor their relative expression of PD1⁺Lag3⁺Tim3⁺ (FIG. 21B), TNFα⁺ (FIG. 21C), INFγ⁺ (FIG. 21D) and IL2⁺ (FIG. 21E). Mean±S.E.M.

FIGS. 22A-22I: Sensory neuron silencing prevents TILs exhaustion. Sensory neuron silencing with QX-314 decreased B16F10 tumor volume (FIG. 22A), and weight (FIG. 22B). QX-314 exposed B16F10-bearing mice show increased intra-tumoral number of total (FIG. 22C), INFγ⁺ (FIG. 22D) Granzyme B⁺ (FIG. 22E) and TNFα⁺ (FIG. 22F) CD8⁺ T-cells as well as total CD4⁺ T-cells (FIG. 22G). QX-314 decreased intra-tumoral proportion of PD1⁺Lag3⁺Tim3⁺ CD4⁺ T-cells (FIG. 22H). Tumor-innervating nociceptor silencing heighten αPDL1 (6 mg/kg, i.p.) decreased in B16F10-OVA tumor growth (FIG. 22I). Mean±S.E.M; Unpaired Student's t-test (FIGS. 22A-22H); p values are showed in figure.

FIGS. 23A-23E: QX-314 do not impact CD8⁺ T-cells function. When compared to vehicle exposed cells, QX-314 (50-150 μM; 24 h) do not impact the survival (FIG. 23A) of cultured cytotoxic CD8⁺ T-cells, nor their proportion of PD1⁺Lag3⁺Tim3⁺ (FIG. 23B), TNFα⁺ (FIG. 23C), INFγ⁺ (FIG. 23D) and IL2⁺ (FIG. 23E). Mean±S.E.M.

FIGS. 24A-24J: RAMP1 antagonism prevents TILs exhaustion. Blockade of the CGRP receptor RAMP1 with BIBN4096 (5 mg/kg, i.p.) decreased B16F10 tumor volume (FIG. 24A), and weight (FIG. 24B). RAMP1 blockade in B16F10-bearing mice increased intra-tumoral number of total (FIG. 24C), Granzyme B⁺ (FIG. 24D) and TNFα⁺ (FIG. 24E) CD8⁺ T-cells. Similar effects were found for total (FIG. 24F), TNFα⁺ (FIG. 24G), INFγ⁺ (FIG. 24H), and Granzyme B⁺ (FIG. 241) CD4⁺ T-cells. BIBN4096 decreased intra-tumoral proportion of PD1⁺ CD4⁺ T-cells (FIG. 24J). Mean±S.E.M; Unpaired Student's t-test; p values are showed in figure.

FIGS. 25A-25E: BIBN4096 do not impact CD8⁺ T-cells function. When compared to vehicle exposed cells, BIBN4096 (1-4 μM; 24 h) do not impact the survival (FIG. 25A) of cultured cytotoxic CD8⁺ T-cells, nor their proportion of PD1⁺Lag3⁺Tim3⁺ (FIG. 25B), TNFα⁺ (FIG. 25C), INFγ⁺ (FIG. 25D) and IL2⁺ (FIG. 25E). Mean±S.E.M.

FIGS. 26A-26D: Sensory neuron silencing prevents tumor-induced pain and itch. B16F10-hindpaw inoculation led to tumor induction (FIG. 26A), mechanical (FIG. 26B) and thermal pain hypersensitivities (FIG. 26C), while flank injection trigger occasional (˜30%) itch (FIG. 26D). These effects were stopped by sensory neuron silencing (QX-314 (0.3%, qd, i.d., FIGS. 26A-26D) and BoNT/a (25 pg/μl, FIGS. 26A-26C) or absent in mice whose sensory neuron are genetically ablated (TRPV1^{cre}::DTA^{fl/wt}, FIG. 26D). Mean±S.E.M; Unpaired Student's t-test (FIGS. 26B-26C) or one-way ANOVA post-hoc Bonferroni (FIG. 26D).

FIGS. 27A-27B: Sensory neuron silencing does not impact B16F10 survival. QX-314 (0.1-1%; 72 h; FIG. 27A) or BoNT/a (1.6-50 pg/μl; 24 h; FIG. 27B) did not impact the in vitro proliferation (FIG. 27A) or apoptosis (FIG. 27B) of cultured B16F10 cells.

FIG. 28: Single-cell RNA sequencing of human melanoma. In-silico analysis of single-cell RNA-sequencing of human melanoma revealed that Ramp1⁺ T-cells lost IL-2 expression but strongly overexpressed several immune checkpoint receptors (PD1, Tim3, Lag3, CTLA-4, CD28, ICOS, BTLA, CD27) in comparison to Ramp1⁻ T-cells. In-silico analysis of Tirosh et al (46).

FIG. 29: Single-cell RNA sequencing of human melanoma. Melanoma, cancer associated fibroblasts, macrophage, endothelial, natural killer, T, and B cells harvested from patients' melanoma do not express Calca. In addition, these cells do not express Snap25, the molecular target of BoNT/A. They also do not express Trp and Nav channels which are the molecular targets of QX-314. In-silico analysis of Tirosh et al (46).

FIG. 30: Single-cell RNA sequencing of human melanoma. Melanoma, cancer associated fibroblasts, macrophage, endothelial, natural killer, T, and B cells harvested from patients' melanoma do not express Calca. In addition, these cells do not express Snap25, the molecular target of BoNT/A. They also do not express Trp and Nav channels which are the molecular targets of QX-314. In-silico analysis of Jerby-Arnon et al (45).

FIGS. 31A-31E: Higher neuronal-associated genes transcript expression worsened patient's survival. Across a population of 458 melanoma patients (40), enhanced RNA-sequencing gene expression (label as high; FIG. 31A) of Na_{V}1.7 (FIG. 31B), Piezo1 (FIG. 31C), Pgp9.5 (FIG. 31D), and Tubb3 (FIG. 31E) in biopsy correlate with decreased patient survival (p≤0.05). Relative gene expression across is shown in blue (FIG. 31A). Mantel-Cox regression (FIGS. 31B-31E).

FIG. 32: Increased transcript expression of neuronal-associated genes were found in melanoma biopsies. Melanoma skin biopsy show heighten expression (heatmap) of Calca, Ramp1, Pouf4f1, Eno2 and Tubb3 as well as other neuronal-enriched genes in comparison to benign skin nevi (41). Unpaired Student's t-test; p value showed in figure.

FIG. 33: Increased transcript expression of neuronal-associated genes were found in melanoma biopsies. Melanoma skin biopsy show heighten expression (heatmap) of Calca, Tubb3, Pouf4f1, and Eno2 as well as other neuronal-enriched genes encoding for neuropeptides and their receptors, ion channels receptors, and transcription factors in comparison to the skin of healthy patient (44). Unpaired Student's t-test; p value showed in figure.

FIG. 34: Increased transcript expression of neuronal-associated genes were found in melanoma biopsies. Melanoma skin biopsy show heighten expression (heatmap) of Ramp1, Calca, and Trpa1 as well as other neuronal-enriched genes encoding for neuropeptides and their receptors, ion channels receptors, and transcription factors in comparison to healthy patients' blood PBMC (42). Unpaired Student's t-test; p value showed in figure.

FIG. 35: PD1⁺ nociceptors decrease in tumor-innervating neurons. In comparison to TRPV1⁺ nociceptor neurons from keratinocytes-injected skin, FACS-sorted B16F10-infiltrating TRPV1⁺ nociceptor neurons express less Pd1 transcript. Mean±S.E.M.

FIG. 36: RNA-Sequencing signatures of human immune cell types. An in-silico analysis of Monaco et al. (49) revealed that immunocytes express Cd45; but not Na_{V}1.7, Na_{V}1.8 and Trpv1 which are relevant for the activity of QX-314. Similarly, immunocytes do not expressed Snap25 which is the molecular target of BoNT/A.

FIGS. 37A-37J: Melanoma sensitizes nociceptors. In comparison to control keratinocyte-injected skin (FIG. 37A), 14 days after B16F10-inoculation TRPV1⁺ nociceptor (red; TRPV1^{Cre}::Tdtomato^{fl/wt}) innervate hyper-proliferating (green; BrdU⁺) cells (FIG. 37B). TRPV1⁺ nociceptors co-cultured with B16F10 have longer neurites (FIGS. 37C, 37E, 37F), reduced arborization (FIG. 37G), often form neuro-neoplastic contacts (FIG. 37C), then when cultured with non-tumorigenic keratinocytes (FIG. 37D). In co-culture, B16F10 cells sensitize the response of nociceptors to channel ligand (Capsaicin (100 nM), AITC (100 uM), ATP (1 uM), (KCl (50 mM) used as control)) measured by calcium influx (FIG. 37H). Low concentration of the ligands induced minimal response in control neurons, while B16F10 show enhanced responses to ATP (FIG. 37H). L3-L5 DRG neurons harvested from mice 2-weeks post implantation with B16F10- or non-tumorigenic keratinocytes in the hindpaw were cultured and calcium flux generated by ligands tested. In comparison to keratinocytes, neurons from tumor-bearing mice were more sensitive to ATP (10 uM) and Capsaicin (1 uM; FIG. 37I). Neurons co-cultured with B16F10 cells release the neuropeptides VIP, SP and CGRP (FIG. 37J).

FIGS. 38A-38O: Nociceptors-released neuropeptide drives cytotoxic T cell exhaustion. Cytotoxic CD8 T cells challenged with conditioned media (CM) from naive and KCl-activated neurons have an increased proportion of PD1⁺Lag3⁺ cells (FIG. 38A), and reduced INFγ⁺ (FIG. 38B) and TNFα⁺ expressing cells (FIG. 38C). For co-cultured cytotoxic CD8 T cells and DRG neurons (FIGS. 38D-38F), capsaicin-stimulation increased the proportion of PD1⁺Lag3⁺Tim3⁺ cells (FIG. 38D) and reduced INFγ⁺ (FIG. 38E) and TNFα⁺ levels (FIG. 38F). Cytotoxic CD8 T cells directly stimulated with CGRP (0.1 uM) and VIP (1 uM) have an increased proportion of PD1⁺Lag3⁺ cells (FIG. 38G), while VIP enhances proportion of INFγ⁺ cells (FIG. 38H). CGRP (0.1 uM) and VIP (1 uM) both reduced the proportion of TNFα⁺ (FIG. 38I). OVA-specific cytotoxic CD8 T cells trigger apoptosis in B16F10-OVA (AnnexinV⁺7AAD⁺) cells (FIGS. 38J-380). B16F10-OVA apoptosis decreases when the cells are challenged with capsaicin-stimulated nociceptor conditioned media (FIG. 38J), co-cultured with DRG neurons (FIG. 38K) and stimulated with neuropeptides (FIG. 38L). Representative image of DRG nociceptors (TRPV1^{cre}td-tomato^{fl/wt}; green) cultured with B16F10-OVA-mCherry (red) cells (FIG. 38M). Representative image of cytotoxic CD8 T cells (blue) cultured with B16F10-OVA-mCherry cells (red) without (FIG. 38N) or with nociceptors (TRPV1^{cre}td-tomato^{fl/wt}; green; FIG. 38O).

FIGS. 39A-39J: Ablation of nociceptors decreases tumor growth. Orthotopic B16F10 tumor growth (FIG. 39A), weight (FIG. 39B), appearance (FIG. 39D), and the infiltration of PD1⁺Lag3⁺TIM3⁺ CD8 T cells (FIG. 39G) were reduced in mice whose nociceptors are genetically ablated (TRPV1^{Cre}::DTA^{fl/wt}) while their survival (FIG. 39C), and intra-tumor number of total (FIG. 39E) and INFγ⁺ CD8 T cells (FIG. 39F) were increased. The reduction in B16F10 tumor growth (FIG. 39H) and volume (FIG. 391) observed nociceptor in ablated mice was absent following systemic CD3 depletion in these mice (FIG. 39J; αCD3, 200 μg/mice; i.p.; every 3 days).

FIGS. 40A-40I: Activating nociceptors increases growth of solid tumors. αPDL1 (6 mg/kg, i.p.; d7, d10, d13) injections further reduced B16F10-OVA tumor growth when given to nociceptor ablated mice (FIG. 40A). In B16F10-OVA inoculated mice, sensory neuron ablation increased the infiltration of tumor-specific CD8 T cells (FIG. 40B) which are also less exhausted (FIG. 40C). Daily optogenetic activation of B16F10-inoculated skin in light sensitive Na_{V}1.8^{Cre}::ChR2^{fl/wt} mice enhanced tumor growth (FIG. 40D), intra-tumor number of CD8 T cells (FIG. 40E) and reduced number of intra-tumor PD1⁺Lag3⁺ CD8 T cells (FIG. 40F). Ablation of NaV1.8+ lineage neurons (Na_{V}1.8^{Cre}::DTA^{fl/wt}) had the opposite effects (FIG. 40D-40F). The tumor growth (FIG. 40G) of flank inoculated lung metastatic HPV⁺ oropharyngeal squamous cell carcinomas mEERL and MLM3 was decrease in sensory neuron ablated mice, while their survival (FIG. 40H) was increased. In contrast, the growth of EG7 lymphoma was enhanced in sensory neuron ablated mice (FIG. 40I).

FIGS. 41A-41P: Blocking vesicle release and silencing tumor-innervating nociceptor neurons rescues anti-tumor immunity. B16F10 tumor growth (FIG. 41A, 41E), and intra-tumor PD1⁺Lag3⁺TIM3⁺ CD8 T cells (FIG. 41C, 41G) were reduced in mice treated with BoNT/a (FIG. 41A-41D; 25 pg/μl; twice, i.d.), or QX-314 (FIG. 41E-41H, 100 μM, i.d., q.d.), while infiltration of total (FIG. 41B, 41F) and INFγ⁺ CD8 T cells (FIG. 41D, 41H) were increased. αPDL1 (6 mg/kg, i.p.) decreased in B16F10-OVA tumor growth (FIG. 411, 41K) and volume (FIG. 41J, 41L) was further enhanced in mice co-exposed to QX-314 (FIG. 41I-41J) or BoNT/a (FIG. 41K-41L). The Ramp1 antagonist BIBN (5 mg/kg) decreased B16F10 tumor growth (FIG. 41M), and intra-tumor number of PD1⁺ CD8 T cells (FIG. 41O), while numbers of total and INFγ⁺ CD8 T cells (FIGS. 41N, 41P, respectively) were enhanced.

FIGS. 42A-42B: Cancer cells control neuron sensitivity and growth. Calcium flux induced by nociceptor activating ligands (capsaicin (100 nM), AITC (100 uM), ATP (1 uM)) in lumbar neurons co-cultured with B16F0 or B16F10 cells (FIG. 42A). TRPV1⁺ nociceptors co-cultured with B16F0 or B16F10 have longer neurites, then when cultured with non-tumorigenic keratinocytes (FIG. 42B).

FIGS. 43A-43G: Neuropeptides drive CD8 exhaustion. In-silico analysis of various leukocyte population revealed their basal expression of various neuropeptide receptors, including the one for CGRP (FIG. 43A). Cytotoxic CD8 T cells either challenged with KCl-stimulated neuron conditioned media (FIGS. 43B-43C), or co-cultured with capsaicin-stimulated DRG neurons (FIGS. 43D-43G) have increased proportion of PD1⁺ (FIGS. 43B, 43D), Lag3⁺ (FIGS. 43C, 43E), Tim3⁺ (FIG. 43F), and decreased level of IL2⁺ (FIG. 43G).

FIGS. 44A-44L: Sensory neuron-released neuropeptides modify CD8 anti-tumor immunity. In comparison to TRPV1^{cre}::DTA^{fl/wt} neurons, cytotoxic CD8 T cells co-cultured with wildtype neurons have increased proportion of PD1⁺Lag3⁺ cells (FIG. 44A), and reduced levels of INFγ⁺ (FIG. 44B) TNFα⁺ (FIG. 44C) and IL2⁺ cells (FIG. 44D). Cytotoxic CD8 T cells co-cultured with naïve lumbar neurons showed increase transcripts expression of Ramp1 (FIG. 44E), Ramp3 (FIG. 44F), and Vpac2 (FIG. 44G). Neuropeptides (FIGS. 44H-44L) increased the proportion of PD1⁺ (FIG. 44H), Lag3⁺ (FIG. 44I), Tim3⁺ (FIG. 44J) cytotoxic CD8 T cells, while it decreased their expression of IL2 (FIG. 44K) and INFγ (FIG. 44L).

FIGS. 45A-45G: CD8 express neuropeptide receptors. Capsaicin-stimulated nociceptor conditioned media (FIGS. 45A-45C), neuron co-culture (FIGS. 45D-45F) or exogenous neuropeptide challenges (FIG. 45G) reduced B16F10-OVA (Annexin V⁺) elimination by OVA-specific cytotoxic CD8 T cells (FIGS. 45A, 45D, 45G), while it increased mean fluorescence intensity of PD1⁺ (FIGS. 45B, 45E) and Lag3⁺ (FIGS. 45C, 45F) on CD8 T cells.

FIGS. 46A-46Q: Sensory neuron ablation prevents intra-tumoral CD8 exhaustion. In comparison to B16F10-inoculated wildtype mice, nociceptor ablated animals have increased intra-tumoral number of total (FIG. 46A), TNFα⁺ (FIGS. 46C, 46H), granzyme B⁺ (FIGS. 46D, 46I), and INFγ⁺ (FIG. 46E) CD4 (FIGS. 46A-46E) and CD8 (FIGS. 46F-46I) T cells. Conversely, these mice display lower intra-tumoral number of PD1⁺Lag3⁺Tim3⁺ CD4 T cells (FIG. 46B) as well as PD1⁺ (FIG. 46F), Lag3⁺ (FIG. 46G) CD8 T cells. The tumor draining lymph node of B16F10-inoculated nociceptor ablated mice also have higher proportion (FIGS. 46J, 46N) and number (FIGS. 46K, 46O) of total (FIGS. 46J, 46K, 46N, 46O), INFγ⁺ (FIGS. 46L, 46P) and TNFα⁺ (FIGS. 46C, 46H, 46M, 46Q) CD4 (FIGS. 46J-46M) and CD8 (FIGS. 46N-46Q) T cells.

FIGS. 47A-47K: Sensory neuron ablation prevents intra-tumoral NK cell exhaustion. In comparison to B16F10-inoculated wildtype mice, nociceptor ablated animals have increased intra-tumoral proportion (FIG. 47A) and number (FIGS. 47B-47F) of total (FIGS. 47A, 47B), INFg+ (FIG. 47C), and granzyme B⁺ (FIG. 47D) NK (FIGS. 47A-47D). We found similar intra-tumoral number of total (FIG. 47G) NK T cells, but lower levels of PD1⁺ (FIG. 47H) and Lag3⁺ (FIG. 47I). Nociceptor ablated animals also showed reduced B16F10 tumor volume (FIG. 47J), an effect that was further enhanced by αPDL1 treatment (FIG. 47K; 6 mg/kg, i.p. day 7, 10 and 13).

FIGS. 48A-48Z: Sensory neuron silencing prevents CD8 exhaustion. Twenty-four hours BoNT/a (1.6-50 pg/μl) or 72 h QX-314 (0.1-1%) exposure had no impact on B16F10 tumor growth in vitro (FIGS. 48A, 48I). B16F10 tumor volume (FIGS. 48B, 48J, 48R), and weight (FIGS. 48C, 48K, 48S) were reduced in mice treated with BoNT/a (FIGS. 48B, 48C; 25 pg/μl), QX-314 (FIGS. 48J, 48K, 100 μM, i.d., q.d.) or BIBN (R-S, 5 mg/kg. i.d., q.d.). When given prior to (d-3, d-1), or immediately after (dl and d3), B16F10 inoculation, BoNT/a (i.d.; 1.6-50 pg/μl) increased intra-tumoral number of Granzyme B⁺ CD4 T cells (FIG. 48G), and reduced numbers of PD1⁺Lag3⁺TIM3⁺ CD4 T cells (FIG. 48H). BoNT/a treatment had no impact on B16F10 infiltration of Granzyme B⁺ CD8 T cells (FIG. 48D), as well as total (FIG. 48E), INFγ⁺ (FIG. 48F), and Granzyme B⁺ (FIG. 48G) CD4 T cells. QX-314 (100 μM, i.d., qd) increased tumor infiltration of Granzyme B+ (FIG. 48L) and TNFα+ (FIG. 48M) CD8 T cells as well as total number of CD4 T cells (FIG. 48N). QX-314 decrease tumor infiltration of Granzyme B+ (FIG. 48O), and PD1⁺Lag3⁺TIM3⁺ (FIG. 48P) CD4 T cells (FIG. 48H), but failed to impact levels of TNFα+ CD4 T cells (FIG. 48Q). In B16F10-bearing mice, BIBN (5 mg/kg, i.d.) increased tumor infiltration of Granzyme B+ CD8 T cells (FIG. 48T), TNFα+ CD8 T cells (FIG. 48U). as well as total (FIG. 48V), INFγ⁺ (FIG. 48W), Granzyme B⁺ (FIG. 48X) CD4 T cells. BIBN (5 mg/kg, i.d.) also decreased B16F10 infiltration of PD1+ (FIG. 48Y) and TNFα+ (FIG. 48Z) CD4 T cells.

FIGS. 49A-49E: Sensory neuron silencing prevents tumor growth. Intradermal inoculation of B16F10 to the mice hindpaw led to tumor growth (FIG. 49A), occasional (˜30%) itch (FIG. 49B), mechanical (FIG. 49C) and thermal pain hypersensitivities (FIG. 49D). These effects were absent in mice whose sensory neuron are genetically ablated (TRPV1^{cre}::DTA^{fl/wt}, FIG. 49D) or pharmacologically silenced (QX-314 (100 μM, qd, i.d., FIGS. 49A-49D) and BoNT/a (25 pg/μl, FIGS. 49A-49C). CGRP levels are shown in B16F10 tumor surrounding skin explant. Capsaicin-induced CGRP release from tumor-inoculated skin was absent in QX-314 and BoNT/A pre-treated skin (FIG. 49E).

### DEFINITIONS

For convenience, certain terms employed herein, in the specification, examples, and claims are collected herein.

Unless otherwise required by context, singular terms shall include pluralities, and plural terms shall include the singular.

The language “in some embodiments” and the language “in certain embodiments” are used interchangeably.

The singular terms “a,” “an,” and “the” include plural references unless the context clearly indicates otherwise. Similarly, the word “or” is intended to include “and” unless the context clearly indicates otherwise.

Other than in the examples, or where otherwise indicated, all numbers expressing quantities of ingredients or reaction conditions used herein should be understood as modified in all instances by the term “about.” “About” and “approximately” shall generally mean an acceptable degree of error for the quantity measured given the nature or precision of the measurements. Exemplary degrees of error are within 20 percent (%), typically, within 10%, or more typically, within 5%, 4%, 3%, 2%, or 1% of a given value or range of values.

When a range of values (“range”) is listed, it encompasses each value and sub-range within the range. A range is inclusive of the values at the two ends of the range unless otherwise provided. For example “C₁₋₆ alkyl” encompasses, C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆ alkyl.

The term “aliphatic” refers to alkyl, alkenyl, alkynyl, and carbocyclic groups. Likewise, the term “heteroaliphatic” refers to heteroalkyl, heteroalkenyl, heteroalkynyl, and heterocyclic groups.

The term “alkyl” refers to a radical of a straight-chain or branched saturated hydrocarbon group having from 1 to 20 carbon atoms (“C₁₋₂₀ alkyl”). In some embodiments, an alkyl group has 1 to 12 carbon atoms (“C₁₋₁₂ alkyl”). In some embodiments, an alkyl group has 1 to 10 carbon atoms (“C₁₋₁₀ alkyl”). In some embodiments, an alkyl group has 1 to 9 carbon atoms (“C₁₋₉ alkyl”). In some embodiments, an alkyl group has 1 to 8 carbon atoms (“C₁₋₈ alkyl”). In some embodiments, an alkyl group has 1 to 7 carbon atoms (“C₁₋₇ alkyl”). In some embodiments, an alkyl group has 1 to 6 carbon atoms (“C₁₋₆ alkyl”). In some embodiments, an alkyl group has 1 to 5 carbon atoms (“C₁₋₅ alkyl”). In some embodiments, an alkyl group has 1 to 4 carbon atoms (“C₁₋₄ alkyl”). In some embodiments, an alkyl group has 1 to 3 carbon atoms (“C₁₋₃ alkyl”). In some embodiments, an alkyl group has 1 to 2 carbon atoms (“C₁₋₂ alkyl”). In some embodiments, an alkyl group has 1 carbon atom (“C₁ alkyl”). In some embodiments, an alkyl group has 2 to 6 carbon atoms (“C₂₋₆ alkyl”). Examples of C₁₋₆ alkyl groups include methyl (C₁), ethyl (C₂), propyl (C₃) (e.g., n-propyl, isopropyl), butyl (C₄) (e.g., n-butyl, tert-butyl, sec-butyl, isobutyl), pentyl (C₅) (e.g., n-pentyl, 3-pentanyl, amyl, neopentyl, 3-methyl-2-butanyl, tert-amyl), and hexyl (C₆) (e.g., n-hexyl). Additional examples of alkyl groups include n-heptyl (C₇), n-octyl (C₈), n-dodecyl (C₁₂), and the like. Unless otherwise specified, each instance of an alkyl group is independently unsubstituted (an “unsubstituted alkyl”) or substituted (a “substituted alkyl”) with one or more substituents (e.g., halogen, such as F). In certain embodiments, the alkyl group is an unsubstituted C₁₋₁₂ alkyl (such as unsubstituted C₁₋₆ alkyl, e.g., —CH₃ (Me), unsubstituted ethyl (Et), unsubstituted propyl (Pr, e.g., unsubstituted n-propyl (n-Pr), unsubstituted isopropyl (i-Pr)), unsubstituted butyl (Bu, e.g., unsubstituted n-butyl (n-Bu), unsubstituted tert-butyl (tert-Bu or t-Bu), unsubstituted sec-butyl (sec-Bu or s-Bu), unsubstituted isobutyl (i-Bu)). In certain embodiments, the alkyl group is a substituted C₁₋₁₂ alkyl (such as substituted C₁₋₆ alkyl, e.g., —CH₂F, —CHF₂, —CF₃, —CH₂CH₂F, —CH₂CHF₂, —CH₂CF₃, or benzyl (Bn)).

The term “haloalkyl” is a substituted alkyl group, wherein one or more of the hydrogen atoms are independently replaced by a halogen, e.g., fluoro, bromo, chloro, or iodo. “Perhaloalkyl” is a subset of haloalkyl, and refers to an alkyl group wherein all of the hydrogen atoms are independently replaced by a halogen, e.g., fluoro, bromo, chloro, or iodo. In some embodiments, the haloalkyl moiety has 1 to 20 carbon atoms (“C₁₋₂₀ haloalkyl”). In some embodiments, the haloalkyl moiety has 1 to 10 carbon atoms (“C₁₋₁₀ haloalkyl”). In some embodiments, the haloalkyl moiety has 1 to 9 carbon atoms (“C₁₋₉ haloalkyl”). In some embodiments, the haloalkyl moiety has 1 to 8 carbon atoms (“C₁₋₈ haloalkyl”). In some embodiments, the haloalkyl moiety has 1 to 7 carbon atoms (“C₁₋₇ haloalkyl”). In some embodiments, the haloalkyl moiety has 1 to 6 carbon atoms (“C₁₋₆ haloalkyl”). In some embodiments, the haloalkyl moiety has 1 to 5 carbon atoms (“C₁₋₅ haloalkyl”). In some embodiments, the haloalkyl moiety has 1 to 4 carbon atoms (“C₁₋₄ haloalkyl”). In some embodiments, the haloalkyl moiety has 1 to 3 carbon atoms (“C₁₋₃ haloalkyl”). In some embodiments, the haloalkyl moiety has 1 to 2 carbon atoms (“C₁₋₂ haloalkyl”). In some embodiments, all of the haloalkyl hydrogen atoms are independently replaced with fluoro to provide a “perfluoroalkyl” group. In some embodiments, all of the haloalkyl hydrogen atoms are independently replaced with chloro to provide a “perchloroalkyl” group. Examples of haloalkyl groups include —CHF₂, —CH₂F, —CF₃, —CH₂CF₃, —CF₂CF₃, —CF₂CF₂CF₃, —CCl₃, —CFCl₂, —CF₂Cl, and the like.

The term “heteroalkyl” refers to an alkyl group, which further includes at least one heteroatom (e.g., 1, 2, 3, or 4 heteroatoms) selected from oxygen, nitrogen, or sulfur within (e.g., inserted between adjacent carbon atoms of) and/or placed at one or more terminal position(s) of the parent chain. In certain embodiments, a heteroalkyl group refers to a saturated group having from 1 to 20 carbon atoms and 1 or more heteroatoms within the parent chain (“heteroC₁₋₂₀ alkyl”). In certain embodiments, a heteroalkyl group refers to a saturated group having from 1 to 12 carbon atoms and 1 or more heteroatoms within the parent chain (“heteroC₁₋₁₂ alkyl”). In some embodiments, a heteroalkyl group is a saturated group having 1 to 11 carbon atoms and 1 or more heteroatoms within the parent chain (“heteroC₁₋₁₁ alkyl”). In some embodiments, a heteroalkyl group is a saturated group having 1 to 10 carbon atoms and 1 or more heteroatoms within the parent chain (“heteroC₁₋₁₀ alkyl”). In some embodiments, a heteroalkyl group is a saturated group having 1 to 9 carbon atoms and 1 or more heteroatoms within the parent chain (“heteroC₁₋₉ alkyl”). In some embodiments, a heteroalkyl group is a saturated group having 1 to 8 carbon atoms and 1 or more heteroatoms within the parent chain (“heteroC₁₋₈ alkyl”). In some embodiments, a heteroalkyl group is a saturated group having 1 to 7 carbon atoms and 1 or more heteroatoms within the parent chain (“heteroC₁₋₇ alkyl”). In some embodiments, a heteroalkyl group is a saturated group having 1 to 6 carbon atoms and 1 or more heteroatoms within the parent chain (“heteroC₁₋₆ alkyl”). In some embodiments, a heteroalkyl group is a saturated group having 1 to 5 carbon atoms and 1 or 2 heteroatoms within the parent chain (“heteroC₁₋₅ alkyl”). In some embodiments, a heteroalkyl group is a saturated group having 1 to 4 carbon atoms and 1 or 2 heteroatoms within the parent chain (“heteroC₁₋₄ alkyl”). In some embodiments, a heteroalkyl group is a saturated group having 1 to 3 carbon atoms and 1 heteroatom within the parent chain (“heteroC₁₋₃ alkyl”). In some embodiments, a heteroalkyl group is a saturated group having 1 to 2 carbon atoms and 1 heteroatom within the parent chain (“heteroC₁₋₂ alkyl”). In some embodiments, a heteroalkyl group is a saturated group having 1 carbon atom and 1 heteroatom (“heteroC₁ alkyl”). In some embodiments, a heteroalkyl group is a saturated group having 2 to 6 carbon atoms and 1 or 2 heteroatoms within the parent chain (“heteroC₂₋₆ alkyl”). Unless otherwise specified, each instance of a heteroalkyl group is independently unsubstituted (an “unsubstituted heteroalkyl”) or substituted (a “substituted heteroalkyl”) with one or more substituents. In certain embodiments, the heteroalkyl group is an unsubstituted heteroC₁₋₁₂ alkyl. In certain embodiments, the heteroalkyl group is a substituted heteroC₁₋₁₂ alkyl.

The term “alkenyl” refers to a radical of a straight-chain or branched hydrocarbon group having from 1 to 20 carbon atoms and one or more carbon-carbon double bonds (e.g., 1, 2, 3, or 4 double bonds). In some embodiments, an alkenyl group has 1 to 20 carbon atoms (“C₁₋₂₀ alkenyl”). In some embodiments, an alkenyl group has 1 to 12 carbon atoms (“C₁₋₁₂ alkenyl”). In some embodiments, an alkenyl group has 1 to 11 carbon atoms (“C₁₋₁₁ alkenyl”). In some embodiments, an alkenyl group has 1 to 10 carbon atoms (“C₁₋₁₀ alkenyl”). In some embodiments, an alkenyl group has 1 to 9 carbon atoms (“C₁₋₉ alkenyl”). In some embodiments, an alkenyl group has 1 to 8 carbon atoms (“C₁₋₈ alkenyl”). In some embodiments, an alkenyl group has 1 to 7 carbon atoms (“C₁₋₇ alkenyl”). In some embodiments, an alkenyl group has 1 to 6 carbon atoms (“C₁₋₆ alkenyl”). In some embodiments, an alkenyl group has 1 to 5 carbon atoms (“C₁₋₅ alkenyl”). In some embodiments, an alkenyl group has 1 to 4 carbon atoms (“C₁₋₄ alkenyl”). In some embodiments, an alkenyl group has 1 to 3 carbon atoms (“C₁₋₃ alkenyl”). In some embodiments, an alkenyl group has 1 to 2 carbon atoms (“C₁₋₂ alkenyl”). In some embodiments, an alkenyl group has 1 carbon atom (“C₁ alkenyl”). The one or more carbon-carbon double bonds can be internal (such as in 2-butenyl) or terminal (such as in 1-butenyl). Examples of C₁₋₄ alkenyl groups include methylidenyl (C₁), ethenyl (C₂), 1-propenyl (C₃), 2-propenyl (C₃), 1-butenyl (C₄), 2-butenyl (C₄), butadienyl (C₄), and the like. Examples of C₁₋₆ alkenyl groups include the aforementioned C₂₋₄ alkenyl groups as well as pentenyl (C₅), pentadienyl (C₅), hexenyl (C₆), and the like. Additional examples of alkenyl include heptenyl (C₇), octenyl (C₈), octatrienyl (C₈), and the like. Unless otherwise specified, each instance of an alkenyl group is independently unsubstituted (an “unsubstituted alkenyl”) or substituted (a “substituted alkenyl”) with one or more substituents. In certain embodiments, the alkenyl group is an unsubstituted C₁₋₂₀ alkenyl. In certain embodiments, the alkenyl group is a substituted C₁₋₂₀ alkenyl. In an alkenyl group, a C═C double bond for which the stereochemistry is not specified (e.g., —CH═CHCH₃ or

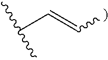

may be in the (E)- or (Z)-configuration.

The term “heteroalkenyl” refers to an alkenyl group, which further includes at least one heteroatom (e.g., 1, 2, 3, or 4 heteroatoms) selected from oxygen, nitrogen, or sulfur within (e.g., inserted between adjacent carbon atoms of) and/or placed at one or more terminal position(s) of the parent chain. In certain embodiments, a heteroalkenyl group refers to a group having from 1 to 20 carbon atoms, at least one double bond, and 1 or more heteroatoms within the parent chain (“heteroC₁₋₂₀ alkenyl”). In certain embodiments, a heteroalkenyl group refers to a group having from 1 to 12 carbon atoms, at least one double bond, and 1 or more heteroatoms within the parent chain (“heteroC₁₋₁₂ alkenyl”). In certain embodiments, a heteroalkenyl group refers to a group having from 1 to 11 carbon atoms, at least one double bond, and 1 or more heteroatoms within the parent chain (“heteroC₁₋₁₁ alkenyl”). In certain embodiments, a heteroalkenyl group refers to a group having from 1 to 10 carbon atoms, at least one double bond, and 1 or more heteroatoms within the parent chain (“heteroC₁₋₁₀ alkenyl”). In some embodiments, a heteroalkenyl group has 1 to 9 carbon atoms at least one double bond, and 1 or more heteroatoms within the parent chain (“heteroC₁₋₉ alkenyl”). In some embodiments, a heteroalkenyl group has 1 to 8 carbon atoms, at least one double bond, and 1 or more heteroatoms within the parent chain (“heteroC₁₋₈ alkenyl”). In some embodiments, a heteroalkenyl group has 1 to 7 carbon atoms, at least one double bond, and 1 or more heteroatoms within the parent chain (“heteroC₁₋₇ alkenyl”). In some embodiments, a heteroalkenyl group has 1 to 6 carbon atoms, at least one double bond, and 1 or more heteroatoms within the parent chain (“heteroC₁₋₆ alkenyl”). In some embodiments, a heteroalkenyl group has 1 to 5 carbon atoms, at least one double bond, and 1 or 2 heteroatoms within the parent chain (“heteroC₁₋₅ alkenyl”). In some embodiments, a heteroalkenyl group has 1 to 4 carbon atoms, at least one double bond, and 1 or 2 heteroatoms within the parent chain (“heteroC₁₋₄ alkenyl”). In some embodiments, a heteroalkenyl group has 1 to 3 carbon atoms, at least one double bond, and 1 heteroatom within the parent chain (“heteroC₁₋₃ alkenyl”). In some embodiments, a heteroalkenyl group has 1 to 2 carbon atoms, at least one double bond, and 1 heteroatom within the parent chain (“heteroC₁₋₂ alkenyl”). In some embodiments, a heteroalkenyl group has 1 to 6 carbon atoms, at least one double bond, and 1 or 2 heteroatoms within the parent chain (“heteroC₁₋₆ alkenyl”). Unless otherwise specified, each instance of a heteroalkenyl group is independently unsubstituted (an “unsubstituted heteroalkenyl”) or substituted (a “substituted heteroalkenyl”) with one or more substituents. In certain embodiments, the heteroalkenyl group is an unsubstituted heteroC₁₋₂₀ alkenyl. In certain embodiments, the heteroalkenyl group is a substituted heteroC₁₋₂₀ alkenyl.

The term “alkynyl” refers to a radical of a straight-chain or branched hydrocarbon group having from 1 to 20 carbon atoms and one or more carbon-carbon triple bonds (e.g., 1, 2, 3, or 4 triple bonds) (“C₁₋₂₀ alkynyl”). In some embodiments, an alkynyl group has 1 to 10 carbon atoms (“C₁₋₁₀ alkynyl”). In some embodiments, an alkynyl group has 1 to 9 carbon atoms (“C₁₋₉ alkynyl”). In some embodiments, an alkynyl group has 1 to 8 carbon atoms (“C₁₋₈ alkynyl”). In some embodiments, an alkynyl group has 1 to 7 carbon atoms (“C₁₋₇ alkynyl”). In some embodiments, an alkynyl group has 1 to 6 carbon atoms (“C₁₋₆ alkynyl”). In some embodiments, an alkynyl group has 1 to 5 carbon atoms (“C₁₋₅ alkynyl”). In some embodiments, an alkynyl group has 1 to 4 carbon atoms (“C₁₋₄ alkynyl”). In some embodiments, an alkynyl group has 1 to 3 carbon atoms (“C₁₋₃ alkynyl”). In some embodiments, an alkynyl group has 1 to 2 carbon atoms (“C₁₋₂ alkynyl”). In some embodiments, an alkynyl group has 1 carbon atom (“C₁ alkynyl”). The one or more carbon-carbon triple bonds can be internal (such as in 2-butynyl) or terminal (such as in 1-butynyl). Examples of C₁₋₄ alkynyl groups include, without limitation, methylidynyl (C₁), ethynyl (C₂), 1-propynyl (C₃), 2-propynyl (C₃), 1-butynyl (C₄), 2-butynyl (C₄), and the like. Examples of C₁₋₆ alkenyl groups include the aforementioned C₂₋₄ alkynyl groups as well as pentynyl (C₅), hexynyl (C₆), and the like. Additional examples of alkynyl include heptynyl (C₇), octynyl (C₈), and the like. Unless otherwise specified, each instance of an alkynyl group is independently unsubstituted (an “unsubstituted alkynyl”) or substituted (a “substituted alkynyl”) with one or more substituents. In certain embodiments, the alkynyl group is an unsubstituted C₁₋₂₀ alkynyl. In certain embodiments, the alkynyl group is a substituted C₁₋₂₀ alkynyl.

The term “heteroalkynyl” refers to an alkynyl group, which further includes at least one heteroatom (e.g., 1, 2, 3, or 4 heteroatoms) selected from oxygen, nitrogen, or sulfur within (e.g., inserted between adjacent carbon atoms of) and/or placed at one or more terminal position(s) of the parent chain. In certain embodiments, a heteroalkynyl group refers to a group having from 1 to 20 carbon atoms, at least one triple bond, and 1 or more heteroatoms within the parent chain (“heteroC₁₋₂₀ alkynyl”). In certain embodiments, a heteroalkynyl group refers to a group having from 1 to 10 carbon atoms, at least one triple bond, and 1 or more heteroatoms within the parent chain (“heteroC₁₋₁₀ alkynyl”). In some embodiments, a heteroalkynyl group has 1 to 9 carbon atoms, at least one triple bond, and 1 or more heteroatoms within the parent chain (“heteroC₁₋₉ alkynyl”). In some embodiments, a heteroalkynyl group has 1 to 8 carbon atoms, at least one triple bond, and 1 or more heteroatoms within the parent chain (“heteroC₁₋₈alkynyl”). In some embodiments, a heteroalkynyl group has 1 to 7 carbon atoms, at least one triple bond, and 1 or more heteroatoms within the parent chain (“heteroC₁₋₇ alkynyl”). In some embodiments, a heteroalkynyl group has 1 to 6 carbon atoms, at least one triple bond, and 1 or more heteroatoms within the parent chain (“heteroC₁₋₆ alkynyl”). In some embodiments, a heteroalkynyl group has 1 to 5 carbon atoms, at least one triple bond, and 1 or 2 heteroatoms within the parent chain (“heteroC₁₋₅ alkynyl”). In some embodiments, a heteroalkynyl group has 1 to 4 carbon atoms, at least one triple bond, and 1 or 2 heteroatoms within the parent chain (“heteroC₁₋₄ alkynyl”). In some embodiments, a heteroalkynyl group has 1 to 3 carbon atoms, at least one triple bond, and 1 heteroatom within the parent chain (“heteroC₁₋₃ alkynyl”). In some embodiments, a heteroalkynyl group has 1 to 2 carbon atoms, at least one triple bond, and 1 heteroatom within the parent chain (“heteroC₁₋₂ alkynyl”). In some embodiments, a heteroalkynyl group has 1 to 6 carbon atoms, at least one triple bond, and 1 or 2 heteroatoms within the parent chain (“heteroC₁₋₆ alkynyl”). Unless otherwise specified, each instance of a heteroalkynyl group is independently unsubstituted (an “unsubstituted heteroalkynyl”) or substituted (a “substituted heteroalkynyl”) with one or more substituents. In certain embodiments, the heteroalkynyl group is an unsubstituted heteroC₁₋₂₀ alkynyl. In certain embodiments, the heteroalkynyl group is a substituted heteroC₁₋₂₀ alkynyl.

The term “carbocyclyl” or “carbocyclic” refers to a radical of a non-aromatic cyclic hydrocarbon group having from 3 to 14 ring carbon atoms (“C₃₋₁₄ carbocyclyl”) and zero heteroatoms in the non-aromatic ring system. In some embodiments, a carbocyclyl group has 3 to 14 ring carbon atoms (“C₃₋₁₄ carbocyclyl”). In some embodiments, a carbocyclyl group has 3 to 13 ring carbon atoms (“C₃₋₁₃ carbocyclyl”). In some embodiments, a carbocyclyl group has 3 to 12 ring carbon atoms (“C₃₋₁₂ carbocyclyl”). In some embodiments, a carbocyclyl group has 3 to 11 ring carbon atoms (“C₃₋₁₁ carbocyclyl”). In some embodiments, a carbocyclyl group has 3 to 10 ring carbon atoms (“C₃₋₁₀ carbocyclyl”). In some embodiments, a carbocyclyl group has 3 to 8 ring carbon atoms (“C₃₋₈ carbocyclyl”). In some embodiments, a carbocyclyl group has 3 to 7 ring carbon atoms (“C₃₋₇ carbocyclyl”). In some embodiments, a carbocyclyl group has 3 to 6 ring carbon atoms (“C₃₋₆ carbocyclyl”). In some embodiments, a carbocyclyl group has 4 to 6 ring carbon atoms (“C₄₋₆ carbocyclyl”). In some embodiments, a carbocyclyl group has 5 to 6 ring carbon atoms (“C₅₋₆ carbocyclyl”). In some embodiments, a carbocyclyl group has 5 to 10 ring carbon atoms (“C₅₋₁₀ carbocyclyl”). Exemplary C₃₋₆ carbocyclyl groups include cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), and the like. Exemplary C₃₋₈ carbocyclyl groups include the aforementioned C₃₋₆ carbocyclyl groups as well as cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), and the like. Exemplary C₃₋₁₀ carbocyclyl groups include the aforementioned C₃₋₈ carbocyclyl groups as well as cyclononyl (C₉), cyclononenyl (C₉), cyclodecyl (C₁₀), cyclodecenyl (C₁₀), octahydro-1H-indenyl (C₉), decahydronaphthalenyl (C₁₀), spiro[4.5]decanyl (C₁₀), and the like. Exemplary C₃₋₈ carbocyclyl groups include the aforementioned C₃₋₁₀ carbocyclyl groups as well as cycloundecyl (C₁₁), spiro[5.5]undecanyl (C₁₁), cyclododecyl (C₁₂), cyclododecenyl (C₁₂), cyclotridecane (C₁₃), cyclotetradecane (C₁₄), and the like. As the foregoing examples illustrate, in certain embodiments, the carbocyclyl group is either monocyclic (“monocyclic carbocyclyl”) or polycyclic (e.g., containing a fused, bridged or spiro ring system such as a bicyclic system (“bicyclic carbocyclyl”) or tricyclic system (“tricyclic carbocyclyl”)) and can be saturated or can contain one or more carbon-carbon double or triple bonds. “Carbocyclyl” also includes ring systems wherein the carbocyclyl ring, as defined above, is fused with one or more aryl or heteroaryl groups wherein the point of attachment is on the carbocyclyl ring, and in such instances, the number of carbons continue to designate the number of carbons in the carbocyclic ring system. Unless otherwise specified, each instance of a carbocyclyl group is independently unsubstituted (an “unsubstituted carbocyclyl”) or substituted (a “substituted carbocyclyl”) with one or more substituents. In certain embodiments, the carbocyclyl group is an unsubstituted C₃₋₁₄ carbocyclyl. In certain embodiments, the carbocyclyl group is a substituted C₃₋₁₄ carbocyclyl.

In some embodiments, “carbocyclyl” or “cycloalkyl” is a monocyclic, saturated carbocyclyl group having from 3 to 14 ring carbon atoms (“C₃₋₁₄ cycloalkyl”). In some embodiments, a cycloalkyl group has 3 to 10 ring carbon atoms (“C₃₋₁₀ cycloalkyl”). In some embodiments, a cycloalkyl group has 3 to 8 ring carbon atoms (“C₃₋₈ cycloalkyl”). In some embodiments, a cycloalkyl group has 3 to 6 ring carbon atoms (“C₃₋₆ cycloalkyl”). In some embodiments, a cycloalkyl group has 4 to 6 ring carbon atoms (“C₄₋₆ cycloalkyl”). In some embodiments, a cycloalkyl group has 5 to 6 ring carbon atoms (“C₅₋₆ cycloalkyl”). In some embodiments, a cycloalkyl group has 5 to 10 ring carbon atoms (“C₅₋₁₀ cycloalkyl”). Examples of C₅₋₆ cycloalkyl groups include cyclopentyl (C₅) and cyclohexyl (C₅). Examples of C₃₋₆ cycloalkyl groups include the aforementioned C₅₋₆ cycloalkyl groups as well as cyclopropyl (C₃) and cyclobutyl (C₄). Examples of C₃₋₈ cycloalkyl groups include the aforementioned C₃₋₆ cycloalkyl groups as well as cycloheptyl (C₇) and cyclooctyl (C₈). Unless otherwise specified, each instance of a cycloalkyl group is independently unsubstituted (an “unsubstituted cycloalkyl”) or substituted (a “substituted cycloalkyl”) with one or more substituents. In certain embodiments, the cycloalkyl group is an unsubstituted C₃₋₁₄ cycloalkyl. In certain embodiments, the cycloalkyl group is a substituted C₃₋₁₄ cycloalkyl. In certain embodiments, the carbocyclyl includes 0, 1, or 2 C═C double bonds in the carbocyclic ring system, as valency permits.

The term “heterocyclyl” or “heterocyclic” or “heterocyclyl” refers to a radical of a 3-to 14-membered non-aromatic ring system having ring carbon atoms and 1 to 4 ring heteroatoms, wherein each heteroatom is independently selected from nitrogen, oxygen, and sulfur (“3-14 membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or polycyclic (e.g., a fused, bridged or spiro ring system such as a bicyclic system (“bicyclic heterocyclyl”) or tricyclic system (“tricyclic heterocyclyl”)), and can be saturated or can contain one or more carbon-carbon double or triple bonds. Heterocyclyl polycyclic ring systems can include one or more heteroatoms in one or both rings. “Heterocyclyl” also includes ring systems wherein the heterocyclyl ring, as defined above, is fused with one or more carbocyclyl groups wherein the point of attachment is either on the carbocyclyl or heterocyclyl ring, or ring systems wherein the heterocyclyl ring, as defined above, is fused with one or more aryl or heteroaryl groups, wherein the point of attachment is on the heterocyclyl ring, and in such instances, the number of ring members continue to designate the number of ring members in the heterocyclyl ring system. Unless otherwise specified, each instance of heterocyclyl is independently unsubstituted (an “unsubstituted heterocyclyl”) or substituted (a “substituted heterocyclyl”) with one or more substituents. In certain embodiments, the heterocyclyl group is an unsubstituted 3-14 membered heterocyclyl. In certain embodiments, the heterocyclyl group is a substituted 3-14 membered heterocyclyl. In certain embodiments, the heterocyclyl is substituted or unsubstituted, 3- to 7-membered, monocyclic heterocyclyl, wherein 1, 2, or 3 atoms in the heterocyclic ring system are independently oxygen, nitrogen, or sulfur, as valency permits.

In some embodiments, a heterocyclyl group is a 5-10 membered non-aromatic ring system having ring carbon atoms and 1-4 ring heteroatoms, wherein each heteroatom is independently selected from nitrogen, oxygen, and sulfur (“5-10 membered heterocyclyl”). In some embodiments, a heterocyclyl group is a 5-8 membered non-aromatic ring system having ring carbon atoms and 1-4 ring heteroatoms, wherein each heteroatom is independently selected from nitrogen, oxygen, and sulfur (“5-8 membered heterocyclyl”). In some embodiments, a heterocyclyl group is a 5-6 membered non-aromatic ring system having ring carbon atoms and 1-4 ring heteroatoms, wherein each heteroatom is independently selected from nitrogen, oxygen, and sulfur (“5-6 membered heterocyclyl”). In some embodiments, the 5-6 membered heterocyclyl has 1-3 ring heteroatoms selected from nitrogen, oxygen, and sulfur. In some embodiments, the 5-6 membered heterocyclyl has 1-2 ring heteroatoms selected from nitrogen, oxygen, and sulfur. In some embodiments, the 5-6 membered heterocyclyl has 1 ring heteroatom selected from nitrogen, oxygen, and sulfur.

Exemplary 3-membered heterocyclyl groups containing 1 heteroatom include azirdinyl, oxiranyl, and thiiranyl. Exemplary 4-membered heterocyclyl groups containing 1 heteroatom include azetidinyl, oxetanyl, and thietanyl. Exemplary 5-membered heterocyclyl groups containing 1 heteroatom include tetrahydrofuranyl, dihydrofuranyl, tetrahydrothiophenyl, dihydrothiophenyl, pyrrolidinyl, dihydropyrrolyl, and pyrrolyl-2,5-dione. Exemplary 5-membered heterocyclyl groups containing 2 heteroatoms include dioxolanyl, oxathiolanyl and dithiolanyl. Exemplary 5-membered heterocyclyl groups containing 3 heteroatoms include triazolinyl, oxadiazolinyl, and thiadiazolinyl. Exemplary 6-membered heterocyclyl groups containing 1 heteroatom include piperidinyl, tetrahydropyranyl, dihydropyridinyl, and thianyl. Exemplary 6-membered heterocyclyl groups containing 2 heteroatoms include piperazinyl, morpholinyl, dithianyl, and dioxanyl. Exemplary 6-membered heterocyclyl groups containing 3 heteroatoms include triazinyl. Exemplary 7-membered heterocyclyl groups containing 1 heteroatom include azepanyl, oxepanyl and thiepanyl. Exemplary 8-membered heterocyclyl groups containing 1 heteroatom include azocanyl, oxecanyl and thiocanyl. Exemplary bicyclic heterocyclyl groups include indolinyl, isoindolinyl, dihydrobenzofuranyl, dihydrobenzothienyl, tetra-hydrobenzothienyl, tetrahydrobenzofuranyl, tetrahydroindolyl, tetrahydroquinolinyl, tetrahydroisoquinolinyl, decahydroquinolinyl, decahydroisoquinolinyl, octahydrochromenyl, octahydroisochromenyl, decahydronaphthyridinyl, decahydro-1,8-naphthyridinyl, octahydropyrrolo[3,2-b]pyrrole, indolinyl, phthalimidyl, naphthalimidyl, chromanyl, chromenyl, 1H-benzo[e][1,4]diazepinyl, 1,4,5,7-tetrahydropyrano[3,4-b]pyrrolyl, 5,6-dihydro-4H-furo[3,2-b]pyrrolyl, 6,7-dihydro-5H-furo[3,2-b]pyranyl, 5,7-dihydro-4H-thieno[2,3-c]pyranyl, 2,3-dihydro-1H-pyrrolo[2,3-b]pyridinyl, 2,3-dihydrofuro[2,3-b]pyridinyl, 4,5,6,7-tetrahydro-1H-pyrrolo[2,3-b]pyridinyl, 4,5,6,7-tetrahydrofuro[3,2-c]pyridinyl, 4,5,6,7-tetrahydrothieno[3,2-b]pyridinyl, 1,2,3,4-tetrahydro-1,6-naphthyridinyl, and the like.

The term “aryl” refers to a radical of a monocyclic or polycyclic (e.g., bicyclic or tricyclic) 4n+2 aromatic ring system (e.g., having 6, 10, or 14 π electrons shared in a cyclic array) having 6-14 ring carbon atoms and zero heteroatoms provided in the aromatic ring system (“C₆₋₁₄ aryl”). In some embodiments, an aryl group has 6 ring carbon atoms (“C₆ aryl”; e.g., phenyl). In some embodiments, an aryl group has 10 ring carbon atoms (“C₁₀ aryl”; e.g., naphthyl such as 1-naphthyl and 2-naphthyl). In some embodiments, an aryl group has 14 ring carbon atoms (“C₁₄ aryl”; e.g., anthracyl). “Aryl” also includes ring systems wherein the aryl ring, as defined above, is fused with one or more carbocyclyl or heterocyclyl groups wherein the radical or point of attachment is on the aryl ring, and in such instances, the number of carbon atoms continue to designate the number of carbon atoms in the aryl ring system. Unless otherwise specified, each instance of an aryl group is independently unsubstituted (an “unsubstituted aryl”) or substituted (a “substituted aryl”) with one or more substituents. In certain embodiments, the aryl group is an unsubstituted C₆₋₁₄ aryl. In certain embodiments, the aryl group is a substituted C₆₋₁₄ aryl.

The term “heteroaryl” refers to a radical of a 5-14 membered monocyclic or polycyclic (e.g., bicyclic, tricyclic) 4n+2 aromatic ring system (e.g., having 6, 10, or 14 π electrons shared in a cyclic array) having ring carbon atoms and 1-4 ring heteroatoms provided in the aromatic ring system, wherein each heteroatom is independently selected from nitrogen, oxygen, and sulfur (“5-14 membered heteroaryl”). In heteroaryl groups that contain one or more nitrogen atoms, the point of attachment can be a carbon or nitrogen atom, as valency permits. Heteroaryl polycyclic ring systems can include one or more heteroatoms in one or both rings. “Heteroaryl” includes ring systems wherein the heteroaryl ring, as defined above, is fused with one or more carbocyclyl or heterocyclyl groups wherein the point of attachment is on the heteroaryl ring, and in such instances, the number of ring members continue to designate the number of ring members in the heteroaryl ring system. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused polycyclic (aryl/heteroaryl) ring system. Polycyclic heteroaryl groups wherein one ring does not contain a heteroatom (e.g., indolyl, quinolinyl, carbazolyl, and the like) the point of attachment can be on either ring, e.g., either the ring bearing a heteroatom (e.g., 2-indolyl) or the ring that does not contain a heteroatom (e.g., 5-indolyl). In certain embodiments, the heteroaryl is substituted or unsubstituted, 5- or 6-membered, monocyclic heteroaryl, wherein 1, 2, 3, or 4 atoms in the heteroaryl ring system are independently oxygen, nitrogen, or sulfur. In certain embodiments, the heteroaryl is substituted or unsubstituted, 9- or 10-membered, bicyclic heteroaryl, wherein 1, 2, 3, or 4 atoms in the heteroaryl ring system are independently oxygen, nitrogen, or sulfur.

In some embodiments, a heteroaryl group is a 5-10 membered aromatic ring system having ring carbon atoms and 1-4 ring heteroatoms provided in the aromatic ring system, wherein each heteroatom is independently selected from nitrogen, oxygen, and sulfur (“5-10 membered heteroaryl”). In some embodiments, a heteroaryl group is a 5-8 membered aromatic ring system having ring carbon atoms and 1-4 ring heteroatoms provided in the aromatic ring system, wherein each heteroatom is independently selected from nitrogen, oxygen, and sulfur (“5-8 membered heteroaryl”). In some embodiments, a heteroaryl group is a 5-6 membered aromatic ring system having ring carbon atoms and 1-4 ring heteroatoms provided in the aromatic ring system, wherein each heteroatom is independently selected from nitrogen, oxygen, and sulfur (“5-6 membered heteroaryl”). In some embodiments, the 5-6 membered heteroaryl has 1-3 ring heteroatoms selected from nitrogen, oxygen, and sulfur. In some embodiments, the 5-6 membered heteroaryl has 1-2 ring heteroatoms selected from nitrogen, oxygen, and sulfur. In some embodiments, the 5-6 membered heteroaryl has 1 ring heteroatom selected from nitrogen, oxygen, and sulfur. Unless otherwise specified, each instance of a heteroaryl group is independently unsubstituted (an “unsubstituted heteroaryl”) or substituted (a “substituted heteroaryl”) with one or more substituents. In certain embodiments, the heteroaryl group is an unsubstituted 5-14 membered heteroaryl. In certain embodiments, the heteroaryl group is a substituted 5-14 membered heteroaryl.

Exemplary 5-membered heteroaryl groups containing 1 heteroatom include pyrrolyl, furanyl, and thiophenyl. Exemplary 5-membered heteroaryl groups containing 2 heteroatoms include imidazolyl, pyrazolyl, oxazolyl, isoxazolyl, thiazolyl, and isothiazolyl. Exemplary 5-membered heteroaryl groups containing 3 heteroatoms include triazolyl, oxadiazolyl, and thiadiazolyl. Exemplary 5-membered heteroaryl groups containing 4 heteroatoms include tetrazolyl. Exemplary 6-membered heteroaryl groups containing 1 heteroatom include pyridinyl. Exemplary 6-membered heteroaryl groups containing 2 heteroatoms include pyridazinyl, pyrimidinyl, and pyrazinyl. Exemplary 6-membered heteroaryl groups containing 3 or 4 heteroatoms include triazinyl and tetrazinyl, respectively. Exemplary 7-membered heteroaryl groups containing 1 heteroatom include azepinyl, oxepinyl, and thiepinyl. Exemplary 5,6-bicyclic heteroaryl groups include indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. Exemplary 6,6-bicyclic heteroaryl groups include naphthyridinyl, pteridinyl, quinolinyl, isoquinolinyl, cinnolinyl, quinoxalinyl, phthalazinyl, and quinazolinyl. Exemplary tricyclic heteroaryl groups include phenanthridinyl, dibenzofuranyl, carbazolyl, acridinyl, phenothiazinyl, phenoxazinyl, and phenazinyl.

The term “alkaryl” refers to an alkyl substituted by an aryl group (e.g., benzyl, phenethyl, or 3,4-dichlorophenethyl).

The term “alkcycloalkyl” refers to an alkyl substituted with a carbocyclic group (e.g., cyclopropylmethyl).

The term “alkheterocyclyl” refers to an alkyl substituted with a heterocyclic group (e.g., 3-furanylmethyl, 2-furanylmethyl, 3-tetrahydrofuranylmethyl, or 2-tetrahydrofuranylmethyl).

The term “unsaturated bond” refers to a double or triple bond.

The term “unsaturated” or “partially unsaturated” refers to a moiety that includes at least one double or triple bond.

The term “saturated” or “fully saturated” refers to a moiety that does not contain a double or triple bond, e.g., the moiety only contains single bonds.

A group is optionally substituted unless expressly provided otherwise. The term “optionally substituted” refers to being substituted or unsubstituted. In certain embodiments, alkyl, alkenyl, alkynyl, heteroalkyl, heteroalkenyl, heteroalkynyl, carbocyclyl, heterocyclyl, aryl, and heteroaryl groups are optionally substituted. “Optionally substituted” refers to a group which is substituted or unsubstituted (e.g., “substituted” or “unsubstituted” alkyl, “substituted” or “unsubstituted” alkenyl, “substituted” or “unsubstituted” alkynyl, “substituted” or “unsubstituted” heteroalkyl, “substituted” or “unsubstituted” heteroalkenyl, “substituted” or “unsubstituted” heteroalkynyl, “substituted” or “unsubstituted” carbocyclyl, “substituted” or “unsubstituted” heterocyclyl, “substituted” or “unsubstituted” aryl or “substituted” or “unsubstituted” heteroaryl group). In general, the term “substituted” means that at least one hydrogen present on a group is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent is either the same or different at each position. The term “substituted” is contemplated to include substitution with all permissible substituents of organic compounds, and includes any of the substituents described herein that results in the formation of a stable compound. The present disclosure contemplates any and all such combinations in order to arrive at a stable compound. For purposes of this disclosure, heteroatoms such as nitrogen may have hydrogen substituents and/or any suitable substituent as described herein which satisfy the valencies of the heteroatoms and results in the formation of a stable moiety. The disclosure is not limited in any manner by the exemplary substituents described herein.

The term “halo” or “halogen” refers to fluorine (fluoro, —F), chlorine (chloro, —Cl), bromine (bromo, —Br), or iodine (iodo, —I).

The term “quaternary amine” refers a cationic amine in which the nitrogen atom has four groups bonded to it and carries a positive charge. In some embodiments, the quaternary amines provided herein have a counterion. A “counterion” or “anionic counterion” or “pharmaceutically acceptable anion” (e.g., X⁻) is a negatively charged group associated with a positively charged group in order to maintain electronic neutrality. An anionic counterion may be monovalent (e.g., including one formal negative charge). An anionic counterion may also be multivalent (e.g., including more than one formal negative charge), such as divalent or trivalent. Exemplary counterions include halide ions (e.g., F⁻, Cl⁻, Br⁻, I⁻), NO₃⁻, ClO₄⁻, OH⁻, H₂PO₄⁻, HCO₃⁻, HSO₄⁻, sulfonate ions (e.g., methansulfonate, trifluoromethanesulfonate, p-toluenesulfonate, benzenesulfonate, 10-camphor sulfonate, naphthalene-2-sulfonate, naphthalene-1-sulfonic acid-5-sulfonate, ethan-1-sulfonic acid-2-sulfonate, and the like), carboxylate ions (e.g., acetate, propanoate, benzoate, glycerate, lactate, tartrate, glycolate, gluconate, and the like), BF₄⁻, PF₄⁻, PF₆⁻, AsF₆⁻, SbF₆⁻, B[3,5-(CF₃)₂C₆H₃]₄]⁻, B(C₆F₅)₄⁻, BPh₄⁻, Al(OC(CF₃)₃)₄⁻, and carborane anions (e.g., CB₁₁H₁₂⁻ or (HCB₁₁Me₅Br₆)⁻). Exemplary counterions which may be multivalent include CO₃²⁻, HPO₄²⁻, PO₄³⁻, B₄O₇²⁻, SO₄²⁻, S₂O₃²⁻, carboxylate anions (e.g., tartrate, citrate, fumarate, maleate, malate, malonate, gluconate, succinate, glutarate, adipate, pimelate, suberate, azelate, sebacate, salicylate, phthalates, aspartate, glutamate, and the like), and carboranes. In some embodiments, the quaternary amine counterion is F⁻, Cl⁻, Br⁻, or I⁻. In some embodiments, the counterion is a non-coordinating anionic counterion. The term “non-coordinating anionic counterion” refers to an anion that interacts weakly with cations. Exemplary non-coordinating anions include, but are not limited to, ClO₄⁻, NO₃⁻, TfO⁻, BF₄⁻, PF₄⁻, PF₆⁻, and SbF₆⁻. Other examples of non-coordinating anions include, but are not limited to, B(C₆F₅)₄⁻, B[3,5-(CF₃)₂C₆H₃]₄]⁻, BPh₄⁻, Sb(OTeF₅)₆⁻, Al(OC(CF₃)₃)₄⁻, or a carborane anion (e.g., CB₁₁H₁₂⁻, CB₁₁(CF₃)₁₂⁻, or (HCB₁₁Me₅Br₆)⁻).

The term “nociceptor” refers to a sensory neuron. Nociceptors respond to damaging or potentially damaging stimuli by sending signals to the spinal cord and brain. Upon the brain recognizing a credible threat, the sensation of pain is created to direct attention to the originating body part in order to mitigate the threat. The action of nociceptors is called nociception.

A “sodium channel” is a membrane protein that form ion channels, conducting sodium ions (Na⁺) through a cell's plasma membrane. In neurons, sodium channels are responsible for the rising phase of action potentials. In some embodiments, the sodium channel is a Na_{V}1.6, Na_{V}1.7, Na_{V}1.8, and Na_{V}1.9 comprising sodium channel.

As used herein, the term “agent” means a molecule, group of molecules, complex, or substance. Agents include small molecules, peptides, proteins, nucleic acids, and the like.

A “neuropeptide modulating agent” is an agent modifies the activity of a neuropeptide. In some embodiments, the neuropeptide modulating agent is an agent that blocks the release of a neuropeptide. In some embodiments, the neuropeptide modulating agent is an agent that modifies the action of a neuropeptide.

The term “neuropeptide” refers to small proteins produced by neurons that act on G protein-coupled receptors and are responsible for slow-onset, long-lasting modulation of synaptic transmission.

The term “vesicle” refers to a structure within or outside a cell, consisting of liquid or cytoplasm enclosed by a lipid bilayer. Vesicles are involved in metabolism, transport, buoyancy, and temporary storage of food and enzymes.

A “nociceptor modulating agent” is an agent that modifies the activity of a nociceptor. In some embodiments, the nociceptor modulating agent inhibits the activity of a nociceptor (e.g., the agent is a nociceptor antagonist).

A “nociceptor antagonist” is an agent that inhibits the activity of a nociceptor. In some embodiments, the nociceptor antagonist is a sodium channel blocker. In some embodiments, the nociceptor antagonist is a calcium channel blocker. In some embodiments, the nociceptor antagonist is a sodium and calcium channel blocker.

A “sodium channel blocker” is an agent impairs the conduction of sodium ions (Na⁺) through sodium channels. In some embodiments, the sodium channel blocker is also a nociceptor antagonist. In some embodiments, the sodium channel blocker is Ranolazine, Phenytoin, Disopyramide, Lidocaine, Mexiletine, Triamterene, Lamotrigine, Amiloride, Moricizine, Oxcarbazepine, Quinidine, Procainamide, Tocainide, Amiodarone, Propafenone, Flecainide, Encainide, Ajmaline, Aprindine, Tetrodotoxin, Eslicarbazepine acetate, Pilsicainide, Eslicarbazepine, Carbamazepine, Ethotoin, Fosphenytoin, Rufinamide, or Lacosamide. In some embodiments, the sodium channel blocker is a compound comprising a quaternary amine (e.g., a quaternary amine of Formula (I), (IA), (II), (III), (IV), (V), (VI), (VII), (VIII), (IX), (X), (XI), (XII), (XIII), or (XIV)).

A “calcium channel blocker” is an agent that disrupts the movement of calcium (Ca₂⁺) through calcium channels. In some embodiments, the calcium channel blocker is also a nociceptor antagonist. In some embodiments, the calcium channel blocker is ziconotide, amlodipine, clevidipine, diltiazem, felodipine, isradipine, nicardipine, nifedipine, nimodipine, nisoldipine, or verapamil. In some embodiments, the calcium channel blocker is ziconotide. In some embodiments, the calcium channel blocker is a compound comprising a quaternary amine (e.g., a quaternary amine of Formula (I), (IA), (II), (III), (IV), (V), (VI), (VII), (VIII), (IX), (X), (XI), (XII), (XIII), or (XIV)).

A “sodium and calcium channel blocker” is an agent that is both a sodium channel blocker as well as a calcium channel blocker. In some embodiments the sodium and calcium channel blocker is CNCB-2 (Lee et al. Elife 2019 Nov. 25;8:e48118. doi: 10.7554/eLife.48118). In some embodiments, the sodium and calcium channel blocker is a compound comprising a quaternary amine (e.g., a quaternary amine of Formula (I), (IA), (II), (III), (IV), (V), (VI), (VII), (VIII), (IX), (X), (XI), (XII), (XIII), or (XIV)).

An “ion channel blocker” or “channel blocker” refers to an agent that is a sodium channel blocker, calcium channel blocker, or sodium and calcium channel blocker.

An “antagonist” is a substance that interferes with or inhibits the physiological action of another.

The terms “calcitonin gene-related peptide modulating agent” and “CGRP modulating agent” are used interchangeably and refer to an agent that modifies the activity of calcitonin gene-related peptide. In some embodiments, the CGRP modulating agent acts through CGRP's receptor activity-modifying proteins (RAMPs). In some embodiments, the RAMP is RAMP1 or RAMP2.

The term “RAMP” or “receptor activity-modifying proteins” refers to a class of protein that interact with and modulate activity of numerous Class B G protein-coupled receptors including the receptors for secretin, calcitonin, glucagon, and vasoactive intestinal peptide. There are three types of RAMPs in mammals: RAMP1, RAMP2, and RAMP3. RAMP1 is a protein that in humans is encoded by the RAMP1 gene. In combination with the RAMP1 protein, calcitonin-receptor-like receptor functions as the CGRP receptor. RAMP2 is a protein which in humans is encoded by the RAMP2 gene. In the presence of RAMP2 protein, calcitonin-receptor-like receptor functions as an adrenomedullin receptor.

The terms “calcitonin gene-related peptide antagonist” and “CGRP antagonist” are used interchangeably to refer to agents that antagonize the CGRP. In some embodiments, the CGRP antagonist is BIBN 4096.

The term “ablate” as used herein refers to removal, especially the removal of a nociceptor. In some embodiments, the nociceptor is ablated via surgical, genetic, or chemical means.

The term “inhibition,” “inhibiting,” “inhibit,” or “inhibitor” refers to the ability of an agent to reduce, slow, halt or prevent activity of a particular biological process (e.g., activity of a nociceptor (e.g., nociception)) in a cell relative to vehicle.

The terms “silence” or “silencing” as used herein refers to preventing the normal activity of something. In certain embodiments, “silencing” means to partially prevent the normal activity of something. In certain embodiments, “silencing” means to completely prevent the normal activity of something (e.g., it is now inactive). In some embodiments, silencing a nociceptor means to prevent the normal activity of a nociceptor (e.g., via use of an agent that inhibits or antagonizes the nociceptor), or by removing the nociceptor (e.g., genetically or surgically ablating the nociceptor). In some embodiments, silencing a sensory neuron means to prevent the normal activity of a sensory neuron (e.g., via use of an agent that inhibits or antagonizes the sensory neuron), or by removing the sensory neuron (e.g., genetically or surgically ablating the sensory neuron).

The term “BIBN 4096”, “BIBN4096” and “BIBN” refer to a small molecule having the following structure:

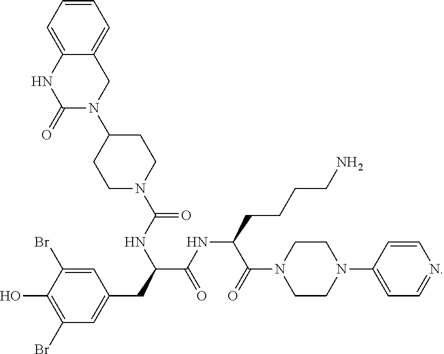 [SMILES: NCCCC[C@H](NC(=O)[C@@H](Cc1cc(Br)c(O)c(Br)c1)NC(=O)N1CCC(N2Cc3ccccc3NC2=O)CC1)C(=O)N1CCN(c2ccncc2)CC1]

The terms “BoNT” or “BONT” refer to botulinum neurotoxins. BoNTs are protein neurotoxins produced by neurotoxigenic strains of anaerobic and spore forming bacteria of the genus Clostridium (Clostridium botulinum, Clostridium butyrricum, Clostridium barati, and Clostridium argentinensis). “BoNT/a” and “BONT/a” refer to botulinum toxin type a.

“TeNT” refers to tetanus neurotoxin produced by Clostridium tetani.

The term “innervated”, “innervate”, or the like refers to nerves being supplied. In some embodiments, the tumors are innervated, such that the tumors are supplied with nerves. In some embodiments, innervated tumors are more aggressive than less innervated one.

A “subject” to which administration is contemplated refers to a human (i.e., male or female of any age group, e.g., pediatric subject (e.g., infant, child, or adolescent) or adult subject (e.g., young adult, middle-aged adult, or senior adult)) or non-human animal. In certain embodiments, the non-human animal is a mammal (e.g., primate (e.g., cynomolgus monkey or rhesus monkey), commercially relevant mammal (e.g., cattle, pig, horse, sheep, goat, cat, or dog), or bird (e.g., commercially relevant bird, such as chicken, duck, goose, or turkey)). In certain embodiments, the non-human animal is a fish, reptile, or amphibian. The non-human animal may be a male or female at any stage of development. The non-human animal may be a transgenic animal or genetically engineered animal. The term “patient” refers to a human subject in need of treatment of a disease.

The term “administer,” “administering,” or “administration” refers to implanting, absorbing, ingesting, injecting, inhaling, or otherwise introducing a compound described herein, or a composition thereof, in or on a subject.

The terms “treatment,” “treat,” and “treating” refer to reversing, alleviating, delaying the onset of, or inhibiting the progress of a disease described herein. In some embodiments, treatment may be administered after one or more signs or symptoms of the disease have developed or have been observed. In other embodiments, treatment may be administered in the absence of signs or symptoms of the disease. For example, treatment may be administered to a susceptible subject prior to the onset of symptoms (e.g., in light of a history of symptoms and/or in light of exposure to a pathogen). Treatment may also be continued after symptoms have resolved, for example, to delay or prevent recurrence.

An “effective amount” of a compound described herein refers to an amount sufficient to elicit the desired biological response. An effective amount of a compound described herein may vary depending on such factors as the desired biological endpoint, severity of side effects, disease, or disorder, the identity, pharmacokinetics, and pharmacodynamics of the particular compound, the condition being treated, the mode, route, and desired or required frequency of administration, the species, age and health or general condition of the subject. In certain embodiments, an effective amount is a therapeutically effective amount. In certain embodiments, an effective amount is a prophylactic treatment. In certain embodiments, an effective amount is the amount of a compound described herein in a single dose. In certain embodiments, an effective amount is the combined amounts of a compound described herein in multiple doses. In certain embodiments, the desired dosage is delivered three times a day, two times a day, once a day, every other day, every third day, every week, every two weeks, every three weeks, or every four weeks. In certain embodiments, the desired dosage is delivered using multiple administrations (e.g., two, three, four, five, six, seven, eight, nine, ten, eleven, twelve, thirteen, fourteen, or more administrations). In certain embodiments, an effective amount of a compound for administration one or more times a day to a 70 kg adult human comprises about 0.0001 mg to about 3000 mg, about 0.0001 mg to about 2000 mg, about 0.0001 mg to about 1000 mg, about 0.001 mg to about 1000 mg, about 0.01 mg to about 1000 mg, about 0.1 mg to about 1000 mg, about 1 mg to about 1000 mg, about 1 mg to about 100 mg, about 10 mg to about 1000 mg, or about 100 mg to about 1000 mg, of a compound per unit dosage form. In certain embodiments, the compounds provided herein may be administered orally or parenterally at dosage levels sufficient to deliver from about 0.001 mg/kg to about 100 mg/kg, from about 0.01 mg/kg to about 50 mg/kg, preferably from about 0.1 mg/kg to about 40 mg/kg, preferably from about 0.5 mg/kg to about 30 mg/kg, from about 0.01 mg/kg to about 10 mg/kg, from about 0.1 mg/kg to about 10 mg/kg, and more preferably from about 1 mg/kg to about 25 mg/kg, of subject body weight per day, one or more times a day, to obtain the desired therapeutic effect. It will be appreciated that dose ranges as described herein provide guidance for the administration of compounds to an adult. The amount to be administered to, for example, a child or an adolescent can be determined by a medical practitioner or person skilled in the art and can be lower or the same as that administered to an adult.

A “therapeutically effective amount” of a compound described herein is an amount sufficient to provide a therapeutic benefit in the treatment of a condition or to delay or minimize one or more symptoms associated with the condition. A therapeutically effective amount of a compound means an amount of therapeutic agent, alone or in combination with other therapies, which provides a therapeutic benefit in the treatment of the condition. The term “therapeutically effective amount” can encompass an amount that improves overall therapy, reduces or avoids symptoms, signs, or causes of the condition, and/or enhances the therapeutic efficacy of another therapeutic agent. In certain embodiments, a therapeutically effective amount is an amount sufficient for silencing tumor-innervating sensory neurons. In certain embodiments, a therapeutically effective amount is an amount sufficient for blocking nociceptors. In certain embodiments, a therapeutically effective amount is an amount sufficient for treating cancer.

A “prophylactically effective amount” of a compound described herein is an amount sufficient to prevent a condition, or one or more symptoms associated with the condition or prevent its recurrence. A prophylactically effective amount of a compound means an amount of a therapeutic agent, alone or in combination with other agents, which provides a prophylactic benefit in the prevention of the condition. The term “prophylactically effective amount” can encompass an amount that improves overall prophylaxis or enhances the prophylactic efficacy of another prophylactic agent. In certain embodiments, a prophylactically effective amount is an amount sufficient for silencing tumor-innervating sensory neurons. In certain embodiments, a prophylactically effective amount is an amount sufficient for blocking nociceptors. In certain embodiments, a prophylactically effective amount is an amount sufficient for preventing cancer.

The term “prevent,” “preventing,” or “prevention” refers to a prophylactic treatment of a subject who is not and was not with a disease but is at risk of developing the disease or who was with a disease, is not with the disease, but is at risk of regression of the disease. In certain embodiments, the subject is at a higher risk of developing the disease or at a higher risk of regression of the disease than an average healthy member of a population.

The terms “neoplasm” and “tumor” are used herein interchangeably and refer to an abnormal mass of tissue wherein the growth of the mass surpasses and is not coordinated with the growth of a normal tissue. A neoplasm or tumor may be “benign” or “malignant,” depending on the following characteristics: degree of cellular differentiation (including morphology and functionality), rate of growth, local invasion, and metastasis. A “benign neoplasm” is generally well differentiated, has characteristically slower growth than a malignant neoplasm, and remains localized to the site of origin. In addition, a benign neoplasm does not have the capacity to infiltrate, invade, or metastasize to distant sites. Exemplary benign neoplasms include, but are not limited to, lipoma, chondroma, adenomas, acrochordon, senile angiomas, seborrheic keratoses, lentigos, and sebaceous hyperplasias. In some cases, certain “benign” tumors may later give rise to malignant neoplasms, which may result from additional genetic changes in a subpopulation of the tumor's neoplastic cells, and these tumors are referred to as “pre-malignant neoplasms.” An exemplary pre-malignant neoplasm is a teratoma. In contrast, a “malignant neoplasm” is generally poorly differentiated (anaplasia) and has characteristically rapid growth accompanied by progressive infiltration, invasion, and destruction of the surrounding tissue. Furthermore, a malignant neoplasm generally has the capacity to metastasize to distant sites. The term “metastasis,” “metastatic,” or “metastasize” refers to the spread or migration of cancerous cells from a primary or original tumor to another organ or tissue and is typically identifiable by the presence of a “secondary tumor” or “secondary cell mass” of the tissue type of the primary or original tumor and not of that of the organ or tissue in which the secondary (metastatic) tumor is located. For example, a prostate cancer that has migrated to bone is said to be metastasized prostate cancer and includes cancerous prostate cancer cells growing in bone tissue. IN some embodiments, the tumor is innervated.

The term “cancer” refers to a class of diseases characterized by the development of abnormal cells that proliferate uncontrollably and have the ability to infiltrate and destroy normal body tissues. See e.g., Stedman's Medical Dictionary, 25th ed.; Hensyl ed.; Williams & Wilkins: Philadelphia, 1990. Exemplary cancers include, but are not limited to, acoustic neuroma; adenocarcinoma; adrenal gland cancer; anal cancer; angiosarcoma (e.g., lymphangiosarcoma, lymphangioendotheliosarcoma, hemangiosarcoma); appendix cancer; benign monoclonal gammopathy; biliary cancer (e.g., cholangiocarcinoma); bladder cancer; breast cancer (e.g., adenocarcinoma of the breast, papillary carcinoma of the breast, mammary cancer, medullary carcinoma of the breast); brain cancer (e.g., meningioma, glioblastomas, glioma (e.g., astrocytoma, oligodendroglioma), medulloblastoma); bronchus cancer; carcinoid tumor; cervical cancer (e.g., cervical adenocarcinoma); choriocarcinoma; chordoma; craniopharyngioma; colorectal cancer (e.g., colon cancer, rectal cancer, colorectal adenocarcinoma); connective tissue cancer; epithelial carcinoma; ependymoma; endotheliosarcoma (e.g., Kaposi's sarcoma, multiple idiopathic hemorrhagic sarcoma); endometrial cancer (e.g., uterine cancer, uterine sarcoma); esophageal cancer (e.g., adenocarcinoma of the esophagus, Barrett's adenocarcinoma); Ewing's sarcoma; ocular cancer (e.g., intraocular melanoma, retinoblastoma); familiar hypereosinophilia; gall bladder cancer; gastric cancer (e.g., stomach adenocarcinoma); gastrointestinal stromal tumor (GIST); germ cell cancer; head and neck cancer (e.g., head and neck squamous cell carcinoma, oral cancer (e.g., oral squamous cell carcinoma), throat cancer (e.g., laryngeal cancer, pharyngeal cancer, nasopharyngeal cancer, oropharyngeal cancer)); hematopoietic cancers (e.g., leukemia such as acute lymphocytic leukemia (ALL) (e.g., B-cell ALL, T-cell ALL), acute myelocytic leukemia (AML) (e.g., B-cell AML, T-cell AML), chronic myelocytic leukemia (CML) (e.g., B-cell CML, T-cell CML), and chronic lymphocytic leukemia (CLL) (e.g., B-cell CLL, T-cell CLL)); lymphoma such as Hodgkin lymphoma (HL) (e.g., B-cell HL, T-cell HL) and non-Hodgkin lymphoma (NHL) (e.g., B-cell NHL such as diffuse large cell lymphoma (DLCL) (e.g., diffuse large B-cell lymphoma), follicular lymphoma, chronic lymphocytic leukemia/small lymphocytic lymphoma (CLL/SLL), mantle cell lymphoma (MCL), marginal zone B-cell lymphomas (e.g., mucosa-associated lymphoid tissue (MALT) lymphomas, nodal marginal zone B-cell lymphoma, splenic marginal zone B-cell lymphoma), primary mediastinal B-cell lymphoma, Burkitt lymphoma, lymphoplasmacytic lymphoma (i.e., Waldenström's macroglobulinemia), hairy cell leukemia (HCL), immunoblastic large cell lymphoma, precursor B-lymphoblastic lymphoma and primary central nervous system (CNS) lymphoma; and T-cell NHL such as precursor T-lymphoblastic lymphoma/leukemia, peripheral T-cell lymphoma (PTCL) (e.g., cutaneous T-cell lymphoma (CTCL) (e.g., mycosis fungoides, Sezary syndrome), angioimmunoblastic T-cell lymphoma, extranodal natural killer T-cell lymphoma, enteropathy type T-cell lymphoma, subcutaneous panniculitis-like T-cell lymphoma, and anaplastic large cell lymphoma); a mixture of one or more leukemia/lymphoma as described above; and multiple myeloma (MM)), heavy chain disease (e.g., alpha chain disease, gamma chain disease, mu chain disease); hemangioblastoma; hypopharynx cancer; inflammatory myofibroblastic tumors; immunocytic amyloidosis; kidney cancer (e.g., nephroblastoma a.k.a. Wilms' tumor, renal cell carcinoma); liver cancer (e.g., hepatocellular cancer (HCC), malignant hepatoma); lung cancer (e.g., bronchogenic carcinoma, small cell lung cancer (SCLC), non-small cell lung cancer (NSCLC), adenocarcinoma of the lung); leiomyosarcoma (LMS); mastocytosis (e.g., systemic mastocytosis); muscle cancer; myelodysplastic syndrome (MDS); mesothelioma; myeloproliferative disorder (MPD) (e.g., polycythemia vera (PV), essential thrombocytosis (ET), agnogenic myeloid metaplasia (AMM) a.k.a. myelofibrosis (MF), chronic idiopathic myelofibrosis, chronic myelocytic leukemia (CML), chronic neutrophilic leukemia (CNL), hypereosinophilic syndrome (HES)); neuroblastoma; neurofibroma (e.g., neurofibromatosis (NF) type 1 or type 2, schwannomatosis); neuroendocrine cancer (e.g., gastroenteropancreatic neuroendoctrine tumor (GEP-NET), carcinoid tumor); osteosarcoma (e.g., bone cancer); ovarian cancer (e.g., cystadenocarcinoma, ovarian embryonal carcinoma, ovarian adenocarcinoma); papillary adenocarcinoma; pancreatic cancer (e.g., pancreatic andenocarcinoma, intraductal papillary mucinous neoplasm (IPMN), Islet cell tumors); penile cancer (e.g., Paget's disease of the penis and scrotum); pinealoma; primitive neuroectodermal tumor (PNT); plasma cell neoplasia; paraneoplastic syndromes; intraepithelial neoplasms; prostate cancer (e.g., prostate adenocarcinoma); rectal cancer; rhabdomyosarcoma; salivary gland cancer; skin cancer (e.g., squamous cell carcinoma (SCC), keratoacanthoma (KA), melanoma, basal cell carcinoma (BCC)); small bowel cancer (e.g., appendix cancer); soft tissue sarcoma (e.g., malignant fibrous histiocytoma (MFH), liposarcoma, malignant peripheral nerve sheath tumor (MPNST), chondrosarcoma, fibrosarcoma, myxosarcoma); sebaceous gland carcinoma; small intestine cancer; sweat gland carcinoma; synovioma; testicular cancer (e.g., seminoma, testicular embryonal carcinoma); thyroid cancer (e.g., papillary carcinoma of the thyroid, papillary thyroid carcinoma (PTC), medullary thyroid cancer); urethral cancer; vaginal cancer; and vulvar cancer (e.g., Paget's disease of the vulva). In some embodiments, cancer includes a benign and malignant tumors. In some embodiments, cancer includes a benign tumor. In some embodiments, the cancer is a tumor. In some embodiments, the cancer is a melanoma. In some embodiments, the melanoma is superficial spreading melanoma, nodular melanoma, acral-lentiginous melanoma, lentigo maligna melanoma, amelanotic melanoma, desmoplastic melanoma, ocular melanoma, or metastatic melanoma. In some embodiments, the cancer is metastatic melanoma.

These and other exemplary substituents are described in more detail in the Detailed Description, Examples, and Claims. The invention is not limited in any manner by the above exemplary listing of substituents. Additional terms may be defined in other sections of this disclosure.

### DETAILED DESCRIPTION OF CERTAIN EMBODIMENTS

Before the disclosed methods and uses are described in more detail, it should be understood that the aspects described herein are not limited to specific embodiments, methods, or uses, and as such can, of course, vary. It is also to be understood that the terminology used herein is for the purpose of describing particular embodiments only and, unless specifically defined herein, is not intended to be limiting.

Described herein are methods of treating and preventing cancer. The data presented herein shows nociceptors play a regulatory role in the immune response to tumor growth, through the regulation of immune checkpoint receptor expression on cytotoxic CD8⁺ T-cells. Silencing tumor-innervating sensory neurons represents an innovative strategy for attenuating the immunomodulatory power of the nervous system and promoting anti-tumor activity.

### Methods and Uses

In some embodiments, provided herein are methods of treating cancer in a subject comprising silencing tumor-innervating sensory neurons. In some embodiments, provided herein are methods of preventing cancer in a subject comprising silencing tumor-innervating sensory neurons.

In some embodiments, provided herein are methods of treating cancer in a subject comprising silencing tumor-innervating nociceptors. In some embodiments, provided herein are methods of preventing cancer in a subject comprising silencing tumor-innervating nociceptors.

In some embodiments, provided herein are methods of treating cancer in a subject comprising administering to the subject a therapeutically effective amount of a nociceptor modulating compound. In some embodiments, provided herein are methods of preventing cancer in a subject, the method comprising administering to the subject a prophylactically effective amount of a nociceptor modulating agent. In some embodiments, the nociceptor modulating agent is a nociceptor antagonist. In some embodiments, the nociceptor antagonist is a sodium channel blocker. In certain embodiments, the sodium channel is selected from Na_{V}1.6, Na_{V}1.7, Na_{V}1.8, and Na_{V}1.9. In some embodiments, the sodium channel is Na_{V}1.8. In some embodiments, the nociceptor antagonist is a calcium channel blocker. In some embodiments, the calcium channel is Ca_{V}1.1-1.4, Ca_{V}2.1, Ca_{V}2.2, Ca_{V}2.3, Ca_{V}3.1-3.3. In some embodiments, the calcium channel is Ca_{V}2.2.

In some embodiments, provided herein are methods of treating cancer in a subject comprising administering to the subject a therapeutically effective amount of a neuropeptide modulating agent. In some embodiments, provided herein are methods of preventing cancer in a subject comprising administering to the subject a prophylactically effective amount of a neuropeptide modulating agent. In some embodiments, the neuropeptide is calcitonin gene-related peptide (CGRP).

In some embodiments, provided herein are methods of treating cancer in a subject, the method comprising administering to the subject a therapeutically effective amount of an agent that blocks the release or action of neuropeptide from tumor-innervating neurons. In certain embodiments, provided herein are methods of treating cancer in a subject comprising administering to the subject a therapeutically effective amount of an agent that blocks the release or action of a neuropeptide from tumor-innervating nociceptors. In some embodiments, provided herein are methods of preventing cancer in a subject comprising administering to the subject a prophylactically effective amount of an agent that blocks the release or action of a neuropeptide from tumor-innervating neurons. In certain embodiments, provided herein are methods of preventing cancer in a subject comprising administering to the subject a prophylactically effective amount of an agent that blocks the release or action of a neuropeptide from tumor-innervating nociceptors. In some embodiments, the neuropeptide is CGRP.

In some embodiments, the agent blocks the release of a neuropeptide. In some embodiments, the agent blocks the action of a neuropeptide. In some embodiments, the neuropeptide is CGRP.

In some embodiments, the nociceptor antagonist prevents release of a neuropeptide. In some embodiments, the neuropeptide is CGRP.

In certain embodiments, the nociceptor modulating agent, nociceptor antagonist, neuropeptide modulating agent, an agent that blocks vesicle release, or agent that blocks the release or action of a neuropeptide from tumor-innervating nociceptor is a compound comprising a quaternary amine. In some embodiments, the nociceptor modulating agent, nociceptor antagonist, neuropeptide modulating agent, an agent that blocks vesicle release, or agent that blocks the release or action of the neuropeptide from tumor-innervating nociceptor is QX-314:

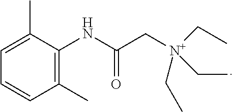

In certain embodiments, the nociceptor modulating agent, nociceptor antagonist, neuropeptide modulating agent, an agent that blocks vesicle release, or agent that blocks the release or action of a neuropeptide from tumor-innervating nociceptor is a compound selected from the group consisting of:

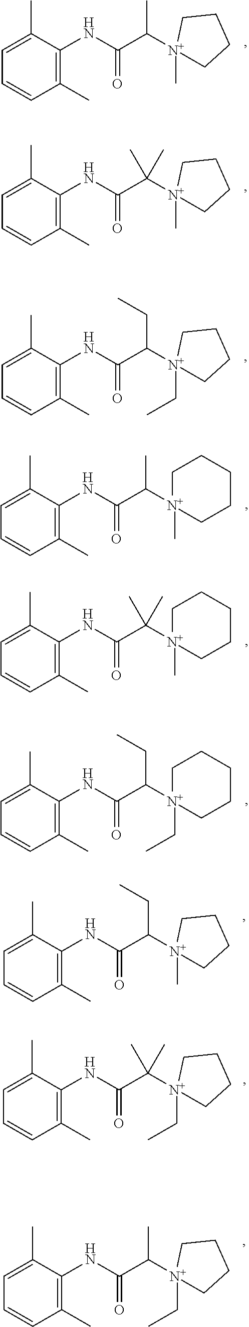

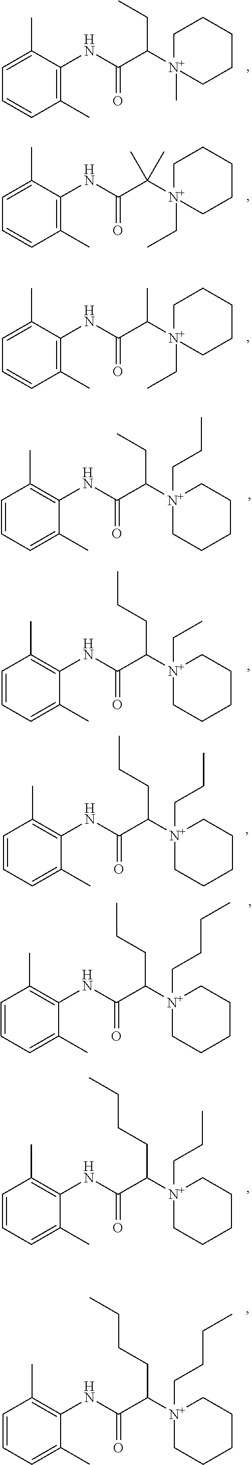

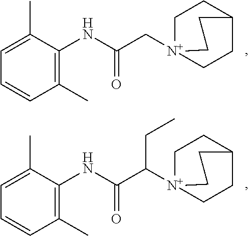 [SMILES: Cc1cccc(C)c1NC(=O)C[N+]12CCC(CC1)CC2 ; CCC(C(=O)Nc1c(C)cccc1C)[N+]12CCC(CC1)CC2]

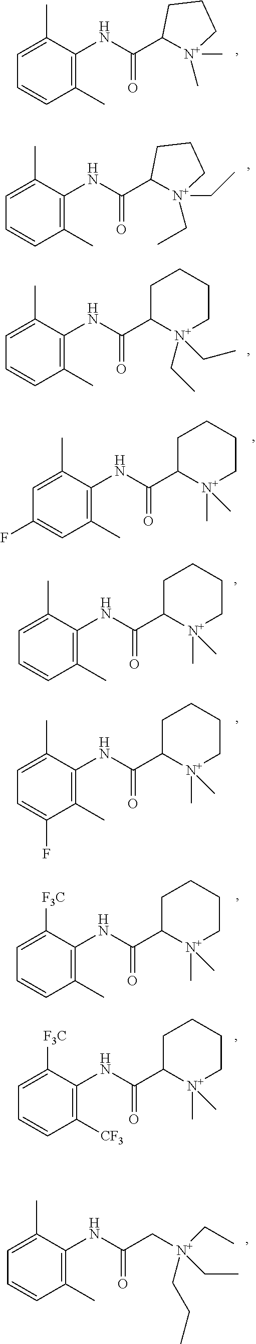

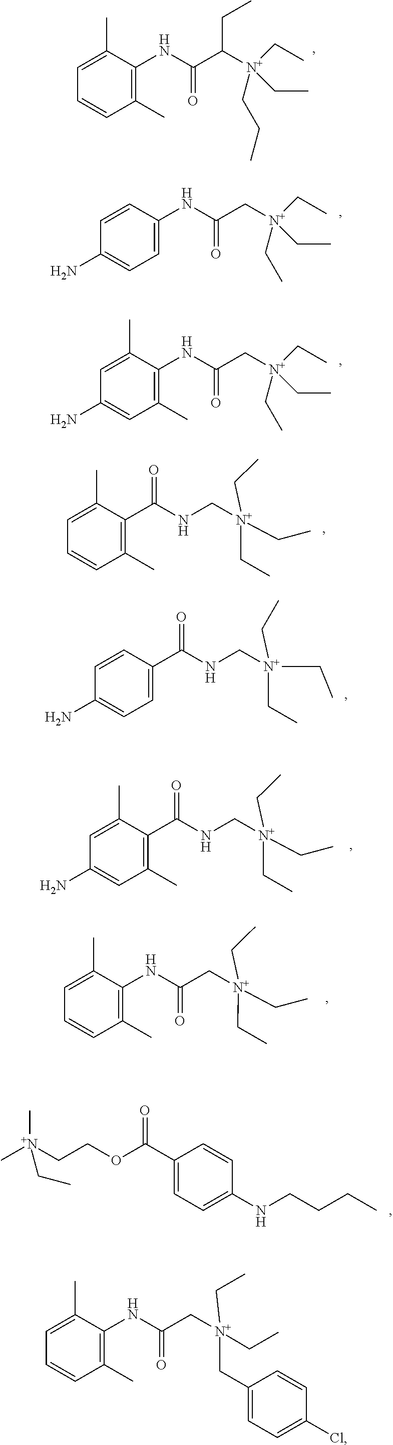

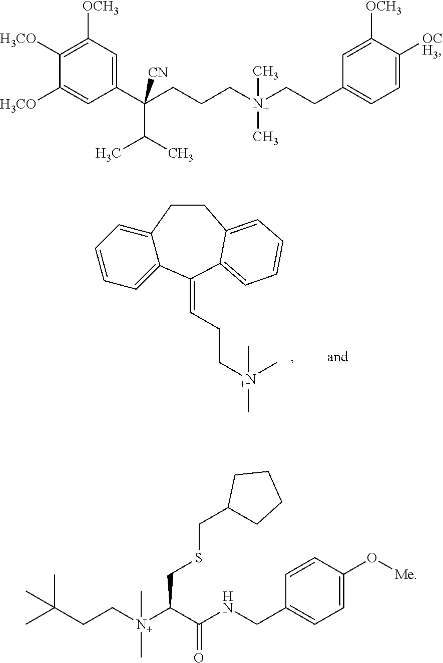 [SMILES: COc1ccc(CC[N+](C)(C)CCC[C@@](C#N)(c2cc(OC)c(OC)c(OC)c2)C(C)C)cc1OC ; C[N+](C)(C)CCC=C1c2ccccc2CCc2ccccc21 ; COc1ccc(CNC(=O)[C@H](CSCC2CCCC2)[N+](C)(C)CCC(C)(C)C)cc1]

In some embodiments, the nociceptor modulating agent, nociceptor antagonist, neuropeptide modulating agent, an agent that blocks vesicle release, or agent that blocks the release or action of a neuropeptide from tumor-innervating nociceptor is a quaternary amine of Formula (I):

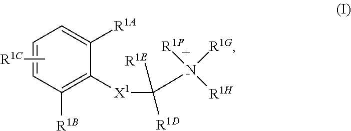

wherein:

R^{1F} and R^{1G} together complete a heterocyclic or heteroaryl ring having at least one nitrogen atom;

each of R^{1A}, R^{1B}, and R^{1C} is independently selected from H, halogen, C₁₋₄ alkyl, C₂₋₄ alkenyl, C₂₋₄ alkynyl, OR^{1I}, NR^{1J}R^{1K}, NR^{1L}C(O)R^{1M}, S(O)R^{1N}, SO₂R^{1O}R^{1P}, SO₂NR^{1Q}R^{1R}, SO₃R^{1S}, CO₂R^{1T}, C(O)R^{1U}, and C(O)NR^{1V}R^{1W};

each of R^{1I}, R^{1J}, R^{1K}, R^{1L}, R^{1M}, R^{1N}, R^{1O}, R^{1P}, R^{1Q}, R^{1R}, R^{1S}, R^{1T}, R^{1U}, R^{1V}, and R^{1W} is, independently, selected from H, C₁₋₄ alkyl, C₂₋₄ alkenyl, C₂₋₄ alkynyl, and C₂₋₄ heteroalkyl;

X¹ is selected from —CR^{1X}R^{1Y}—, —NR^{1Z}C(O)—, —OC(O)—, —SC(O)—, —C(O)NR^{1AA}—, —CO₂—, and —OC(S)—;

each of R^{1X}, R^{1Y}, R^{1Z}, and R^{1AA} is independently selected from H, C₁₋₄ alkyl, C₂₋₄ alkenyl, C₂₋₄ alkynyl, and C₂₋₄ heteroalkyl;

each of R^{1D} and R^{1E} is independently selected from H, C₁₋₄ alkyl, C₂₋₄ alkenyl, C₂₋₄ alkynyl, C₂₋₄ heteroalkyl, and C₃₋₆ carbocyclyl, wherein each R^{1D} and R^{1E} is optionally substituted with halogen, C₃₋₈ carbocyclyl, aryl, or heteroaryl; or R^{1D} and R^{1E} together form a 3-6-membered heterocyclic or carbocyclic ring; and

R^{1H} is selected from C₁₋₄ alkyl, C₂₋₄ alkenyl, C₂₋₄ alkynyl, C₂₋₄ heteroalkyl, and C₃₋₆ carbocyclyl, wherein R^{1H} is optionally substituted with halogen, C₃₋₈ carbocyclyl, aryl, or heteroaryl.

In some embodiments, R^{1F} and R^{1G} together complete a 4-8-membered heterocyclic ring. In some embodiments, R^{1F} and R^{1G} together complete a 5-, 6-, or 7-membered heterocyclic ring. In some embodiments, R^{1F} and R^{1G} together complete a pyrrolidine, piperidine, or azepane ring. In some embodiments, the heterocyclic ring formed by R^{1F} and R^{1G} is optionally substituted with C₁₋₄ alkyl, halogen, C₃₋₈ carbocyclyl, aryl, or heteroaryl.

In some embodiments, R^{1A} and R^{1B} are each independently H, halogen, C₁₋₄ alkyl, or CO₂R^{1T}. In some embodiments, R^{1A} and R^{1B} are each independently H, C₁₋₄ alkyl, or CO₂R^{1T}. In some embodiments, R^{1A} and R^{1B} are each independently C₁₋₄ alkyl or CO₂R^{1T} In some embodiments, R^{1A} and R^{1B} are each methyl.

In some embodiments, X¹ is —NHC(O)—.

In some embodiments, each of R^{1D} and R^{1E} is independently selected from H, C₁₋₄ alkyl, C₂₋₄ alkenyl, C₂₋₄ alkynyl, C₂₋₄ heteroalkyl, and C₃₋₆ carbocyclyl, wherein each R^{1D} and R^{1E} is optionally substituted with halogen, C₃₋₈ carbocyclyl, aryl, or heteroaryl. In some embodiments, each of R^{1D} and R^{1E} is hydrogen. In some embodiments, each of R^{1D} and R^{1E} is independently C₁₋₄ alkyl, wherein each R^{1D} and R^{1E} is optionally substituted with halogen, C₃₋₈ carbocyclyl, aryl, or heteroaryl. In some embodiments, each of R^{1D} and R^{1E} is independently C₁₋₄ alkyl, wherein each R^{1D} and R^{1E} is substituted with halogen, C₃₋₈ carbocyclyl, aryl, or heteroaryl.

In some embodiments, R^{1H} is selected from C₁₋₄ alkyl, wherein R^{1H} is optionally substituted with halogen, C₃₋₈ carbocyclyl, aryl, or heteroaryl. In some embodiments, R^{1H} is selected from C₁₋₄ alkyl, wherein R^{1H} is substituted with halogen, C₃₋₈ carbocyclyl, aryl, or heteroaryl.

In some embodiments, the nociceptor modulating agent, nociceptor antagonist, neuropeptide modulating agent, an agent that blocks vesicle release, or agent that blocks the release or action of a neuropeptide from tumor-innervating nociceptor is a compound or agent described in (i) PCT publication WO2008/063603, WO2011/006073, WO2017/024037, WO2020/142657, WO2020/185830, WO2020/185928, WO2020/185915, or WO2020/185881, (ii) U.S. patent Ser. No. 10/780,083, 10927096, 10842798, 10828287, 10925865, or 10786485, or (iii) US patent publication US 2020/0290953, US 2020/0290965, or US 2020/0290979.

In some embodiments, the nociceptor modulating agent, nociceptor antagonist, neuropeptide modulating agent, an agent that blocks vesicle release, or agent that blocks the release or action of a neuropeptide from tumor-innervating nociceptor is a quaternary amine derivative or other permanently charged derivative of a compound selected from riluzole, mexilitine, phenytoin, carbamazepine, procaine, articaine, bupivicaine, mepivicaine, tocainide, prilocaine, diisopyramide, bencyclane, quinidine, bretylium, lifarizine, lamotrigine, flunarizine, and fluspirilene.

In some embodiments, the nociceptor modulating agent, nociceptor antagonist, neuropeptide modulating agent, an agent that blocks vesicle release, or agent that blocks the release or action of a neuropeptide from tumor-innervating nociceptor is QX-314, N-methyl-procaine, QX-222, N-octyl-guanidine, 9-aminoacridine, pancuronium, or another low molecular weight, charged molecule that inhibits voltage-gated sodium channels when present inside of said nociceptor. In some embodiments, the nociceptor modulating agent, nociceptor antagonist, neuropeptide modulating agent, an agent that blocks vesicle release, or agent that blocks the release or action of a neuropeptide from tumor-innervating nociceptor is D-890, CERM 11888, N-methyl-verapamil, N-methylgallopamil, N-methyl-devapamil, dodecyltrimethylammonium, or a quaternary amine derivative of verapamil, gallopamil, devapamil, diltiazem, fendiline, mibefradil, or farnesol amine.

In some embodiments, the nociceptor modulating agent, nociceptor antagonist, neuropeptide modulating agent, an agent that blocks vesicle release, or agent that blocks the release or action of a neuropeptide from tumor-innervating nociceptor is of Formula IA:

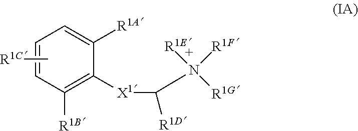

wherein:

each of R^{1A′}, R^{1B′}, and R^{1C′} is, independently, selected from H, halogen, C₁₋₄ alkyl, C₂₋₄ alkenyl, C₂₋₄ alkynyl, OR^{1H′}, NR^{1I′}R^{1J′}, NR^{1K′}C(O)R^{1L′}, S(O)R^{1M′}, SO₂R^{1N′}R^{1O′}, SO₂NR^{1P′}R^{1Q′}, SO₃R^{1R′}, CO₂R^{1S′}, C(O)R^{1T′}, and C(O)NR^{1U′}R^{1V′};

each of R^{1H′}, R^{1I′}, R^{1J′}, R^{1K′}, R^{1L′}, R^{1M′}, R^{1N′}, R^{1O′}, R^{1P′}, R^{1Q′}, R^{1R′}, R^{1S′}, R^{1T′}, R^{1U′}, and R^{1V′} is, independently, selected from H, C₁₋₄ alkyl, C₂₋₄ alkenyl, C₂₋₄ alkynyl, and C₂₋₄ heteroalkyl

X^{1′} is selected from —CR^{1W′}R^{1X′}—, —NR^{1Y′}C(O)—, —OC(O)—, —SC(O)—, —C(O)NR^{1Z′}—, —CO₂—, and —OC(S)—;

each of R^{1W′}, R^{1X′}, R^{1Y′}, and R^{1Z′} is, independently, selected from H, C₁₋₄ alkyl, C₂₋₄ alkenyl, C₂₋₄ alkynyl, and C₂₋₄ heteroalkyl;

R^{1D′} is selected from H, C₁₋₄ alkyl, C₂₋₄ alkenyl, C₂₋₄ alkynyl, and C₂₋₄ heteroalkyl;

each of R^{1E′}, R^{1F′}, and R^{1G′} is, independently, selected from C₁₋₄ alkyl, C₂₋₄ alkenyl, C₂₋₄ alkynyl, and C₂₋₄ heteroalkyl; or

R^{1D′} and R^{1G′} together complete a heterocyclic ring having at least one nitrogen atom.

In a preferred embodiment, X^{1′} is —NHC(O)—. Exemplary compounds of Formula IA include methylated quaternary ammonium derivatives of anesthetic drugs, such as N-methyl lidocaine, N,N-dimethyl prilocaine, N,N,N-trimethyl tocainide, N-methyl etidocaine, N-methyl ropivacaine, N-methyl bupivacaine, N-methyl levobupivacaine, N-methyl mepivacaine. These derivatives can be prepared using methods analogous to those described in Scheme 1. Compounds of Formula IA include

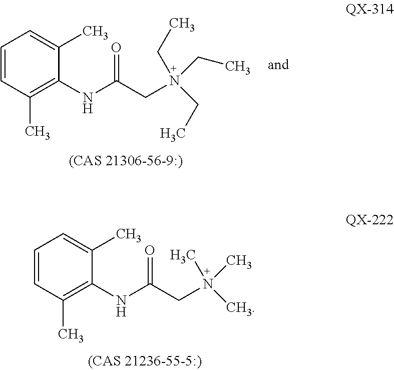

In some embodiments, the nociceptor modulating agent, nociceptor antagonist, neuropeptide modulating agent, an agent that blocks vesicle release, or agent that blocks the release or action of a neuropeptide from tumor-innervating nociceptor is of Formula II:

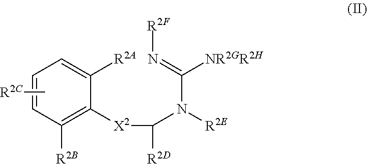

wherein:

each of R^{2A}, R^{2B}, and R^{2C} is, independently, selected from H, halogen, C₁₋₄ alkyl, C₂₋₄ alkenyl, C₂₋₄ alkynyl, OR^{2I}, NR^{2J}R^{2K}, NR^{2L}C(O)R^{2M}, S(O)R^{2N}, SO₂R^{2O}R^{2P}, SO₂NR^{2Q}R^{2R}, SO₃R^{2S}, CO₂R^{2T}, C(O)R^{2U}, and C(O)NR^{2V}R^{2W};

each of R^{2I}, R^{2J}, R^{2K}, R^{2L}, R^{2M}, R^{2N}, R^{2O}, R^{2P}, R^{2Q}, R^{2R}, R^{2S}, R^{2T}, R^{2U}, R^{2V}, R^{2W} is, independently, selected from H, C₁₋₄ alkyl, C₂₋₄ alkenyl, C₂₋₄ alkynyl, and C₂₋₄ heteroalkyl;

X² is selected from —CR^{2X}R^{2Y}—, —NR^{2Z}C(O)—, —OC(O)—, —SC(O)—, —C(O)NR^{2AA}—, —CO₂—, and —OC(S)—;

each of R^{2X}, R^{2Y}, R^{2Z}, and R^{2AA} is, independently, selected from H, C₁₋₄ alkyl, C₂₋₄ alkenyl, C₂₋₄ alkynyl, and C₂₋₄ heteroalkyl;

R^{2D} is selected from H, C₁₋₄ alkyl, C₂₋₄ alkenyl, C₂₋₄ alkynyl, and C₂₋₄ heteroalkyl;

R^{2E} is H or C₁₋₄ alkyl; and

each of R^{2F}, R^{2G}, and R^{2H} is, independently, selected from H, C₁₋₄ alkyl, C₂₋₄ alkenyl, C₂₋₄ alkynyl, and C₂₋₄ heteroalkyl;

or R^{2F} and R^{2G} together complete a heterocyclic ring having two nitrogen atoms.

In some embodiments, when R^{2F} and R^{2G} form a heterocyclic ring having two nitrogen atoms, the resulting guanidine group is selected from

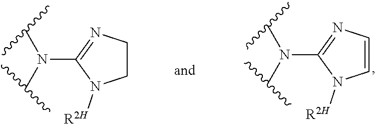

where R^{2H} is H or CH₃. In some embodiments, R^{2F} and R^{2G} combine to form an alkylene or alkenylene of from 2 to 4 carbon atoms, e.g., ring systems of 5, 6, and 7-membered rings. In a preferred embodiment, X² is —NHC(O)—. Exemplary compounds of formula II include N-guanidyl derivatives (e.g., —C(NH)NH₂ derivatives) of anesthetic drugs, such as desethyl-N-guanidyl lidocaine, N-guanidyl prilocaine, N-guanidyl tocainide, desethyl-N-guanidyl etidocaine, desbutyl-N-guanidyl ropivacaine, desbutyl-N-guanidyl bupivacaine, desbutyl-N-guanidyl levobupivacaine, desmethyl-N-guanidyl mepivacaine. These derivatives can be prepared using methods analogous to those described in Schemes 2-5.

The guanidyl derivatives described herein (e.g., the compounds of formula II) are presented in their uncharged base form. These compounds can be administered either as a salt (i.e., an acid addition salt) or in their uncharged base form, which undergoes protonation in situ to form a charged moiety.

The synthesis of parent drugs of formulas I and II are described in the literature. See, for example, U.S. Pat. No. 2,441,498 (synthesis of lidocaine), U.S. Pat. No. 3,160,662 (synthesis of prilocaine), DE Patent No. 2235745 (synthesis of tocainide), DE Patent No. 2162744 (synthesis of etidocaine), PCT Publication No. WO85/00599 (synthesis of ropivacaine), U.S. Pat. No. 2,955,111 (synthesis of bupivacaine and levobupivacaine), and U.S. Pat. No. 2,799,679 (synthesis of mepivacaine).

In some embodiments, the nociceptor modulating agent, nociceptor antagonist, neuropeptide modulating agent, an agent that blocks vesicle release, or agent that blocks the release or action of a neuropeptide from tumor-innervating nociceptor is of Formula III:

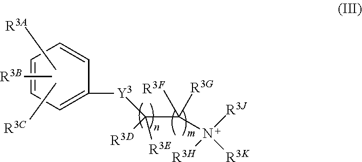

wherein:

n=0-3;

m=0-3; wherein (n+m)=0-6;

each of R^{3A}, R^{3B}, and R^{3C} is, independently, selected from H, halogen, C₁₋₄ alkyl, C₂₋₄ alkenyl, C₂₋₄ alkynyl, C₂₋₄ heteroalkyl, OR^{3L}, NR^{3M}R^{3N}, NR^{3O}C(O)R^{3P}, S(O)R^{3Q}, SO₂R^{3R}R^{3S}, SO₂NR^{3T}R^{3U}, SO₃R^{3V}, CO₂R^{3W}, C(O)R^{3X}, and C(O)NR^{3Y}R^{3Z};

each of R^{3L}, R^{3M}, R^{3N}, R^{3O}, R^{3P}, R^{3Q}, R^{3R}, R^{3S}, R^{3T}, R^{3U}, R^{3V}, R^{3W}, R^{3X}, R^{3Y}, R^{3Z} is, independently, selected from H, C₁₋₄ alkyl, C₂₋₄ alkenyl, C₂₋₄ alkynyl, and C₂₋₄ heteroalkyl;

Y³ is selected from —CR^{3AA}R^{3AB}—, NR^{3AC}C(O)—, —OC(O)—, —SC(O)—, —C(O)NR^{3AD}—, —CO₂—, and —OC(S)—;

each of R^{3AA}, R^{3AB}, R^{3AC}, and R^{3AD} is, independently, selected from H, C₁₋₄ alkyl, C₂₋₄ alkenyl, C₂₋₄ alkynyl, and C₂₋₄ heteroalkyl;

each of R^{3D}, R^{3E}, R^{3F}, and R^{3G} is, independently, selected from H, C₁₋₄ alkyl, C₂₋₄ alkenyl, C₂₋₄ alkynyl, C₂₋₄ heteroalkyl, C₂₋₆ heterocyclyl, C₆₋₁₂ aryl, C₇₋₁₄ alkaryl, and C₃₋₁₀ alkheterocyclyl; and

each of R^{3H}, R^{3J}, and R^{3K} is, independently, selected from C₁₋₄ alkyl, C₂₋₄ alkenyl, C₂₋₄ alkynyl, and C₂₋₄ heteroalkyl.

The quaternary nitrogen in formula III is identified herein as N′. Exemplary compounds of formula III include methylated quaternary ammonium derivatives of anesthetic drugs, such as N′-methyl procaine, N′-methyl proparacaine, N′-methyl allocain, N′-methyl encainide, N′-methyl procainamide, N′-methyl metoclopramide, N′-methyl stovaine, N′-methyl propoxycaine, N′-methyl chloroprocaine, N′,N′-dimethyl flecainide, and N′-methyl tetracaine. These derivatives can be prepared using methods analogous to those described in Scheme 1.

In some embodiments, the nociceptor modulating agent, nociceptor antagonist, neuropeptide modulating agent, an agent that blocks vesicle release, or agent that blocks the release or action of a neuropeptide from tumor-innervating nociceptor is of Formula IV:

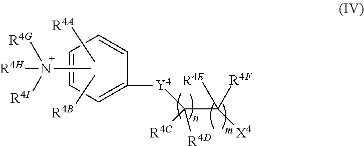

wherein:

n=0-3;

m=0-3; wherein with (n+m)=0-6;

each of R^{4A} and R^{4B} is, independently, selected from H, halogen, C₁₋₄ alkyl, C₂₋₄ alkenyl, C₂₋₄ alkynyl, C₂₋₄ heteroalkyl, OR^{4L}, NR^{4M}R^{4N}, NR^{4O} C(O)R^{4P}, S(O)R^{4Q}, SO₂R^{4R}R^{4S}, SO₂NR^{4T}R^{4U}, SO₃R^{4V}, CO₂R^{4W}, C(O)R^{4X}, and C(O)NR^{4Y}R^{4Z};

each of R^{4L}, R^{4M}R^{4N}, R^{4O}, R^{4P}, R^{4Q}, R^{4R}, R^{4S}, R^{4T}, R^{4U}, R^{4V}, R^{4W}, R^{4X}, R^{4Y}, and R^{4Z} is, independently, selected from H, C₁₋₄ alkyl, C₂₋₄ alkenyl, C₂₋₄ alkynyl, and C₂₋₄ heteroalkyl;

Y⁴ is selected from —CR^{4AA}R^{4AB}—, —NR^{4AC}C(O)—, —OC(O)—, —SC(O)—, —C(O)NR^{4AD}—, —CO₂—, and —OC(S)—;

each of R^{4AA}, R^{4AB}, R^{4AC}, and R^{4AD} is, independently, selected from H, C₁₋₄ alkyl, C₂₋₄ alkenyl, C₂₋₄ alkynyl, and C₂₋₄ heteroalkyl;

each of R^{4C}, R^{4D}, R^{4E}, and R^{4F} is, independently, selected from H, C₁₋₄ alkyl, C₂₋₄ alkenyl, C₂₋₄ alkynyl, C₂₋₄ heteroalkyl, C₂₋₆ heterocyclyl, C₆₋₁₂ aryl, C₇₋₁₄ alkaryl, and C₃₋₁₀ alkheterocyclyl;

X⁴ is selected from H, C₁₋₄ alkyl, C₂₋₄ alkenyl, C₂₋₄ alkynyl, and NR^{4J}R^{4K};

each of R^{4J} and R^{4K} is, independently, selected from H, C₁₋₄ alkyl, C₂₋₄ alkenyl, C₂₋₄ alkynyl, and C₂₋₄ heteroalkyl; and

each of R^{4G}, R^{4H}, and R^{4I} is, independently, selected from C₁₋₄ alkyl, C₂₋₄ alkenyl, C₂₋₄ alkynyl, and C₂₋₄ heteroalkyl.

The quaternary nitrogen in formula IV is identified herein as N″. Exemplary compounds of formula III include methylated quaternary ammonium derivatives of anesthetic drugs, such as N″,N″,N″-trimethyl procaine, N″,N″,N″-trimethyl proparacaine, N″,N″,N″-trimethyl procainamide, N″,N″,N″-trimethyl metoclopramide, N″,N″,N″-trimethyl propoxycaine, N″,N″,N″-trimethyl chloroprocaine, N″,N″-dimethyl tetracaine, N″,N″,N″-trimethyl benzocaine, and N″,N″,N″-trimethyl butamben. These derivatives can be prepared using methods analogous to those described in Scheme 1.

In some embodiments, the nociceptor modulating agent, nociceptor antagonist, neuropeptide modulating agent, an agent that blocks vesicle release, or agent that blocks the release or action of a neuropeptide from tumor-innervating nociceptor is of Formula V:

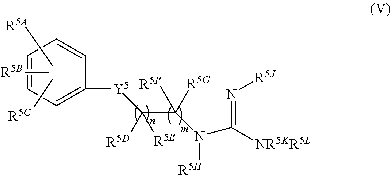

wherein:

n=0-3;

m=0-3; with (n+m)=0-6;

each of R^{5A}, R^{5B}, and R^{5C} is, independently, selected from H, halogen, C₁₋₄ alkyl, C₂₋₄ alkenyl, C₂₋₄ alkynyl, C₂₋₄ heteroalkyl, OR^{5M}, NR^{5N}R^{5O}, NR^{5P}C(O)R^{5Q}, S(O)R^{5R}, SO₂R^{5S}R^{5T}, SO₂NR^{5U}R^{5V}, SO₃R^{5W}, CO₂R^{5X}, C(O)R^{5Y}, and C(O)NR^{5Z}R^{5AA};

each of R^{5M}, R^{5N}, R^{5O}, R^{5P}, R^{5Q}, R^{5R}, R^{5S}, R^{5T}, R^{5U}, R^{5V}, R^{5W}, R^{5X}, R^{5Y}, R^{5Z}, and R^{5AA} is, independently, selected from H, C₁₋₄ alkyl, C₂₋₄ alkenyl, C₂₋₄ alkynyl, and C₂₋₄ heteroalkyl;

Y⁵ is selected from —CR^{5AB}R^{5AC}, —NR^{5AD}C(O)—, —OC(O)—, —SC(O)—, —C(O)NR^{5AE}—, —CO₂—, and —OC(S)—;

each of R^{5AB}, R^{5AC}, R^{5AD}, and R^{5AE} is, independently, selected from H, C₁₋₄ alkyl, C₂₋₄ alkenyl, C₂₋₄ alkynyl, and C₂₋₄ heteroalkyl;

each of R^{5D}, R^{5E}, R^{5F}, and R^{5G} is, independently, selected from H, C₁₋₄ alkyl, C₂₋₄ alkenyl, C₂₋₄ alkynyl, C₂₋₄ heteroalkyl, C₂₋₆ heterocyclyl, C₆₋₁₂ aryl, C₇₋₁₄ alkaryl, and C₃₋₁₀ alkheterocyclyl;

R^{5H} is H or C₁₋₄ alkyl; and

each of R^{5J}, R^{5K}, and R^{5L} is, independently, selected from H, C₁₋₄ alkyl, C₂₋₄ alkenyl, C₂₋₄ alkynyl, and C₂₋₄ heteroalkyl;

or R^{5J} and R^{5K} together complete a heterocyclic ring having two nitrogen atoms.

In some embodiments, R^{5J} and R^{5K} form a heterocyclic ring having two nitrogen atoms, the resulting guanidine group is selected from:

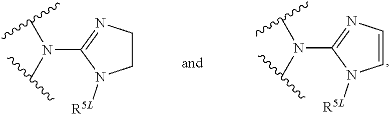

where R^{5L} is H or CH₃. In certain embodiments, R^{5J} and R^{5K} combine to form an alkylene or alkenylene of from 2 to 4 carbon atoms, e.g., ring systems of 5, 6, and 7-membered rings.

The guanylated nitrogen in formula V is identified herein as N′. Exemplary compounds of formula V include N-guanidyl derivatives (e.g., —C(NH)NH₂ derivatives) of anesthetic drugs, such as such as desethyl-N′-guanidyl procaine, desethyl-N′-guanidyl proparacaine, desethyl-N′-guanidyl allocain, desmethyl-N′-guanidyl encainide, desethyl-N′-guanidyl procainamide, desethyl-N′-guanidyl metoclopramide, desmethyl-N′-guanidyl stovaine, desethyl-N′-guanidyl propoxycaine, desethyl-N′-guanidyl chloroprocaine, N′-guanidyl flecainide, and desethyl-N′-guanidyl tetracaine. These derivatives can be prepared using methods analogous to those described in Schemes 2-5.

In some embodiments, the nociceptor modulating agent, nociceptor antagonist, neuropeptide modulating agent, an agent that blocks vesicle release, or agent that blocks the release or action of a neuropeptide from tumor-innervating nociceptor is of Formula VI:

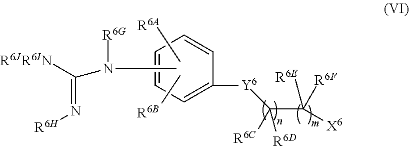

wherein:

n=0-3;

m=0-3; wherein with (n+m)=0-6;

each of R^{6A} and R^{6B} is, independently, selected from H, halogen, C₁₋₄ alkyl, C₂₋₄ alkenyl, C₂₋₄ alkynyl, C₂₋₄ heteroalkyl, OR^{6K}, NR^{6L}R^{6M}, NR^{6N}C(O)R^{6O}, S(O)R^{6P}, SO₂R^{6Q}R^{6R}, SO₂NR^{6S}R^{6T}, SO₃R^{6U}, CO₂R^{6V}, C(O)R^{6W}, and C(O)NR^{6X}R^{6Y};

each of R^{6K}, R^{6L}, R^{6M}, R^{6N}, R^{6O}, R^{6P}, R^{6Q}, R^{6R}, R^{6S}, R^{6T}, R^{6U}, R^{6V}, R^{6W}, R^{6X}, and R^{6Y} is, independently, selected from H, C₁₋₄ alkyl, C₂₋₄ alkenyl, C₂₋₄ alkynyl, and C₂₋₄ heteroalkyl;

Y⁶ is selected from —CR^{6Z}R^{6AA}—, NR^{6AB}C(O)—, —OC(O)—, —SC(O)—, —C(O)NR^{6AC}—, —CO₂—, and —OC(S)—;

each of R^{6Z}, R^{6AA}, R^{6AB}, and R^{6AC} is, independently, selected from H, C₁₋₄ alkyl, C₂₋₄ alkenyl, C₂₋₄ alkynyl, and C₂₋₄ heteroalkyl;

each of R^{6C}, R^{6D}, R^{6E}, and R^{6F} is, independently, selected from H, C₁₋₄ alkyl, C₂₋₄ alkenyl, C₂₋₄ alkynyl, C₂₋₄ heteroalkyl, C₂₋₆ heterocyclyl, C₆₋₁₂ aryl, C₇₋₁₄ alkaryl, and C₃₋₁₀ alkheterocyclyl;

X⁶ is selected from H, C₁₋₄ alkyl, C₂₋₄ alkenyl, C₂₋₄ alkynyl, and NR^{6AD}R6^{AE}; each of R^{6AD} and R^{6AE} is, independently, selected from H, C₁₋₄ alkyl, C₂₋₄ alkenyl, C₂₋₄ alkynyl, and C₂₋₄ heteroalkyl;

R^{6G} is H or C₁₋₄ alkyl; and

each of R^{6H}, R^{6I}, and R^{6J} is, independently, selected from H, C₁₋₄ alkyl, C₂₋₄ alkenyl, C₂₋₄ alkynyl, and C₂₋₄ heteroalkyl;

or R^{6H} and R^{6I} together complete a heterocyclic ring having two nitrogen atoms.

In some embodiments, R^{6H} and R^{6I} form a heterocyclic ring having two nitrogen atoms, the resulting guanidine group is selected from:

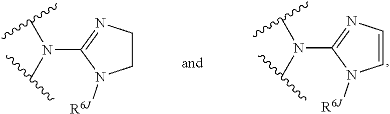

where R^{6J} is H or CH₃. In some embodiments, R^{6H} and R^{6I} combine to form an alkylene or alkenylene of from 2 to 4 carbon atoms, e.g., ring systems of 5, 6, and 7-membered rings.

The guanylated nitrogen in formula V is identified herein as N″. Exemplary compounds of formula VI include N-guanidyl derivatives (e.g., —C(NH)NH₂ derivatives) of anesthetic drugs, such as such as N″-guanidyl procaine, N″-guanidyl proparacaine, N″-guanidyl procainamide, N″-guanidyl metoclopramide, N″-guanidyl propoxycaine, N″-guanidyl chloroprocaine, N″-guanidyl tetracaine, N″-guanidyl benzocaine, and N″-guanidyl butamben. These derivatives can be prepared using methods analogous to those described in Schemes 2-5.

In some embodiments, the nociceptor modulating agent, nociceptor antagonist, neuropeptide modulating agent, an agent that blocks vesicle release, or agent that blocks the release or action of a neuropeptide from tumor-innervating nociceptor is of Formula VII:

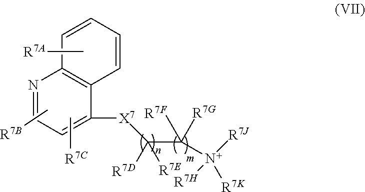

wherein:

n=0-3;

m=0-3; wherein with (n+m)=0-6;

each of R^{7A}, R^{7B}, and R^{7C} is, independently, selected from H, halogen, C₁₋₄ alkyl, C₂₋₄ alkenyl, C₂₋₄ alkynyl, C₂₋₄ heteroalkyl, OR^{7L}, NR^{7M}R^{7N}, NR^{7O}C(O)R^{7P}, S(O)R^{7Q}, SO₂R^{7R}R^{7S}, SO₂NR^{7T}R^{7U}, SO₃R^{7V}, CO₂R^{7W}, C(O)R^{7X}, and C(O)NR^{7Y}R^{7Z};

each of R^{7L}, R^{7M}, R^{7N}, R^{7O}, R^{7P}, R^{7Q}, R^{7R}, R^{7S}, R^{7T}, R^{7U}, R^{7V}, R^{7W}, R^{7X}, R^{7Y}, and R^{7Z} is, independently, selected from H, C₁₋₄ alkyl, C₂₋₄ alkenyl, C₂₋₄ alkynyl, and C₂₋₄ heteroalkyl;

X⁷ is selected from —CR^{7AA}R^{7AB}—, —NR^{7AC}C(O)—, —OC(O)—, —SC(O)—, —C(O)NR^{7AD}—, —CO₂—, and —OC(S)—;

each of R^{7AA}, R^{7AB}, R^{7AC}, and R^{7AD} is, independently, selected from H, C₁₋₄ alkyl, C₂₋₄ alkenyl, C₂₋₄ alkynyl, and C₂₋₄ heteroalkyl;

each of R^{7D}, R^{7E}, R^{7F}, and R^{7G} is, independently, selected from H, C₁₋₄ alkyl, C₂₋₄ alkenyl, C₂₋₄ alkynyl, C₂₋₄ heteroalkyl, C₂₋₆ heterocyclyl, C₆₋₁₂ aryl, C₇₋₁₄ alkaryl, and C₃₋₁₀ alkheterocyclyl;

each of R^{7H}, R^{7J}, and R^{7K} is, independently, selected from C₁₋₄ alkyl, C₂₋₄ alkenyl, C₂₋₄ alkynyl, and C₂₋₄ heteroalkyl.

In a preferred embodiment, X⁷ is —C(O)NH—. Exemplary compounds of formula VII include methylated quaternary ammonium derivatives of anesthetic drugs, such as N′-methyl dibucaine. These derivatives can be prepared using methods analogous to those described in Scheme 1.

In some embodiments, the nociceptor modulating agent, nociceptor antagonist, neuropeptide modulating agent, an agent that blocks vesicle release, or agent that blocks the release or action of a neuropeptide from tumor-innervating nociceptor is of Formula VIII:

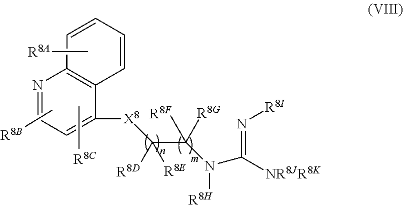

wherein:

n=0-3;

m=0-3; wherein (n+m)=0-6;

each of R^{8A}, R^{8B}, and R^{8C} is, independently, selected from H, halogen, C₁₋₄ alkyl, C₂₋₄ alkenyl, C₂₋₄ alkynyl, C₂₋₄ heteroalkyl, OR^{8L}, NR^{8M}R^{8N}, NR^{8O}C(O)R^{8P}, S(O)R^{8Q}, SO₂R^{8R}R^{8S}, SO₂NR^{8T}R^{8U}, SO₃R^{8V}, CO₂R^{8W}, C(O)R^{8X}, and C(O)NR^{8Y}R^{8Z};

each of R^{8L}, R^{8M}, R^{8N}, R^{8O}, R^{8P}, R^{8Q}, R^{8R}, R^{8S}, R^{8T}, R^{8U}, R^{8V}, R^{8W}, R^{8X}, R^{8Y}, and R^{8Z} is, independently, selected from H, C₁₋₄ alkyl, C₂₋₄ alkenyl, C₂₋₄ alkynyl, and C₂₋₄ heteroalkyl;

X⁸ is selected from —CR^{8AA}R^{8AB}—, —NR^{8AC}C(O)—, —OC(O)—, —SC(O)—, —C(O)NR^{8AD}—, —CO₂—, and —OC(S)—;

each of R^{8AA}, R^{8AB}, R^{8AC}, and R^{8AD} is, independently, selected from H, C₁₋₄ alkyl, C₂₋₄ alkenyl, C₂₋₄ alkynyl, and C₂₋₄ heteroalkyl;

each of R^{8D}, R^{8E}, R^{8F}, and R^{8G} is, independently, selected from H, C₁₋₄ alkyl, C₂₋₄ alkenyl, C₂₋₄ alkynyl, C₂₋₄ heteroalkyl, C₂₋₆ heterocyclyl, C₆₋₁₂ aryl, C₇₋₁₄ alkaryl, and C₃₋₁₀ alkheterocyclyl;

R^{8H} is H or C₁₋₄ alkyl;

each of R^{8I}, R^{8J}, and R^{8K} is, independently, selected from H, C₁₋₄ alkyl, C₂₋₄ alkenyl, C₂₋₄ alkynyl, and C₂₋₄ heteroalkyl; or R^{8I} and R^{8J} together complete a heterocyclic ring having two nitrogen atoms.

In some embodiments, R^{8I} and R^{8J} form a heterocyclic ring having two nitrogen atoms, the resulting guanidine group is selected from

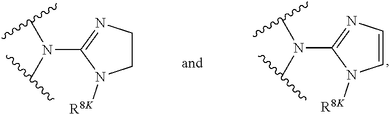

where R^{8K} is H or CH₃. In some embodiments, R^{8I} and R^{8J} combine to form an alkylene or alkenylene of from 2 to 4 carbon atoms, e.g., ring systems of 5, 6, and 7-membered rings.

The guanylated nitrogen in formula V is identified herein as N′. In a preferred embodiment, X⁸ is —C(O)NH—. Exemplary compounds of formula VIII include N-guanidyl derivatives (e.g., —C(NH)NH₂ derivatives) of anesthetic drugs, such as such as desethyl-N-guanidyl dibucaine. These derivatives can be prepared using methods analogous to those described in Schemes 2-5.

In some embodiments, the nociceptor modulating agent, nociceptor antagonist, neuropeptide modulating agent, an agent that blocks vesicle release, or agent that blocks the release or action of a neuropeptide from tumor-innervating nociceptor is of Formula IX:

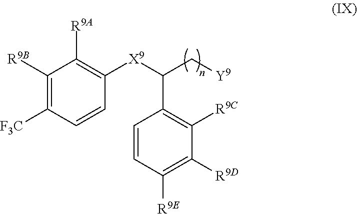

wherein:

n=0-6;

each of R^{9A}, R^{9B}, R^{9C}, R^{9D}, and R^{9E} is, independently, selected from H, halogen, C₁₋₄ alkyl, C₂₋₄ alkenyl, C₂₋₄ alkynyl, OR^{9I}, NR^{9J}R^{9K}, NR^{9L}C(O)R^{9M}, S(O)R^{9N}, SO₂R^{9O}R^{9P}, SO₂NR^{9Q}R^{9R}, SO₃R^{9S}, CO₂R^{9T}, C(O)R^{9U}, and C(O)NR^{9V}R^{9W};

each of R^{9I}, R^{9J}, R^{9K}, R^{9L}, R^{9M}, R^{9N}, R^{9O}, R^{9P}, R^{9Q}, R^{9R}, R^{9S}, R^{9T}, R^{9U}, R^{9V}, and R^{9W} is, independently, selected from H, C₁₋₄ alkyl, C₂₋₄ alkenyl, C₂₋₄ alkynyl, and C₂₋₄ heteroalkyl;

X⁹ is selected from —CR^{9X}R^{9Y}—, —O—, —S—, and —NR^{9Z}—; and each of R^{9X}, R^{9Y}, and R^{9Z} is, independently, selected from H, C₁₋₄ alkyl, C₂₋₄ alkenyl, C₂₋₄ alkynyl, and C₂₋₄ heteroalkyl;

Y⁹ is NR^{9AA}NR^{9AB}NR^{9AC} or NR^{9AD}Z⁹;

each of R^{9AA}, R^{9AB}, and R^{9AC} is, independently, selected from H, C₁₋₄ alkyl, C₂₋₄ alkenyl, and C₂₋₄ alkynyl;

R^{9AD} is H or C₁₋₄ alkyl;

Z⁹ is

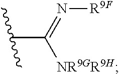

each of R^{9F}, R^{9G}, and R^{9H} is, independently, selected from H, C₁₋₄ alkyl, C₂₋₄ alkenyl, and C₂₋₄ alkynyl, or R^{9F} and R^{9G} together complete a heterocyclic ring having two nitrogen atoms.

In some embodiments, R^{9F} and R^{9G} form a heterocyclic ring having two nitrogen atoms, the resulting guanidine group is selected from

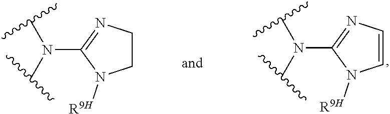

where R^{9H} is H or CH₃. In some embodiments, R^{9F} and R^{9G} combine to form an alkylene or alkenylene of from 2 to 4 carbon atoms, e.g., ring systems of 5, 6, and 7-membered rings. In a preferred embodiment, X⁹═—O—. Exemplary compounds of formula IX include N-guanidyl derivatives (e.g., —C(NH)NH₂ derivatives), such as N-guanidyl fluoxetine, and methylated quaternary ammonium derivatives, such as N,N-dimethyl fluoxetine. These derivatives can be prepared using methods analogous to those described in Schemes 1-5.

In some embodiments, the nociceptor modulating agent, nociceptor antagonist, neuropeptide modulating agent, an agent that blocks vesicle release, or agent that blocks the release or action of a neuropeptide from tumor-innervating nociceptor is of Formula X:

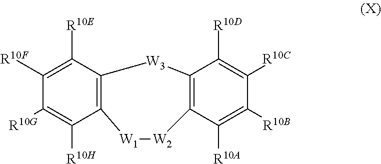

wherein:

W₃ is O, NH, NCH₂R^{10J}, NC(O)CH₂R^{10J}, CHCH₂R^{10J}, C═CHR^{10J}, or C═CHR^{10K};

W₁-W₂ is S, O, OCHR^{10K}, SCHR^{10K}, N═CR^{10K}, CHR^{10L}—CHR^{10K}, or CR^{10L}═CR^{10K};

each of R^{10A}, R^{10B}, R^{10C}, R^{10D}, R^{10E}, R^{10F}, R^{10G}, and R^{10H} is, independently, selected from H, OH, halide, C₁₋₄ alkyl, and C₂₋₄ heteroalkyl;

R^{10J} is CH₂CH₂X^{10A} or CH(CH₃)CH₂X^{10A};

R^{10L} is H or OH; R^{10K} is H, OH, or the group:

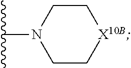

X^{10A} is NR^{10M}R^{10N}R^{10P}, or NR^{10Q}X^{10C}; X^{10B} is NR^{10R}R^{10S} or NX^{10C};

each of R^{10M}, R^{10N}, R^{10P}, R^{10R}, and R^{10S} is, independently, selected from C₁₋₄ alkyl, C₂₋₄ alkenyl, C₂₋₄ alkynyl, and C₂₋₄ heteroalkyl, or R^{10R}, and R^{10S} together complete a heterocyclic ring having at least one nitrogen atom;

R^{10Q} is H or C₁₋₄ alkyl;

X^{10C} is

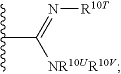

and

each of R^{10T}, R^{10U}, and R^{10V} is, independently, selected from H, C₁₋₄ alkyl, C₂₋₄ alkenyl, and C₂₋₄ alkynyl, or R^{10T} and R^{10V} together complete a heterocyclic ring having two nitrogen atoms.

In some embodiments, R^{10T} and R^{10V} form a heterocyclic ring having two nitrogen atoms, the resulting guanidine group is selected from

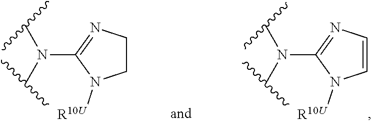

where R^{10U} is H or CH₃. In some embodiments, R^{10T} and R^{10V} combine to form an alkylene or alkenylene of from 2 to 4 carbon atoms, e.g., ring systems of 5, 6, and 7-membered rings.

Exemplary compounds of formula X include N-guanidyl derivatives (e.g., —C(NH)NH₂ derivatives) and methylated quaternary ammonium derivatives. N-guanidyl derivatives of formula X include, without limitation, N-guanidyl amoxapine, desmethyl-N-guanidyl trimipramine, desmethyl-N-guanidyl dothiepin, desmethyl-N-guanidyl doxepin, desmethyl-N-guanidyl amitriptyline, N-guanidyl protriptyline, N-guanidyl desipramine, desmethyl-N-guanidyl clomipramine, desmethyl-N-guanidyl clozapine, desmethyl-N-guanidyl loxapine, N-guanidyl nortriptyline, desmethyl-N-guanidyl cyclobenzaprine, desmethyl-N-guanidyl cyproheptadine, desmethyl-N-guanidyl olopatadine, desmethyl-N-guanidyl promethazine, desmethyl-N-guanidyl trimeprazine, desmethyl-N-guanidyl chlorprothixene, desmethyl-N-guanidyl chlorpromazine, desmethyl-N-guanidyl propiomazine, desmethyl-N-guanidyl prochlorperazine, desmethyl-N-guanidyl thiethylperazine, desmethyl-N-guanidyl trifluoperazine, desethyl-N-guanidyl ethacizine, and desmethyl-N-guanidyl imipramine. Methylated quaternary ammonium derivatives of formula X include, without limitation, N,N-dimethyl amoxapine, N-methyl trimipramine, N-methyl dothiepin, N-methyl doxepin, N-methyl amitriptyline, N,N-dimethyl protriptyline, N,N-dimethyl desipramine, N-methyl clomipramine, N-methyl clozapine, N-methyl loxapine, N,N-dimethyl nortriptyline, N-methyl cyclobenzaprine, N-methyl cyproheptadine, N-methyl olopatadine, N-methyl promethazine, N-methyl trimeprazine, N-methyl chlorprothixene, N-methyl chlorpromazine, N-methyl propiomazine, N-methyl moricizine, N-methyl prochlorperazine, N-methyl thiethylperazine, N-methyl fluphenazine, N-methyl perphenazine, N-methyl flupenthixol, N-methyl acetophenazine, N-methyl trifluoperazine, N-methyl ethacizine, and N-methyl imipramine. These derivatives can be prepared using methods analogous to those described in Schemes 1-5.

In some embodiments, the nociceptor modulating agent, nociceptor antagonist, neuropeptide modulating agent, an agent that blocks vesicle release, or agent that blocks the release or action of a neuropeptide from tumor-innervating nociceptor is a quaternary amine derived from orphenadrine, phenbenzamine, bepridil, pimozide, penfluridol, flunarizine, fluspirilene, propiverine, disopyramide, methadone, tolterodine, tridihexethyl salts, tripelennamine, mepyramine, brompheniramine, chlorpheniramine, dexchlorpheniramine, carbinoxamine, levomethadyl acetate, gallopamil, verapamil, devapamil, tiapamil, emopamil, dyclonine, pramoxine, lamotrigine, fendiline, mibefradil, gabapentin, amiloride, diltiazem, nifedipine, nimodipine, nitrendipine, cocaine, mexiletine, propafenone, quinidine, oxethazaine, articaine, riluzole, bencyclane, lifarizine, and strychnine. Still other compounds can be modified to incorporate a nitrogen atom suitable for quaternization or guanylation (e.g., fosphenytoin, ethotoin, phenytoin, carbamazepine, oxcarbazepine, topiramate, zonisamide, and salts of valproic acid).

In some embodiments, the nociceptor modulating agent, nociceptor antagonist, neuropeptide modulating agent, an agent that blocks vesicle release, or agent that blocks the release or action of a neuropeptide from tumor-innervating nociceptor is a quaternized or guanylated derivative of a compound as described in Table 1.

TABLE 1

| No. | Compound | Exemplary References |
|---|---|---|
| 1 | orphenadrine | U.S. Pat. No. 2,567,351 (see, e.g., the |
| | | compounds of Examples 1-6 and the formula |
| | | described at col.1, lines 10-24). |
| | | U.S. Pat. No. 2,991,225 (see, e.g., the |
| | | structure shown at col. 1, line 25). |
| 2 | phenbenzamine (RP- | Passalacqua et al., “Structure and |
| | 2339; Antergan ®), | Classification of Hi-Antihistamines and |
| | | Overview of Their Activities,” in Histamine |
| | | and Hi -antihistamines in Allergic Disease, |
| | | F.E.R. Simons, Ed., Informa Health Care |
| | | (2002). |
| 3 | bepridil | U.S. Pat. No. 3,962,238 (see, e.g., Formulas |
| | | I-V and compounds 1-6 of Table 1). |
| | | U.S. RE30577 |
| 4 | pimozide | See, e.g., Janssen et al., Arzneimittel-Forsch. |
| | | 18:261, 279, 282 (1968), and Journal of |
| | | Neuroscience, 22(2):396-403 (2002) |
| 5 | penfluridol | U.S. Pat. No. 3,575,990 (see, e.g., the |
| | | compounds of Formula (I), claims 1-7, and |
| | | Examples I-XXXIII). |
| 6 | flunarizine | U.S. Pat. No. 3,773,939 (see, e.g., Formula |
| | | (1) and the compound described at col. 5, line |
| | | 40). |
| 7 | fluspirilene | U.S. Pat. No. 3,238,216 (see, e.g., the |
| | | compounds recited in any of claims 1-34). |
| 8 | propiverine | DD 106643 |
| 9 | disopyramide | U.S. Pat. No. 3,225,054 (see, e.g., the |
| | | compounds of Examples 1-15 and claims 1-3) |
| 10 | methadone | DE711069 |
| | | U.S. Pat. No. 2,983,757 |
| 11 | tolterodine | U.S. Pat. No. 5,382,600 (see, e.g., Formula |
| | | (I), the compounds described at col.3, lines |
| | | 20-39, in Table 1, and in claims 1-7) |
| 12 | tridihexethyl salts | U.S. Pat. No. 2,913,494 (see, e.g., col. 1, |
| | | lines 15-22) |
| 13 | tripelennamine | U.S. Pat. No. 2,502,151 (see, e.g., Formula |
| | | (1) and the compounds recited in claims 1-13) |
| 14 | mepyramine | U.S. Pat. No. 2,502,151 |
| | (pyrilamine) | |
| 15 | brompheniramine | U.S. Pat. No. 2,567,245 (see, e.g., the |
| | | formula described at col. 1, lines 30-45, the |
| | | compounds of Examples I-XXI, and the |
| | | compounds recited in claims 1-15) |
| | | U.S. Pat. No. 2,676,964 (see, e.g., the |
| | | formula described at col.1, lines 5-28, the |
| | | compounds of Examples I-XLIV, and the |
| | | compounds recited in claims 1-14) |
| | | U.S. Pat. No. 3,061,517 (see, e.g., the |
| | | formula at col.1, lines 49-67, and the |
| | | compounds described at col. 2, lines 17-19, |
| | | col. 2, lines 40-43, col. 4, lines 2-7, and claims |
| | | 1-6) |
| 16 | chlorpheniramine | |
| 17 | dexchlorphenir amine | U.S. Pat. No. 2,567,245 (see, e.g., the |
| | | formula described at col. 1, lines 30-45, the |
| | | compounds of Examples I-XXI, and the |
| | | compounds recited in claims 1-15) |
| | | U.S. Pat. No. 2,676,964 (see, e.g., the |
| | | formula described at col.1, lines 5-28, the |
| | | compounds of Examples I-XLIV, and the |
| | | compounds recited in claims 1-14) |
| | | U.S. Pat. No. 3,061,517 (see, e.g., the |
| | | formula at col.1, lines 49-67, and the |
| | | compounds described at col. 2, lines 17-19, |
| | | col. 2, lines 40-43, col. 4, lines 2-7, and claims |
| | | 1-6)U.S. Pat. No. 2,766,174 (see, e.g., the |
| | | formula described at col. 1, lines 41-72) |
| 18 | carbinoxamine | U.S. Pat. No. 2,606,195 (see, e.g., the |
| | | formula described at col. 1, lines 7-24, |
| | | Examples I-VIII, and in claims 1-3) |
| | | U.S. Pat. No. 2,800,485 |
| | | GB 905993 |
| 19 | levomethadyl acetate | Pohland et al., J. Am. Chem. Soc. 71:460 |
| | | (1949) |
| 20 | gallopamil | U.S. Pat. No. 3,261,859 (see, e.g., Formula |
| | | (1), Examples 1-28, and claims 1-19) |
| | | Theodore et al., J. Org. Chem. 52:1309 (1987) |
| 21 | verapamil | U.S. Pat. No. 3,261,859 (see, e.g., Formulas |
| | | (I) and (IV), Examples 1-28, and claims 1-19) |
| 22 | devapamil | Godfraind, Calcium Channel Blockers, |
| 23 | tiapamil | Birkhauser Verlag (January 2004). |
| 24 | emopamil | |
| 25 | dyclonine | Pofft, Chem. Tech. (Berlin) 4:241 (1952) |
| 26 | pramoxine | U.S. Pat. No. 2,870,151 (see, e.g., the |
| | | formula described at col.1, lines 18-25, and |
| | | the compounds of Examples I-XII and claims |
| | | 1-13). |
| 27 | lamotrigine | EP21121 |
| | | U.S. Pat. No. 4,602,017 (see, e.g., Formulas |
| | | (I)-(III) and the compounds described at col. |
| | | 2, line 63-col. 3, line 12, Examples 1-5, and |
| | | claims 1-2) |
| 28 | mibefradil | U.S. Pat. No. 4,808,605 (see, e.g., Formula 1 |
| | | described at col.1, lines 10-33 and the |
| | | compounds described at col. 3, line 58-col. 7, |
| | | line 6, Examples 1-41, and claims 1-15). |
| 29 | gabapentin | U.S. Pat. No. 4,024,175 (see, e.g., Formula |
| | | (I) described at col.1, lines 5-17, Examples 1- |
| | | 12, and claims 1-11) |
| 30 | amiloride | U.S. Pat. No. 3,313,813 (see, e.g., the |
| | | compounds described at col. 1, line 13-col.2, |
| | | line 55, Examples 1-205, and claims 1-31) |
| 31 | diltiazem | U.S. Pat. No. 3,562,257 (see, e.g., Formula |
| | | (1) described at col.1, lines 39-64, and the |
| | | compounds described at col. 2, lines 15-30, |
| | | Tables 1-3, and claims 1-43) |
| | | U.S. Pat. No. 4,552,695 (see, e.g., the |
| | | compound of Formula (I)) |
| 32 | nifedipine | U.S. Pat. No. 3,485,847 (see, e.g., the |
| | | Formula described at col. 1, line 40-col. 2, line |
| | | 6, the compounds of Examples 1-6, and claims |
| | | 1-27) |
| 33 | nimodipine | U.S. Pat. No. 3,799,934 (see, e.g., the |
| | | Formula described at col. 1, lines 39-69, the |
| | | compounds described at col. 4, line 50-col. 5, |
| | | line 16, Examples 1-53, and claims 1-13) |
| 34 | nitrendipine | |
| 35 | mexiletine | U.S. Pat. No. 3,954,872 (see, e.g., Formula |
| | | (1) described at col.1, lines 14-35, and the |
| | | compounds of Examples 1-6 and claims 1-4) |
| 36 | propafenone | DE2001431 (see, e.g., claims 1-4) |
| 37 | quinidine | Turner et al., The Alkaloids, Vol. 3, 1-63 |
| | | (1953) |
| | | Mason et al., Ann. N.Y. Acad. Sci. 432:162- |
| | | 176(1984) |
| 38 | oxethazaine | U.S. Pat. No. 2,780,646 (see, e.g., the |
| | | formula described at col. 1, lines 18-42, and |
| | | the compounds of Examples 1-14 and claims |
| | | 1-8) |
| 39 | articaine | Becker et al., Anesth Prog. 53(3): 98-109 |
| | | (Fall 2006) |
| 40 | riluzole | U.S. Pat. No. 4,370,338 (see, e.g., the |
| | | compound described at col. 1, line 15) |
| 41 | bencyclane | HU 151865 |
| 42 | lifarizine | Grauert et al., J. Med. Chem. 45(17):3755- |
| | | 3764 (2002) |
| 43 | strychnine | Makarevich et al., “Quaternary salts of |
| | | alkaloids,” Vol. 42, pages 473-476, Chemistry |
| | | of Natural Compounds, Springer New York: |
| | | 2006. |
| 44 | fendiline | U.S. Pat. No. 3,262,977 (see, e.g., Formula |
| | | (I), Examples 1-9, and the compounds of |
| | | claims 1-9) |

Exemplary calcium channel blockers include D-890, CERM 11888, N-methyl-verapamil, N-methylgallopamil, N-methyl-devapamil, and dodecyltrimethylammonium. Other exemplary compounds include any charged derivative, e.g., a quaternary amine derivative, of verapamil, gallopamil, devapamil, diltiazem, fendiline, mibefradil, terpene compounds (e.g., sesquiterpenes) such as those described in Norman et al. Agricultural and Biological Chemistry 49(10):2893-8 (1985), and other inhibitors of calcium channels (see, for example, Triggle, European Journal of Pharmacology, 375:311-325 (1999), Eller et al., British Journal of Pharmacology, 130:669-677 (2000), and Yamamoto et al., Current Topics in Medicinal Chemistry, 9:377-395 (2009), which can be prepared according to the methods described herein.

For example, Yamamoto et al. provides the following N-type calcium channel blockers (Table 2), which can be modified (e.g., quaternized or guanylated) according to the methods described herein.

In some embodiments, the nociceptor modulating agent, nociceptor antagonist, neuropeptide modulating agent, an agent that blocks vesicle release, or agent that blocks the release or action of a neuropeptide from tumor-innervating nociceptor is a quaternized or guanylated derivative of a compound as described in Table 2.

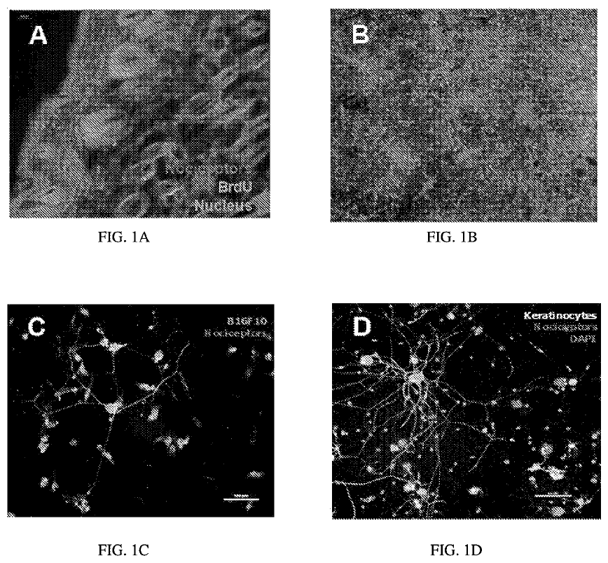

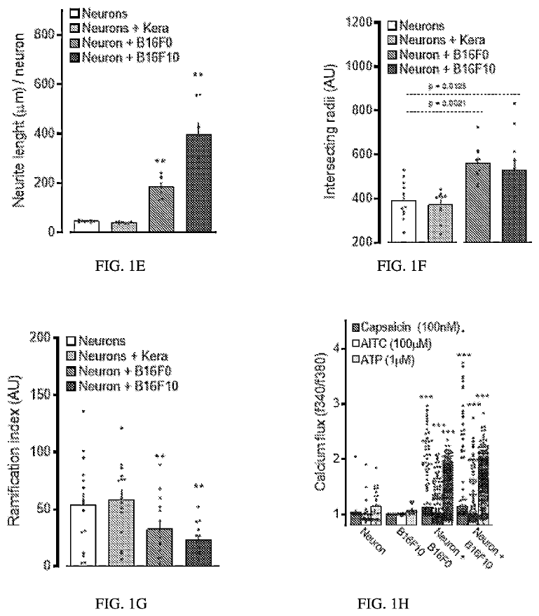

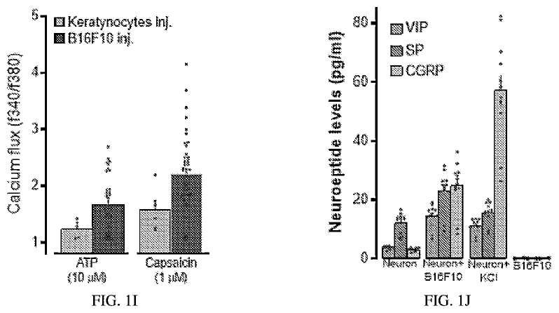

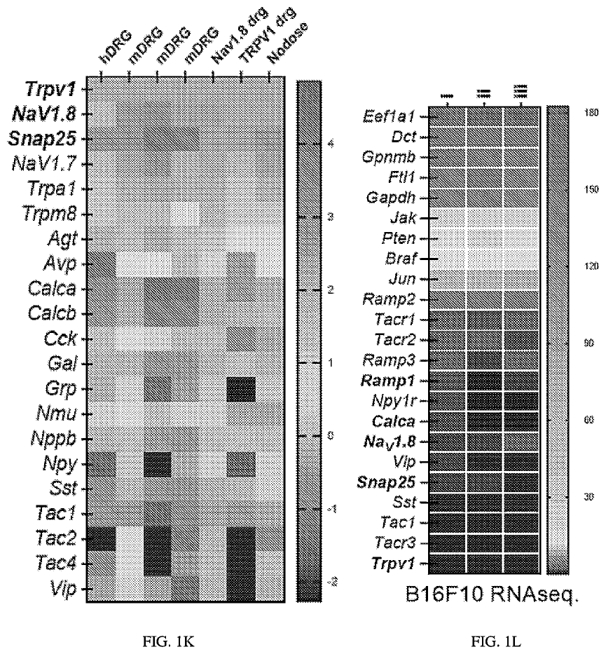

TABLE 2

| No. | Compound | Exemplary References |
|---|---|---|
| 45 | 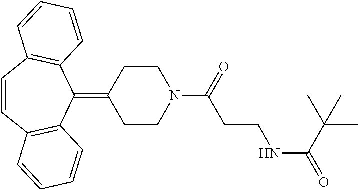 [SMILES: CC(C)(C)C(=O)NCCC(=O)N1CCC(=C2c3ccccc3C=Cc3ccccc32)CC1] | Yamamoto et al., Bioorg. Med. Chem. 14:5333-5339 (2006). |
| 46 | 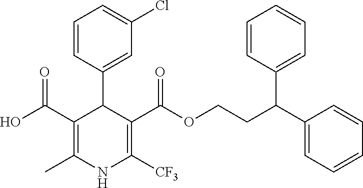 [SMILES: CC1=C(C(=O)O)C(c2cccc(Cl)c2)C(C(=O)OCCC(c2ccccc2)c2ccccc2)=C(C(F)(F)F)N1] | Yamamoto et al., Bioorg. Med. Chem. Lett. 16:798-802 (2006). |
| 47 | 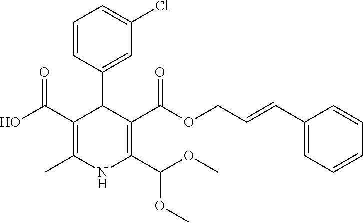 [SMILES: COC(OC)C1=C(C(=O)OC/C=C/c2ccccc2)C(c2cccc(Cl)c2)C(C(=O)O)=C(C)N1] | Yamamoto et al., Bioorg. Med. Chem. Lett. 18:4813-4815 (2008). |
| 48 | 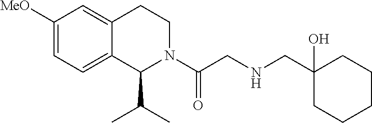 [SMILES: COc1ccc2c(c1)CCN(C(=O)CNCC1(O)CCCCC1)[C@H]2C(C)C] | See, e.g., WO08143263 and EP2149560 (e.g., Formula (I), the compounds of Tables 6-35, 43-110, 126-127, and the compounds of claims 1-6) |
| 49 | 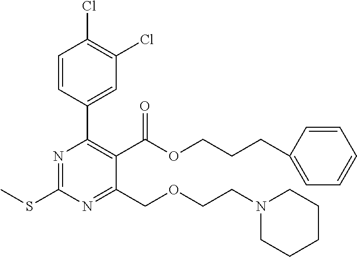 [SMILES: CSc1nc(COCCN2CCCCC2)c(C(=O)OCCCc2ccccc2)c(-c2ccc(Cl)c(Cl)c2)n1] | Miller et al., Soc. Neurosci. Abstr. 25(Part2):896.3 (1999) |
| 50 | 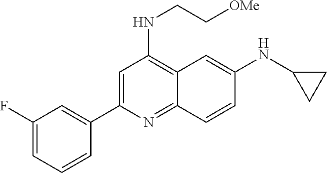 [SMILES: COCCNc1cc(-c2cccc(F)c2)nc2ccc(NC3CC3)cc12] | WO0236567 (see, e.g., formulas I-IV, the compounds of Table 2 (Examples 1-111), and claims 1-5) |
| 51 | 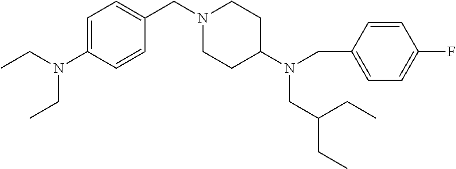 [SMILES: CCC(CC)CN(Cc1ccc(F)cc1)C1CCN(Cc2ccc(N(CC)CC)cc2)CC1] | Zhang et al., Eur. J. Pharmacol. 587:24-47 (2008) |
| 52 | 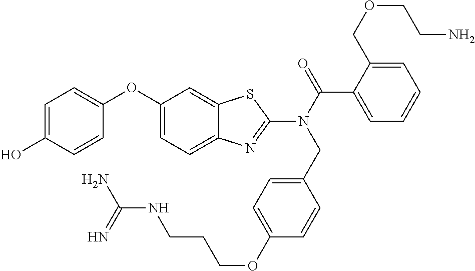 [SMILES: N=C(N)NCCCOc1ccc(CN(C(=O)c2ccccc2COCCN)c2nc3ccc(Oc4ccc(O)cc4)cc3s2)cc1] | Baell et al., Bioorg. Med. Chem. 12:4025-4037 (2004) |
| 53 | 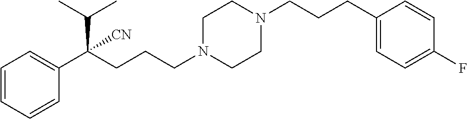 [SMILES: CC(C)[C@@](C#N)(CCCN1CCN(CCCc2ccc(F)cc2)CC1)c1ccccc1] | Yamamoto et al., 22^{nd} National Meeting of American Chemical Society, American Chemical Society: Washington, DC: Chicago, IL 2001; Kaneda et al, Soc. Neurosci. Abstr. 2T.332A5 (2001); Niidome et al., Soc. Neurosci. |
| | | Abstr. 27:332.14 (2001); and |
| | | Suzuki et al., Bioorg. Med. |
| | | Chem. Eett. 13:919-922 (2003). |
| 54 | E-2051 | Kaneda, Soc. Neurosci. Abstr. |
| | | 28:490.1 (2002) |
| 55 | 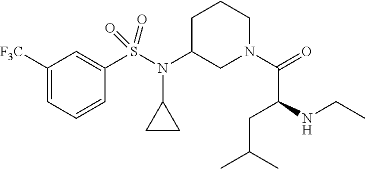 [SMILES: CCN[C@@H](CC(C)C)C(=O)N1CCCC(N(C2CC2)S(=O)(=O)c2cccc(C(F)(F)F)c2)C1] | WO07110449 (see, e.g., Formulas I-XIII, the compounds described at Paragraphs [0181]- [0183] and Examples 1-14, and claims 1-72) |
| 56 | 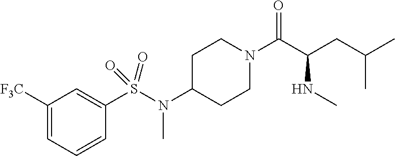 [SMILES: CN[C@H](CC(C)C)C(=O)N1CCC(N(C)S(=O)(=O)c2cccc(C(F)(F)F)c2)CC1] | WO06040181 (see, e.g., Formulas I-X, the compounds described at Paragraphs [0105]- [0109] and Examples 1-37, and in claims 1-56) |
| 57 | 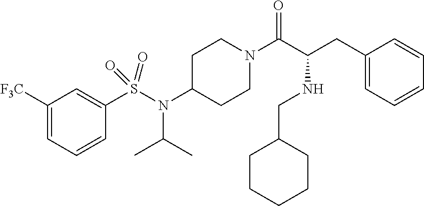 [SMILES: CC(C)N(C1CCN(C(=O)[C@H](Cc2ccccc2)NCC2CCCCC2)CC1)S(=O)(=O)c1cccc(C(F)(F)F)c1] | WO07118853 (see, e.g., Formulas I-XIII, the compounds described at Paragraph [0320] and Examples 1-19, and the compounds of claims 1-165) |
| 58 | 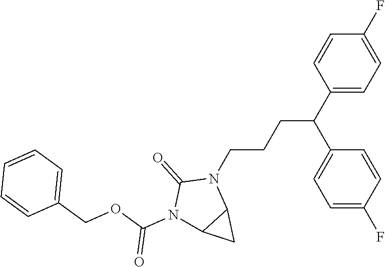 [SMILES: O=C(OCc1ccccc1)N1C(=O)N(CCCC(c2ccc(F)cc2)c2ccc(F)cc2)C2CC21] | WO07085357 (see, e.g., Formulas I-VII, the compounds described at Paragraphs [0065]- [0067], Examples 1-6, and claims 1-16) |
| 59 | 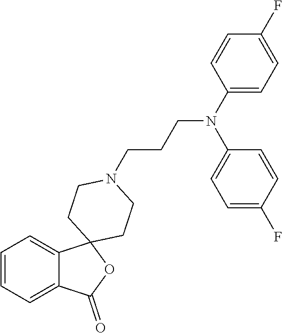 [SMILES: O=C1OC2(CCN(CCCN(c3ccc(F)cc3)c3ccc(F)cc3)CC2)c2ccccc21] | WO07028638 (see, e.g., Formulas XXVI, the compounds described a Paragraphs [0119]-[0123], Examples 1-24, and claims 1-20) |
| 60 | 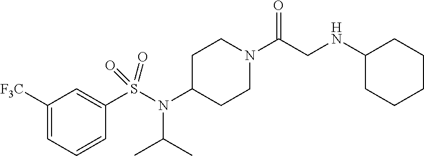 [SMILES: CC(C)N(C1CCN(C(=O)CNC2CCCCC2)CC1)S(=O)(=O)c1cccc(C(F)(F)F)c1] | WO07118854 (see, e.g., Formulas I-VII and the compounds of Examples 1-11 and claims 1-36) |
| 61 | 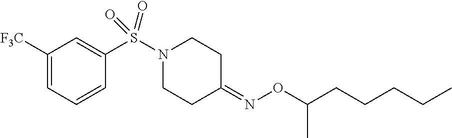 [SMILES: CCCCCC(C)ON=C1CCN(S(=O)(=O)c2cccc(C(F)(F)F)c2)CC1] | WO08008398 (see, e.g., Formulas I, I′, I″, II, and II′; Examples 1-377, and claims 1-7) |
| 62 | 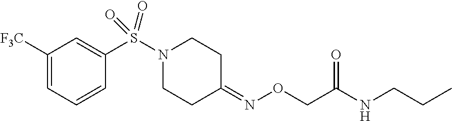 [SMILES: CCCNC(=O)CON=C1CCN(S(=O)(=O)c2cccc(C(F)(F)F)c2)CC1] | WO08150447 (see, e.g., Formulas I, I′, I″, and the compounds of Examples 1-135 and claims 1-5 |
| 63 | 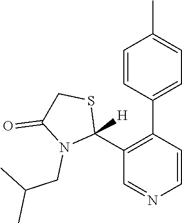 [SMILES: Cc1ccc(-c2ccncc2[C@H]2SCC(=O)N2CC(C)C)cc1] | Knutsen et al., Bioorg. Med. Chem. Lett. 17: 662-667 (2007) |
| 64 | 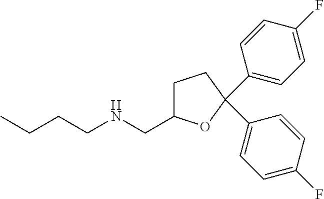 [SMILES: CCCCNCC1CCC(c2ccc(F)cc2)(c2ccc(F)cc2)O1] | O’Neill, Brain Res. 888:138-149 (2001); Hicks et al., Eur. J. Pharmacol. 408:241-248 (2000) |
| 65 | 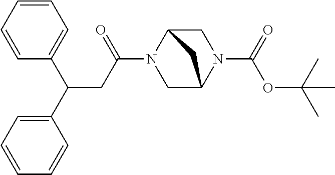 [SMILES: CC(C)(C)OC(=O)N1C[C@@H]2C[C@H]1CN2C(=O)CC(c1ccccc1)c1ccccc1] | WO07084394 (see, e.g., the compounds of Formulas I and Ia-Ig, and the compounds of Examples 1-11 and claims 1 and 2) |
| 66 | 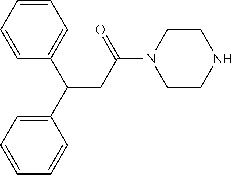 [SMILES: O=C(CC(c1ccccc1)c1ccccc1)N1CCNCC1] | WO08066803 (see, e.g., Formulas I and II, the compound of Example 1, and claims 1-11) |
| 67 | 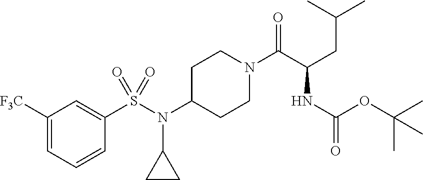 [SMILES: CC(C)C[C@@H](NC(=O)OC(C)(C)C)C(=O)N1CCC(N(C2CC2)S(=O)(=O)c2cccc(C(F)(F)F)c2)CC1] | WO07075524 (see, e.g., Formulas (I), (Ia)-(Ie), the compounds of Examples 1-184, and claims 1-16) |
| 68 | 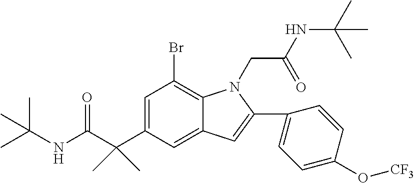 [SMILES: CC(C)(C)NC(=O)Cn1c(-c2ccc(OC(F)(F)F)cc2)cc2cc(C(C)(C)C(=O)NC(C)(C)C)cc(Br)c21] | WO08133867 (see, e.g., Formulas (I) and (II), the compounds of Examples 1-163, and claims 1-16) |
| 69 | 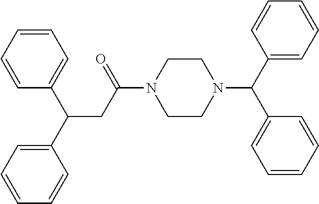 [SMILES: O=C(CC(c1ccccc1)c1ccccc1)N1CCN(C(c2ccccc2)c2ccccc2)CC1] | WO01045709 (see, e.g., Formula (1), the compounds of Example 4, and claims 24-38) WO06105670 (see, e.g., Formula (1), the compounds described at Paragraphs [0065] and [0066], and claims 1-13) |
| 70 | 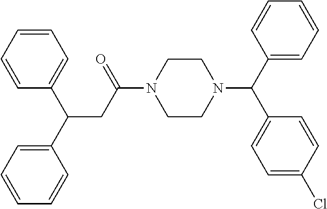 [SMILES: O=C(CC(c1ccccc1)c1ccccc1)N1CCN(C(c2ccccc2)c2ccc(Cl)cc2)CC1] | WO04089377 (see, e.g., Formula (1), Examples 1-5, original claims 1-13, and amended claims 1-17) |
| 71 | 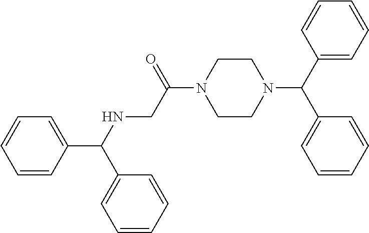 [SMILES: O=C(CNC(c1ccccc1)c1ccccc1)N1CCN(C(c2ccccc2)c2ccccc2)CC1] | WO07071035 (see, e.g., Formula (1), the compounds of Examples 1-18, and claims 20-35) |
| 72 | 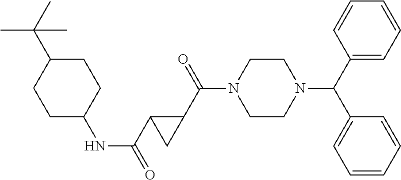 [SMILES: CC(C)(C)C1CCC(NC(=O)C2CC2C(=O)N2CCN(C(c3ccccc3)c3ccccc3)CC2)CC1] | WO08043183 (see, e.g., Formulas (1) and (2), the compounds of Examples 1-16, and claims 16-28) |
| 73 | 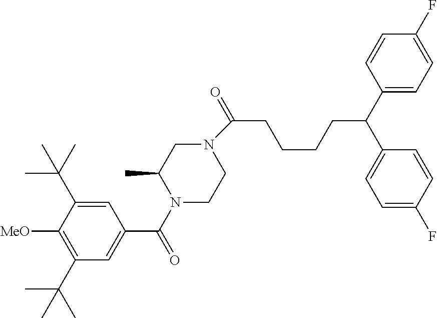 [SMILES: COc1c(C(C)(C)C)cc(C(=O)N2CCN(C(=O)CCCCC(c3ccc(F)cc3)c3ccc(F)cc3)C[C@@H]2C)cc1C(C)(C)C] | WO04089922 (see, e.g., Formulas (l)-(4), the compounds of Examples 1-9, claims 1-17, and the compounds of Figure 1) |
| 74 | 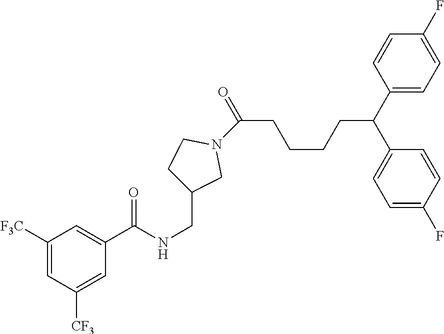 [SMILES: O=C(NCC1CCN(C(=O)CCCCC(c2ccc(F)cc2)c2ccc(F)cc2)C1)c1cc(C(F)(F)F)cc(C(F)(F)F)c1] | WO04105750 (see, e.g., Formulas (l)-(8), the compounds of Examples 1-10, claims 1-23, and Figure 1) |
| 75 | 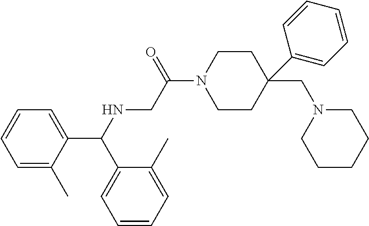 [SMILES: Cc1ccccc1C(NCC(=O)N1CCC(CN2CCCCC2)(c2ccccc2)CC1)c1ccccc1C] | WO08031227 (see, e.g., Formulas (1) and (2), the compounds of Examples 1-20, and claims 21-37) |
| 76 | 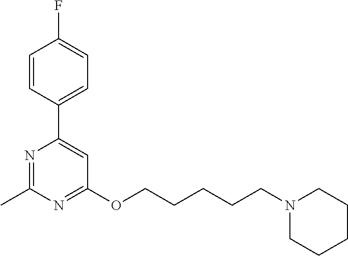 [SMILES: Cc1nc(OCCCCCN2CCCCC2)cc(-c2ccc(F)cc2)n1] | Tatsumi et al., Jpn. J. Pharmacol. 73:193 (1997);Aoki et al., Brain Res. 890:162-169 (2001); Katsumata et al., Brain Res. 969:168-174 (2003); Tamura et al., Brain Res. 890:170-176 (2001); Shi et al., J. Thorac. Cardiovasc Surg. 129:364-371 (2005); Small, IDrugs, 3:460-465 (2000); Suma et al., Jpn. J. Pharmacol. 73: 193 (1997); Shimidzu et al., Naunyn Schmiedebergs Arch. Pharamcol. 355:601-608 (1997); and Suma et al., Eur. J. Pharmacol. |
| | | 336:283-290(1997). |
| 77 | 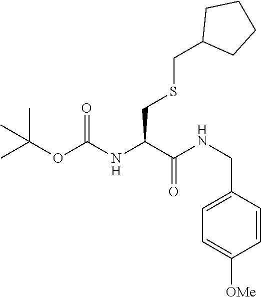 [SMILES: COc1ccc(CNC(=O)[C@H](CSCC2CCCC2)NC(=O)OC(C)(C)C)cc1] | Seko et al, Bioorg. Med. Chem. Lett. 11:2067-2070(2001) |
| 78 | 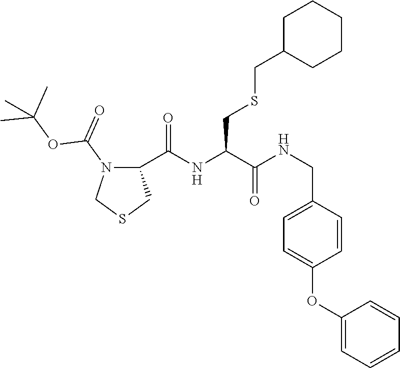 [SMILES: CC(C)(C)OC(=O)N1CSC[C@H]1C(=O)N[C@@H](CSCC1CCCCC1)C(=O)NCc1ccc(Oc2ccccc2)cc1] | Seko et al., Bioorg. Med. Chem. 11:1901-1913 (2003). Seko et al., Bioorg. Med. Chem. Lett. 12:915-918 (2002) |
| 79 | 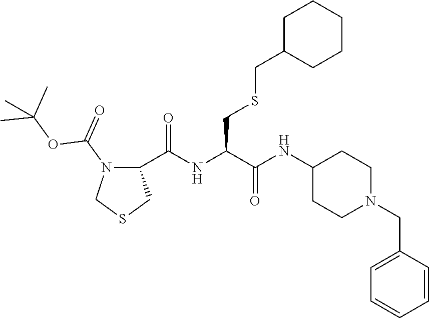 [SMILES: CC(C)(C)OC(=O)N1CSC[C@H]1C(=O)N[C@@H](CSCC1CCCCC1)C(=O)NC1CCN(Cc2ccccc2)CC1] | Seko et al., Bioorg. Med. Chem. Lett. 12:2267-2269 (2002) |
| 80 | 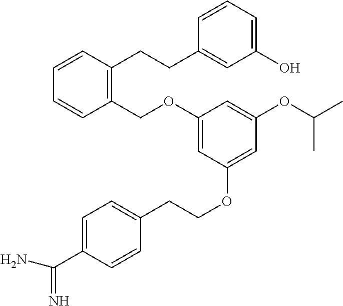 [SMILES: CC(C)Oc1cc(OCCc2ccc(C(=N)N)cc2)cc(OCc2ccccc2CCc2cccc(O)c2)c1] | Menzler et al., Bioorg. Med. Chem. Lett. 10:345-347 (2000) |
| 81 | 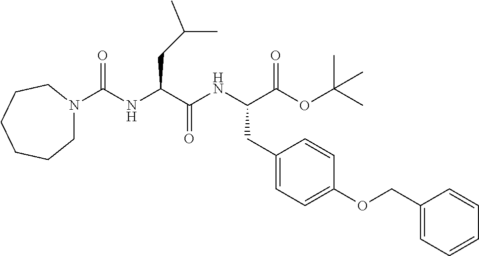 [SMILES: CC(C)C[C@H](NC(=O)N1CCCCCC1)C(=O)N[C@@H](Cc1ccc(OCc2ccccc2)cc1)C(=O)OC(C)(C)C] | Malone et al., 217^{th} National Meeting of the American Chemical Society, American Chemical Society: Washington DC: Anaheim CA 1999; Hu et al., J. Med. Chem. 42:4239-4249 (1999) |
| 82 | 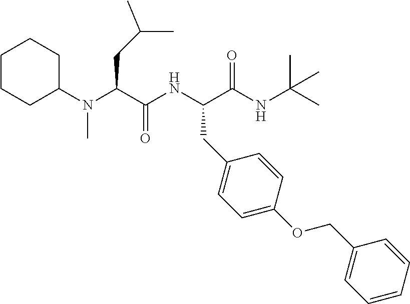 [SMILES: CC(C)C[C@@H](C(=O)N[C@@H](Cc1ccc(OCc2ccccc2)cc1)C(=O)NC(C)(C)C)N(C)C1CCCCC1] | Hu et al., Bioorg. Med. Chem. Lett. 9:907-912 (1999) |
| 83 | 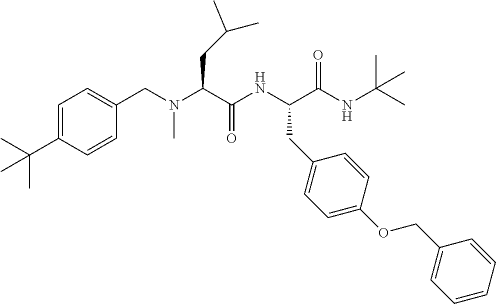 [SMILES: CC(C)C[C@@H](C(=O)N[C@@H](Cc1ccc(OCc2ccccc2)cc1)C(=O)NC(C)(C)C)N(C)Cc1ccc(C(C)(C)C)cc1] | Hu et al., Bioorg. Med. Chem. Lett. 9:2151-2156 (1999) Ryder et al., Bioorg. Med. Chem. Lett. 9:1813-1818 (1999) |
| 84 | 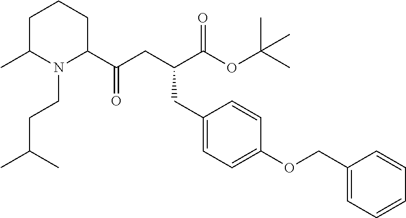 [SMILES: CC(C)CCN1C(C)CCCC1C(=O)C[C@@H](Cc1ccc(OCc2ccccc2)cc1)C(=O)OC(C)(C)C] | Hu et al., Bioorg. Med. Chem. Lett. 9:1121-1126 (1999) |
| 85 | 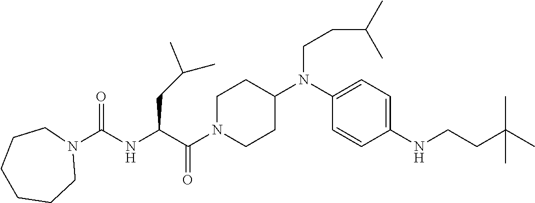 [SMILES: CC(C)CCN(c1ccc(NCCC(C)(C)C)cc1)C1CCN(C(=O)[C@H](CC(C)C)NC(=O)N2CCCCCC2)CC1] | Bennett et al., Pain 33:87-107 (1988) |
| 86 | 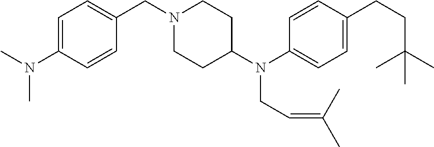 [SMILES: CC(C)=CCN(c1ccc(CCC(C)(C)C)cc1)C1CCN(Cc2ccc(N(C)C)cc2)CC1] | Hu et al., Bioorg. Med. Chem. 8:1203-1212 (2000) |
| 87 | 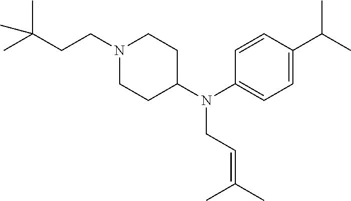 [SMILES: CC(C)=CCN(c1ccc(C(C)C)cc1)C1CCN(CCC(C)(C)C)CC1] | Hu et al., Bioorg. Med. Chem. 8:1203-1212 (2000) |
| 88 | 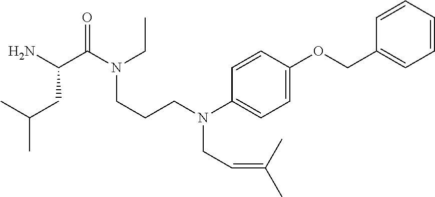 [SMILES: CCN(CCCN(CC=C(C)C)c1ccc(OCc2ccccc2)cc1)C(=O)[C@@H](N)CC(C)C] | Hu et al., J. Med. Chem. 42:4239-4249 (1999) |
| 89 | 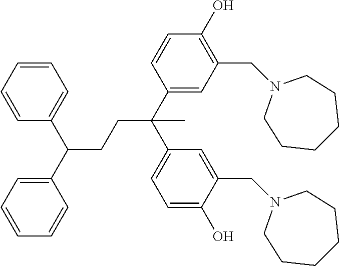 [SMILES: CC(CCC(c1ccccc1)c1ccccc1)(c1ccc(O)c(CN2CCCCCC2)c1)c1ccc(O)c(CN2CCCCCC2)c1] | Schelkun et al., Bioorg. Med. Chem. Lett. 9:2447-2452 (1999). |
| 90 | 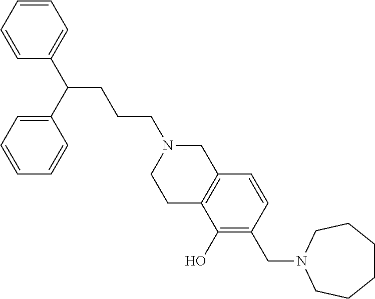 [SMILES: Oc1c(CN2CCCCCC2)ccc2c1CCN(CCCC(c1ccccc1)c1ccccc1)C2] | Yuen et al., Bioorg. Med. Chem. Lett. 8:2415-2418 (1998) |
| 91 | 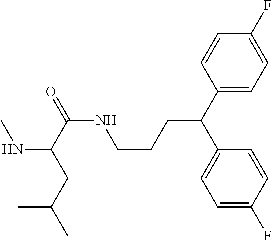 [SMILES: CNC(CC(C)C)C(=O)NCCCC(c1ccc(F)cc1)c1ccc(F)cc1] | Song et al., J. Med. Chem. 43:3474-3477 (2000) |
| 92 | 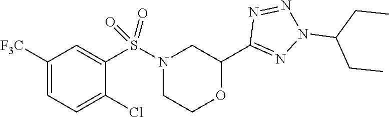 [SMILES: CCC(CC)n1nnc(C2CN(S(=O)(=O)c3cc(C(F)(F)F)ccc3Cl)CCO2)n1] | WO07125398 (see, e.g., Formula (1), the compounds of Examples 1-29, and claims 1-9) |
| 93 | 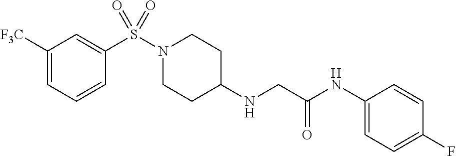 [SMILES: O=C(CNC1CCN(S(=O)(=O)c2cccc(C(F)(F)F)c2)CC1)Nc1ccc(F)cc1] | WO08124118 (see, e.g., Formula I-VI, the compounds of Paragraphs [0129] and Examples 1-5, and claims 1-42) |
| 94 | 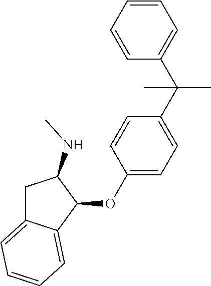 [SMILES: CN[C@@H]1Cc2ccccc2[C@@H]1Oc1ccc(C(C)(C)c2ccccc2)cc1] | Campbell et al., Eur. J. Pharmacol. 401:419-428 (2000) |
| 95 | 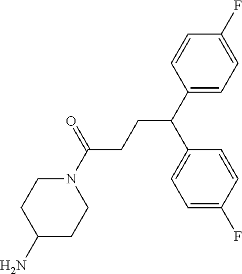 [SMILES: NC1CCN(C(=O)CCC(c2ccc(F)cc2)c2ccc(F)cc2)CC1] | Teodori et al., J. Med. Chem. 47:6070-6081 (2004) |
| 96 | 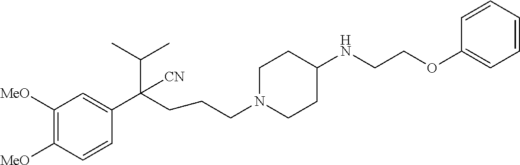 [SMILES: COc1ccc(C(C#N)(CCCN2CCC(NCCOc3ccccc3)CC2)C(C)C)cc1OC] | Teodori et al., J. Med. Chem. 47:6070-6081 (2004) |
| 97 | 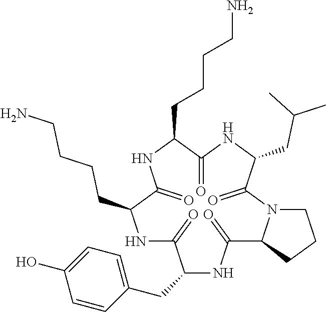 [SMILES: CC(C)C[C@H]1NC(=O)[C@H](CCCCN)NC(=O)[C@H](CCCCN)NC(=O)[C@@H](Cc2ccc(O)cc2)NC(=O)[C@@H]2CCCN2C1=O] | Schroeder et al., Mol. Divers. 8:127-134 (2004). |
| 98 | 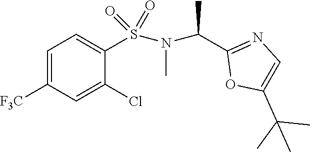 [SMILES: C[C@@H](c1ncc(C(C)(C)C)o1)N(C)S(=O)(=O)c1ccc(C(F)(F)F)cc1Cl] | WO06030211 (see, e.g., Formula (I), the compounds described at page 9, line 17-page 15, line 12, Examples 1-99, and claims 1-12) |

In some embodiments, the nociceptor modulating agent, nociceptor antagonist, neuropeptide modulating agent, an agent that blocks vesicle release, or agent that blocks the release or action of a neuropeptide from tumor-innervating nociceptor is of Formula XI:

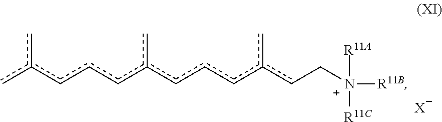

wherein:

each R^{11A}, R^{11B}, and R^{11C} is selected, independently, from H or C₁₋₄ alkyl, and 0, 1, 2, or 3 of the dashed bonds ( [symbol] ) represents a carbon-carbon double bond (i.e., compounds of Formula (XI) can include 0, 1, 2, or 3 double bonds), provided that when 2 or 3 carbon-carbon double bonds are present, the double bonds are not adjacent to one another. Compounds that include 0, 1, or 2 double bonds can be prepared according to methods known in the literature, e.g., partial or total hydrogenation of the parent triene.

In some embodiments, compounds of Formula (XI) can be represented by the following formula (XI-A),

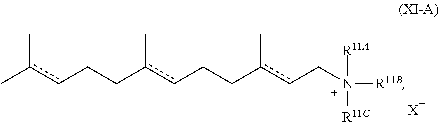

where each R^{11A}, R^{11B}, R^{11C}, and X is according to Formula (XI), and where each dashed bond represents an optional carbon-carbon double bond.

In some embodiments, compounds of Formula (XI) include those compounds that have a structure according to Formula (XI-B),

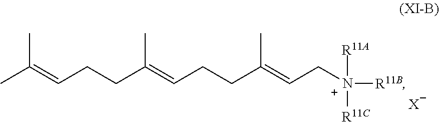

where each R^{11A}, R^{11B}, R^{11C}, and X is according to Formula (XI).

Exemplary compounds of Formula (XI) include

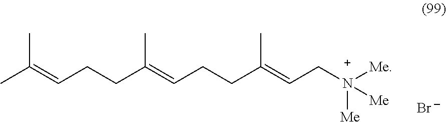 [SMILES: CC(C)=CCC/C(C)=C/CC/C(C)=C/C[N+](C)(C)C ; [Br-]]

Amino acid derivatives, e.g., those described in U.S. Pat. No. 7,166,590 or in Seko et al., Bioorg. Med. Chem. Lett. 11(16):2067-2070 (2001), each of which is herein incorporated by reference, can also be used herein. For example, compounds having a structure according to Formula (XII) can be N-type calcium channel blockers.

In some embodiments, the nociceptor modulating agent, nociceptor antagonist, neuropeptide modulating agent, an agent that blocks vesicle release, or agent that blocks the release or action of a neuropeptide from tumor-innervating nociceptor is of Formula XII:

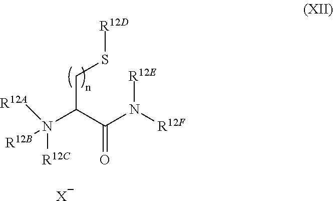

wherein:

each of R^{12A}, R^{12B}, R^{12C}, and R^{12D} is, independently, selected from C₁₋₄ alkyl, C₂₋₄ alkenyl, C₂₋₄ alkynyl, C₂₋₄ heteroalkyl, C₇₋₁₄ alkaryl, C₃₋₁₀ alkcycloalkyl, and C₃₋₁₀ alkheterocyclyl; or R^{12A} and R^{12B} together complete a heterocyclic ring having at least one nitrogen atom;

n is an integer between 1-5;

each of R^{12E} and R^{12F} is, independently, selected from H, C₁₋₄ alkyl, C₂₋₄ alkenyl, C₂₋₄ alkynyl, C₂₋₄ heteroalkyl, C₇₋₁₄ alkaryl, C₃₋₁₀ alkcycloalkyl, or C₃₋₁₀ alkheterocyclyl; and

X is any pharmaceutically acceptable anion.

Exemplary compounds of Formula (XII) include

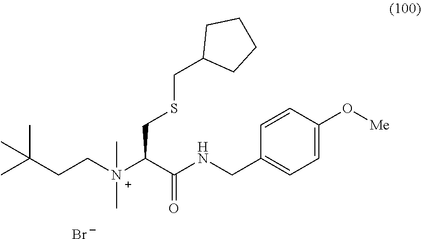 [SMILES: COc1ccc(CNC(=O)[C@H](CSCC2CCCC2)[N+](C)(C)CCC(C)(C)C)cc1 ; [Br-]]

Still other compounds that can be used herein are quaternary amine derivatives of flunarizine and related compounds (see, e.g., U.S. Pat. Nos. 2,883,271 and 3,773,939, as well as Zamponi et al., Bioorg. Med. Chem. Lett. 19: 6467 (2009)), each of which is hereby incorporated by reference. For example, compounds according to Formulas (XIII-A), (XIII-B), and (XIII-C) can be prepared according to, e.g., Zamponi et al., and used herein.

In some embodiments, the nociceptor modulating agent, nociceptor antagonist, neuropeptide modulating agent, an agent that blocks vesicle release, or agent that blocks the release or action of a neuropeptide from tumor-innervating nociceptor is of Formula XIII-A, XIII-B, or XIII-C:

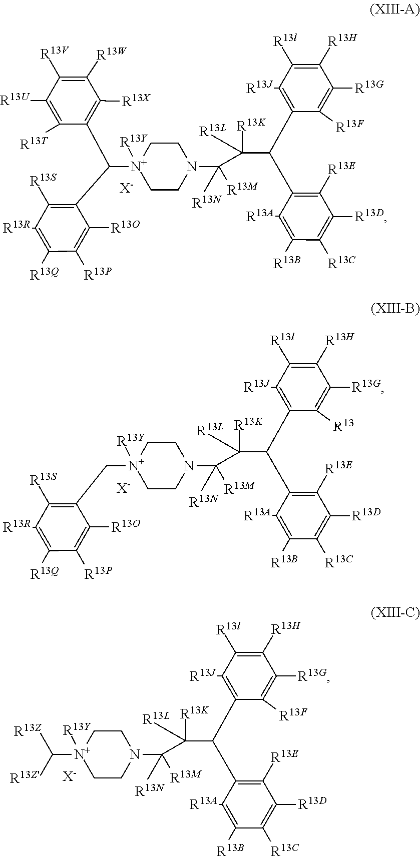

wherein:

each R^{13A}-R^{13J} and R^{13O}-R^{13T} is selected, independently, from H, halogen, C₁₋₄ alkyl, C₂₋₄ alkenyl, C₂₋₄ alkynyl, C₂₋₄ heteroalkyl, C₇₋₁₄ alkaryl, C₃₋₁₀ alkcycloalkyl, and C₃₋₁₀ alkheterocyclyl, OR^{13AA}, NR^{13AB}R^{13AC}, NR^{13AD}C(O)R^{13AE}, S(O)R^{13AF}, SO₂R^{13AG}R^{13AH}, SO₂NR^{13AI}R^{13AJ}, SO₃R^{13AK}, CO₂R^{13AL}, C(O)R^{13AM}, and C(O)NR^{13AN}R^{13AO};

each of R^{13AA}-R^{13AO} is, independently, selected from H, C₁₋₄ alkyl, C₂₋₄ alkenyl, C₂₋₄ alkynyl, and C₂₋₄ heteroalkyl;

each R^{13K}, R^{13L}, R^{13M} and R^{13N} is, independently, H or C₁₋₄ alkyl, or R^{13K} and R^{13L}, or R^{13M} and R^{13N}, combine to form C═O, or R^{13K} and R^{13M} combine to form C═C;

R^{13Y} is H or C₁₋₄ alkyl;

R^{13Z} and R^{13Z′} are, independently, selected from H, halogen, C₁₋₄ alkyl, C₂₋₄ alkenyl, C₂₋₄ alkynyl, C₂₋₄ heteroalkyl, C₇₋₁₄ alkaryl, C₃₋₁₀ alkcycloalkyl, and C₃₋₁₀ alkheterocyclyl; and

X⁻ is any pharmaceutically acceptable anion.

Exemplary compounds according to Formulas (XIII-A)-(XIII-C) include

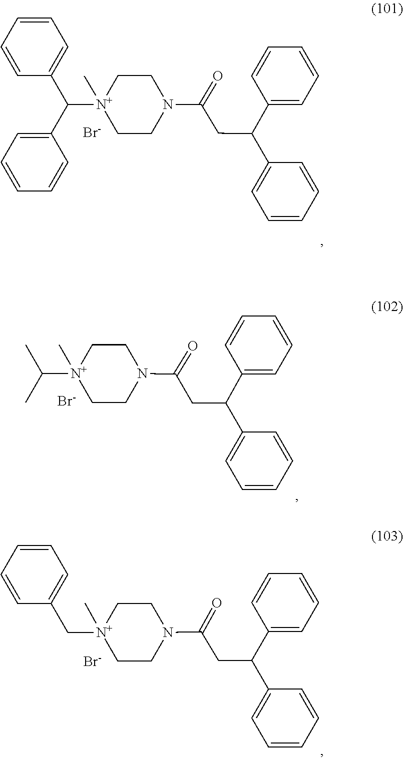 [SMILES: C[N+]1(C(c2ccccc2)c2ccccc2)CCN(C(=O)CC(c2ccccc2)c2ccccc2)CC1 ; [Br-] ; CC(C)[N+]1(C)CCN(C(=O)CC(c2ccccc2)c2ccccc2)CC1 ; [Br-] ; C[N+]1(Cc2ccccc2)CCN(C(=O)CC(c2ccccc2)c2ccccc2)CC1 ; [Br-]]

Derivatives of mibrefradil, such as those described in U.S. Pat. No. 4,808,605, hereby incorporated by reference can also be used. Exemplary mibrefadil derivatives include compounds of Formula (XIV). In some embodiments, the nociceptor modulating agent, nociceptor antagonist, neuropeptide modulating agent, an agent that blocks vesicle release, or agent that blocks the release or action of a neuropeptide from tumor-innervating nociceptor is of Formula XIV:

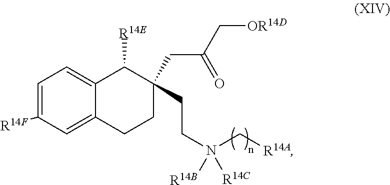

wherein:

n is an integer between 0-5;

R^{14A} is heterocyclyl (e.g., a heteroaryl such as benzimidazole),

each of R^{14B}, R^{14C}, R^{14D}, and R^{14E} is, independently, C₁₋₄ alkyl, C₂₋₄ alkenyl, C₂₋₄ alkynyl, C₂₋₄ heteroalkyl, C₇₋₁₄ alkaryl, C₃₋₁₀ alkcycloalkyl, and C₃₋₁₀ alkheterocyclyl; and

R^{14F} is selected from H, halogen, C₁₋₄ alkyl, C₂₋₄ alkenyl, C₂₋₄ alkynyl, C₂₋₄ heteroalkyl, C₇₋₁₄ alkaryl, C₃₋₁₀ alkcycloalkyl, and C₃₋₁₀ alkheterocyclyl, OR^{14G}, NR^{14H}R^{14I}, NR^{14J}C(O)R^{14K}, S(O)R^{14L}, SO₂R^{14M}R^{14N}, SO₂NR^{14O}R^{14P}, SO₃R^{14Q}, CO₂R^{14R}, C(O)R^{14S}, and C(O)NR^{14T}R^{14V}; and

each of R^{14G}-R^{13AO} is, independently, selected from H, C₁₋₄ alkyl, C₂₋₄ alkenyl, C₂₋₄ alkynyl, and C₂₋₄ heteroalkyl.

An exemplary compound of Formula (XIV) is

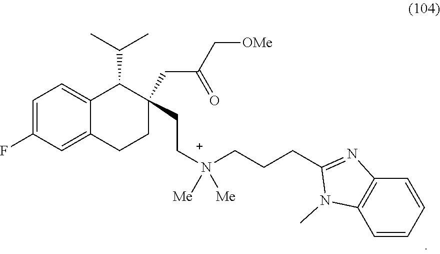 [SMILES: COCC(=O)C[C@]1(CC[N+](C)(C)CCCc2nc3ccccc3n2C)CCc2cc(F)ccc2[C@@H]1C(C)C]

Charged derivatives of 4-piperidinylaniline compounds (e.g., Compounds (86)-(88) of Table 2) can be prepared according to methods known in the literature and described herein. For example, charged N-alkyl derivatives (e.g., N-methyl) of Compounds (86)-(88) can be prepared and used in the compositions, methods, and kits described herein.

Still other channel blockers that can be quaternized or guanylated according to the methods described herein are described, for example, in PCT Publication No. WO 2004/093813 (see, e.g., Tables 5, 6 and 8), which is herein incorporated by reference.

In some embodiments, the nociceptor modulating agent, nociceptor antagonist, neuropeptide modulating agent, an agent that blocks vesicle release, or agent that blocks the release or action of a neuropeptide from tumor-innervating nociceptor is a quaternized or guanylated derivative of a compound as described in Table 3.

TABLE 3

| No. | Compound | Exemplary References |
|---|---|---|
| 105 | Isradipine | |
| 106 | Nickel Chloride | |
| 107 | A-53930A | JP 08208690 |
| 108 | AE-0047 Watanidipine | EP 00424901 |
| | dihydrochloride | |
| 109 | AGN-190604 | Inflammation, 19(2):261-275 |
| | | (1995) |
| 110 | AGN-190744 | EP372940 |
| 111 | AH-1058 | European Journal of |
| | | Pharmacology, 398(1): 107-112 |
| | | (2000) |
| 112 | AHR 5360C | European Journal of |
| | | Pharmacology 146(2-3): 215-22 |
| | | (1988) |
| 113 | AHR 12234 | Archives Internationales de |
| | | Pharamcodynamie et de Therapie |
| | | 301:131-50(1989) |
| 114 | AHR-12742 | ZA 08604522 |
| 115 | AHR-16303B | Journal of Cardiovascular |
| | | Pharmacology 17(1): 134-44 |
| | | (1991) |
| 116 | AHR-16462B | Drug Development Research, |
| | | 22(3): 259-271 (1991) |
| 117 | AIT 110 | |
| 118 | AIT 111 | |
| 119 | AJ2615 | WO 8601203 A1 |
| 120 | AJ-3941 | Arzneimittel Forschung |
| | | 46(6):567-71 (1996) |
| 121 | (+)-alismol | JP 04077420 A2 |
| 122 | AM-336 (synthetic version of | WO9954350 |
| | CVID marine cone snail venom) | |
| 123 | AM 543 | |
| 124 | amlodipine | U.S. Pat. No. 4572902 |
| 125 | S-(−)amlodipine | GB 2233974 A1 |
| 126 | AN 132 | EP 196648 |
| 127 | animpamil LU 42668 | EP 64158 A1 |
| 128 | antioquine (alkaloid from stem | Journal of natural Products |
| | bark) | 55(9):1281-6 (1992) |
| 129 | AP-1067 | IDDB 268934 |
| 130 | AQ-AH-208 | CH 645628 A |
| 131 | AR 12456 (derivative of trapidil) | BE 902218 A1 |
| | | Cardiovascular Drug Reviews |
| | | 9(4):385-397 (1991) |
| 132 | aranidipine | U.S. Pat. No. 4446325 |
| 133 | atosiban | EP 00112809 |
| 134 | azenidipine CS 905 | EP 88266922 |
| 135 | B 84439 | EP 240828 |
| 136 | barnidipine (derivative of | U.S. Pat. No. 4220649 |
| | nicardipine) | DE 02904552 |
| 137 | BAY-E-6927 | DE 2117571 |
| 138 | BAY-K-9320 | EP 9206 |
| 139 | BAY-T-7207 | |
| 140 | BBR-2160 | EP 28204 A2 |
| 141 | BDF 8784 | EP 25111 |
| 142 | belfosdil/BMY 21891/SR7037 | EP 173041 A1 |
| 143 | Bencylcalne/EGYT-201 | FR 151193 |
| 144 | benipidine/KW3049/N akadipine | U.S. Pat. No. 4448964 |
| 145 | bepridil | U.S. Pat. No. 3962238 |
| 146 | bisaramil/RGH 2957 | WO 9622096 |
| 147 | BK 129 | Methods and Findings in |
| | | Experimental and Clinical |
| | | Pharamcology 14(3): 175-81 |
| | | (1992) |
| 148 | BMS-181102 | EP 559569 |
| 149 | BMS-188107 | U.S. Pat. No. 5070088 |
| 150 | BMY 20014 | DE 3512995 A1 |
| 151 | BMY 20064 | DE 3512995 A1 |
| 152 | BMY-43011 | Bioorganic and Medicinal |
| | | Chemistry Letters, 3(12):2817- |
| | | 2820 (1993) |
| 153 | BN 50149 | WO 9323082 |
| 154 | BN 50175 | WO 9323082 |
| 155 | BN 50394 | WO 9323082 |
| 156 | BR 1022 | Current Science 83(4):426-431 |
| | | (2002) |
| 157 | BRL 3287A | WO 9323082 |
| 158 | BRL-32872 | WO 09323024 |
| 159 | buflomedil | U.S. Pat. No. 4326083 |
| 160 | butoprozine | DE 2707048 |
| 161 | CAF 603 | Organic and Bioorganic |
| | | Chemistry, 22:3349:52 (1994) |
| 162 | calciseptine (venom polypeptide) | WO 2000 069900 |
| 163 | calcium antagonists | WO 9205165 |
| 164 | calcium channel antagonists | WO 00236586 |
| | | WO 0236567 |
| 165 | calcium channel blocker (L-type) | Journal of Medicinal Chemistry, |
| | | 39(15):2922-2938 (1996) |
| 166 | calcium channel blockers | EP 400665 A2 |
| | | U.S. Pat. No. 4965356 |
| 167 | calcium channel blockers | WO 9526325 |
| 168 | carvedilol | U.S. Pat. No. 4503067 |
| 169 | caryachine | British Journal of Pharmacology, |
| | | 116(8):3211-8 (1995) |
| 170 | CD-349 | EP 92936 A1 |
| 171 | CD-832 | EP 00370821 |
| 172 | CER-2 metabolite of furnipidine | WO 9919302 |
| 173 | cerebrocrast | DE 3534385 |
| 174 | CERM 11956 | EP 138684 |
| 175 | CERM-12816 | IDDB 283075 |
| 176 | CGP 22442 | WO 9323082 |
| 177 | CGP 26797 | WO 9323082 |
| 178 | CGP 28727 | WO 9323082 |
| 179 | CGP 32413 | WO 9323082 |
| 180 | changrolin | Sci. Sin. (Engl. Ed.) 22(10): 1220-8 |
| | | (1979) |
| 181 | CHF-1521 (combination of | |
| | delapril and manidipine) | |
| 182 | cilnidipine | U.S. Pat. No. 4672068 |
| 183 | cinnarizine | U.S. Pat. No. 3799934 |
| 184 | civamide | WO 9640079 |
| | | U.S. Pat. No. 5840762 |
| 185 | clentiazem/T A3090 | EP 00127882 |
| | | U.S. Pat. No. 4567175 |
| 186 | clevidipine | WO 9512578 |
| 187 | CNS-1067 | IDdb 211675 |
| 188 | CNS-1237 | Annals of the New York Academy |
| | | of Sciences, 765 (Neuroprotective |
| | | Agents):210-29 (1995) |
| 189 | CNS-2103 (from spider venom) | WO 9214709 A2 |
| 190 | COR 28-22 | WO 9323082 |
| 191 | COR 2707C | WO 9323082 |
| 192 | COR 3752C | WO 9323082 |
| 193 | CP-060S | WO 9500471 A1 |
| 194 | CPC-301 | IDdb 231888 |
| 195 | CPC 304 | IDdb 185705 |
| 196 | CPC-317 | IDdb 185700 |
| 197 | CPU 23 | Yaoxue Xuebao, 25(11): 815-23 |
| | | (1990) |
| | | CAN 114:143097 |
| 198 | CPU-86017 | EP 00538844 |
| 199 | CRE 202 | WO 9323082 |
| 200 | CRE 204 | WO 9323082 |
| 201 | CRE 1005 | WO 9323082 |
| 202 | CRL-42752 | WO 00003987 |
| 203 | cronidipine (LF 2-0254) | EP 240398 A1 |
| 204 | CV 159 | FR 2511370 A1 |
| 205 | D-2024 (verapamil(S)) | WO 09509150 |
| 206 | D2603 | WO 9323082 |
| 207 | dagapamil | WO 9323082 |
| | | EP 64158 A1 |
| 208 | darodipine PY108068 | EP 00000150 |
| 209 | dauricine NSC 36413 | Acta Pharmacologica Sinica 7(6): |
| | | 543-7(1986) |
| 210 | desmethyl verapamil | |
| 211 | DHM9 | WO 8604581 A1 |
| 212 | DHP218/PAK9 | EP 00121117 |
| 213 | diclofurime | DE 79-29227999 |
| 214 | dihydropyridine calcium channel | Journal of Medicinal Chemistry |
| | blockers | 41(4):509-514 (1998) |
| 215 | diltiazem | U.S. Pat. No. 3562257 |
| 216 | diperdipine | EP 00218996 |
| 217 | dipfluzine | DE 3318577 A1 |
| 218 | diproteverine BRL 40015 | BE 866208 |
| 219 | dopropidil | EP 00031771 |
| 220 | dotarizine/FI 6026 | U.S. Pat. No. 4883797 |
| 221 | DTZ-323 | Molecular Pharmacology, |
| | | 51(2):262-268 (1997) |
| 222 | E-2050 | JP 2001199949 A2 |
| 223 | E4080 | EP 344577 A2 |
| 224 | efonidipine hydrochloride | U.S. Pat. No. 4885284 |
| 225 | EG 1088 | EP 56637 A1 |
| 226 | EGIS 3966 | DE 4027052 A1 |
| 227 | eglodipine | DE 3825962 A1 |
| 228 | emopamil (racemic) SZ 45 | DE 3344755 A1 |
| 229 | (S)-emopamil | DE 3344755 A1 |
| 230 | enalapril_nitrendipine, Vita- | EP 00884054 |
| | Inveest | |
| 231 | etafenonee LG 11457 | DE 1265758 |
| 232 | ethosuximide | |
| 233 | eugenodilol | JP 11255719 A2 |
| 234 | evodiamine | JP 52077098 |
| 235 | F-0401 | EP 00320984 |
| 236 | falipamil AQA 39 | Journal of Medicinal Chemistry, |
| | | 33(5): 1496-504 (1990) |
| 237 | fantofarone SR 33557 | EP 235111 A1 |
| | | U.S. Pat. No. 4957925 |
| 238 | fasudil (iv formulation), Asahi | U.S. Pat. No. 4678783 |
| 239 | FCE-24265 | EP 373645 A1 |
| 240 | FCE-26262 | |
| 241 | FCE-27335 | |
| 242 | FCE-27892 | |
| 243 | FCE-28718 | EP 00755931 |
| 244 | fedopamil | |
| 245 | felodipine | U.S. Pat. No. 4264611 |
| 246 | felodipine + ramipril | WO 09607400 |
| | (Astra/Aventis) | |
| 247 | fendiline | U.S. Pat. No. 3262977 |
| 248 | feniline | |
| 249 | flezelastine, D 18024 | EP 590551 A2 |
| 250 | flordipine | |
| 251 | fluodipine | U.S. Pat. No. 3773939 |
| 252 | fluphenazine, S94 | Journal of Medicinal Chemistry, |
| | SQ 4918 | 19(6):850-2 (1976) |
| | Triflumethazine | |
| | Vespazine | |
| 253 | fostedil KB 944 | EP 10120 |
| 254 | FPL 62129 | EP 125803 A2 |
| 255 | FR 46171 | |
| 256 | FR-172516 | JP 09040647 |
| 257 | FRC9411 | |
| 258 | FRG 8653 | |
| 259 | FRG-8701 | |
| 260 | furaldipine | |
| 261 | furnidipine (CRE 319) | Journal of Medicinal Chemistry, |
| | | 38(15):2830-41 (1995) |
| 262 | GOE 5057 | |
| 263 | GOE 5584 A | EP 173933 A1 |
| 264 | GOE 93007 | |
| 265 | GR 60139 | |
| 266 | GR 55234A (R-enantiomer of | Haemotalogica, 79(4):328-33 |
| | telupidine) | (1994) |
| 267 | GR 55235A (L-enantiomer of | Haemotalogica, 79(4):328-33 |
| | telupidine) | (1994) |
| 268 | GS-386 | |
| 269 | GYKI 46544 | |
| 270 | H32438 | |
| 271 | HA 22 | U.S. Pat. No. 5240947 |
| 272 | HA 23 | U.S. Pat. No. 5240947 |
| 273 | HA 1004 | |
| 274 | GA 1077 | |
| 275 | HE 30346 | |
| 276 | HNS 32 | JP 08311007 A2 |
| 277 | HOE 166 | Molecular Pharmacology |
| | | 33(4):363-9(1988) |
| 278 | HOE 263 | |
| 279 | HP 406 | U.S. Pat. No. 4521537 |
| 280 | ICI206970 | EP 293170 A1 |
| | | 19881130 |
| 281 | iganidipine | JP 63225355 A2 |
| | | 19880920 |
| 282 | IHC 72 | Acta Pharmaceutica Sinica, |
| | | 27(6):407-11 (1992) |
| 283 | ipenoxazone | |
| 284 | isradipine | U.S. Pat. No. 4466972 |
| 285 | JTV-519 | WO 09212148 |
| 286 | KB 2796 | |
| 287 | KP-840 | Yakubutsu, Seishin, Kodo, |
| | | 12(6):353 (1992) |
| 288 | KP873 | |
| 289 | KT-362 | Archiv Der Pharmazie, |
| | | 328(4):313-6(1995) |
| 290 | KT 2230 | General Pharmacology, 22(3):443- |
| | | 8(1991) |
| 291 | KW 3049 (see benipidine) | |
| 292 | L-366682 | EP 00444898 |
| 293 | L-651582 | |
| 294 | L735821 | WO 9514471 A1 |
| | | 19950601 |
| | | British Journal of Pharmacology, |
| | | 132(1):101-110 (2001) |
| 295 | lacidipine GR 43659 Sn305 | U.S. Pat. No. 4801599 |
| | | DE 03529997 |
| 296 | LAS 30356 | |
| 297 | LAS 30398 | |
| 298 | LAS 30538 | Journal of Pharmacy and |
| | | Pharmacology, 44(10:830-5 |
| | | (1992) |
| 299 | LAS Z077 | |
| 300 | LCB-2514 | |
| 301 | lemildipine | P59152373A2 |
| 302 | lercanidipine | U.S. Pat. No. 4705797 |
| 303 | leualacin | EP 00358418 |
| 304 | levosemotiadil SA 3212 | WO 08700838 |
| 305 | lidoflazine R7904 | U.S. Pat. No. 3267104 |
| 306 | lifarizine RS 87476 | U.S. Pat. No. 0435417 |
| 307 | LOE-908 | |
| 308 | lomerizine KB 2796 | U.S. Pat. No. 4663325 |
| | | EP 00158566 |
| 309 | LU 49700 (main metabolite of | DE 3642331 A1 |
| | gallopamil) | |
| 310 | LU 49938 | |
| 311 | LY-042826 | European Journal of |
| | | Pharmacology, 408(3):241-248 |
| | | (2000) |
| 312 | LY-393615 | European Journal of |
| | | Pharmacology, 408(3):241-248 |
| | | (2000) |
| 313 | manidipine/CV 4093/franidipine | U.S. Pat. No. 4892875 |
| | | EP 00094159 |
| 314 | MCI 176 (MY7674) | EP 169537 A2 |
| 315 | McN 5691 (see RWJ 26240) | |
| 316 | McN-6186 | |
| 317 | MCN 6497 | |
| 318 | MD 260792 | |
| 319 | MDL 143 | |
| 320 | MDL 12330A | |
| 321 | MDL 16582A | WO 9323082 |
| 322 | MDL 72567 | GB 2137622 A1 |
| | | 19841010 |
| | | CAN 102:95548 |
| 323 | MEM 1003/nimopidine | |
| | analog/BAY Z 4406 | |
| 324 | mepirodipine | |
| 325 | mesudipine | |
| 326 | mibefradil | EP 00268148 |
| | | U.S. Pat. No. 4808605 |
| 327 | minodipine | |
| 328 | mioflazine | |
| 329 | MJ 14712 | |
| 330 | monatepil maleate (AD 2615) | WO 08601203 |
| | | U.S. Pat. No. 4749703 |
| 331 | MPC 1304 | |
| 332 | MPC 2101 | FR 2514761 A1 |
| 333 | MR-14134 | Pharmacology, 51(2):84-95 (1995) |
| 334 | N-3601 | EP 254322 A1 |
| 335 | N 20776 | |
| 336 | N-allyl secoboldine | |
| 337 | naltiazem Ro 23-6152 | U.S. Pat. No. 4652561 |
| 338 | NB 818 | |
| 339 | NC 1100 | |
| 340 | NC O 700 | |
| 341 | NCC 09-0026 | |
| 342 | nexopamil | EP 00271013 |
| 343 | NH 2250 | |
| 344 | NH2716 | |
| 345 | nicainoprol RU 42924 | DE 2934609 |
| 346 | nicardipine (nifelan) | U.S. Pat. No. 3985847 |
| 347 | nictiazem | |
| 348 | nifedipine | U.S. Pat. No. 3485847 |
| 349 | nigulipine | WO 8807525 A1 |
| 350 | niludipine | |
| 351 | nilvadipine FK 235 | U.S. Pat. No. 4338322 |
| | | DE 02940833 |
| 352 | nimodipine | U.S. Pat. No. 3842096 |
| 353 | misoldipine Bay y 5552 | U.S. Pat. No. 4154839 |
| 354 | nitrendipine Bay k 5009 | U.S. Pat. No. 3799934 |
| 355 | NMDA/calcium channel | WO 09745115 |
| | antagonists, Allelix | |
| 356 | NKY 722 | |
| 357 | NMED 126 (MC-34D) | WO 0145709 A1 |
| | | U.S. Pat. No. 6387897 |
| 358 | NMED 427 | WO 0145709 A1 |
| | | U.S. Pat. No. 6387897 |
| 359 | NMED 724 | WO 0145709 A1 |
| | | U.S. Pat. No. 6387897 |
| 360 | NMED 826 | WO 0145709 A1 |
| | | U.S. Pat. No. 6387897 |
| 361 | NMED JM-G-10 | WO 0145709 A1 |
| | | U.S. Pat. No. 6387897 |
| 362 | NMED 157 39-1B4 | WO 0145709 A1 |
| | | U.S. Pat. No. 6387897 |
| 363 | NMED 160 39-45-3 | WO 0145709 A1 |
| | | U.S. Pat. No. 6387897 |
| 364 | NNC-09-0026 | WO 9201672 |
| 365 | NP 252 | Life Sciences, 48(2): 183-8 (1991) |
| 366 | NS 626 | |
| 367 | NS-638 | U.S. Pat. No. 5314903 |
| | | EP 545845 A1 |
| 368 | NS-649 | EP 520200 A2 |
| 369 | NS-696 | |
| 370 | NS-7 | WO 09607641 |
| 371 | NS 3034 | |
| 372 | NZ 105 | |
| 373 | olradipine S 11568 | FR 2602231 A1 |
| 374 | ONO-2921 | WO 0000470 A1 |
| 375 | OPC 13340 | |
| 376 | OPC 88117 | EP 236140 A2 |
| 377 | ORG 13020 | |
| 378 | Org-13061 | Fundamental & Clinical |
| | | Pharmacology, 11(5):416-426 |
| | | (1997) |
| 379 | OS AT (nifedipine) | |
| 380 | osthole | JP 47000430 |
| 381 | oxodipine IQB 837V | ES 531033 A1 |
| 382 | P 0825 | |
| 383 | P 1268 | |
| 384 | palonidipine hydrochloride | Ep 128010 A2 |
| 385 | PCA-50922 | |
| 386 | PCA-50938 | Brain Research 772(1,2):57-62 |
| | | (1997) |
| 387 | PCA-50941 | |
| 388 | PCA 50982 | |
| 389 | PD-0204318 | WO 9943658 A1 |
| 390 | PD-029361 | IDdb 300520 |
| 391 | PD 122860 | Ep 206747 A2 |
| 392 | PD 151307 | U.S. Pat. No. 6423689 |
| | | J. Med. Chern. 43:3472 (2000) |
| 393 | PD-157667 | U.S. Pat. No. 5767129 |
| 394 | PD-158143 | WO 9705125 A1 |
| 395 | PD 173212 | |
| 396 | PD 175069 | WO 9854123 A1 |
| 397 | PD 176078 | WO 9955688 |
| | | J. Med. Chem. 43:3474 (2000) |
| 398 | PD 181283 | Bioorganic & Medicinal Chemistry |
| | | Letters, 9(16):2453-2458 (1999) |
| 399 | pelanserin | |
| 400 | perhexiline | GB 1025578 |
| 401 | petro synol | Tetrahedron, 49(45): 10435-8 |
| | | (1993) |
| 402 | PF 244 | |
| 403 | PFS 1144 (EO 122) | DE 2802208 |
| 404 | pirmenol | U.S. Pat. No. 4112103 |
| 405 | pirprofurol | |
| 406 | | |
| 407 | PN 200110 | |
| 408 | PNU 156654E | WO 9705102 A1 |
| 409 | pranidipine | EP 00145434 |
| 410 | prenylamine | |
| 411 | propiverine | DD 106643 |
| 412 | ptilomycalin AM | |
| 413 | QM 96233 | |
| 414 | QM 96159 | |
| 415 | QM 96127 | |
| 416 | QX-314 | Biophysical Journal, 27(1):39-55 |
| | | (1979) |
| 417 | R56865 | EP 184257 A1 |
| 418 | R 59494 | Ep 184257 A1 |
| 419 | R71811 | |
| 420 | Rec 152288 | |
| 421 | Rec 152375, Rec 15/375 | |
| 422 | RGH-2716 (TDN 345) | EP 414421 A2 |
| 423 | RGH 2970 | |
| 424 | riodipine | |
| 425 | Ro-11-2933 | EP 00523493 |
| 426 | Ro 18-3981 | |
| 427 | Ro 40-5967 | |
| 428 | RO 445912 dithiane | Biochemical Pharmacology, |
| | derivatives of tiapamil | 50(2): 187-96 (1995) |
| 429 | ronipamil | |
| 430 | RS-5773 | EP 00353032 |
| 431 | RS 93007 | |
| 432 | RS 93522 | U.S. Pat. No. 4595690 |
| 433 | RU-43945 | WO 9323082 Al |
| 434 | RWJ-22108 | U.S. Pat. No. 04845225 |
| 435 | RWJ-22726 | U.S. Pat. No. 04845225 |
| 436 | RWJ 26240 McN 5691 | EP 146721 A2 |
| 437 | RWJ 26899 | EP 237191 A1 |
| 438 | RJW-26902 | |
| 439 | RWJ-29009 | EP 00493048 |
| 440 | RWJ-37868 | WO 0048584 |
| 441 | ryanodine | |
| 442 | S-(−)-amlodipine | |
| 443 | S 11568 | |
| 444 | S 12967 | ZA 9000231 A |
| 445 | S-12968 | EP 00406502 |
| 446 | S-2150 | Ep 00615971 |
| 447 | S-312-d | JP 03052890 |
| 448 | S 830327 | |
| 449 | SA 2572 | JP 63104969 A2 |
| 450 | SA 2995 | |
| 451 | SA 3212 | |
| 452 | sabeluzole | Ep 184257 A1 |
| 453 | safinamide | EP 400495 A1 |
| 454 | sagandipine | |
| 455 | salicylaldoxime | Clinical and Experimental |
| | | Pharmacology and Physiology |
| | | 26(12):964-9 (1999) |
| 456 | SANK-71996 | |
| 457 | SB-201823A | WO 09202502 |
| 458 | SB-206284A | |
| 459 | SB 221420A | WO 9002494 A1 |
| 460 | SB-237376 | WO 0209761 A2 |
| 461 | SB 262470 | WO 0183546 A1 |
| 462 | SC 30552 | |
| 463 | SDZ-249482 | |
| 464 | selodipine | |
| 465 | semotiadil (SD 3211) | U.S. Pat. No. 4786635 |
| | | JP 09012576 |
| 466 | SIM 6080 | Ep 293925 A2 |
| 467 | sipatrigine | EP 372934 A2 |
| 468 | sinomenine (active from | WO 0269971 A1 |
| | a Chinese medicinal plant) | |
| 469 | siratiazem | WO 09117153 |
| 470 | SKF-45675 | |
| 471 | SKF-96365 | European Journal of |
| | | Pharmacology 188(6):417-21 |
| | | (1990) |
| 472 | SKT-M-26 | |
| 473 | SL-34.0829 | WO 0209761 A2 |
| 474 | SL 651708 | |
| 475 | SL 851016 | |
| 476 | SL-870495 | |
| 477 | SM-6586 | EP 00177965 |
| 478 | S NX-124 | |
| 479 | SNX 185 | WO 9310145 A1 |
| 480 | S NX-236 | WO 09313128 |
| 481 | S NX-239 | Pain, 60(1):83-90 (1995) |
| 482 | SNX-483 (peptides from tarantula | WO 9805780 A2 |
| | venom) | |
| 483 | sornidipine | |
| 484 | SQ 31486 | EP 205334 A2 |
| 485 | SQ 31727 | |
| 486 | SQ 31765 | |
| 487 | SQ 32321 | |
| 488 | SQ 32324 | |
| 489 | SQ 32547 | EP 400665 A2 |
| 490 | SQ 32926 | EP 400665 A2 |
| 491 | SQ-33351 | WO 09006118 |
| 492 | SQ 33537 | |
| 493 | SQ 34399 | |
| 494 | SR-33805 | EP 576347 A1 |
| 495 | SUN 5647 | |
| 496 | SUN 6087 | |
| 497 | SUN-N8075 | WO 9923072 A2 |
| 498 | T-477 | EP 00441539 |
| 499 | TA-993 | JP 01050872 |
| 500 | taludipine | |
| 501 | tamolarizine | EP 00354068 |
| 502 | TDN-345 | |
| 503 | Teczem | |
| 504 | temiverine | CAN 131:193592 |
| 505 | terflavoxate | EP 72620 A1 |
| 506 | terodiline TD 758 | U.S. Pat. No. 3371014 |
| 507 | tetrandrine | Clinical and Experimental |
| | | Pharmacology and Physiology, |
| | | 23(8):715-753 (1996) |
| 508 | TH-1177 | |
| 509 | TH-9229 | WO 09607415 |
| 510 | thapsigargin | British Journal of Pharmacology, |
| | | 95(3):705-712 (1985) |
| 511 | tiapamil | |
| 512 | tinctormine | Chemical & Pharmaceutical |
| | | Bulletin 40(12):3355-7 (1992) |
| 513 | TJN 220 (O-ethylfangchinoline) | JP 63179878 A2 |
| 514 | TMB 8 | Journal of Cell Science 79:151-160 |
| | | (1985) |
| 515 | TN-871 | European Journal of |
| | | Pharmacology 342 (2/3): 167-175 |
| | | (1998) |
| 516 | TR 2957 | |
| 517 | trapidil | |
| 518 | trimetazidine | U.S. Pat. No. 3262852 |
| 519 | TY-10835 | Pharmacometrics, 1998, 54:3 |
| | | (153) |
| 520 | U-88999 | WO 9204338 |
| 521 | U-92032 | WO 09204338 |
| 522 | U-92798 | WO 9204338 A1 |
| 523 | UK 1745 | EP 653426 A1 |
| 524 | UK-51656 | EP 00089167 |
| 525 | UK 52831 | JP 59118782 A2 |
| 526 | UK 55444 | EP 00132375 |
| 527 | UK 56593 | |
| 528 | UK-84149 | EP 404359 A1 |
| 529 | ULAH 99 | European Journal of |
| | | Pharmacology, 229(1):55-62 |
| | | (1992) |
| 530 | vantanipidine | EP 257616 A2 |
| 531 | verapamil, verelan | U.S. Pat. No. 3261859 |
| 532 | S-verapamil, D-2024, | WO 09509150 |
| | levo verapamil | |
| 533 | vexibinol | Chemical and Pharmaceutical |
| | Sophoraflavanone G | Bulletin 38(4): 1039-44 (1990) |
| 534 | vinigrol | |
| 535 | vintoperol RGH 2981 RT 303 | WO 9207851 |
| 536 | vingrol | |
| 537 | vintoperol/RGH 2981/RT 303 | WO 9207851 |
| 538 | VUF-8929 | EP 467435 A2 |
| 539 | VULM 993 | |
| 540 | vantanipidine | Ep 257616 A2 |
| 541 | W787 | |
| 542 | WAS 4206 | |
| 543 | WK 269 | |
| 544 | WY 27569 | |
| 545 | WY 44644 | |
| 546 | WY 44705 | |
| 547 | WY 46622 | |
| 548 | WY 47324 | |
| 549 | xanthonolol | U.S. Pat. No. 5495005 |
| 550 | Y19638 | |
| 551 | Y-22516 | WO 9323082 |
| 552 | Y208835 | |
| 553 | YC 114 | |
| 554 | YH-334 | EP 00366548 |
| 555 | YM 15430-1 (see YM 430) | |
| 556 | YM-16151-4 (YM 151) | EP 00167371 |
| 557 | YM-430 (YM 15430) | WO 0209761 A2 |
| 558 | YS 035 | BE 897244 |
| 559 | YS 161 | |
| 560 | Z-6568 | Journal of Mass Spectrometry, |
| | | 31(1):37-46 (1996) |
| 561 | ziconotiide omega | WO 9107980 |
| | conotoxin/MVIIA/SNX-111 | |
| 562 | ZM-224832 | EP 00343865 |
| 563 | zonisamide | U.S. Pat. No. 4172896 |

In some embodiments, the nociceptor modulating agent, nociceptor antagonist, neuropeptide modulating agent, an agent that blocks vesicle release, or agent that blocks the release or action of a neuropeptide from tumor-innervating nociceptor is a capsaicinoid. In some embodiments, the nociceptor modulating agent, nociceptor antagonist, neuropeptide modulating agent, an agent that blocks vesicle release, or agent that blocks the release or action of a neuropeptide from tumor-innervating nociceptor is capsaicin or resiniferatoxin.

In some embodiments, the nociceptor modulating agent, nociceptor antagonist, neuropeptide modulating agent, an agent that blocks vesicle release, or agent that blocks the release or action of a neuropeptide from tumor-innervating nociceptor is a neurotoxic protein. In some embodiments, the agent is a neurotoxic protein. In some embodiments, the neurotoxic protein is a clostridial neurotoxin. In some embodiments, the neurotoxic protein is produced by a clostridium. In certain embodiments, the neurotoxic protein is produced by Clostridium botulinum, C. argentinense, C. butyricum, C. baratii spp, or Clostridium tetani. In some embodiments, the neurotoxic protein is produced by Chryseobacterium piperi. In some embodiments, the neurotoxic protein is produced by Enterococcus faecium (BoNT/En). In some embodiments, the neurotoxic protein is produced by Weissella oryzae. In certain embodiments, the neurotoxic protein is a botulinum neurotoxin. In certain embodiments, the neurotoxic protein is a botulinum neurotoxin other than BoNT/a. In certain embodiments, the neurotoxic protein is a botulinum neurotoxin serotype selected from A, B, C, D, E, F, G, H, and, X, or variant or subtype thereof (e.g., BoNT/a subtype A1-A5 (i.e., BoNT/A1, BoNT/A2)). In certain embodiments, the neurotoxic protein is a botulinum neurotoxin serotype selected from A, B, C, D, E, F, and G, or variant or subtype thereof (e.g., BoNT/a subtype A1-A5 (i.e., BoNT/A1, BoNT/A2)). In certain embodiments, the neurotoxic protein is a botulinum neurotoxin serotype selected from B, C, D, E, F, G, H, and, X, or variant or subtype thereof. In certain embodiments, the neurotoxic protein is a botulinum neurotoxin serotype selected from A, B, E, and F. In some embodiments, the botulinum neurotoxin is a BoNT variant. In some embodiments, the BoNT variant is selected from BoNT/A1-A5, B1-B7, E1-E11, and F1-F7. In some embodiments, the botulinum neurotoxin is BoNT/a. In some embodiments, the neurotoxic protein is a BoNT-like toxin. In some embodiments, the neurotoxic protein is tetanus neurotoxin (TeNT). In some embodiments the agent is abobotulinumtoxinA, incobotulinumtoxinA, onabotulinumtoxinA, or rimabotulinumtoxinB.

In some embodiments, provided herein are methods of treating cancer in a subject comprising administrating to the subject a therapeutically effective amount of an agent that blocks vesicle release from tumor-innervating neurons. In some embodiments, provided herein are methods of treating cancer in a subject comprising administrating to the subject a therapeutically effective amount of an agent that blocks vesicle release from tumor-innervating nociceptors. In some embodiments, provided herein are methods of preventing cancer in a subject comprising administrating to the subject a prophylactically effective amount of an agent that blocks vesicle release from tumor-innervating neurons. In some embodiments, provided herein are methods of preventing cancer in a subject comprising administrating to the subject a prophylactically effective amount of an agent that blocks vesicle release from tumor-innervating nociceptors. In some embodiments, the agent is a neurotoxic protein. In some embodiments, the neurotoxic protein is a clostridial neurotoxin. In some embodiments, the neurotoxic protein is produced by a clostridium. In certain embodiments, the neurotoxic protein is produced by Clostridium botulinum, C. argentinense, C. butyricum, C. baratii spp, or Clostridium tetani. In some embodiments, the neurotoxic protein is produced by Chryseobacterium piperi. In some embodiments, the neurotoxic protein is produced by Enterococcus faecium (BoNT/En). In some embodiments, the neurotoxic protein is produced by Weissella oryzae. In certain embodiments, the neurotoxic protein is a botulinum neurotoxin. In certain embodiments, the neurotoxic protein is a botulinum neurotoxin other than BoNT/a. In certain embodiments, the neurotoxic protein is a botulinum neurotoxin serotype selected from A, B, C, D, E, F, G, H, and, X, or variant or subtype thereof (e.g., BoNT/a subtype A1-A5 (i.e., BoNT/A1, BoNT/A2)). In certain embodiments, the neurotoxic protein is a botulinum neurotoxin serotype selected from A, B, C, D, E, F, and G, or variant or subtype thereof (e.g., BoNT/a subtype A1-A5 (i.e., BoNT/A1, BoNT/A2)). In certain embodiments, the neurotoxic protein is a botulinum neurotoxin serotype selected from B, C, D, E, F, G, H, and, X, or variant or subtype thereof. In certain embodiments, the neurotoxic protein is a botulinum neurotoxin serotype selected from A, B, E, and F. In some embodiments, the botulinum neurotoxin is a BoNT variant. In some embodiments, the BoNT variant is selected from BoNT/A1-A5, B1-B7, E1-E11, and F1-F7. In some embodiments, the botulinum neurotoxin is BoNT/a. In some embodiments, the neurotoxic protein is a BoNT-like toxin. In some embodiments, the neurotoxic protein is tetanus neurotoxin (TeNT). In some embodiments the agent is abobotulinumtoxinA, incobotulinumtoxinA, onabotulinumtoxinA, or rimabotulinumtoxinB.

In certain embodiments, provided herein are methods of treating cancer in a subject comprising administering to a subject a therapeutically effective amount of a calcitonin gene-related peptide (CGRP) modulating agent. In certain embodiments, provided herein are methods of preventing cancer in a subject comprising administering to a subject a prophylactically effective amount of a calcitonin gene-related peptide (CGRP) modulating agent. In some embodiments, the CGRP modulating agent is a CGRP receptor antagonist. In certain embodiments the CGRP receptor antagonist is a RAMP1, RAMP3, or Vpac1 blocker. In some embodiments, the CGRP receptor antagonist is a RAMP1 blocker. In certain embodiments, the CGRP receptor antagonist is erenumab, fremanezumab, fremanezumab, eptinezumab, ubrogepant, or rimegepant. In certain embodiments, the CGRP receptor antagonist is BIBN 4096.

In one aspect, provided herein are methods of treating cancer in a subject comprising administering to a subject a therapeutically effective amount of one or more of QX-314, BoNT/a, and BIBN 4096. In one aspect, provided herein are methods of treating cancer in a subject comprising administering to a subject a therapeutically effective amount of QX-314 and BoNT/a. In one aspect, provided herein are methods of treating cancer in a subject comprising administering to a subject a therapeutically effective amount of QX-314. In one aspect, provided herein are methods of treating cancer in a subject, the method comprising administering to a subject a therapeutically effective amount of BIBN 4096.

In one aspect, provided herein are methods of preventing cancer in a subject comprising administering to a subject a prophylactically effective amount of one or more of QX-314, BoNT/a, and BIBN 4096. In one aspect, provided herein are methods of preventing cancer in a subject comprising administering to a subject a prophylactically effective amount of QX-314 and BoNT/a. In one aspect, provided herein are methods of preventing cancer in a subject comprising administering to a subject a prophylactically effective amount of QX-314. In one aspect, provided herein are methods of preventing cancer in a subject comprising administering to a subject a prophylactically effective amount of BIBN 4096.

In certain embodiments, provided herein are methods of treating cancer in a subject comprising ablating an ion channel in a subject, wherein the ion channel is a sodium ion channel or TRPV ion channel. In certain embodiments, provided herein are methods of preventing cancer in a subject comprising ablating an ion channel in a subject, wherein the ion channel is a sodium ion channel or TRPV ion channel. In certain embodiments, the sodium channel is selected from Na_{V}1.6, Na_{V}1.7, Na_{V}1.8, and Na_{V}1.9. In certain embodiments, the sodium channel is Na_{V}1.8. In some embodiments, the calcium channel is Ca_{V}2.2. In certain embodiments, the TRPV ion channel is TRPV1. In some embodiments, the ion channel is a calcium ion channel. In some embodiments, the calcium channel is Ca_{V}1.1-1.4, Ca_{V}2.1, Ca_{V}2.2, Ca_{V}2.3, Ca_{V}3.1-3.3. In some embodiments, the ion channel is genetically ablated. In some embodiments, the ion channel is ablated through genetic mutation. In some embodiments, the ion channel is ablated through genetic mutation during the development of the subject. In some embodiments, the ion channel is ablated through use of a denervating agent. In some embodiments, the denervating agent is capsaicin. In some embodiments, the denervating agent is resiniferatoxin.

In certain embodiments, provided herein are methods of treating cancer in a subject. In some embodiments, the cancer is skin cancer, breast cancer, prostate cancer, ovarian cancer, pancreatic cancer, gastric cancer, or a tumor. In certain embodiments, the cancer is skin cancer. In some embodiments, the cancer is breast cancer. In certain embodiments, the cancer is prostate cancer. In some embodiments, the cancer is ovarian cancer. In certain embodiments, the cancer is pancreatic cancer. In some embodiments, the cancer is gastric cancer. In some embodiments, the cancer is a melanoma. In some embodiments, the melanoma is superficial spreading melanoma, nodular melanoma, acral-lentiginous melanoma, lentigo maligna melanoma, amelanotic melanoma, desmoplastic melanoma, ocular melanoma, or metastatic melanoma. In some embodiments, the cancer is metastatic melanoma. In certain embodiments, the cancer is a tumor. In some embodiments, the cancer is a benign tumor. In certain embodiments, the cancer is a malignant tumor. In certain embodiments, the is innervated.

In certain embodiments, the method decreases tumor growth, volume, and/or size.

In some embodiments, the method inhibits or decreases cancer cell proliferation.

In certain embodiments, the method increases subject survival.

In some embodiments, the method promotes anti-tumor activity.

In certain embodiments, the method increases lymphocyte numbers.

In some embodiments, the method improves response to chemotherapeutics.

In some embodiments, the method improves efficacy of immunotherapy.

In some embodiments, the method increases efficacy of αPDL1 treatment. In some embodiments, the method increases efficacy of PDL1 treatment. In some embodiments, when QX-314 or BoNT/a is used to treat cancer, the method increases efficacy of αPDL1 treatment. In some embodiments, when QX-314 or BoNT/a is used to treat cancer, the method increases efficacy of PDL1 treatment.

In some embodiments, the method leads to exhaustion of tumor-infiltrating lymphocytes.

In certain embodiments, the method decreases tumor comorbidities. In some embodiments, the comorbidity is pain or itch. In some embodiments, the comorbidity is pain. In certain embodiments, the comorbidity is itch.

In some embodiments, one or more additional therapies are administered to the subject. In some embodiments, an additional therapy is administered to the subject. In some embodiments, the additional therapy is chemotherapy, radioimmunotherapy, surgical therapy, immunotherapy, radiation therapy, or targeted therapy, or any combination thereof. In some embodiments, the additional therapy is chemotherapy. In some embodiments, the additional therapy is radioimmunotherapy. In some embodiments, the additional therapy is surgical therapy. In some embodiments, the additional therapy is immunotherapy. In some embodiments, the additional therapy is radiation therapy. In some embodiments, the additional therapy is targeted therapy. In some embodiments, the additional therapy is an anti-cancer agent.

In another aspect, provided herein is the use of a nociceptor modulating agent for treating or preventing cancer in a subject. In some embodiments, the nociceptor modulating agent is a nociceptor antagonist.

In a further aspect, provided herein is the use of a neuropeptide modulating agent for treating or preventing cancer in a subject.

In one aspect, provided herein is the use of an agent that blocks the release or action of a neuropeptide from tumor-innervating neurons for treating cancer in a subject.

In another aspect, provided herein is the use of an agent that blocks vesicle release from tumor-innervating nociceptors for treating cancer in a subject.

In a further aspect, provided herein is the use of a calcitonin gene-related peptide (CGRP) modulating agent for treating cancer in a subject. In some embodiments, the CGRP modulating agent is a CGRP receptor antagonist. In some embodiments, the CGRP receptor antagonist is a RAMP1 blocker.

In one aspect, provided herein is the use of QX-314, BoNT/a, and/or BIBN 4096 for treating cancer in a subject. In one aspect, provided herein is the use of QX-314 for treating cancer in a subject. In one aspect, provided herein is the use of BoNT/a for treating cancer in a subject. In one aspect, provided herein is the use of BIBN 4096 for treating cancer in a subject.

In another aspect, provided herein is the use of genetic mutation to ablate an ion channel in a subject, wherein the ion channel is a sodium ion channel or TRPV ion channel, for treating cancer. In some embodiments, the ion channel is Na_{V}1.8 and/or TRPV1.

Any of the methods or uses provided herein can be carried out employing a composition of the agent, blocker, protein, peptide, antagonist, or compound described herein (e.g., a composition comprising a neuropeptide modulating agent, an agent that blocks the release or action of a neuropeptide from tumor-innervating neurons, a nociceptor modulating agent such as a nociceptor antagonist, a sodium channel blocker, a calcium channel blocker, a sodium and calcium channel blocker, a compound comprising a quaternary amine, an agent that blocks vesicle release from tumor-innervating nociceptors, a neurotoxic protein, a calcitonin gene-related peptide (CGRP) modulating agent, a CGRP receptor antagonist, or RAMP1 blocker).

### Compositions, Combinations, and Kits

In one aspect, also provided herein are compositions comprising (i) an anti-cancer agent, (ii) a nociceptor modulating agent, nociceptor antagonist, neuropeptide modulating agent, an agent that blocks vesicle release, or agent that blocks the release or action of a neuropeptide from tumor-innervating nociceptor described herein, and (iii) optionally a pharmaceutically acceptable excipient. In certain embodiments, the pharmaceutical composition described herein comprises (i) an anti-cancer agent, (ii) a nociceptor modulating agent, nociceptor antagonist, neuropeptide modulating agent, an agent that blocks vesicle release, or agent that blocks the release or action of a neuropeptide from tumor-innervating nociceptor described herein, and (iii) a pharmaceutically acceptable carrier or excipient.

In certain embodiments, provided herein are compositions comprising an anti-cancer agent and a nociceptor modulating agent, nociceptor antagonist, neuropeptide modulating agent, an agent that blocks vesicle release, or agent that blocks the release or action of a neuropeptide from tumor-innervating nociceptor which is as described herein. In certain embodiments, provided herein are compositions comprising an anti-cancer agent and a nociceptor modulating agent, nociceptor antagonist, neuropeptide modulating agent, an agent that blocks vesicle release, or agent that blocks the release or action of a neuropeptide from tumor-innervating nociceptor which comprises a quaternary amine. In some embodiments, the composition comprises an anti-cancer agent and a nociceptor modulating agent, nociceptor antagonist, neuropeptide modulating agent, an agent that blocks vesicle release, or agent that blocks the release or action of a neuropeptide from tumor-innervating nociceptor is selected from:

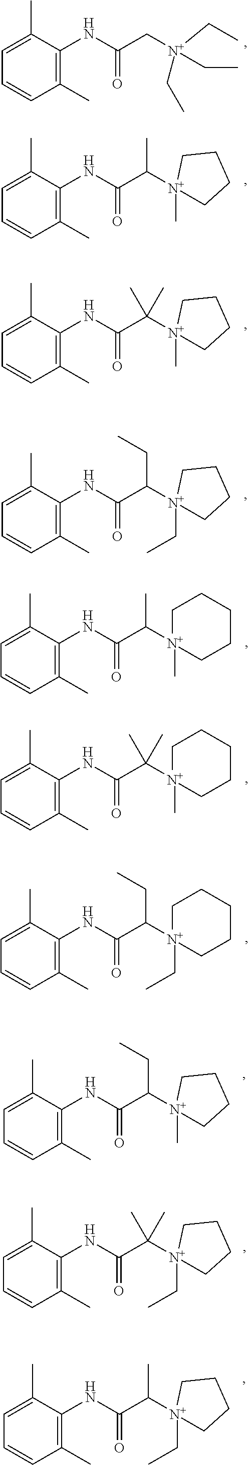

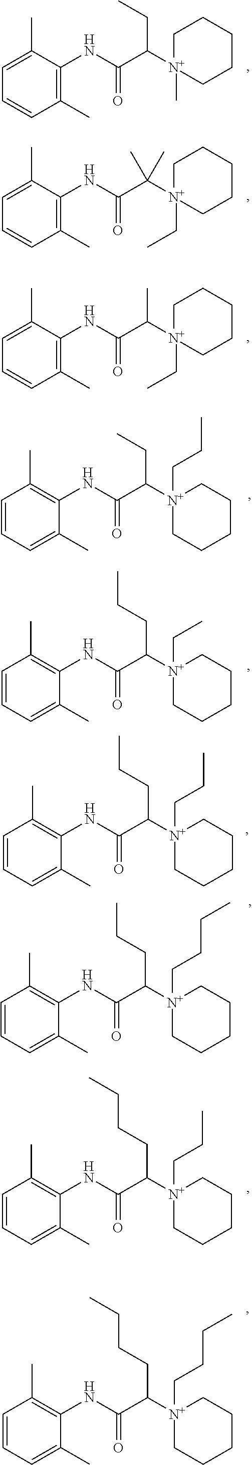

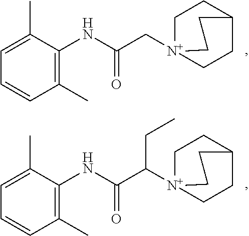 [SMILES: Cc1cccc(C)c1NC(=O)C[N+]12CCC(CC1)CC2 ; CCC(C(=O)Nc1c(C)cccc1C)[N+]12CCC(CC1)CC2]

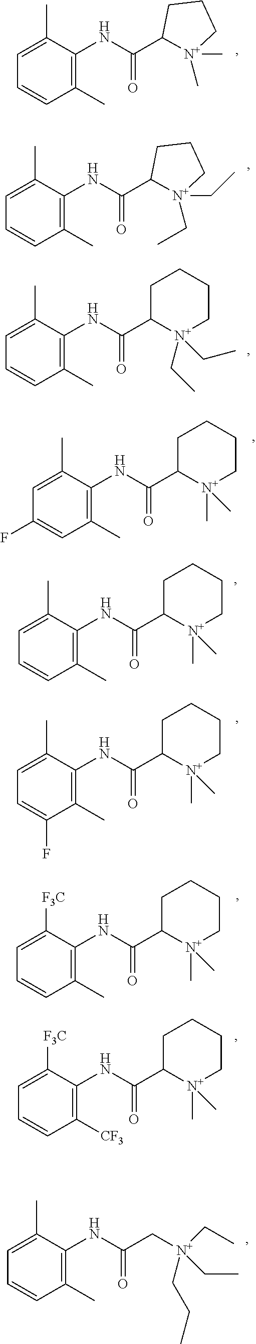

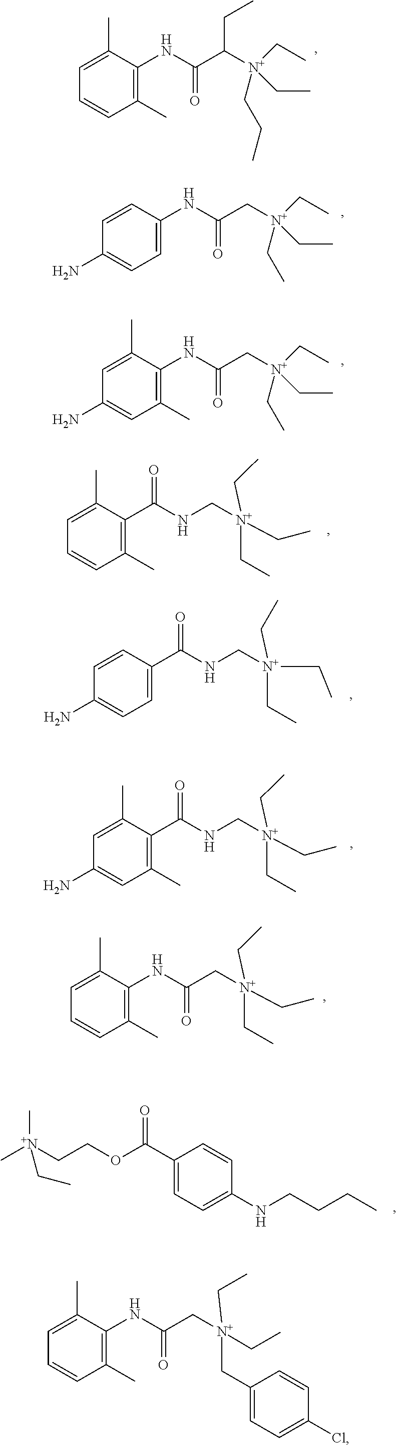

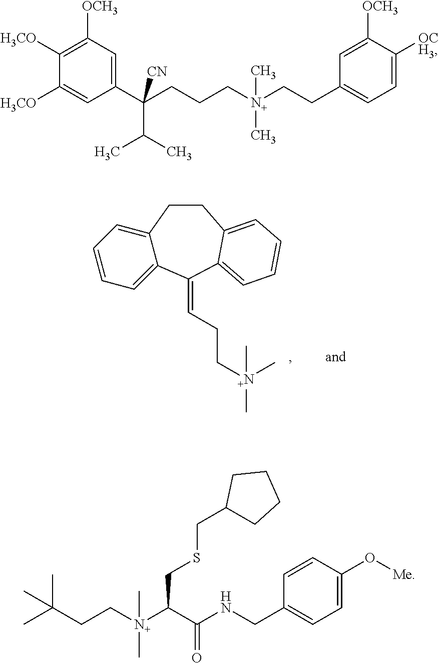 [SMILES: COc1ccc(CC[N+](C)(C)CCC[C@@](C#N)(c2cc(OC)c(OC)c(OC)c2)C(C)C)cc1OC ; C[N+](C)(C)CCC=C1c2ccccc2CCc2ccccc21 ; COc1ccc(CNC(=O)[C@H](CSCC2CCCC2)[N+](C)(C)CCC(C)(C)C)cc1]

In some embodiments, the composition comprises an anti-cancer agent and a nociceptor modulating agent, nociceptor antagonist, neuropeptide modulating agent, an agent that blocks vesicle release, or agent that blocks the release or action of a neuropeptide from tumor-innervating nociceptor of Formula (I), (IA), (II), (III), (IV), (V), (VI), (VII), (VIII), (IX), (X), (XI), (XII), (XIII), or (XIV). In some embodiments, the composition comprises an anti-cancer agent and a nociceptor modulating agent, nociceptor antagonist, neuropeptide modulating agent, an agent that blocks vesicle release, or agent that blocks the release or action of a neuropeptide from tumor-innervating nociceptor which is a quaternized or guanylated derivative of a compound as described in any of Tables 1-3. In some embodiments, the composition comprises an anti-cancer agent and a quaternary amine derivative or other permanently charged derivative of a compound selected from riluzole, mexilitine, phenytoin, carbamazepine, procaine, articaine, bupivicaine, mepivicaine, tocainide, prilocaine, diisopyramide, bencyclane, quinidine, bretylium, lifarizine, lamotrigine, flunarizine, and fluspirilene. In some embodiments, the composition comprises an anti-cancer agent and BIBN 4096. In some embodiments, the composition comprises an anti-cancer agent and a botulinum toxin. In some embodiments, the composition comprises an anti-cancer agent and BoNT/a.

In some embodiments, the additional pharmaceutical agent is an anti-cancer agent. Anti-cancer agents encompass biotherapeutic anti-cancer agents as well as chemotherapeutic agents.

Exemplary biotherapeutic anti-cancer agents include, but are not limited to, interferons, cytokines (e.g., tumor necrosis factor, interferon α, interferon γ), vaccines, hematopoietic growth factors, monoclonal serotherapy, immunostimulants and/or immunodulatory agents (e.g., IL-1, 2, 4, 6, or 12), immune cell growth factors (e.g., GM-CSF) and antibodies and fragments and variants thereof (e.g. HERCEPTIN (trastuzumab), T-DM1, AVASTIN (bevacizumab), ERBITUX (cetuximab), VECTIBIX (panitumumab), RITUXAN (rituximab), BEXXAR (tositumomab)).

Exemplary chemotherapeutic agents include, but are not limited to, anti-estrogens (e.g., tamoxifen, raloxifene, and megestrol), LHRH agonists (e.g., goscrclin and leuprolide), anti-androgens (e.g. flutamide and bicalutamide), photodynamic therapies (e.g. vertoporfin (BPD-MA), phthalocyanine, photosensitizer Pc4, and demethoxy-hypocrellin A (2BA-2-DMHA)), nitrogen mustards (e.g. cyclophosphamide, ifosfamide, trofosfamide, chlorambucil, estramustine, and melphalan), nitrosoureas (e.g. carmustine (BCNU) and lomustine (CCNU)), alkylsulphonates (e.g. busulfan and treosulfan), triazenes (e.g., dacarbazine, temozolomide), platinum containing compounds (e.g., cisplatin, carboplatin, oxaliplatin), vinca alkaloids (e.g. vincristine, vinblastine, vindesine, and vinorelbine), taxoids (e.g., paclitaxel or a paclitaxel equivalent such as nanoparticle albumin-bound paclitaxel (ABRAXANE), docosahexaenoic acid bound-paclitaxel (DHA-paclitaxel, Taxoprexin), polyglutamate bound-paclitaxel (PG-paclitaxel, paclitaxel poliglumex, CT-2103, XYOTAX), the tumor-activated prodrug (TAP) ANG1005 (Angiopep-2 bound to three molecules of paclitaxel), paclitaxel-EC-1 (paclitaxel bound to the erbB2-recognizing peptide EC-1), and glucose-conjugated paclitaxel, e.g., 2′-paclitaxel methyl 2-glucopyranosyl succinate; docetaxel, taxol), epipodophyllins (e.g. etoposide, etoposide phosphate, teniposide, topotecan, 9-aminocamptothecin, camptoirinotecan, irinotecan, crisnatol, mytomycin C), anti-metabolites, DHFR inhibitors (e.g. methotrexate, dichloromethotrexate, trimetrexate, edatrexate), IMP dehydrogenase inhibitors (e.g., mycophenolic acid, tiazofurin, ribavirin, and EICAR), ribonuclotide reductase inhibitors (e.g. hydroxyurea and deferoxamine), uracil analogs (e.g. 5-fluorouracil (5-FU), floxuridine, doxifluridine, ratitrexed, tegafur-uracil, capecitabine), cytosine analogs (e.g., cytarabine (ara C), cytosine arabinoside, and fludarabine), purine analogs (e.g. mercaptopurine and Thioguanine), Vitamin D3 analogs (e.g. EB 1089, CB 1093, and KH 1060), isoprenylation inhibitors (e.g. lovastatin), dopaminergic neurotoxins (e.g. 1-methyl-4-phenylpyridinium ion), cell cycle inhibitors (e.g. staurosporine), actinomycin (e.g. actinomycin D, dactinomycin), bleomycin (e.g. bleomycin A2, bleomycin B2, peplomycin), anthracycline (e.g. daunorubicin, doxorubicin, pegylated liposomal doxorubicin, idarubicin, epirubicin, pirarubicin, zorubicin, mitoxantrone), MDR inhibitors (e.g. verapamil), Ca²⁺ ATPase inhibitors (e.g. thapsigargin), imatinib, thalidomide, lenalidomide, tyrosine kinase inhibitors (e.g., axitinib (AG013736), bosutinib (SKI-606), cediranib (RECENTIN™, AZD2171), dasatinib (SPRYCEL®, BMS-354825), erlotinib (TARCEVA®), gefitinib (IRESSA®), imatinib (Gleevec®, CGP57148B, STI-571), lapatinib (TYKERB®, TYVERB®), lestaurtinib (CEP-701), neratinib (HKI-272), nilotinib (TASIGNA®), semaxanib (semaxinib, SU5416), sunitinib (SUTENT®, SU11248), toceranib (PALLADIA®), vandetanib (ZACTIMA®, ZD6474), vatalanib (PTK787, PTK/ZK), trastuzumab (HERCEPTIN®), bevacizumab (AVASTIN®), rituximab (RITUXAN®), cetuximab (ERBITUX®), panitumumab (VECTIBIX®), ranibizumab (Lucentis®), nilotinib (TASIGNA®), sorafenib (NEXAVAR®), everolimus (AFINITOR®), alemtuzumab (CAMPATH®), gemtuzumab ozogamicin (MYLOTARG®), temsirolimus (TORISEL®), ENMD-2076, PCI-32765, AC220, dovitinib lactate (TK1258, CHIR-258), BIBW 2992 (TOVOK™), SGX523, PF-04217903, PF-02341066, PF-299804, BMS-777607, ABT-869, MP470, BIBF 1120 (VARGATEF®), AP24534, JNJ-26483327, MGCD265, DCC-2036, BMS-690154, CEP-11981, tivozanib (AV-951), OSI-930, MM-121, XL-184, XL-647, and/or XL228), proteasome inhibitors (e.g., bortezomib (VELCADE)), mTOR inhibitors (e.g., rapamycin, temsirolimus (CCI-779), everolimus (RAD-001), ridaforolimus, AP23573 (Ariad), AZD8055 (AstraZeneca), BEZ235 (Novartis), BGT226 (Norvartis), XL765 (Sanofi Aventis), PF-4691502 (Pfizer), GDC0980 (Genetech), SF1126 (Semafoe) and OSI-027 (OSI)), oblimersen, gemcitabine, carminomycin, leucovorin, pemetrexed, cyclophosphamide, dacarbazine, procarbizine, prednisolone, dexamethasone, campathecin, plicamycin, asparaginase, aminopterin, methopterin, porfiromycin, melphalan, leurosidine, leurosine, chlorambucil, trabectedin, procarbazine, discodermolide, carminomycin, aminopterin, and hexamethyl melamine.

In some embodiments, the anti-cancer agent is dacarbazine. In some embodiments, the anti-cancer agent is cisplatin.

In some embodiments, the composition comprises dacarbazine, QX-314, and optionally a pharmaceutically acceptable excipient. In some embodiments, the composition comprises dacarbazine, BoNT/a, and optionally a pharmaceutically acceptable excipient. In some embodiments, the composition comprises dacarbazine, BIBN4096, and optionally a pharmaceutically acceptable excipient.

In some embodiments, the composition comprises cisplatin, QX-314, and optionally a pharmaceutically acceptable excipient. In some embodiments, the composition comprises cisplatin, BoNT/a, and optionally a pharmaceutically acceptable excipient. In some embodiments, the composition comprises cisplatin, BIBN4096, and optionally a pharmaceutically acceptable excipient.

In some embodiments, the composition comprises dacarbazine, QX-314, and a pharmaceutically acceptable carrier or excipient. In some embodiments, the composition comprises dacarbazine, BoNT/a, and a pharmaceutically acceptable carrier or excipient. In some embodiments, the composition comprises dacarbazine, BIBN4096, and a pharmaceutically acceptable carrier or excipient.

In some embodiments, the composition comprises cisplatin, QX-314, and a pharmaceutically acceptable carrier or excipient. In some embodiments, the composition comprises cisplatin, BoNT/a, and a pharmaceutically acceptable carrier or excipient. In some embodiments, the composition comprises cisplatin, BIBN4096, and a pharmaceutically acceptable carrier or excipient.

In some embodiments, dacarbazine is used in combination with QX-314. In some embodiments, dacarbazine is used in combination with BoNT/a. In some embodiments, dacarbazine is used in combination with BIBN4096. In some embodiments, cisplatin is used in combination with QX-314. In some embodiments, cisplatin is used in combination with BoNT/a. In some embodiments, cisplatin is used in combination with BIBN4096.

Pharmaceutical compositions described herein can be prepared by any method known in the art of pharmacology. In general, such preparatory methods include bringing the compound described herein (i.e., the “active ingredient”) into association with a carrier or excipient, and/or one or more other accessory ingredients, and then, if necessary and/or desirable, shaping, and/or packaging the product into a desired single- or multi-dose unit.

Pharmaceutical compositions can be prepared, packaged, and/or sold in bulk, as a single unit dose, and/or as a plurality of single unit doses. A “unit dose” is a discrete amount of the pharmaceutical composition comprising a predetermined amount of the active ingredient. The amount of the active ingredient is generally equal to the dosage of the active ingredient which would be administered to a subject and/or a convenient fraction of such a dosage, such as one-half or one-third of such a dosage.

Relative amounts of the active ingredient, the pharmaceutically acceptable excipient, and/or any additional ingredients in a pharmaceutical composition described herein will vary, depending upon the identity, size, and/or condition of the subject treated and further depending upon the route by which the composition is to be administered. The composition may comprise between 0.1% and 100% (w/w) active ingredient.

Pharmaceutically acceptable excipients used in the manufacture of provided pharmaceutical compositions include inert diluents, dispersing and/or granulating agents, surface active agents and/or emulsifiers, disintegrating agents, binding agents, preservatives, buffering agents, lubricating agents, and/or oils. Excipients such as cocoa butter and suppository waxes, coloring agents, coating agents, sweetening, flavoring, and perfuming agents may also be present in the composition.

Exemplary diluents include calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, and mixtures thereof.

Exemplary granulating and/or dispersing agents include potato starch, corn starch, tapioca starch, sodium starch glycolate, clays, alginic acid, guar gum, citrus pulp, agar, bentonite, cellulose, and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (Veegum), sodium lauryl sulfate, quaternary ammonium compounds, and mixtures thereof.

Exemplary surface active agents and/or emulsifiers include natural emulsifiers (e.g., acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g., bentonite (aluminum silicate) and Veegum (magnesium aluminum silicate)), long chain amino acid derivatives, high molecular weight alcohols (e.g., stearyl alcohol, cetyl alcohol, oleyl alcohol, triacetin monostearate, ethylene glycol distearate, glyceryl monostearate, and propylene glycol monostearate, polyvinyl alcohol), carbomers (e.g., carboxy polymethylene, polyacrylic acid, acrylic acid polymer, and carboxyvinyl polymer), carrageenan, cellulosic derivatives (e.g., carboxymethylcellulose sodium, powdered cellulose, hydroxymethyl cellulose, hydroxypropyl cellulose, hydroxypropyl methylcellulose, methylcellulose), sorbitan fatty acid esters (e.g., polyoxyethylene sorbitan monolaurate (Tween® 20), polyoxyethylene sorbitan (Tween® 60), polyoxyethylene sorbitan monooleate (Tween® 80), sorbitan monopalmitate (Span® 40), sorbitan monostearate (Span® 60), sorbitan tristearate (Span® 65), glyceryl monooleate, sorbitan monooleate (Span® 80), polyoxyethylene esters (e.g., polyoxyethylene monostearate (Myrj® 45), polyoxyethylene hydrogenated castor oil, polyethoxylated castor oil, polyoxymethylene stearate, and Solutol®), sucrose fatty acid esters, polyethylene glycol fatty acid esters (e.g., Cremophor®), polyoxyethylene ethers, (e.g., polyoxyethylene lauryl ether (Brij® 30)), poly(vinyl-pyrrolidone), diethylene glycol monolaurate, triethanolamine oleate, sodium oleate, potassium oleate, ethyl oleate, oleic acid, ethyl laurate, sodium lauryl sulfate, Pluronic® F-68, poloxamer P-188, cetrimonium bromide, cetylpyridinium chloride, benzalkonium chloride, docusate sodium, and/or mixtures thereof.

Exemplary binding agents include starch (e.g., cornstarch and starch paste), gelatin, sugars (e.g., sucrose, glucose, dextrose, dextrin, molasses, lactose, lactitol, mannitol, etc.), natural and synthetic gums (e.g., acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (Veegum®), and larch arabogalactan), alginates, polyethylene oxide, polyethylene glycol, inorganic calcium salts, silicic acid, polymethacrylates, waxes, water, alcohol, and/or mixtures thereof.

Exemplary preservatives include antioxidants, chelating agents, antimicrobial preservatives, antifungal preservatives, antiprotozoan preservatives, alcohol preservatives, acidic preservatives, and other preservatives. In certain embodiments, the preservative is an antioxidant. In other embodiments, the preservative is a chelating agent.

Exemplary antioxidants include alpha tocopherol, ascorbic acid, ascorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and sodium sulfite.

Exemplary chelating agents include ethylenediaminetetraacetic acid (EDTA) and salts and hydrates thereof (e.g., sodium edetate, disodium edetate, trisodium edetate, calcium disodium edetate, dipotassium edetate, and the like), citric acid and salts and hydrates thereof (e.g., citric acid monohydrate), fumaric acid and salts and hydrates thereof, malic acid and salts and hydrates thereof, phosphoric acid and salts and hydrates thereof, and tartaric acid and salts and hydrates thereof. Exemplary antimicrobial preservatives include benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and thimerosal.

Exemplary antifungal preservatives include butyl paraben, methyl paraben, ethyl paraben, propyl paraben, benzoic acid, hydroxybenzoic acid, potassium benzoate, potassium sorbate, sodium benzoate, sodium propionate, and sorbic acid.

Exemplary alcohol preservatives include ethanol, polyethylene glycol, phenol, phenolic compounds, bisphenol, chlorobutanol, hydroxybenzoate, and phenylethyl alcohol.

Exemplary acidic preservatives include vitamin A, vitamin C, vitamin E, beta-carotene, citric acid, acetic acid, dehydroacetic acid, ascorbic acid, sorbic acid, and phytic acid.

Other preservatives include tocopherol, tocopherol acetate, deteroxime mesylate, cetrimide, butylated hydroxyanisol (BHA), butylated hydroxytoluened (BHT), ethylenediamine, sodium lauryl sulfate (SLS), sodium lauryl ether sulfate (SLES), sodium bisulfite, sodium metabisulfite, potassium sulfite, potassium metabisulfite, Glydant® Plus, Phenonip®, methylparaben, Germall® 115, Germaben® II, Neolone®, Kathon®, and Euxyl®.

Exemplary buffering agents include citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, and mixtures thereof.

Exemplary lubricating agents include magnesium stearate, calcium stearate, stearic acid, silica, talc, malt, glyceryl behanate, hydrogenated vegetable oils, polyethylene glycol, sodium benzoate, sodium acetate, sodium chloride, leucine, magnesium lauryl sulfate, sodium lauryl sulfate, and mixtures thereof.

Exemplary natural oils include almond, apricot kernel, avocado, babassu, bergamot, black current seed, borage, cade, camomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils. Exemplary synthetic oils include, but are not limited to, butyl stearate, caprylic triglyceride, capric triglyceride, cyclomethicone, diethyl sebacate, dimethicone 360, isopropyl myristate, mineral oil, octyldodecanol, oleyl alcohol, silicone oil, and mixtures thereof.

Liquid dosage forms for oral and parenteral administration include pharmaceutically acceptable emulsions, microemulsions, solutions, suspensions, syrups and elixirs. In addition to the active ingredients, the liquid dosage forms may comprise inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (e.g., cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can include adjuvants such as wetting agents, emulsifying and suspending agents, sweetening, flavoring, and perfuming agents. In certain embodiments for parenteral administration, the conjugates described herein are mixed with solubilizing agents such as Cremophor®, alcohols, oils, modified oils, glycols, polysorbates, cyclodextrins, polymers, and mixtures thereof.

Injectable preparations, for example, sterile injectable aqueous or oleaginous suspensions can be formulated according to the known art using suitable dispersing or wetting agents and suspending agents. The sterile injectable preparation can be a sterile injectable solution, suspension, or emulsion in a nontoxic parenterally acceptable diluent or solvent, for example, as a solution in 1,3-butanediol. Among the acceptable vehicles and solvents that can be employed are water, Ringer's solution, U.S.P., and isotonic sodium chloride solution. In addition, sterile, fixed oils are conventionally employed as a solvent or suspending medium. For this purpose, any bland fixed oil can be employed including synthetic mono- or di-glycerides. In addition, fatty acids such as oleic acid are used in the preparation of injectables.

The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a drug, it is often desirable to slow the absorption of the drug from subcutaneous or intramuscular injection. This can be accomplished by the use of a liquid suspension of crystalline or amorphous material with poor water solubility. The rate of absorption of the drug then depends upon its rate of dissolution, which, in turn, may depend upon crystal size and crystalline form. Alternatively, delayed absorption of a parenterally administered drug form may be accomplished by dissolving or suspending the drug in an oil vehicle.

Compositions for rectal or vaginal administration are typically suppositories which can be prepared by mixing the conjugates described herein with suitable non-irritating excipients or carriers such as cocoa butter, polyethylene glycol, or a suppository wax which are solid at ambient temperature but liquid at body temperature and therefore melt in the rectum or vaginal cavity and release the active ingredient.

Solid dosage forms for oral administration include capsules, tablets, pills, powders, and granules. In such solid dosage forms, the active ingredient is mixed with at least one inert, pharmaceutically acceptable excipient or carrier such as sodium citrate or dicalcium phosphate and/or (a) fillers or extenders such as starches, lactose, sucrose, glucose, mannitol, and silicic acid, (b) binders such as, for example, carboxymethylcellulose, alginates, gelatin, polyvinylpyrrolidinone, sucrose, and acacia, (c) humectants such as glycerol, (d) disintegrating agents such as agar, calcium carbonate, potato or tapioca starch, alginic acid, certain silicates, and sodium carbonate, (e) solution retarding agents such as paraffin, (f) absorption accelerators such as quaternary ammonium compounds, (g) wetting agents such as, for example, cetyl alcohol and glycerol monostearate, (h) absorbents such as kaolin and bentonite clay, and (i) lubricants such as talc, calcium stearate, magnesium stearate, solid polyethylene glycols, sodium lauryl sulfate, and mixtures thereof. In the case of capsules, tablets, and pills, the dosage form may include a buffering agent.

Solid compositions of a similar type can be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the art of pharmacology. They may optionally comprise opacifying agents and can be of a composition that they release the active ingredient(s) only, or preferentially, in a certain part of the intestinal tract, optionally, in a delayed manner. Examples of encapsulating compositions which can be used include polymeric substances and waxes. Solid compositions of a similar type can be employed as fillers in soft and hard-filled gelatin capsules using such excipients as lactose or milk sugar as well as high molecular weight polethylene glycols and the like.

The active ingredient can be in a micro-encapsulated form with one or more excipients as noted above. The solid dosage forms of tablets, dragees, capsules, pills, and granules can be prepared with coatings and shells such as enteric coatings, release controlling coatings, and other coatings well known in the pharmaceutical formulating art. In such solid dosage forms the active ingredient can be admixed with at least one inert diluent such as sucrose, lactose, or starch. Such dosage forms may comprise, as is normal practice, additional substances other than inert diluents, e.g., tableting lubricants and other tableting aids such a magnesium stearate and microcrystalline cellulose. In the case of capsules, tablets and pills, the dosage forms may comprise buffering agents. They may optionally comprise opacifying agents and can be of a composition that they release the active ingredient(s) only, or preferentially, in a certain part of the intestinal tract, optionally, in a delayed manner. Examples of encapsulating agents which can be used include polymeric substances and waxes.

Dosage forms for topical and/or transdermal administration of a compound described herein may include ointments, pastes, creams, lotions, gels, powders, solutions, sprays, inhalants, and/or patches. Generally, the active ingredient is admixed under sterile conditions with a pharmaceutically acceptable carrier or excipient and/or any needed preservatives and/or buffers as can be required. Additionally, the present disclosure contemplates the use of transdermal patches, which often have the added advantage of providing controlled delivery of an active ingredient to the body. Such dosage forms can be prepared, for example, by dissolving and/or dispensing the active ingredient in the proper medium. Alternatively or additionally, the rate can be controlled by either providing a rate controlling membrane and/or by dispersing the active ingredient in a polymer matrix and/or gel.

Suitable devices for use in delivering intradermal pharmaceutical compositions described herein include short needle devices. Intradermal compositions can be administered by devices which limit the effective penetration length of a needle into the skin. Alternatively or additionally, conventional syringes can be used in the classical mantoux method of intradermal administration. Jet injection devices which deliver liquid formulations to the dermis via a liquid jet injector and/or via a needle which pierces the stratum corneum and produces a jet which reaches the dermis are suitable. Ballistic powder/particle delivery devices which use compressed gas to accelerate the compound in powder form through the outer layers of the skin to the dermis are suitable.

Formulations suitable for topical administration include, but are not limited to, liquid and/or semi-liquid preparations such as liniments, lotions, oil-in-water and/or water-in-oil emulsions such as creams, ointments, and/or pastes, and/or solutions and/or suspensions. Topically administrable formulations may, for example, comprise from about 1% to about 10% (w/w) active ingredient, although the concentration of the active ingredient can be as high as the solubility limit of the active ingredient in the solvent. Formulations for topical administration may further comprise one or more of the additional ingredients described herein.

A pharmaceutical composition described herein can be prepared, packaged, and/or sold in a formulation suitable for pulmonary administration via the buccal cavity. Such a formulation may comprise dry particles which comprise the active ingredient and which have a diameter in the range from about 0.5 to about 7 nanometers, or from about 1 to about 6 nanometers. Such compositions are conveniently in the form of dry powders for administration using a device comprising a dry powder reservoir to which a stream of propellant can be directed to disperse the powder and/or using a self-propelling solvent/powder dispensing container such as a device comprising the active ingredient dissolved and/or suspended in a low-boiling propellant in a sealed container. Such powders comprise particles wherein at least 98% of the particles by weight have a diameter greater than 0.5 nanometers and at least 95% of the particles by number have a diameter less than 7 nanometers. Alternatively, at least 95% of the particles by weight have a diameter greater than 1 nanometer and at least 90% of the particles by number have a diameter less than 6 nanometers. Dry powder compositions may include a solid fine powder diluent such as sugar and are conveniently provided in a unit dose form.

Low boiling propellants generally include liquid propellants having a boiling point of below 65° F. at atmospheric pressure. Generally, the propellant may constitute 50 to 99.9% (w/w) of the composition, and the active ingredient may constitute 0.1 to 20% (w/w) of the composition. The propellant may further comprise additional ingredients such as a liquid non-ionic and/or solid anionic surfactant and/or a solid diluent (which may have a particle size of the same order as particles comprising the active ingredient).

Pharmaceutical compositions described herein formulated for pulmonary delivery may provide the active ingredient in the form of droplets of a solution and/or suspension. Such formulations can be prepared, packaged, and/or sold as aqueous and/or dilute alcoholic solutions and/or suspensions, optionally sterile, comprising the active ingredient, and may conveniently be administered using any nebulization and/or atomization device. Such formulations may further comprise one or more additional ingredients including, but not limited to, a flavoring agent such as saccharin sodium, a volatile oil, a buffering agent, a surface active agent, and/or a preservative such as methylhydroxybenzoate. The droplets provided by this route of administration may have an average diameter in the range from about 0.1 to about 200 nanometers.

Formulations described herein as being useful for pulmonary delivery are useful for intranasal delivery of a pharmaceutical composition described herein. Another formulation suitable for intranasal administration is a coarse powder comprising the active ingredient and having an average particle from about 0.2 to 500 micrometers. Such a formulation is administered by rapid inhalation through the nasal passage from a container of the powder held close to the nares.

Formulations for nasal administration may, for example, comprise from about as little as 0.1% (w/w) to as much as 100% (w/w) of the active ingredient, and may comprise one or more of the additional ingredients described herein. A pharmaceutical composition described herein can be prepared, packaged, and/or sold in a formulation for buccal administration. Such formulations may, for example, be in the form of tablets and/or lozenges made using conventional methods, and may contain, for example, 0.1 to 20% (w/w) active ingredient, the balance comprising an orally dissolvable and/or degradable composition and, optionally, one or more of the additional ingredients described herein. Alternately, formulations for buccal administration may comprise a powder and/or an aerosolized and/or atomized solution and/or suspension comprising the active ingredient. Such powdered, aerosolized, and/or aerosolized formulations, when dispersed, may have an average particle and/or droplet size in the range from about 0.1 to about 200 nanometers, and may further comprise one or more of the additional ingredients described herein.

A pharmaceutical composition described herein can be prepared, packaged, and/or sold in a formulation for ophthalmic administration. Such formulations may, for example, be in the form of eye drops including, for example, a 0.1-1.0% (w/w) solution and/or suspension of the active ingredient in an aqueous or oily liquid carrier or excipient. Such drops may further comprise buffering agents, salts, and/or one or more other of the additional ingredients described herein. Other ophthalmically-administrable formulations which are useful include those which comprise the active ingredient in microcrystalline form and/or in a liposomal preparation. Ear drops and/or eye drops are also contemplated as being within the scope of this disclosure.

Although the descriptions of pharmaceutical compositions provided herein are directed to pharmaceutical compositions which are suitable for administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and/or perform such modification with ordinary experimentation.

Compounds described herein are typically formulated in dosage unit form for ease of administration and uniformity of dosage. It will be understood, however, that the total daily usage of the compositions described herein will be decided by a physician within the scope of sound medical judgment. The specific therapeutically effective dose level for any particular subject or organism will depend upon a variety of factors including the disease being treated and the severity of the disorder; the activity of the specific active ingredient employed; the specific composition employed; the age, body weight, general health, sex, and diet of the subject; the time of administration, route of administration, and rate of excretion of the specific active ingredient employed; the duration of the treatment; drugs used in combination or coincidental with the specific active ingredient employed; and like factors well known in the medical arts.

The compounds described herein and compositions provided herein can be administered by any route, including enteral (e.g., oral), parenteral, intravenous, intramuscular, intra-arterial, intramedullary, intrathecal, subcutaneous, intraventricular, transdermal, interdermal, rectal, intravaginal, intraperitoneal, topical (as by powders, ointments, creams, and/or drops), mucosal, nasal, bucal, sublingual; by intratracheal instillation, bronchial instillation, and/or inhalation; and/or as an oral spray, nasal spray, and/or aerosol. Specifically contemplated routes are oral administration, intravenous administration (e.g., systemic intravenous injection), regional administration via blood and/or lymph supply, and/or direct administration to an affected site. In general, the most appropriate route of administration will depend upon a variety of factors including the nature of the agent (e.g., its stability in the environment of the gastrointestinal tract), and/or the condition of the subject (e.g., whether the subject is able to tolerate oral administration). In certain embodiments, the compound or pharmaceutical composition described herein is suitable for topical administration to the eye of a subject.

The exact amount of a compound required to achieve an effective amount will vary from subject to subject, depending, for example, on species, age, and general condition of a subject, severity of the side effects or disorder, identity of the particular compound, mode of administration, and the like. An effective amount may be included in a single dose (e.g., single oral dose) or multiple doses (e.g., multiple oral doses). In certain embodiments, when multiple doses are administered to a subject, any two doses of the multiple doses include different or substantially the same amounts of a compound described herein. In certain embodiments, when multiple doses are administered to a subject, the frequency of administering the multiple doses to the subject is three doses a day, two doses a day, one dose a day, one dose every other day, one dose every third day, one dose every week, one dose every two weeks, one dose every three weeks, or one dose every four weeks. In certain embodiments, the frequency of administering the multiple doses to the subject is one dose per day. In certain embodiments, the frequency of administering the multiple doses to the subject is two doses per day. In certain embodiments, the frequency of administering the multiple doses to the subject is three doses per day. In certain embodiments, when multiple doses are administered to a subject, the duration between the first dose and last dose of the multiple doses is one day, two days, four days, one week, two weeks, three weeks, one month, two months, three months, four months, six months, nine months, one year, two years, three years, four years, five years, seven years, ten years, fifteen years, twenty years, or the lifetime of the subject. In certain embodiments, the duration between the first dose and last dose of the multiple doses is three months, six months, or one year. In certain embodiments, the duration between the first dose and last dose of the multiple doses is the lifetime of the subject. In certain embodiments, a dose (e.g., a single dose, or any dose of multiple doses) described herein includes independently between 0.1 μg and 1 μg, between 0.001 mg and 0.01 mg, between 0.01 mg and 0.1 mg, between 0.1 mg and 1 mg, between 1 mg and 3 mg, between 3 mg and 10 mg, between 10 mg and 30 mg, between 30 mg and 100 mg, between 100 mg and 300 mg, between 300 mg and 1,000 mg, or between 1 g and 10 g, inclusive, of a compound described herein. In certain embodiments, a dose described herein includes independently between 1 mg and 3 mg, inclusive, of a compound described herein. In certain embodiments, a dose described herein includes independently between 3 mg and 10 mg, inclusive, of a compound described herein. In certain embodiments, a dose described herein includes independently between 10 mg and 30 mg, inclusive, of a compound described herein. In certain embodiments, a dose described herein includes independently between 30 mg and 100 mg, inclusive, of a compound described herein.

Dose ranges as described herein provide guidance for the administration of provided pharmaceutical compositions to an adult. The amount to be administered to, for example, a child or an adolescent can be determined by a medical practitioner or person skilled in the art and can be lower or the same as that administered to an adult.

A compound, as described herein, can be administered in combination with one or more additional pharmaceutical agents (e.g., therapeutically and/or prophylactically active agents). In some embodiments, a compound disclosed herein is administered with an anti-cancer agent. The compounds can be administered in combination with additional pharmaceutical agents that improve their activity (e.g., activity (e.g., potency and/or efficacy) in treating a disease in a subject in need thereof, in preventing a disease in a subject in need thereof, in reducing the risk to develop a disease in a subject in need thereof, improve bioavailability, improve safety, reduce drug resistance, reduce and/or modify metabolism, inhibit excretion, and/or modify distribution in a subject. It will also be appreciated that the therapy employed may achieve a desired effect for the same disorder, and/or it may achieve different effects. In some embodiments, the additional pharmaceutical agent achieves a desired effect for the same disorder. In some embodiments, the additional pharmaceutical agent achieves different effects.

The compound or composition can be administered concurrently with, prior to, or subsequent to one or more additional pharmaceutical agents, which may be useful as, e.g., combination therapies.

Pharmaceutical agents include therapeutically active agents. Pharmaceutical agents also include prophylactically active agents. Pharmaceutical agents include small organic molecules such as drug compounds (e.g., compounds approved for human or veterinary use by the U.S. Food and Drug Administration as provided in the Code of Federal Regulations (CFR)), peptides, proteins, carbohydrates, monosaccharides, oligosaccharides, polysaccharides, nucleoproteins, mucoproteins, lipoproteins, synthetic polypeptides or proteins, small molecules linked to proteins, glycoproteins, steroids, nucleic acids, DNAs, RNAs, nucleotides, nucleosides, oligonucleotides, antisense oligonucleotides, lipids, hormones, vitamins, and cells. In certain embodiments, the additional pharmaceutical agent is a pharmaceutical agent useful for treating and/or preventing a disease (e.g., proliferative disease, hematological disease, neurological disease, painful condition, psychiatric disorder, or metabolic disorder). Each additional pharmaceutical agent may be administered at a dose and/or on a time schedule determined for that pharmaceutical agent. The additional pharmaceutical agents may also be administered together with each other and/or with the compound or composition described herein in a single dose or composition or administered separately in different doses or compositions. The particular combination to employ in a regimen will take into account compatibility of the compound described herein with the additional pharmaceutical agent(s) and/or the desired therapeutic and/or prophylactic effect to be achieved. In general, it is expected that the additional pharmaceutical agent(s) in combination be utilized at levels that do not exceed the levels at which they are utilized individually. In some embodiments, the levels utilized in combination will be lower than those utilized individually.

Also encompassed by the disclosure are kits (e.g., pharmaceutical packs). The kits provided may comprise a pharmaceutical composition or compound described herein and instructions for use. Also encompassed by the disclosure are kits (e.g., pharmaceutical packs). The kits provided may comprise a pharmaceutical composition or compound described herein and instructions for administration of the composition or compound to a subject. The kits provided may comprise a pharmaceutical composition or compound described herein and instructions for administration of the composition or compound to a cancer patient.

Also encompassed by the disclosure are kits (e.g., pharmaceutical packs). The kits provided may comprise a pharmaceutical composition or compound described herein and a container (e.g., a vial, ampule, bottle, syringe, and/or dispenser package, or other suitable container). In some embodiments, provided kits may optionally further include a second container comprising a pharmaceutical excipient for dilution or suspension of a pharmaceutical composition or compound described herein. In some embodiments, the pharmaceutical composition or compound described herein provided in the first container and the second container are combined to form one unit dosage form.

Thus, in one aspect, provided are kits including a first container comprising a compound or pharmaceutical composition described herein. In certain embodiments, the kits are useful for treating a disease (e.g., cancer) in a subject in need thereof. In certain embodiments, the kits are useful for preventing a disease (e.g., cancer) in a subject in need thereof.

In certain embodiments, a kit described herein further includes instructions for using the kit. A kit described herein may also include information as required by a regulatory agency such as the U.S. Food and Drug Administration (FDA). In certain embodiments, the information included in the kits is prescribing information. In certain embodiments, the kits and instructions provide for treating a disease (e.g., cancer) in a subject in need thereof. In certain embodiments, the kits and instructions provide for preventing a disease (e.g., cancer) in a subject in need thereof. A kit described herein may include one or more additional pharmaceutical agents described herein as a separate composition.

### EXAMPLES

In order that the present disclosure may be more fully understood, the following examples are set forth. The synthetic and biological examples described in this application are offered to illustrate the compounds, pharmaceutical compositions, methods, and uses provided herein and are not to be construed in any way as limiting their scope.

### Synthesis

The synthesis of compounds described herein may involve the selective protection and deprotection of alcohols, amines, ketones, sulfhydryls or carboxyl functional groups of the parent compound, the linker, the bulky group, and/or the charged group. For example, commonly used protecting groups for amines include carbamates, such as tert-butyl, benzyl, 2,2,2-trichloroethyl, 2-trimethylsilylethyl, 9-fluorenylmethyl, allyl, and m-nitrophenyl. Other commonly used protecting groups for amines include amides, such as formamides, acetamides, trifluoroacetamides, sulfonamides, trifluoromethanesulfonyl amides, trimethylsilylethanesulfonamides, and tert-butylsulfonyl amides. Examples of commonly used protecting groups for carboxyls include esters, such as methyl, ethyl, tert-butyl, 9-fluorenylmethyl, 2-(trimethylsilyl)ethoxy methyl, benzyl, diphenylmethyl, O-nitrobenzyl, ortho-esters, and halo-esters. Examples of commonly used protecting groups for alcohols include ethers, such as methyl, methoxymethyl, methoxyethoxymethyl, methylthiomethyl, benzyloxymethyl, tetrahydropyranyl, ethoxyethyl, benzyl, 2-napthylmethyl, O-nitrobenzyl, P-nitrobenzyl, P-methoxybenzyl, 9-phenylxanthyl, trityl (including methoxy-trityls), and silyl ethers. Examples of commonly used protecting groups for sulfhydryls include many of the same protecting groups used for hydroxyls. In addition, sulfhydryls can be protected in a reduced form (e.g., as disulfides) or an oxidized form (e.g., as sulfonic acids, sulfonic esters, or sulfonic amides). Protecting groups can be chosen such that selective conditions (e.g., acidic conditions, basic conditions, catalysis by a nucleophile, catalysis by a Lewis acid, or hydrogenation) are required to remove each, exclusive of other protecting groups in a molecule. The conditions required for the addition of protecting groups to amine, alcohol, sulfhydryl, and carboxyl functionalities and the conditions required for their removal are provided in detail in T. W. Green and P. G. M. Wuts, Protective Groups in Organic Synthesis (2^{nd} Ed.), John Wiley & Sons, 1991 and P. J. Kocienski, Protecting Groups, Georg Thieme Verlag, 1994.

Charged ion channel blockers (e.g., quaternary amines) can be prepared using techniques familiar to those skilled in the art. The modifications can be made, for example, by alkylation of the parent compound using the techniques described by J. March, Advanced Organic Chemistry: Reactions, Mechanisms and Structure, John Wiley & Sons, Inc., 1992, page 617. The conversion of amino groups to guanidine groups can be accomplished using standard synthetic protocols. For example, Mosher has described a general method for preparing mono-substituted guanidines by reaction of aminoiminomethanesulfonic acid with amines (Kim et al., Tetrahedron Lett. 29:3183 (1988)). A more convenient method for guanylation of primary and secondary amines was developed by Bernatowicz employing 1H-pyrazole-1-carboxamidine hydrochloride; 1-H-pyrazole-1-(N,N′-bis(tert-butoxycarbonyl) carboxamidine; or 1-H-pyrazole-1-(N,N′-bis(benzyloxycarbonyl)carboxamidine. These reagents react with amines to give mono-substituted guanidines (see Bernatowicz et al., J. Org. Chem. 57:2497 (1992); and Bernatowicz et al., Tetrahedron Lett. 34:3389 (1993)). In addition, thioureas and S-alkyl-isothioureas have been shown to be useful intermediates in the syntheses of substituted guanidines (Poss et al., Tetrahedron Lett. 33:5933 (1992)). In certain embodiments, the guanidine is part of a heterocyclic ring having two nitrogen atoms (see, for example, the structures below). The ring system can include an alkylene or

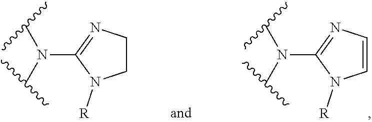

alkenylene of from 2 to 4 carbon atoms, e.g., ring systems of 5, 6, and 7-membered rings. Such ring systems can be prepared, for example, using the methods disclosed by Schlama et al., J. Org. Chem. 62:4200 (1997).

Compounds described herein (e.g., in Tables 1-3) can be prepared by alkylation of an amine nitrogen in the parent compound as shown in Scheme 1.

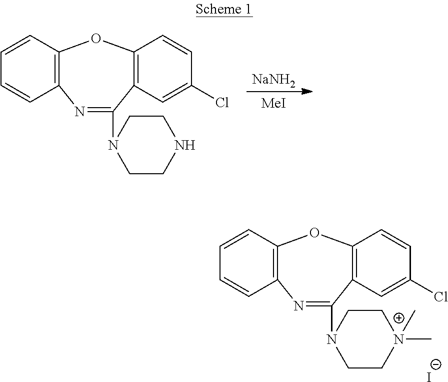

Alternatively, charged ion channel blockers can be prepared by introduction of a guanidine group. The parent compound can be reacted with a cynamide, e.g., methylcyanamide, as shown in Scheme 2 or pyrazole-1-carboxamidine derivatives as shown in Scheme 3 where Z is H or a suitable protecting group. Alternatively, the parent compound can be reacted with cyanogens bromide followed by reaction with methylchloroaluminum amide as shown in Scheme 4. Reagents such as 2-(methylthio)-2-imidazoline can also be used to prepare suitably functionalized derivatives (Scheme 5).

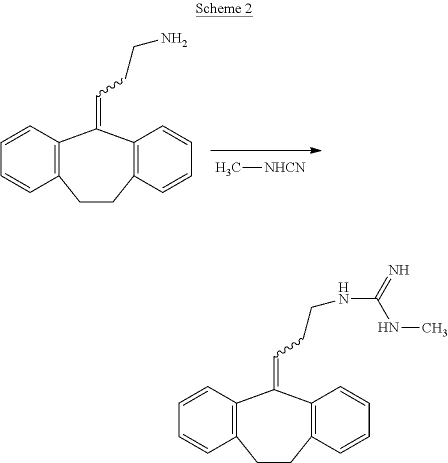

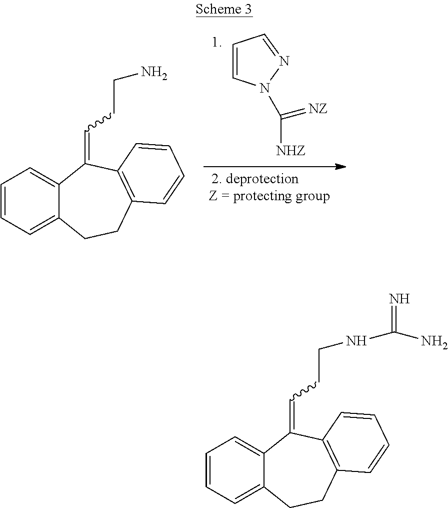

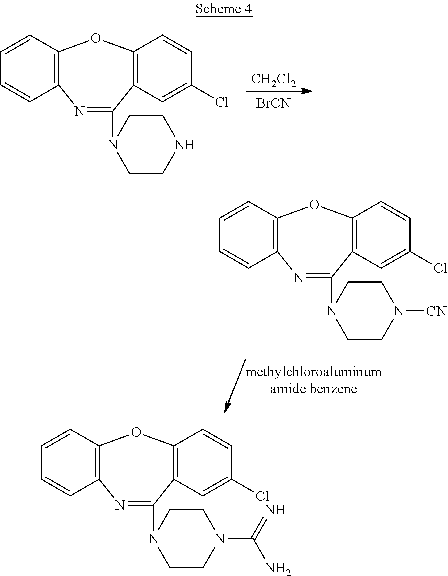

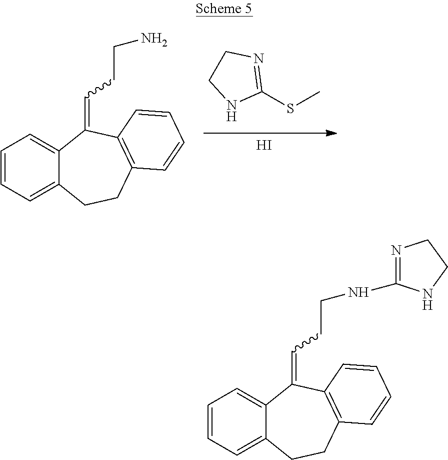

Any compounds containing an amine nitrogen atom (e.g., a compound selected from Compounds (1)-(563) or a compound according to Formulas (IA)-(XIV)) can be modified as shown in Schemes 1-5.

### Example 1

Doublecortin-expressing neural progenitors initiate the neurogenesis found in prostate cancer (1). These autonomic neurons facilitate tumor development and dissemination (2) in part via nerve-derived noradrenaline which activates an angiogenic switch that fuels cancer growth (3, 4). While head and neck tumor-associated sensory nerves appear to transdifferentiate into adrenergic neurons following loss of TP53 (5), the overall impact of tumor neo-innervation by pain-initiating sensory neurons remains unclear. Here, it was found that malignant melanoma skin cancer cells directly interact with nociceptors by increasing neurite outgrowth, responsiveness to noxious ligands and neuropeptide release. In turn, CGRP, one such nociceptor-produced neuropeptide, directly increase exhaustion of cytotoxic T cells (PD1⁺Lag3⁺Tim3⁺INFγ⁻), limiting their capacity to eliminate melanoma cells. Genetic TRPV1 or Na_{V}1.8 lineage ablation, local pharmacological silencing or blockade of neuropeptide release from tumor-innervating nociceptors, as well as the antagonism of the CGRP receptor RAMP1, blunt tumor-infiltrating leukocyte exhaustion and tumor growth, nearly tripling B16F10-inoculated mouse survival. Single-cell RNA sequencing of human melanoma has revealed that Ramp1⁺ CD8⁺ T-cells are severely exhausted and increased in biopsy expression of Calca, the gene encoding for CGRP, correlated with worsened patient′ prognosis. It was concluded that reducing CGRP release from tumor-innervating nociceptors, by eliminating their immunomodulatory action on cytotoxic CD8⁺ T-cells, constitute an unexpected strategy to safeguard anti-tumor immunity.

Cytotoxic T-cells express a variety of receptors, including PD-1 (Programmed Death-1), Tim-3 (T cell immunoglobulin and mucin domain-containing protein 3), and Lag-3 (Lymphocyte Activation Gene-3)(6-9), which inhibit T-cell function after being activated by their cognate ligands. These checkpoint receptors ensure that immune responses to damage or infection are kept in check, preventing overly intense responses that might damage healthy cells(10). Tumor cells express ligands for these immune checkpoints, which, when activated, block the cytolytic functions of T-cells, favoring cancer cells survival (10-12). The posit role for neo-innervation in cancer as well as the known actions of neuropeptides on immune cells (13-22), led us to postulate that the local release of neuropeptides from activated nociceptors favors cancer growth by suppressing immune surveillance.

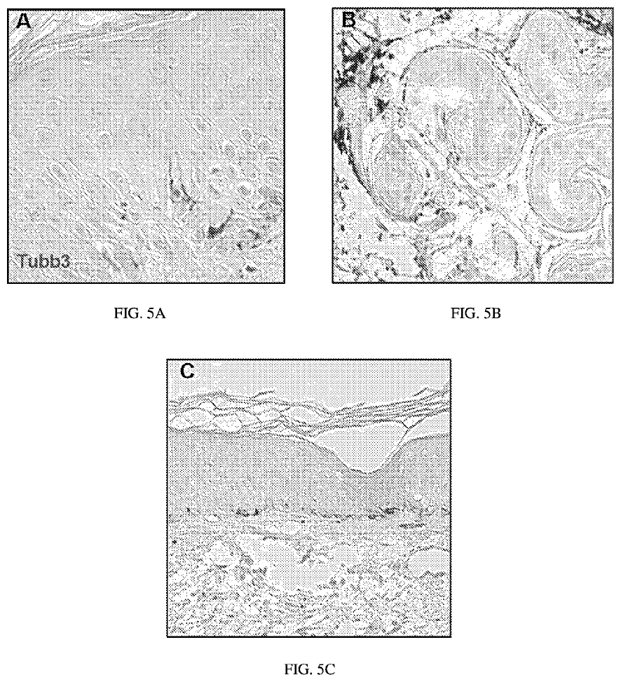

Breast cancer has been found to present increased sympathetic and decreased parasympathetic nerve densities (23), whereas prostate cancers are infiltrated with cholinergic fibers and are surrounded by adrenergic fibers (2). Here, it was sought to probe the extent of melanoma innervation and confirmed the presence of Tubb3 expressing fibers in each of the three melanoma patients' biopsies tested, whom were diagnosed with aggressive diseases (FIGS. 5A-5C). Next, CLARITY was used to obtain a 3-dimensional representation of the innervation present in 22d-B16F10 mouse tumors and confirmed the abundant presence of Tubb3⁺ neurons infiltrating the tumor and its border. To characterize the nature of this innervation, 10⁵ B16F10 cells or 10⁵ non-cancerous keratinocytes (MPEK-BL6) were inoculated (i.d.) into 8-week-old male and female nociceptor neurons reporter mice (TRPV1^{Cre}::Tdtomato^{fl/wt}) (24). Fourteen days post-inoculation, the B16F10 tumor showed nociceptor axonal innervation and outgrowth near the cancer cells (FIGS. 1A-1B).

When co-cultured with eGFP-B16F10 cells, fluorescently labeled (TRPV1^{Cre}::Tdtomato^{fl/wt}) DRG nociceptor neurites were found to largely extend toward the B16F10 cells, often forming physical contacts (FIGS. 1C-1D). In addition, the average neurite length of nociceptor neurons increased (number of intersecting radii; FIGS. 1E-1F) while the overall neuronal arborisation decreased (ramification index; FIG. 1G) when co-cultured with B16F10 cells. Cultured L3-L5 DRG nociceptors harvested from tumor-inoculated mice (on d14) extended longer neurites than their counterparts harvested from keratinocyte-injected mice (not shown). These findings indicate that nociceptors neurite outgrowth is enhanced when in proximity to melanoma cells, as opposed to keratinocytes.

Given that melanoma promotes axonogenesis, which leads to tumor innervation, it was next aimed to examine whether such physical proximity modulates nociceptor sensitivity. To test this, changes in calcium flux were measured, induced in response to sub-threshold concentrations of capsaicin (agonist of the heat sensing channel TRPV1 (Transient Receptor Potential Vanilloid-1)), mustard oil (agonist of the chemical sensing channel TRPA1 (Transient Receptor Potential Anykrin-1)), and ATP (agonist of the proton-sensing channel P2X3R). When nociceptors are cultured without melanoma cells, few DRG neurons responded to the ligands at the concentrations selected. However, the number of responsive neurons increased when co-cultured with B16F10 or B16F0 cancer cells (FIG. 1H). Similarly, the amplitude of calcium flux responses to the ligands was greater in ipsilateral L3-L5 DRG neurons harvested from tumor-inoculated mice (on d14) compared to those harvested from keratinocyte-injected mice (FIG. 1I). As opposed to B16F10 cells alone, DRG neurons co-cultured with B16F10 cells (5×10⁴ cells, 24 h) actively release neuropeptides in the media (e.g. CGRP; FIG. 1J). Unlike DRG neurons (25) (FIG. 1K), B16F10 cells (26) do not express transcripts for Calca, Tac1 or Vip (FIG. 1L); confirming their intrinsic incapacity to produce neuropeptides.

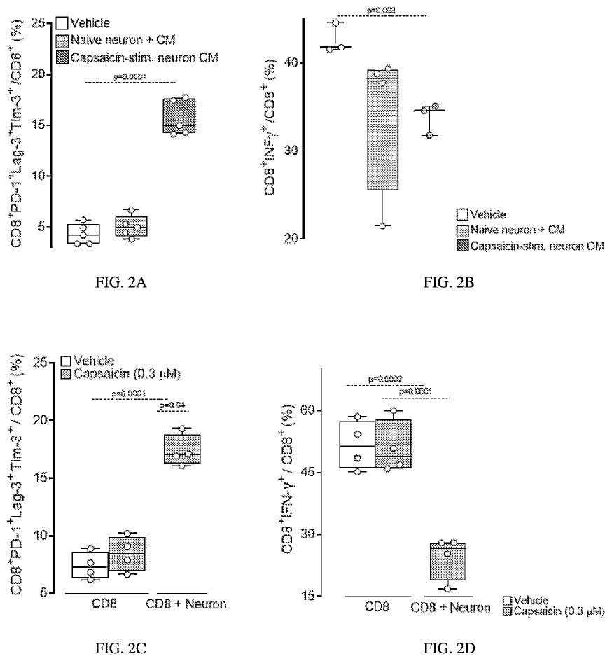

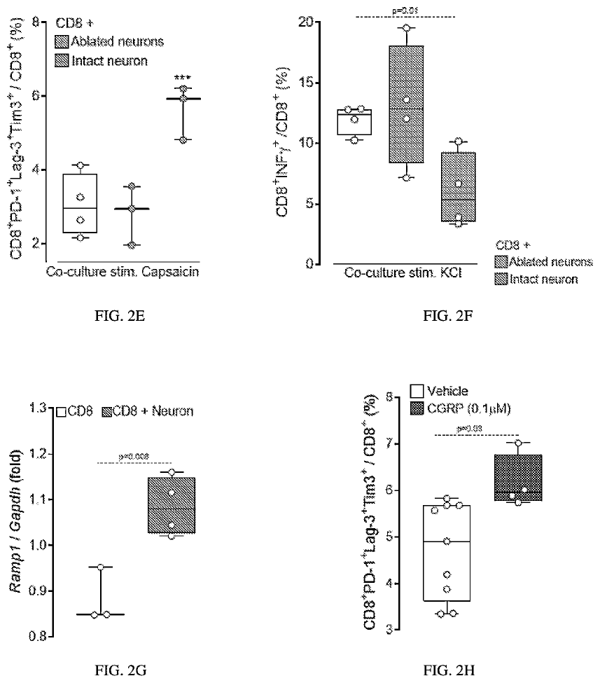

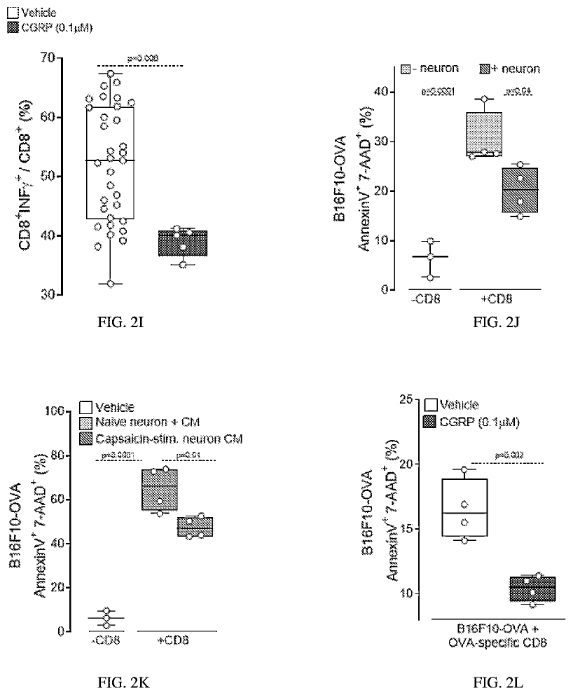

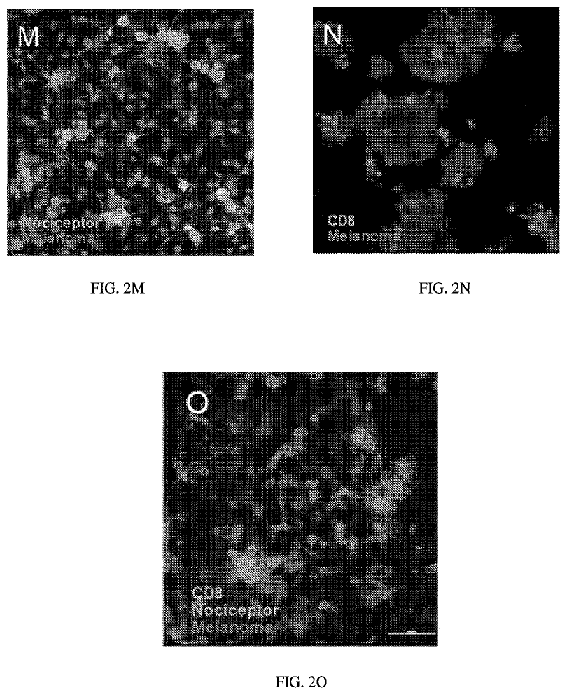

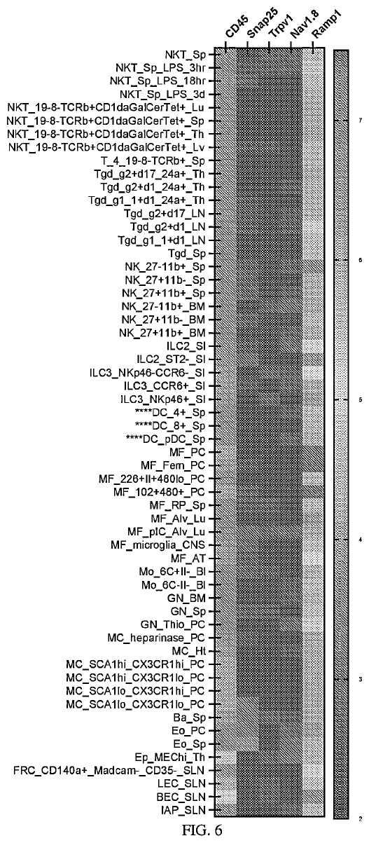

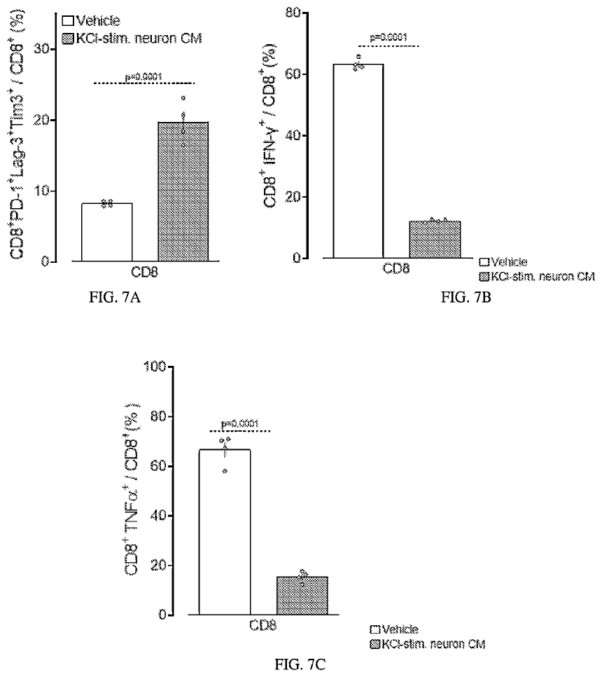

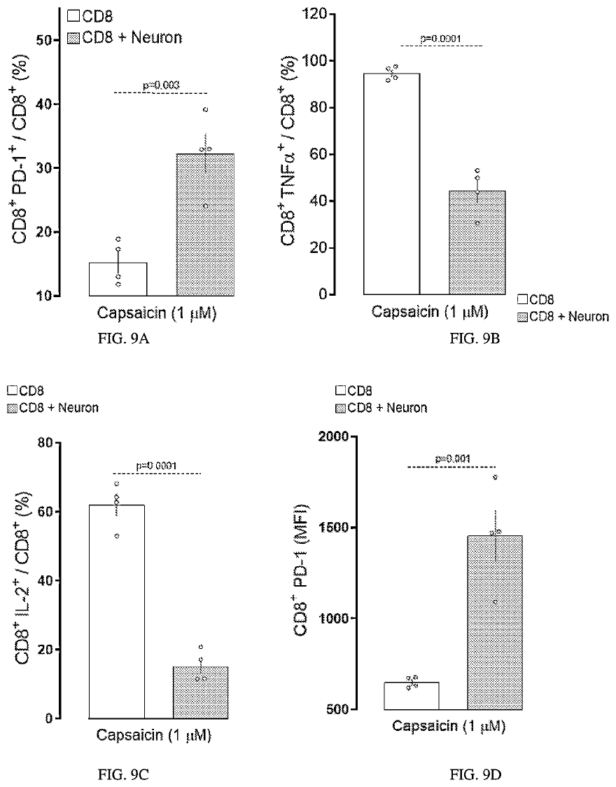

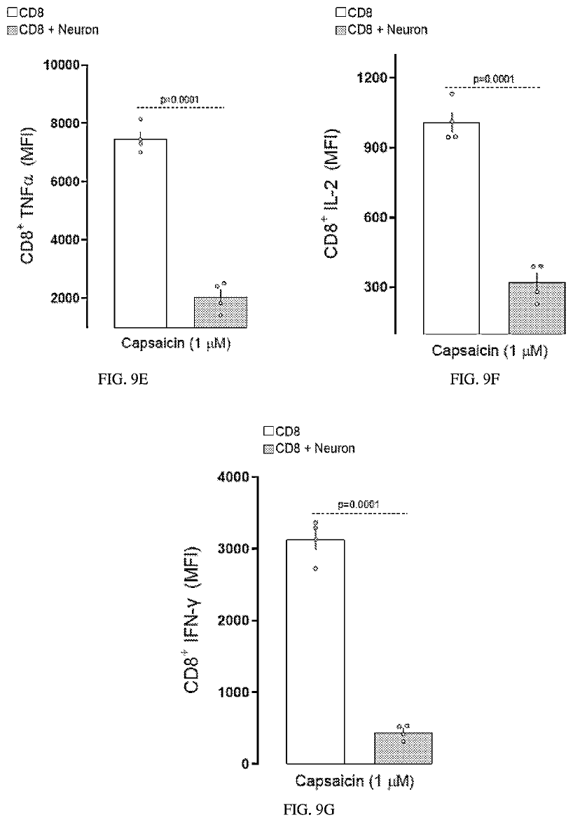

Cytotoxic CD8⁺ T cells express a wide variety (≥10) of neuropeptide receptors (FIG. 6). Given that nociceptors readily interact with CD8⁺ T cells in culture and that the neuropeptides they release block T_{H}1 immunity(27-30), it was tested whether these neuropeptides have direct effects on the expression of immune checkpoint receptors. First, splenocyte-isolated CD8⁺ T cells were cultured under type 1 CD8⁺ T-cell-stimulating conditions for 48 h and were then exposed to conditioned medium harvested from capsaicin-stimulated cultured DRG neurons (30 min). In comparison with naïve neuron media supplemented with capsaicin after harvesting, the conditioned medium containing nociceptor-produced neuropeptides increased the proportion of CD8⁺ T cells expressing PD1⁺Lag3⁺Tim3⁺ (FIG. 2A) and decreased the levels of INFγ⁺ (FIG. 2B) and TNFα⁺ (FIGS. 9A-9G) cells. The conditioned medium from potassium chloride (KCl)-stimulated neurons induced a similar phenotype (FIGS. 7A-7C).

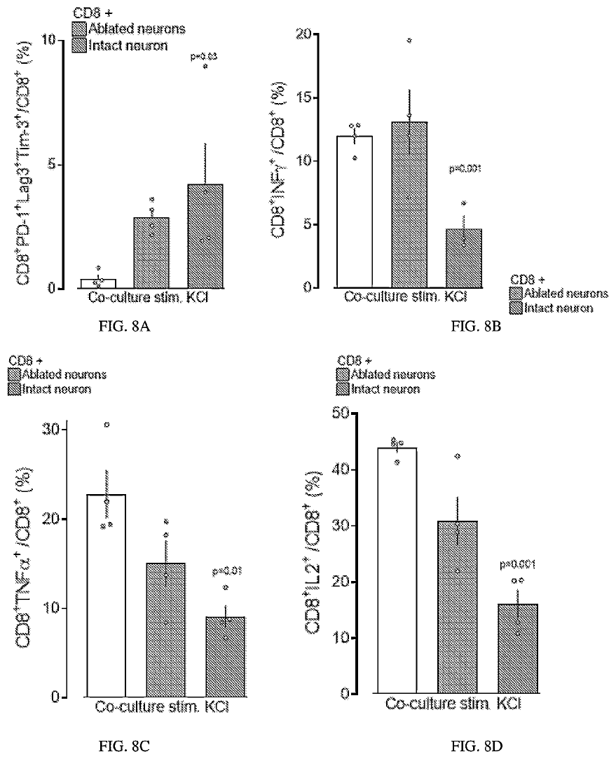

To mimic their interactions within the tumor microenvironment, T_{C1}-stimulated CD8⁺ T-cells were co-cultured with DRG neurons (48 h) and the immunomodulatory role of peptidergic nociceptors were probed using gain-(capsaicin stimulation) and loss-of-function (TRPV1^{Cre}::DTA^{fl/wt}; genetically-engineered nociceptor ablated mice) approaches. Neuron stimulation with capsaicin (FIGS. 2C-2D; FIGS. 9A-9G) or KCl (FIGS. 7A-7C) increased the proportion of PD1⁺Lag3⁺Tim3⁺ (FIG. 2C) and decreased the levels of INFγ⁺ (FIG. 2D) and TNFα⁺ (FIGS. 9A-9G) CD8⁺ T-cells. These effects were absent when CD8⁺ T-cells were co-cultured in absence of TRPV1⁺ neurons (TRPV1^{cre}DTA^{fl/wt}); confirming that peptidergic neurons drive CD8⁺ T-cells exhaustion (FIGS. 2E-2F; FIGS. 8A-8D). In contrast, capsaicin had no measurable impacts on CD8⁺ T-cells in the absence of neurons. These data suggest that neuron-secreted factors, rather than cell-cell contact, are responsible for the exhaustion of cytotoxic CD8 T cells.

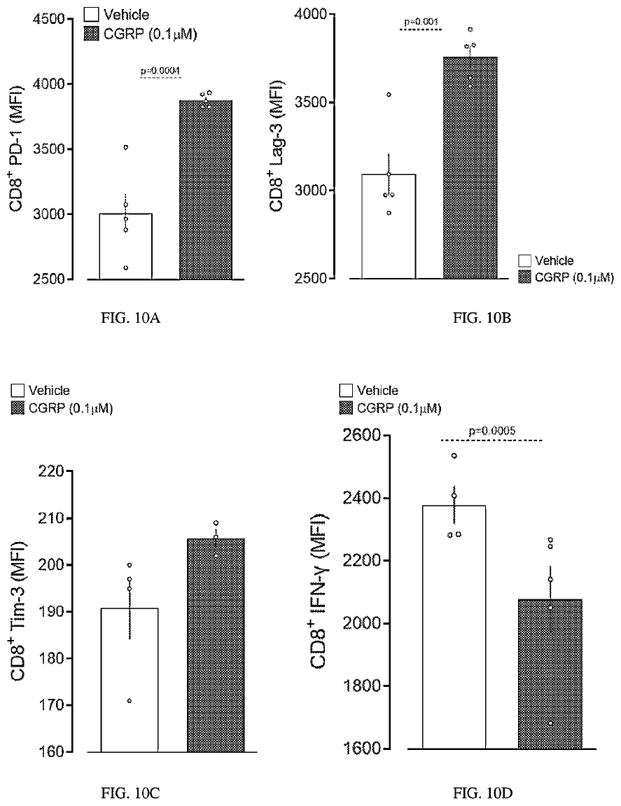

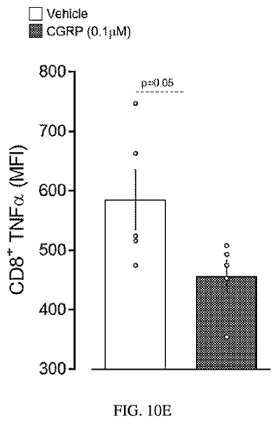

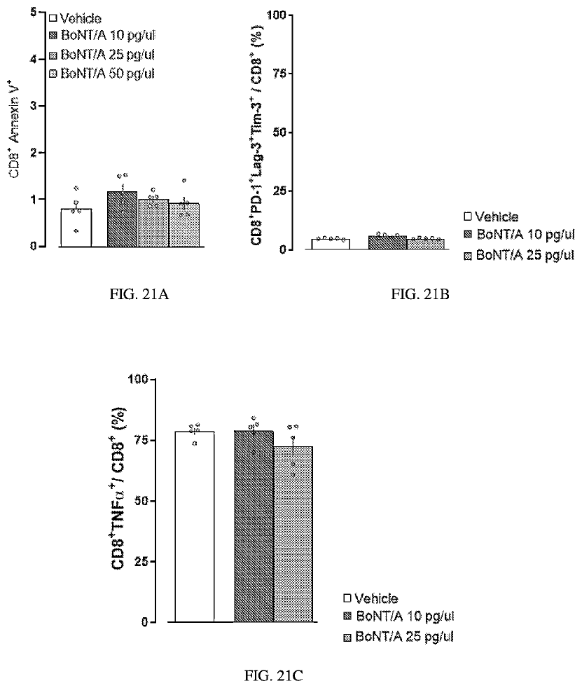

When co-cultured, crosstalk occurs between neurons and cytotoxic CD8⁺ T-cells via cytokines and neuropeptides (22). Such prolong interactions (48 h culture) prompt CD8⁺ T-cells to overexpress the neuropeptide receptor Ramp1(FIG. 2G). Therefore, it was examined whether CGRP has direct immunomodulatory effects on cytotoxic CD8⁺ T-cells and found that it enhanced the proportion of PD1⁺Lag3⁺Tim3⁺ cells (FIG. 2H) and decreased the proportion of INFγ⁺ (FIG. 21) and TNFα⁺ (FIGS. 10A-10E) cells. Along with CGRP blocking CD8⁺ T-cells proliferation (31), the data highlight CGRP as a novel driver of CD8⁺ T-cells exhaustion.

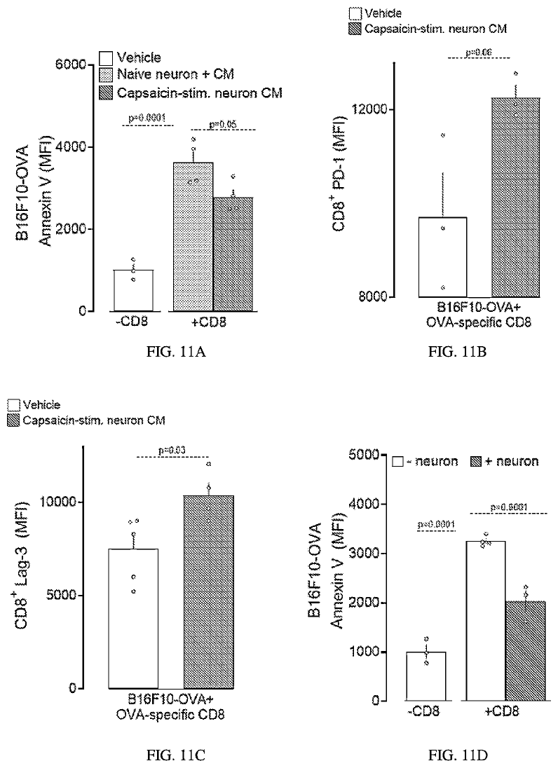

As a litmus test for CD8⁺ T-cell exhaustion, it was probed whether nociceptive neuron-released neuropeptides can blunt the antitumor responses of cytotoxic CD8⁺ T-cells, by culturing B16F10-OVA and OT1 cytotoxic CD8⁺ T-cells, either with or without DRG neurons. OT1 cytotoxic T-cells drastically increased cancer cell apoptosis (annexinV⁺7AAD⁺ B16F10-OVA; FIGS. 2J-2K). B16F10-OVA apoptosis decreased when challenged with capsaicin-stimulated neuron conditioned medium (FIG. 2J), when co-cultured with nociceptors (FIG. 2K) or when stimulated with CGRP (FIG. 2L; FIGS. 11A-11G). Decreased tumor apoptosis correlated with OT1 cytotoxic T-cell exhaustion (FIGS. 11A-11G), which was also confirmed by live-cell imaging (FIGS. 2M-20).

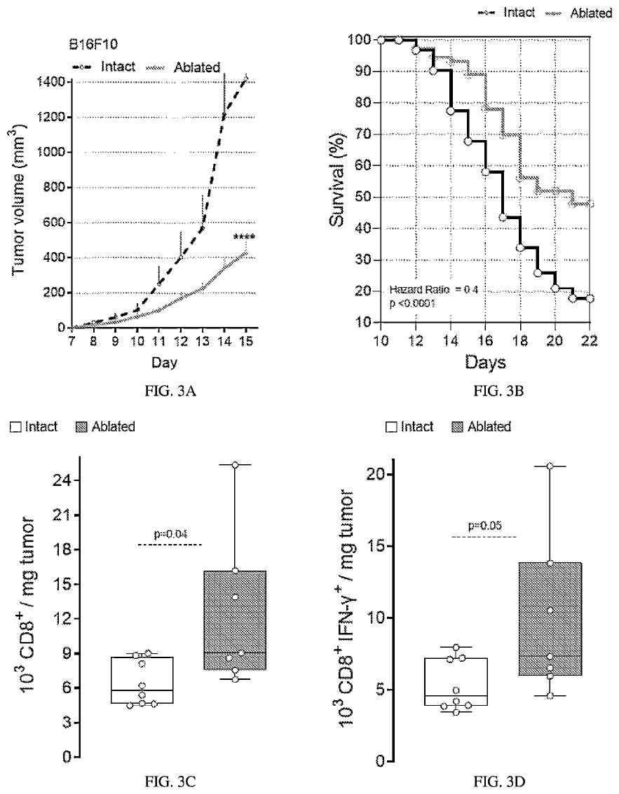

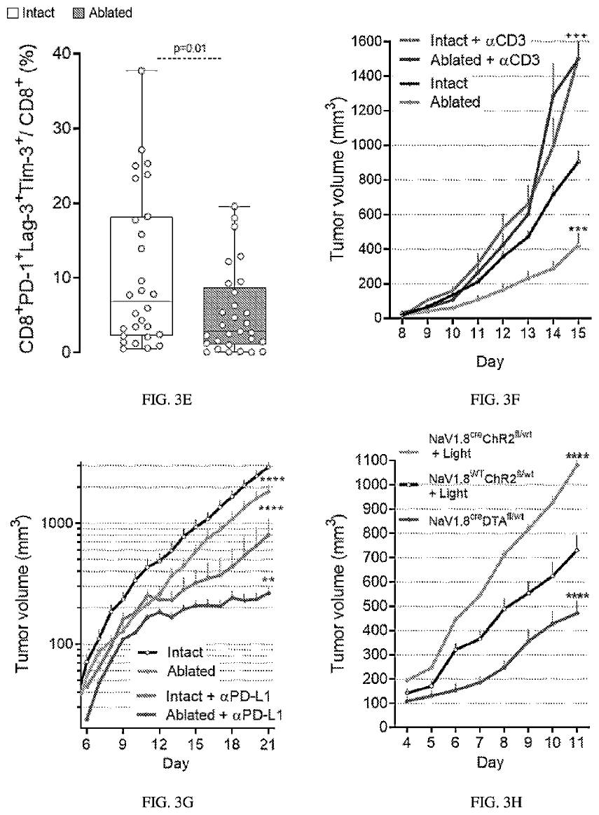

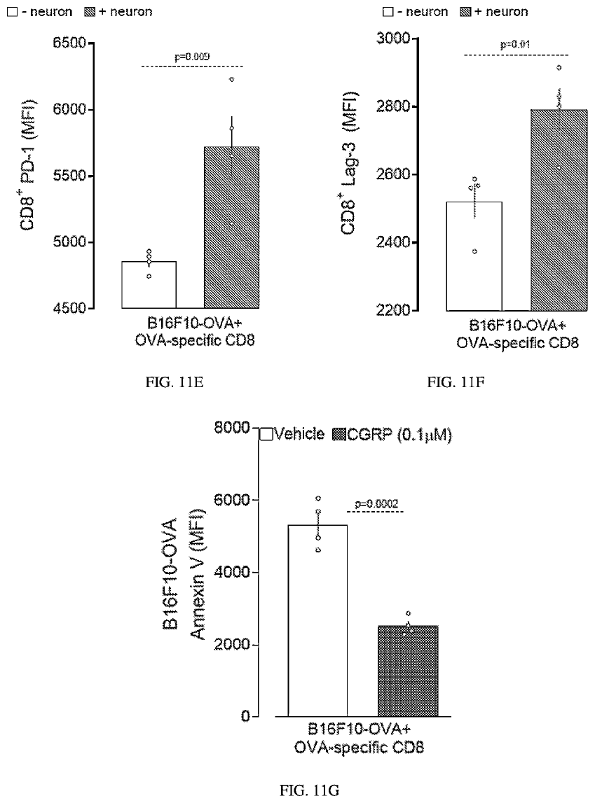

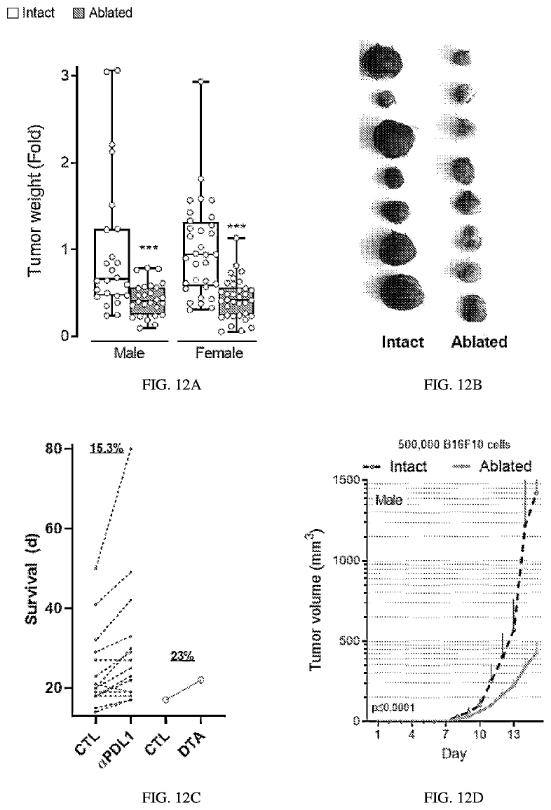

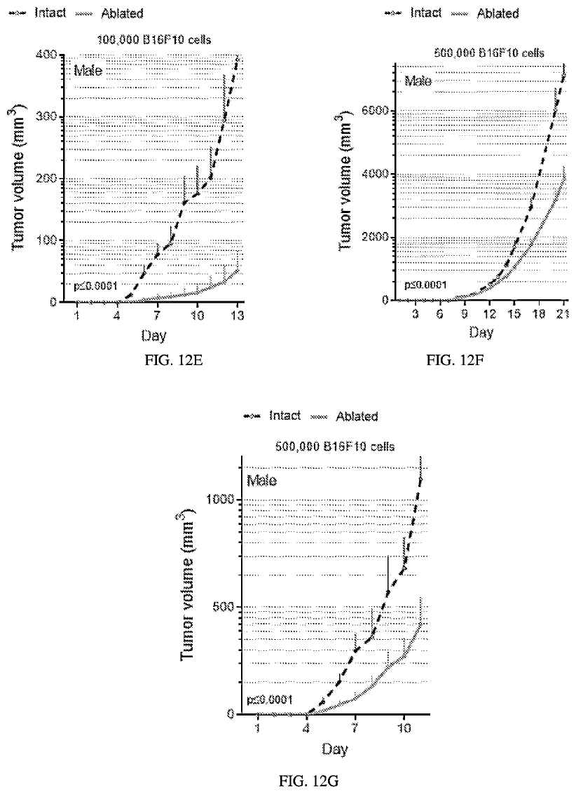

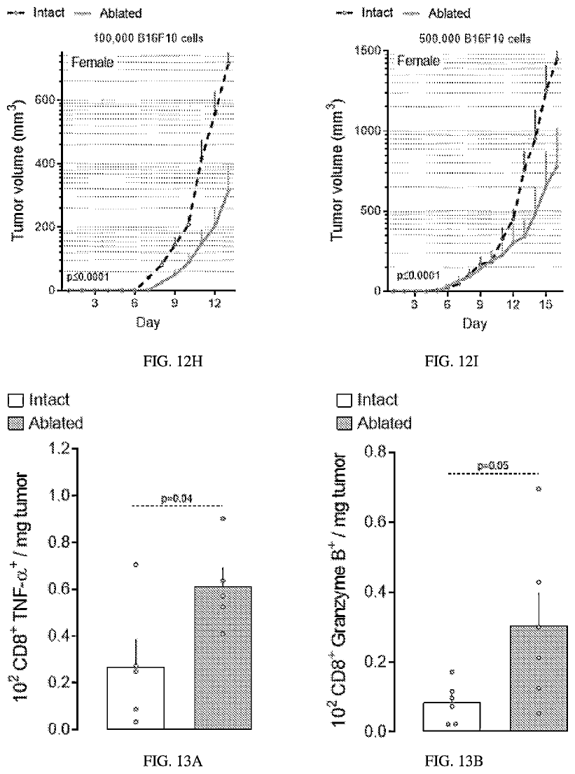

Gastric tumor denervation limits growth, and vagotomised patients have lower mortality rates associated with intestinal cancer (2, 21, 32, 33). Nociceptor-produced neuropeptides have been shown to reduce immunity against bacteria (28) and fungi (34), and to promote the expression of immune checkpoint receptors on cytotoxic CD8⁺ T-cells (FIGS. 2A-2O) (13-18); therefore, it was sought to examine the interaction between cancer-nociceptor-CD8⁺ using a xenograft mouse model of triple-negative melanoma skin cancer, which is an established model of immunosurveillance (10). B16F10 cells were inoculated (i.d., 10⁵) into 8-week-old male and female nociceptor ablated (TRPV1^{Cre}::DTA^{fl/wt}) or intact mice (littermate controls). In both males (n=50) and females (n=68), tumor growth (˜350%) and weight (˜240%) were reduced in mice lacking nociceptors (FIG. 3A; FIGS. 12A-12I), whereas the median length of survival increased by 23% (p≤0.0001; FIG. 3B). This effect represents a 150% increase relative to the survival rate observed following αPDL1 blockade in B16F10 mice (meta-analysis of 16 publications; FIGS. 12A-12I). Consequently, intact mice succumb at a 2.5-fold higher rate than nociceptor ablated mice (0.4 Mantel-Haenszel hazard ratio; FIG. 3B).

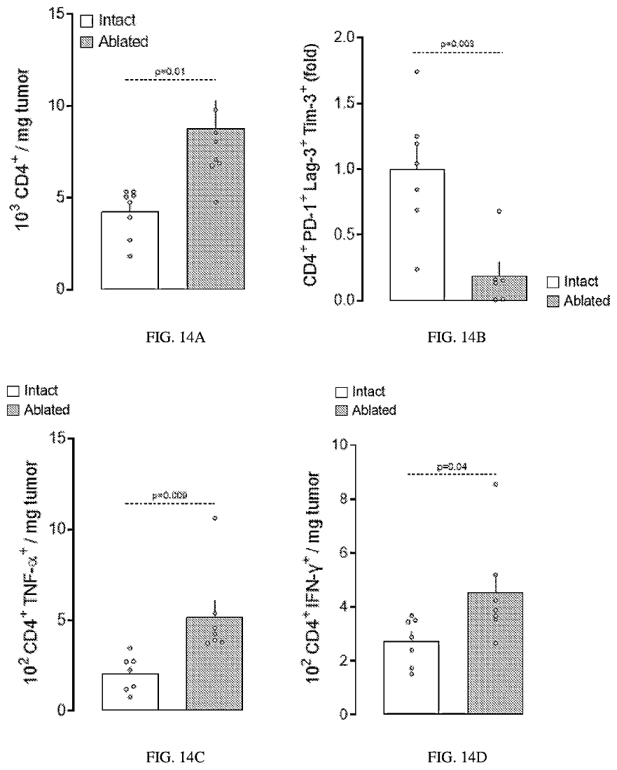

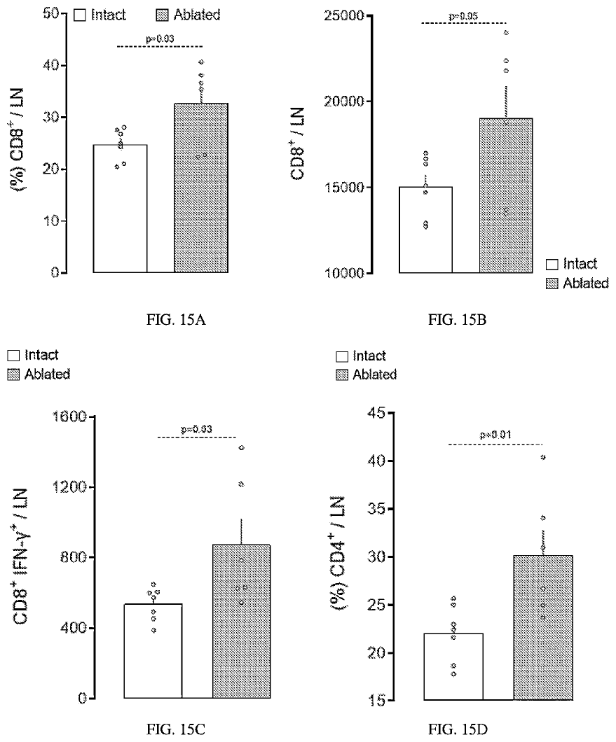

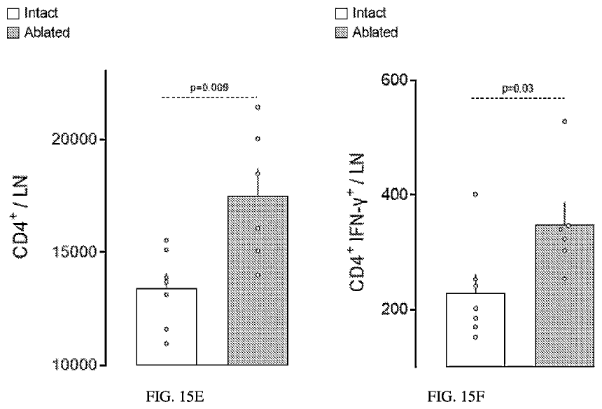

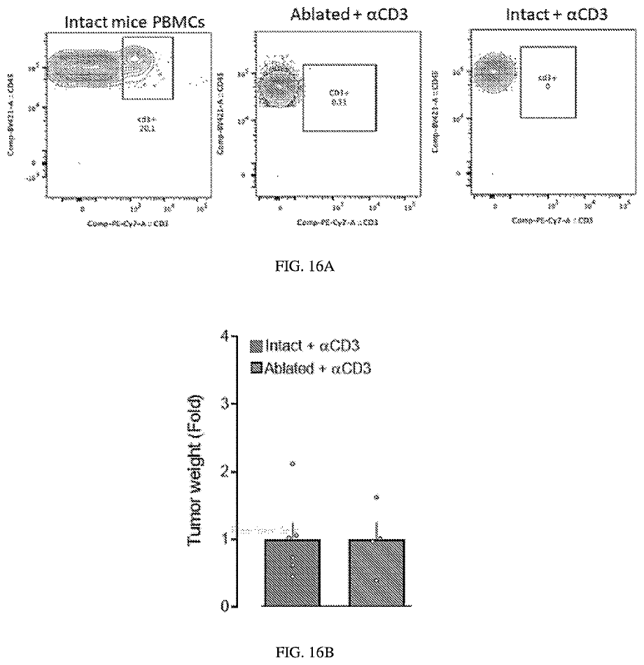

Tumor-specific sympathetic denervation downregulates the expression of PDL1, PD1 and FOXP3 in tumor infiltrating lymphocytes (TIL), whereas parasympathetic innervation decreases the expression of PD1 and PDL1. TIL exhaustion was also correlated with relative distance from sympathetic terminals (23). In B16F10-bearing mice, the genetic ablation of nociceptor neurons increased the numbers of tumor-infiltrating CD8⁺ (FIG. 3C), which were characterized by reduced exhaustion (PD1⁺Lag3⁺Tim3⁺; FIG. 3D) and increased cytotoxic potential (INFγ⁺, TNFα⁺, Granzyme B⁺; FIG. 3D, FIGS. 13A-13B). Similar effects were observed for intra-tumoral CD4⁺ T-cells (FIGS. 14A-14D) as well as for T-cells found in tumor-draining lymph node (FIGS. 15A-15F). Therefore, it was next attempted to test whether the slower B16F10 growth observed in the TRPV1^{Cre}::DTA^{fl/wt} mice depended on lymphocytes. Upon the systemic depletion of CD3⁺ T cells (using αCD3), no differences in B16F10 tumor growth and volume were observed between TRPV1^{Cre}::DTA^{fl/wt} and littermate control mice (FIG. 3E; FIGS. 16A-16B), implying that slower tumor growth depended on nociceptors-induced lymphocyte exhaustion.

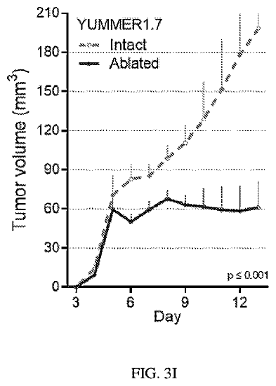

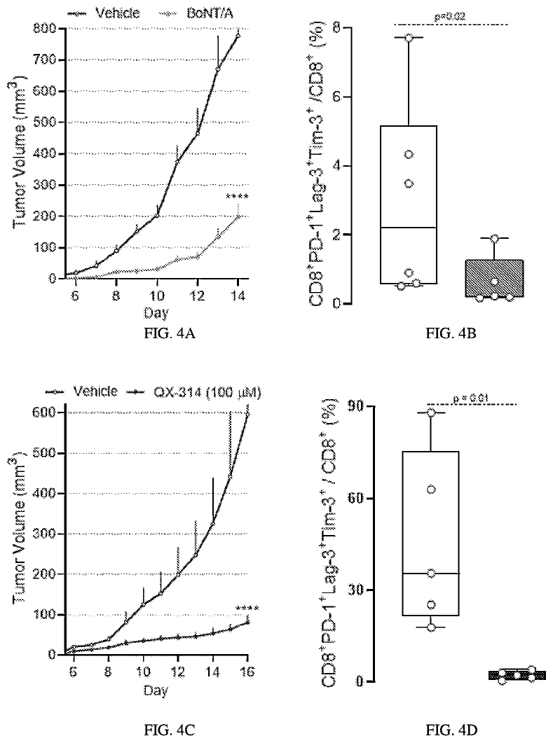

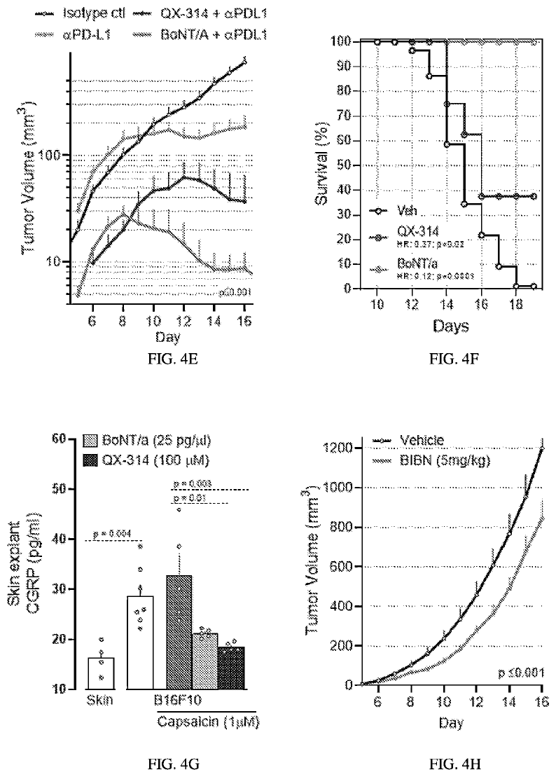

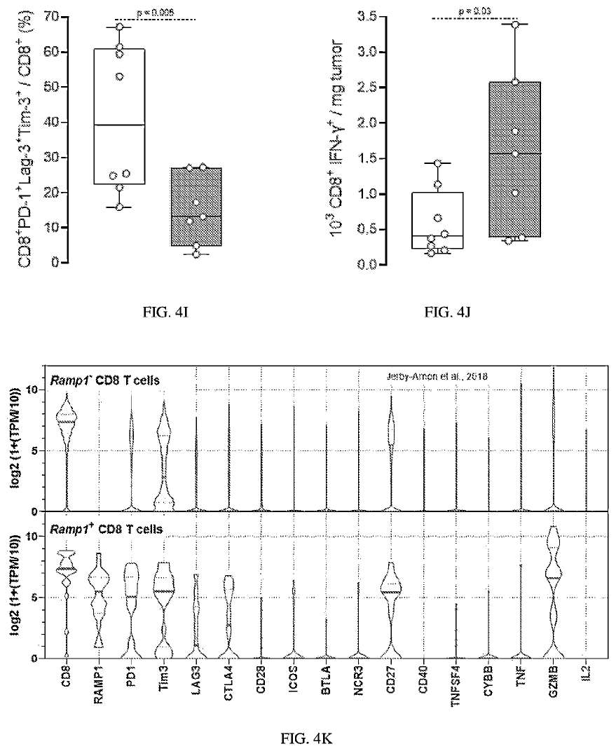

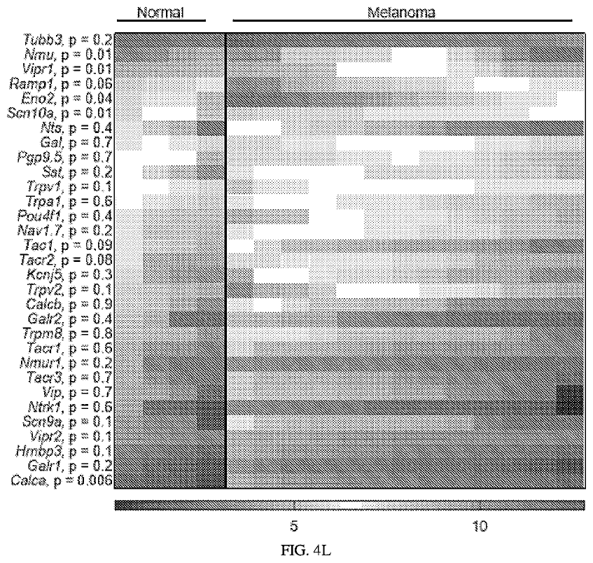

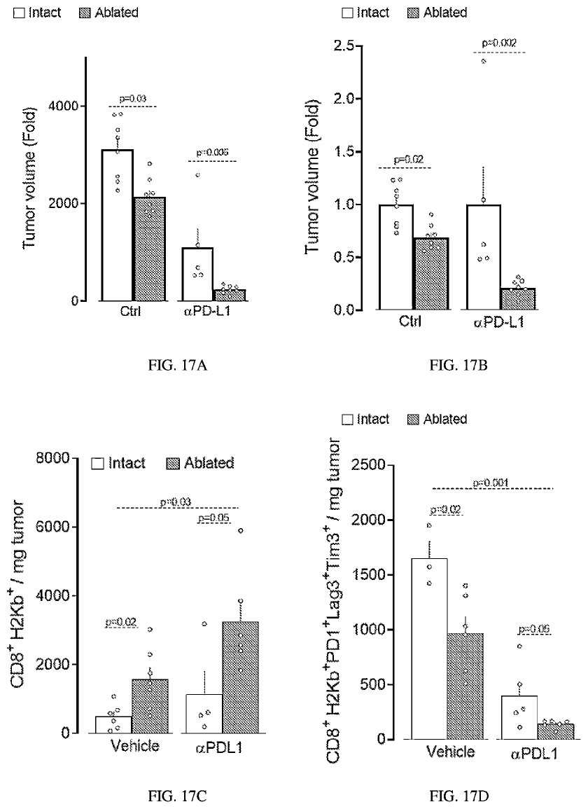

Blocking the activity of immune checkpoint proteins releases the cancer cell-induced “brake” on the immune system, increasing the ability of the system to eliminate tumor (6-8, 10). Immune checkpoint inhibitors, including those targeting PDL1, improve the clinical outcomes associated with metastatic melanoma (8, 35, 36). Given that nociceptor neurons drive CD8⁺ T cell exhaustion, the impacts of nociceptor absence on tumor elimination were assessed following PDL1 blockade. When used after tumor-establishment, αPDL1 (i.p; day 7, 10, 13) treatment resulted in the relative reduction of tumor growth which was enhanced by ˜2.5-fold in nociceptor ablated mice (FIG. 4G). B16F10-OVA tumor size was correlated with increased infiltration of tumor-specific CD8⁺ T-cells which displayed reduced exhaustion (PD1⁺Lag3⁺Tim3⁺), an effect exacerbated by αPDL1 treatment (FIGS. 17A-17D).

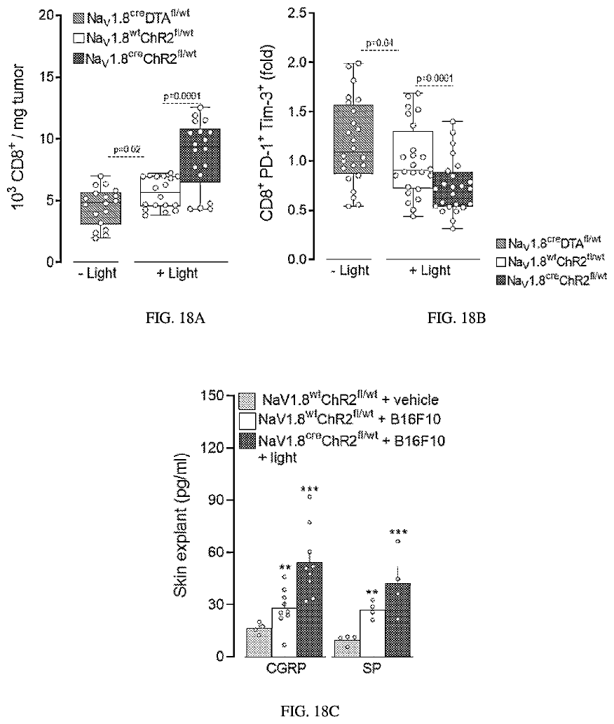

It was next sought to confirm the data using a second line of nociceptor ablated mice. Na_{V}1.8 is a sodium channel expressed by mechano- and thermos-sensitive neurons and ˜80% of nociceptors (28, 37). The ablation of Na_{V}1.8⁺ sensory neurons (Na_{V}1.8^{Cre}::DTA^{fl/wt}) slowed B16F10 tumor growth (FIG. 3H), reduced the proportion of PD1⁺Tim3⁺ CD8⁺ T cells within the tumor but increased the infiltration of total CD8⁺ T-cells (FIGS. 18A-18C). Note that unbiased RNA sequencing data unequivocally showed that TRPV1 and Na_{V}1.8 are not expressed by immune cells (FIG. 6).

Next, a gain-of-function approach was used, in which daily transdermal illumination (3.5 ms, 10 Hz, 478 nm, 100 mW, delivering ˜2-6 mW/mm² to a 0.39 NA fiber placed 5-10 mm from the skin, for 20 min) was used to stimulate optogenetically-controlled tumor-innervating Na_{V}1.8⁺ neurons. In comparison with light-insensitive littermate control mice, blue light-stimulated Na_{V}1.8^{Cre}::ChR2^{fl/wt} mice enhanced B16F10 tumor growth (FIG. 3H), increased proportion of PD1⁺Tim3⁺ CD8⁺ T-cells, and decreased infiltration of total CD8 T-cells (FIGS. 18A-18C). Tumor growth and CD8⁺ T-cells exhaustion correlated with secreted CGRP levels measured in the tumor-surrounding skin after optogenetic stimulation (FIG. 18C).

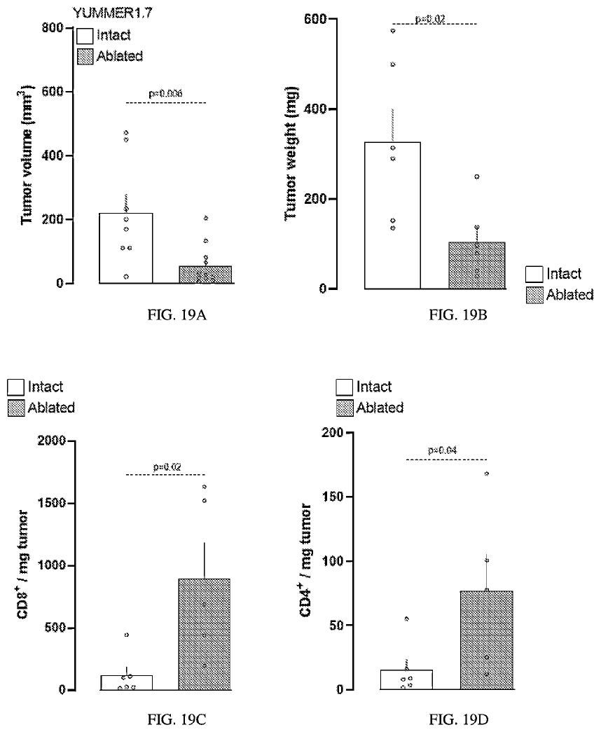

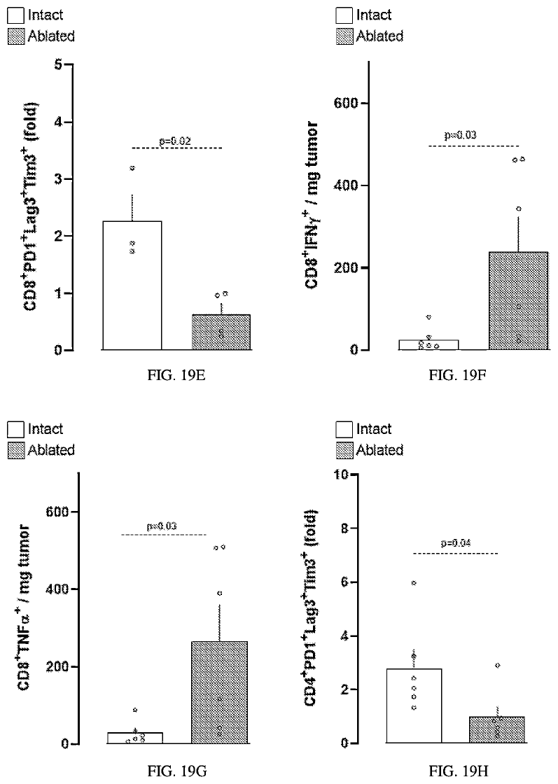

Sensory neuron depleted mice were then inoculated with the YUMMER1.7 cell line; which is a highly immunogenic form of the Braf^{V600E}Cdkn2a^{-/-} Pten^{-/-} cell line that has been modified by UV exposure and represents a clinically relevant melanoma model. In comparison to mice whose sensory neurons are intact, slower tumor growth (˜3.3 fold; FIG. 3I) and reduced tumor weights (˜3.1 fold) were found in the absence of TRPV1⁺ neurons which correlated with a reduced (˜3.6-fold) proportion of PD1⁺Lag3⁺Tim3⁺ CD8⁺ T-cells (FIGS. 19A-19H). The ablation of sensory neurons also increased the infiltration of total (˜7.2-fold), INFγ⁺ (˜9.1-fold) and TNFα⁺ (˜9 fold) CD8 T-cells (FIGS. 19A-19H). Similar effects were observed for CD4⁺ T-cells (FIGS. 19A-19H).

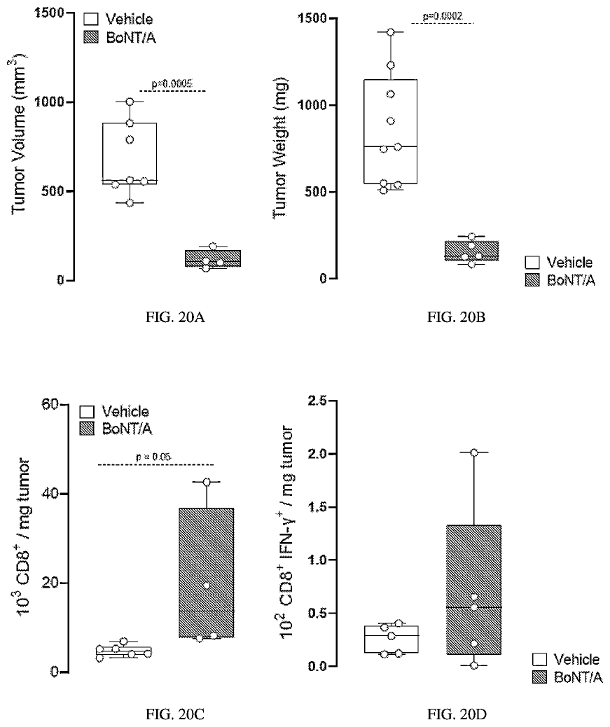

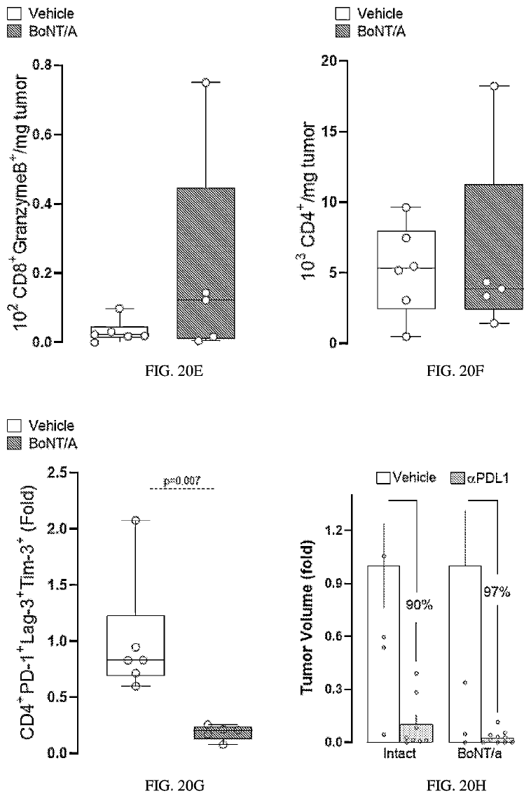

The neonatal/embryonic ablation of neuronal subsets may lead to compensatory mechanisms. To circumvent this potential shortcoming, neurons were silenced using the FDA-approved cervical dystonia treatment Botulinum toxin A (BoNT/A), a neurotoxic protein produced by Clostridium botulinum, which acts by cleaving SNAP25 (38). This strategy caused the long-lasting (20 days) abolition of neurotransmitter′ release from skin-innervating autonomic fibers and somatosensory neurons (CGRP) and blocked the neuro-immune interplay occurring during skin infection (29). In addition, BoNT/a blockade of synaptic vesicle release from neurons reduce tumor growth in prostate cancer (2). BoNTA administration (25 pg/μl; 501; 5 i.d. sites) prior to tumor inoculation, reduced subsequent tumor growth (FIG. 4A) and the proportion of PD1⁺Lag3⁺Tim3⁺ CD8 T-cells (FIG. 4B), resulting in the survival of BoNT/A-treated B16F10-bearing mice (0.12 hazard ratio; FIG. 4F). BoNT/A treatment also increased the intra-tumor CD8⁺ and CD4⁺ T-cells counts and preserved their cytotoxic potential (INFγ, TNFα, Granzyme B; FIGS. 20A-20H).

A proven nociceptor-blocking strategy (39) was then used to silence tumor-innervating nociceptors. This protocol uses large pore ion channels (TRPV1) as cell-specific drug-entry ports to deliver QX-314, a charged and membrane-impermeable form of lidocaine, to block voltage-gated sodium (Na_{V}) channels. During inflammation, similar to that observed in tumor microenvironments, these ion channels open, allowing QX-314 to permeate into these neurons resulting in a long-lasting electrical blockade (37). Compared with vehicle, QX-314-mediated sensory neuron silencing (0.3%; daily i.d. surrounding the tumor) reduced melanoma growth (˜3.3-fold; FIG. 4C), tumor weight (˜5.2-fold; FIGS. 22A-22I) and the proportion of PD1⁺Lag3⁺Tim3⁺ CD8⁺ T-cells (˜15-fold; FIG. 4D). Sensory neuron silencing increased the intra-tumor numbers of CD8⁺ and CD4⁺ T-cells and preserved their cytotoxic potential (INFγ, TNFα, Granzyme B; FIGS. 22A-22I). Vehicle exposed B16F10-bearing mice succumb at a 2.7-fold higher rate (p≤0.0001) than QX-314-exposed mice (0.37 hazard ratio; FIG. 2F).

Nociceptor neurons express PD1, a level found to be reduced in tumor-innervating neuron (FIG. 35). Nevertheless, cell-cell interplay between PDL1⁺ TILs and PD1⁺ neurons may result in immune exhaustion. Here, it was found that αPDL1-mediated tumor reductions were increased by ˜2.5-fold in nociceptor ablated mice (FIG. 3G; FIGS. 17A-17D), ˜10 fold when used in BoNT/A pre-treated mice (FIG. 4E; FIGS. 20A-20H) or ˜5 fold when used in combination with QX-314 (FIG. 4E; FIGS. 22A-22I). Given that αPDL1 induces allodynia by increasing nociceptor activity, it is therefore likely to promote neuropeptide release and subsequent TIL exhaustion. The silencing of nociceptors may augment αPDL1 efficacy by limiting its pro-nociceptive effects, safeguarding the anti-tumor immunity of the host (FIGS. 17A-17D). Overall, these data indicated that silencing nociceptors (QX-314 or BoNT/A) may be a potent adjuvant treatment for immune checkpoint blockers.

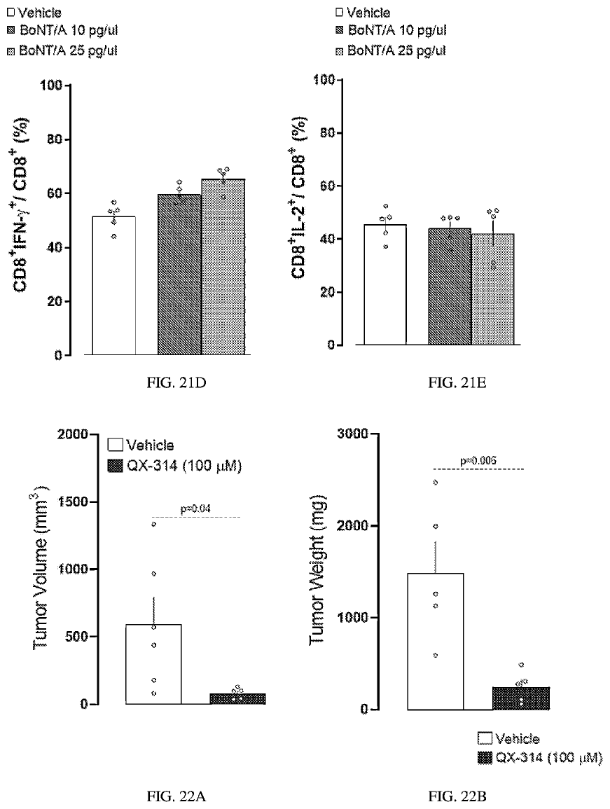

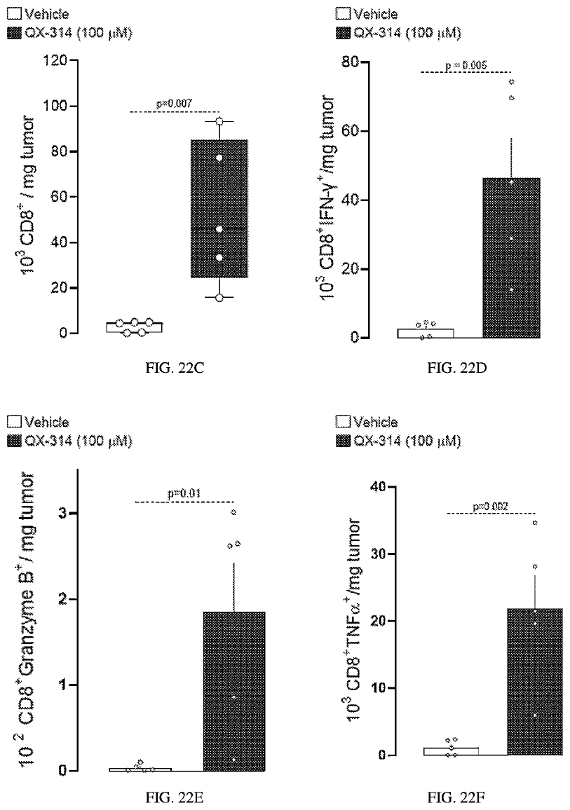

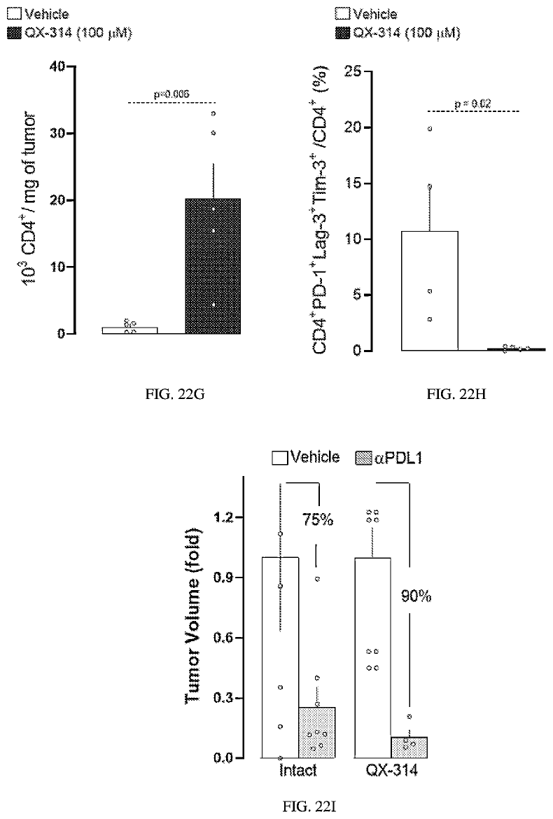

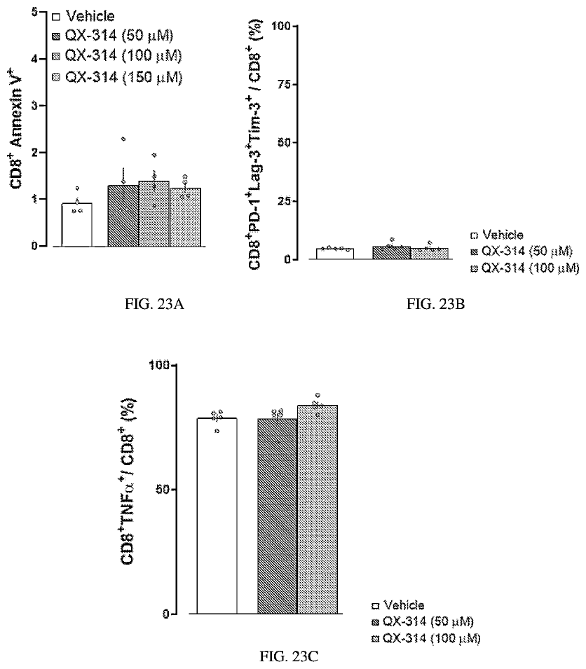

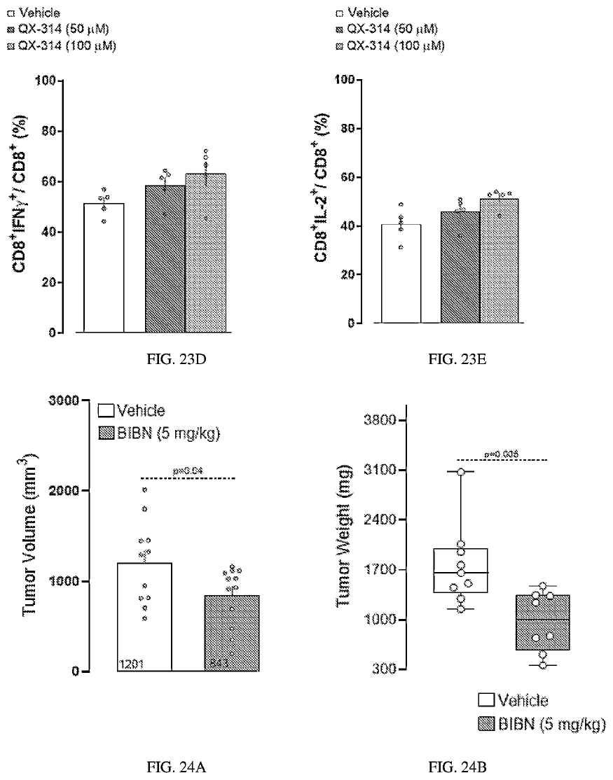

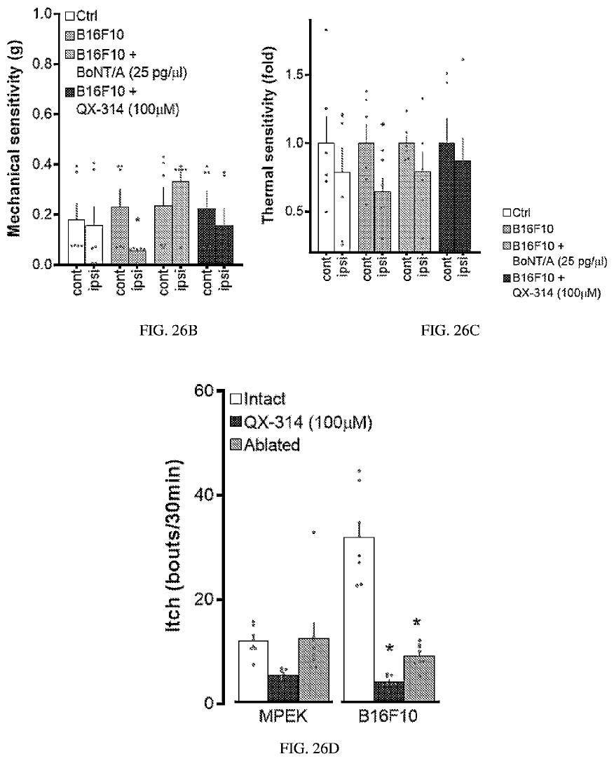

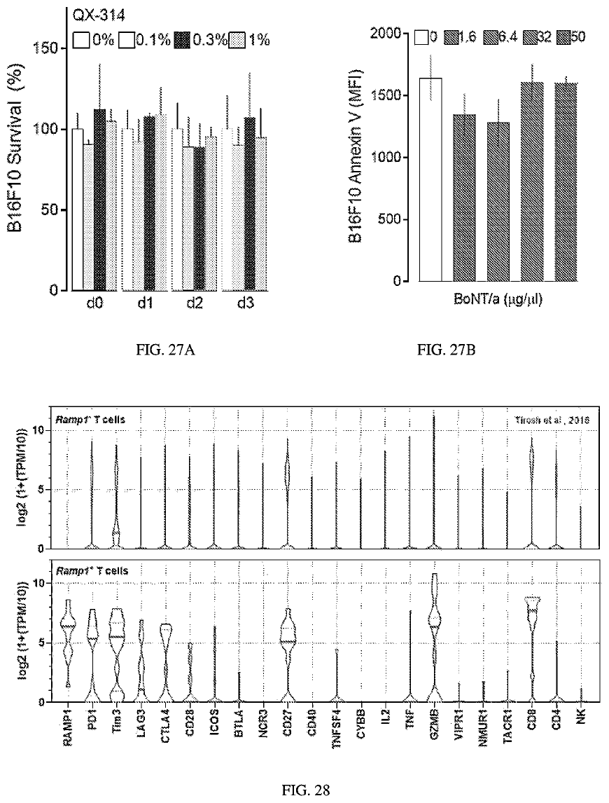

In support of the neuronal specificity of BoNT/A and QX-314, unbiased RNA-sequencing analysis has shown that in contrast to nociceptors (FIG. 1K), B16F10 (FIG. 1L) and immune cells (FIG. 6) do not express Snap25, Trpv1, Na_{V}1.7 or Na_{V}1.8. In addition, BoNT/A and QX-314 have no impacts on B16F10 survival (FIGS. 27A-27B) or CD8⁺ T-cells function (FIGS. 21A-21E and FIGS. 23A-23E). Tumor growth, mechanical and thermal pain-related hypersensitivity measured in hindpaw-inoculated B16F10 mice, and itching (observed in ˜30% of mice with flank inoculated tumor) were absent in mice with genetically ablated or pharmacologically silenced nociceptors (FIGS. 26A-26D). Capsaicin-induced CGRP release from tumor-inoculated skin was also blunted in QX-314 and BoNT/A pre-treated skin (FIG. 4G). These data support the capacity of QX-314 and BoNT/a to i) silence tumor-innervating neurons and ii) prevent the release of CGRP and its subsequent immunomodulation of TILs.

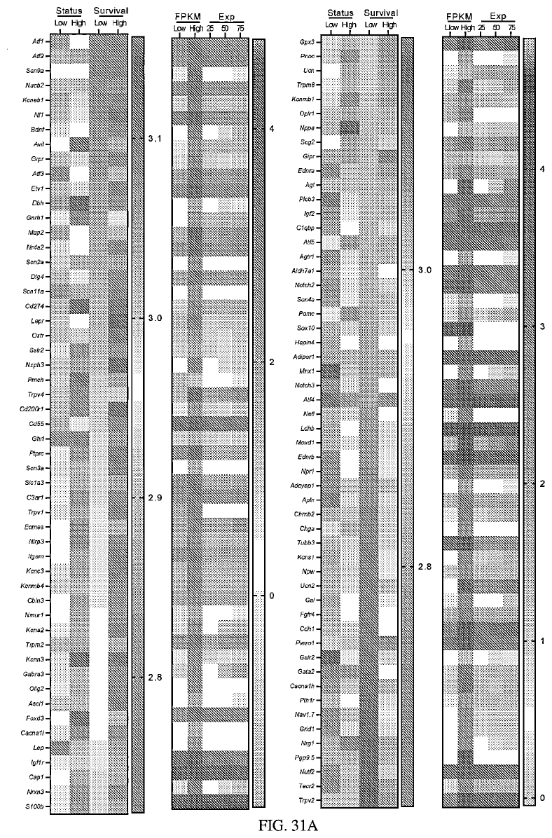

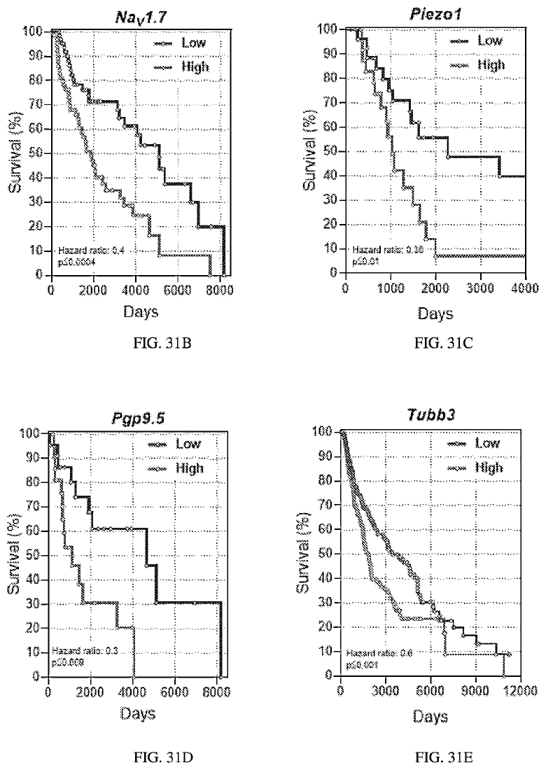

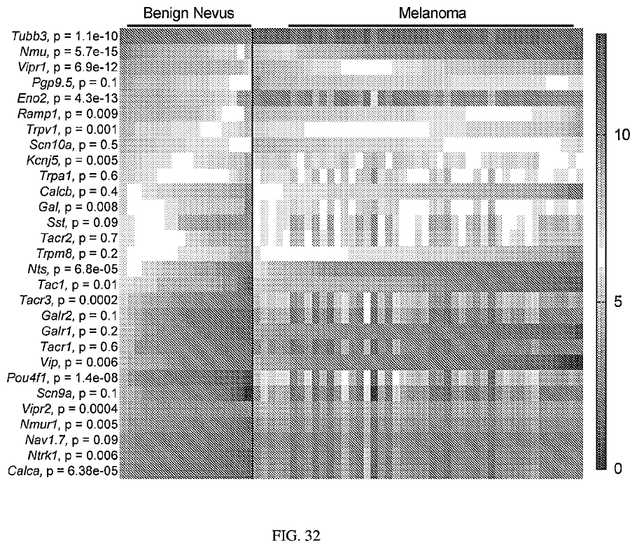

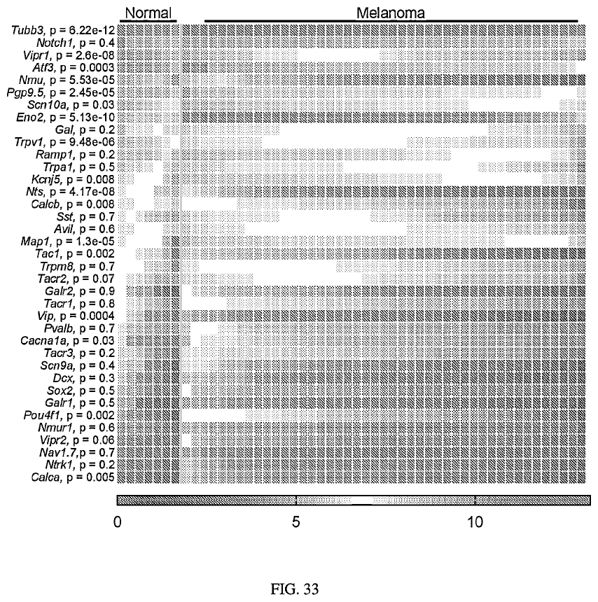

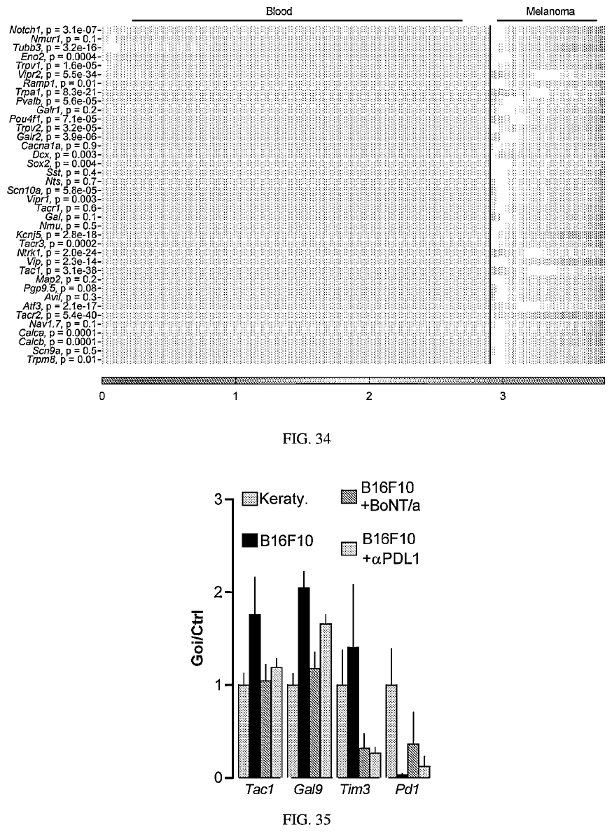

Adrenergic and cholinergic nerve fiber densities in tumors have been correlated with poor clinical outcomes (2). Whether nociceptor innervation of melanomas has a similar effect remains unclear. Here, it was found that patient′ survival (oncolnc database; 459 patients (40)) was negatively correlated (p≤0.05) with increased tumor expression of NaV1.7, Piezo1, Pgp9.5 and Tubb3 (FIGS. 31A-31E). Patient′ biopsies also showed an increased expression (p≤0.05) of Calca, Ramp1 and Na_{V}1.8 expression compared with healthy skin (FIG. 4L; FIG. 33), PBMCs (FIG. 34) or benign nevi (FIG. 32) (41-44).

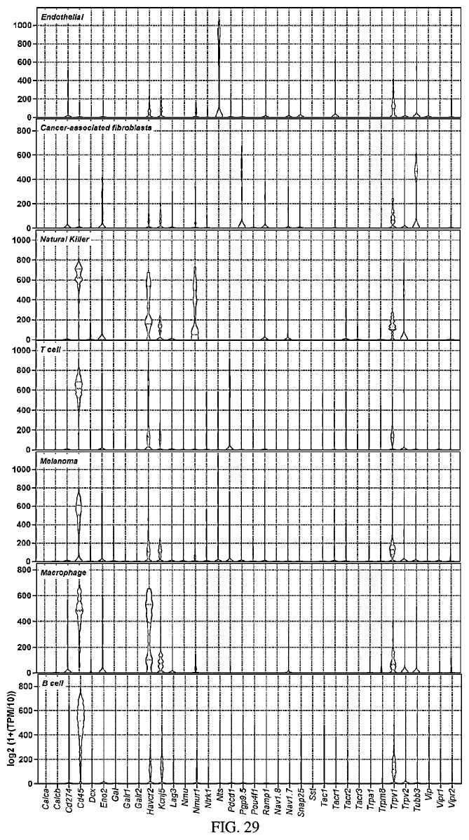

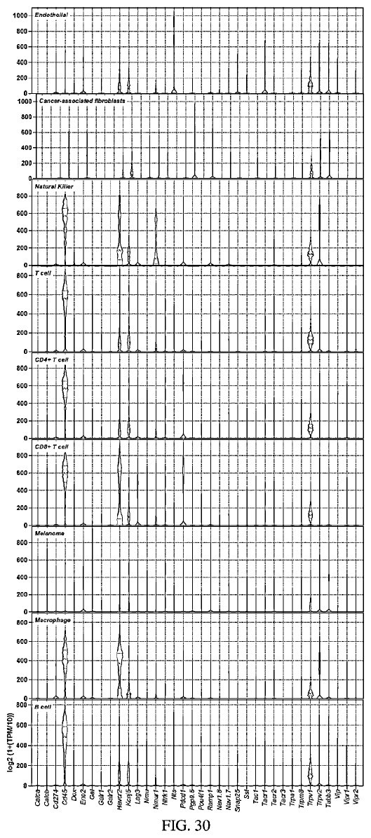

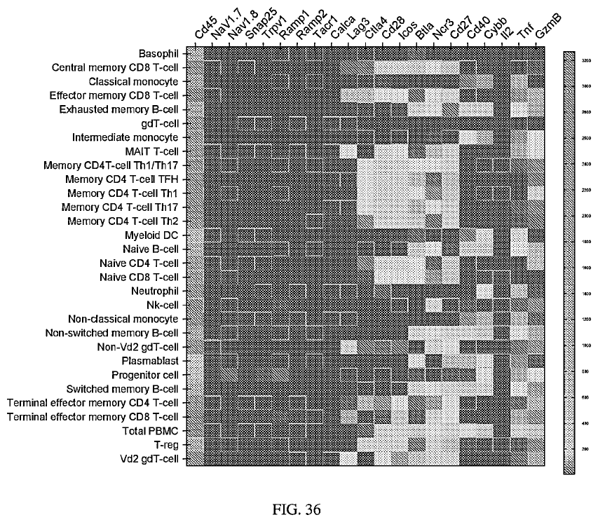

In support of the neuronal origin of these genes and in contrast with doublecortin (1), it was found that Calca, Na_{V}1.7, Piezo1, Pgp9.5 as well as Snap25, Trpv1 and Na_{V}1.8, are not expressed by TILs, cancer-associated fibroblasts, endothelial cells or melanoma cells according to the in-silico analysis of two independent single-cell RNA-sequencing performed on melanoma biopsies (45, 46) (FIGS. 29, 30) as well as the Immgen (47) (FIG. 6) and human protein atlas (48, 49) (FIG. 36) databases. Overall, these data, in addition to the presence of Tubb3⁺ neurons in biopsy samples (FIGS. 5A-5C), support a link between melanoma innervation, the heightened expression of neuropeptides or their cognate receptors and the worsening of clinical prognosis. These data are once more indicative of the translational potential of BoNT/A and QX-314, to silence tumor-innervating nociceptors.

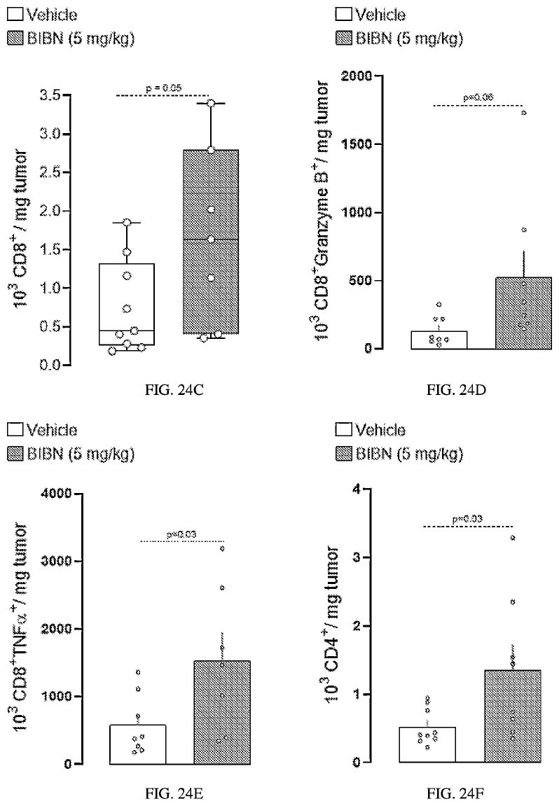

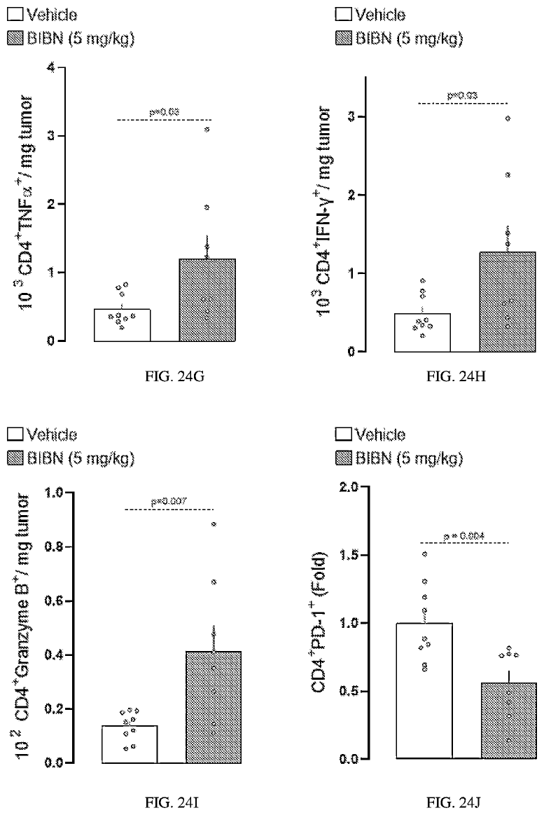

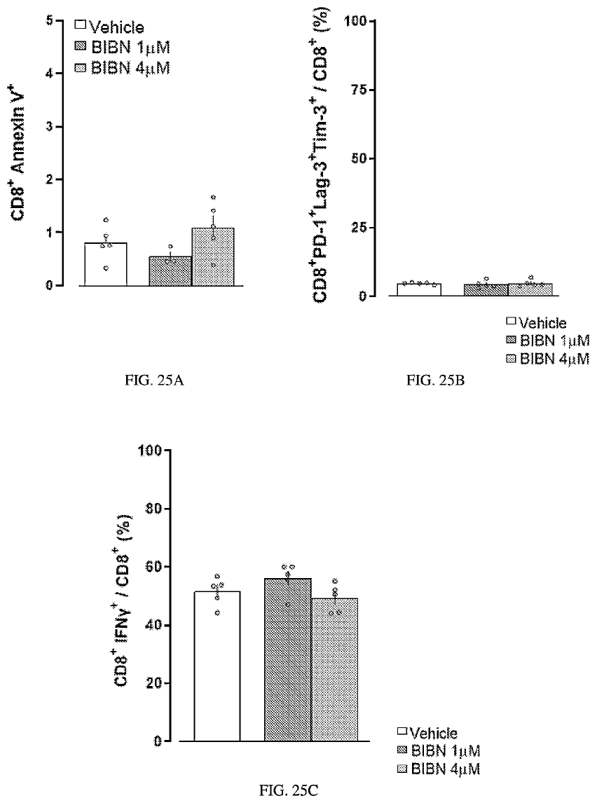

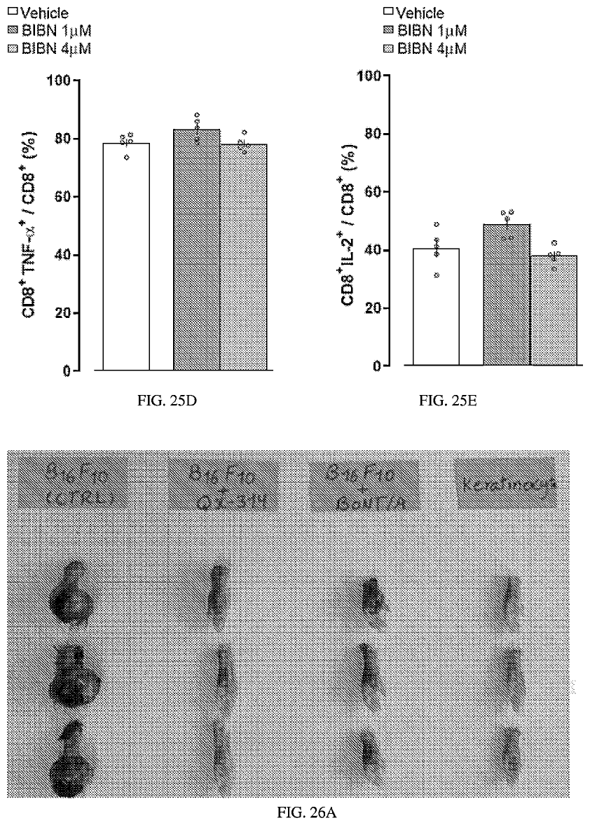

CD8⁺ T-cells overexpress Ramp1when co-cultured with sensory neurons (FIG. 2G), and CGRP increases CD8⁺ T-cells expression of immune checkpoint receptors (FIG. 2H), reduces their production of cytotoxic granules (FIG. 2I), and blunts the OT1-CD8⁺ T-cells capacity to eliminate B16F10-OVA melanomas (FIG. 2L; FIGS. 10A-10E; FIGS. 11A-11G). CGRP receptor antagonism using BIBN4096 blocked the deleterious neuro-immune interplay during microbe infections and rescued host anti-bacterial activity (27). Here, it was found that BIBN4096, when administered systemically once every two days, reduced B16F10 growth (˜1.5-fold; FIG. 2H), tumor weight (˜1.9-fold; FIGS. 24A-24J) and the proportion (˜2.5-fold) of PD1⁺Lag3⁺Tim3⁺ CD8 T cells (FIG. 2I). RAMP1 blockade also increased the intra-tumor number of total (˜3-fold; FIGS. 24A-24J) and INFγ⁺ (˜3-fold; FIG. 2J) CD8⁺ T-cells. Similar effects were observed for intra-tumor CD4⁺ T-cells (FIGS. 24A-24J). It is worth noting that BIBN4096 (1-4 μM) have no impact on cultured B16F10 survival and do not prevent the effects of αCD3/αCD28 stimulation on CD8⁺ T-cells (FIGS. 25A-25E).

Single-cell RNA-sequencing of human melanomas revealed that ˜1% of tumor-infiltrating CD8⁺ T-cells expressed Ramp1 (45). When clustered, these Ramp1⁺ CD8⁺ T-cells revealed their drastic overexpression of the immune checkpoint receptors PD1, Tim3, Lag3, CTLA4, CD27 coupled with a loss of the pro-proliferative cytokine IL-2 (FIG. 4K). Similar findings were obtained from an independent single cell RNA-sequencing analysis of melanoma-infiltrating T cells (46) (FIG. 28). Reminiscent of severe exhaustion (45, 46) (FIG. 4K; FIG. 28), the expression profile of Ramp1⁺ tumor-infiltrating CD8⁺ T-cells support the concept that nociceptor-released CGRP promotes CD8⁺ T-cells exhaustion (FIG. 2H, 2L) and the use of BIBN4096 as a targeted therapy to safeguard anti-tumor immunity (FIG. 4H-4I; FIGS. 24A-24J).

In summary, the data support a regulatory role for nociceptors in the immune responses to tumor growth, through the regulation of immune checkpoint receptors expression on cytotoxic CD8⁺ T-cells. Silencing tumor-innervating sensory neurons represents an innovative strategy for attenuating the immunomodulatory power of the nervous system and promoting anti-tumor activity.

### Methods for Example 1

### Animals

The Institutional Animal Care and Use Committees of Boston Children's Hospital, and the Université de Montréal (CDEA #19027; #19028) approved animal procedures. Mice were housed in standard environmental conditions (12 h light/dark cycle; 23° C.; food and water ad libitum) at facilities accredited by the Association for Assessment and Accreditation of Laboratory Animal Care. 8-week old C57BL6 (Jax, #000664); OT1 (Jax, #003831) (50), TRPV1^{cre} (Jax, #017769) (51), ChR2^{fl/fl} (Jax, #012567) (52), td-tomato^{fl/fl} (Jax, #007908) (53), DTA^{fl/fl} (Jax, #009669) (54), and QuASR2^{fl/fl} (Jax, #028678) (55) mice were purchased from Jackson Laboratory. Na_{V}1.8^{Cre} mice (56) were generously supplied by Professor Rohini Kuner (Heidelberg University). The cre/lox toolbox was used to genetically-engineered the various mice lines used (TRPV1^{cre}::DTA^{fl/wt}, TRPV1^{cre}:: QuASR2^{fl/wt}, TRPV1^{cre}::Tdtomato^{fl/wt}, NaV1.8^{cre}::DTA^{fl/wt}, NaV1.8^{cre}::ChR2^{fl/wt} and littermate control) by crossing male heterozygote Cre mice to female homozygous loxP mice. Cre driver lines used are viable and fertile and abnormal phenotypes were not detected. Offspring were tail clipped; tissue was used to assess the presence of transgene by standard PCR, as described by Jackson Laboratory. Offspring were used at 8 weeks of age.

### Cell Line

B16F0 (#CRL-6322) (57), B16F10 (#CRL-6475) (58), YUMMER1.7 (Marcus Bosenberg, Yale University)(59), EG7-OVA (#CRL-2113) (60) were purchased from ATCC. B16F10-OVA (61) and B16F10-OVA-mCherry2 (62) were kindly supplied by Dr. Matthew F. Krummel (University of California San Francisco) while mEERL (63) and MLM3 (63) cell lines were generated and used by Dr. Paola Vermeer (University of South Dakota). These cell lines were cultured in complete Dulbecco's Modified Eagle's Medium high glucose (DMEM, Gibco #D5796) supplemented with 10% fetal bovine serum (Biochrom, #12483020) and 1% penicillin/sterptomycin (Gibco, #15140163), and maintained at 37° C. in a humidified incubator with 5% CO₂.

### Cancer Inoculation

Cancer cells (1×10⁵) were resuspended in PBS and injected (i.d., 100 μl) to the mice right flank. Growth was daily assessed using a handheld caliper. Mice were euthanized when tumor reached 1000-1500 mm (57, 58, 62). Tumor and their draining lymph node (tdLN) were harvested. Tumors were enzymatically digested in DMEM (Gibco, #D5796)+2 mg/ml collagenase D (Sigma, #C5138)+0.03 mg/ml DNAse I (Sigma, #10104159001) under constant shaking (30 min, 37° C.). Tumor draining lymph nodes were dissected in PBS, mechanically dissociated using a plunger, strained (70 μm), washed with PBS. The cell suspensions were then strained (70 μm), washed and RBC were lysed (Life Technologies, #A1049201; 2 min).

### Drugs

QX-314 (64) (Tocris, #2313; 0.3%) was injected (i.d.) daily in 5 points around the tumor (treatment began once tumor was visible). BIBN4096 (Tocris, #4561; 5 mg/kg) was injected (i.p.) on day 6, 8, 10, 12 and 14. Botulinum neurotoxin A (65) (List biological labs, #130B; 25 pg/μl) was injected (i.d.) three and one day prior to, or one and three days after, tumor inoculation. αPD-L1 (66) (Bioxcell, #BE0101, 6 mg/kg) was injected (i.p.) on day 7, 10 and 13.

### In Vivo Depletion of CD3

200 μg/mice of anti-mouse CD3 (67) (Bioexcell, #BE0001) was injected (i.p.) 3 days prior to B16F10 inoculation (1×10⁵; i.d.) and continued every 3 days. Blood samples were taken twice weekly to confirm depletion and tumor growth measured daily, as previously described.

### Immunophenotyping

Single cells were resuspended in FACS buffer (PBS, 2% FCS, EDTA), Fc blocked (0.5 mg/ml, 10 min; BD Biosciences, #553141) and stained (30 min, 4° C.) with ZombieAqua (Biolegend, #423102) and monoclonal antibodies (anti-CD45-BV421 (1:100, Biolegend, #103134), anti-CD11b-APC/Cy7 (1:100, Biolegend, #101226), anti-CD8-AF700 (1:100, Biolegend, #100730), anti-CD4-PerCP/Cyanine5.5 (1:100, Biolegend, #100540), anti-NK-FITC (1:100, Biolegend, #108706), anti-PD-1-PE-Cy7 (1:100, Biolegend, #109110), anti-Lag3-PE (1:100, Biolegend, #125208), anti-Tim-3-APC (1:100, Biolegend, #119706).

### Intracellular Cytokine Staining

Cells were stimulated (4 h) with Brefeldin A (Biolegend, #423303), washed, fixed/permeabilized (BD Biosciences; #554714) and stained with anti-IFN-7-BV650 (Biolegend, #505832), anti-TNFα-FITC (Biolegend, #506304), anti-Granzyme B-APC (Biolegend, #396407).

### Skin Explant

3 h post-exposure to vehicle (100 al), QX-314 (0.3%, 100 μl) or BoNT/a (25 pg/μL, 100 μl), tumor-surrounding skin was harvested using 10 mm punch biopsies. The biopsies were transferred into 24-well plates and cultured into DMEM containing 1 μl/ml of protease inhibitor (Sigma, #P1860) and capsaicin (1 μM. Sigma, #M2028). After 30 min incubation (37° C.), the supernatant was collected and CGRP release analyzed (65) using a commercial ELISA (Cayman Chemical, #589001).

### Neuron Culture

Mice were sacrificed and dorsal root ganglia (DRG) were dissected out into DMEM medium (Corning, #10-013-CV), completed with 50 U/mL penicillin and 50 μg/ml streptomycin (Fisher, #MT-3001-C1), and 10% FBS (Seradigm, #3100). Cells were then dissociated in HEPES buffered saline (Sigma, #51558) completed with 1 mg/mL collagenase IV (Sigma, #C0130)+2.4 U/mL dispase II (Sigma, #04942078001) and incubated for 80 minutes at 37° C. Ganglia were triturated with glass Pasteur pipettes of decreasing size in supplemented DMEM medium, then centrifuged over a 10% BSA gradient, plated on laminin (Sigma, #L2020) coated cell culture dishes. Cells were cultured with Neurobasal-A medium (Gibco, #21103-049) completed with 0.05 ng/μL NGF (Life Technologies, #13257-019), 0.002 ng/μL GDNF (Peprotech, #450-51-10), 0.01 mM AraC (Sigma, #C6645) and 200 mM L-Glutamine (VWR, #02-0131) (68).

### Calcium Imaging

L3-L5 DRG neurons were harvested and co-cultured with B16F10, B16F0 or MPEK-BL6 for 24-48 h. The cells were then loaded with 5 mM Fura-2 AM (BioVision, #2243) in complete Neurobasal-A medium for 30 min at 37° C., washed into Standard Extracellular Solution (SES, 145 mM NaCl, 5 mM KCl, 2 mM CaCl₂), 1 mM MgCl₂, 10 mm glucose, 10 mM HEPES, pH 7.5), and response to noxious ligands (100 nM capsaicin; 100 μM AITC; 1 μM ATP) analyzed at room temperature. Ligands were flowed (15 s) directly onto neurons using perfusion barrels followed by buffer washout (105-sec minimum) (68). Cells were illuminated by a UV light source (Xenon lamp, 75 watts, Nikon), 340 nm and 380 nm excitation alternated by an LEP MAC 5000 filter wheel (Spectra services), and fluorescence emission captured by Cool SNAP ES camera (Princeton Instruments). 340/380 ratiometric images were processed, background corrected and analyzed (IPLab software; Scientific Analytics) and Microsoft Excel used for post-hoc analyses.

### Immunofluorescence

2×10³ DRG neurons were co-cultured with 2×10⁴ B16F10-mCherry-OVA for 24-48 h. The cells were fixed (4% paraformaldehyde; 30 min), permeabilized (0.1% Triton X-100, 20 min), and blocked (PBS, 0.1% Triton X-100, 5% BSA, 30 min). The cells were rinsed (PBS), stained and mounted with vectashield containing DAPI (Vector Laboratories, #H-1000). Images were acquired using a Ti2 Nikon fluorescent microscope.

### Clarity

Passive CLARITY protocol (69) was used to cleared 14d-inoculated B16F10 tumors. Peripheral nerves were stained with an A647 conjugated anti-tubulin β3 (1:100, Biolegend, #801209) polyclonal antibody. Samples were then submerged in PROTOS index-matching imaging medium (LifeCanvas, #SHIELF), imaged (5×) with lightsheet microscope (Zeiss Z.1) and data showed as 2 mm-thick maximum projection (scale=500 μm).

### CD8 Isolation

6-8 weeks-old male and female mice were euthanized, spleen harvested in ice-cold PBS (5% FBS), and mechanically dissociated. The cells were strained (70 μm), RBC lysed (Life Technologies, #A1049201; 2 min), and counted using a hemocytometer. Naïve CD8⁺ T cells were magnet sorted (Stem cell, #19853A) and cultured (DMEM+FBS 10%, Pen/Strep +non-essential amino acid (Corning, #25-025-C1)+vitamin+β-mercaptoethanol (Gibco, #21985-023)+L-Glutamine (VWR, #02-0131)+sodium pyruvate (Corning, #25-000-C1)). Cell purity was confirmed after magnet sorting by labeling cells against CD62L and the numbers of CD8⁺CD62^{hi} immunophenotyped by flow cytometry. To generate cytotoxic T lymphocytes, 1×10⁶ naïve CD8⁺ T cells were seeded and stimulated for 48 h under Tc1 inflammatory condition (2 μg/ml plate bounded αCD3/αCD28 (Bioxcell, #BE00011, #BE00151)+10 ng/ml rIL-12 (Biolegend, #577008)+10 μg/ml of anti-IL-4 (Bioxcell, #BE0045).

In Vitro Cytotoxic CD8 T Cell Stimulation with Neuropeptides

CD8 T cells were isolated and stimulated for 48 h in 96 wells plate. In the presence of peptidase inhibitor, the cells were treated with either of CGRP (0.1 uM), VIP (1 uM) and SST (0.1 uM), or a combination of these. Expression of PD-1, Lag-3 and Tim-3 as well as INFγ, TNFα and IL-2 were immunophenotyped by flow cytometry.

### Co-Culture

Naïve DRG neurons (1×10⁴) were seeded in T cell media (supplemented with 0.05 ng/μL NGF (Life Technologies, #13257-019), 0.002 ng/μL GDNF (Peprotech, #450-51-10), and co-cultured with Tc1 CD8 T cells (1×10⁵) in presence of IL-2 (575408). In some instances, co-cultures were stimulated with either of capsaicin (300 nM, twice/day) or KCl (40 mM). After 48 h, the cells were collected by centrifugation (5 min at 1300 rpm), stained and immunophenotyped by flow cytometry.

In Vitro Cytotoxic CD8 T Cell Stimulation with Neurons' Conditioned Media

DRG neurons were cultured (24 h) in complete T cell media supplemented with 1 μl/ml protease inhibitor (Sigma, #P1860), 0.05 ng/μL NGF (Life Technologies, #13257-019), and 0.002 ng/μL GDNF (Peprotech, #450-51-10) and stimulated with KCl (40 mM). The conditioned media or vehicle were collected after 10 min and added to CD8 T cells for 48 h. The CD8 T cells expression of exhaustion markers (PD1, Lag3 and Tim3) and cytokine (INFγ, TNFα, IL-2) were analyzed by flow cytometry.

### Apoptosis

1×10⁴ naïve TRPV1^{Cre}::QuASR2^{fl/wt} DRG neurons were co-cultured (24-72 h) with 1×10⁵ OVA-specific cytotoxic CD8 T cells harvested from OT1 mice and 1×10⁵ B16F10-mCherry-OVA in T cell media (supplemented with 0.05 ng/μL NGF (Life Technologies, #13257-019), 0.002 ng/μL GDNF (Peprotech, #450-51-10). After 48 h co-culture, the cells were collected by centrifugation (5 min at 1300 rpm), stained using anti-Annexin V-APC, and 7-AAD, (Biolegend, #640930) and immunophenotyped by flow cytometry. Alternatively, co-cultures were imaged every 6 h using a fluorescent microscope (Nikon, Ti2).

### Pain Behavioral

10⁵ B16F10 cells or 10⁵ non-cancerous keratinocytes (MPEK-BL6) were inoculated intradermally in the mice left hindpaw. Once every two-day, the thermal and mechanical sensitivity was evaluated using a plantar test (Hargreaves' method) analgesia meter (70) (IITC Life Science, Model 390G,) and von Frey monofilaments (70) (UgoBasile, #52-37450-275), respectively.

### B16F10 Survival

2×10⁵ B16F10 cells were cultured in 6-well-plate and challenged with BoNT/a (0-50 pg/μl) for 24 h, QX-314 (0-1%) for 72 h or vehicle. B16F10 cells survival was assessed using anti-annexin V-APC and 7-AAD (Biolegend #640930) using flow cytometry.

qPCR

After 48 h in co-culture with DRG neurons, live CD8 T cells were purified by flow cytometry (FACSAria), RNA extracted in Trizol (Sigma-Aldrich, #93289), cDNA generated (SuperScript® VILO™ cDNA Synthesis Kit) and transcript expression (Ramp1, Ramp3, Vpac2, GAPDH) quantified (Power SYBR Green PCR Master mix) by qPCR (BMS; Mic thermocycler), as instructed by the manufacturer (70).

### Neurite Outgrowth Assessment

2×10³ DRG neurons from TRPV1^{Cre}::Tdtomato^{fl/wt} mice were harvested and co-cultured (2×10⁴) with non-tumorigenic keratinocytes or B16F10-GFP. The cells were fixed, imaged (Nikon, Ti2 fluorescent microscope) and neurite outgrowth analyzed using ImageJ.

### In-Silico Analysis

The OncoLnc database (www.oncolnc.org) was used to assess transcript expression of 333 neuronal-enriched genes (neuronal membrane proteins, neural stem cell markers, transcription factors, ion channel receptors, and neuropeptides) in 459 skin cancer (SKCM) tumor biopsies from the Cancer Genome Atlas (TCGA) database. 206 of these genes were expressed, and 108 selected based on their negative Cox coefficient value, indicating a link between lower gene expression and improved patient survival. The cBioPortal (www.cbioportal.org) allowed us to link gain and loss-of-function mutations to 333 neuronal-enriched genes and survival of 1517 skin cancer patient. The Oncomine database (www.oncomine.org), was used to browse previously published sequencing datasets (71-74), to compare the expression of 30 nociceptor-enriched genes in normal skin or benign nevus with melanoma biopsies.

### Patients Biopsy

Melanoma biopsies were collected at Sanford Health and classified by a board-certified pathologist. Given that patient samples were de-identified and no information other than tumor type was provided, the Sanford Health IRB company established that these samples were not considered human subjects research and no IRB number was assigned. DERM103 patient sample classified as a malignant melanoma from the left posterior shoulder, MART-1 and HMB45 positive, melanocytic in nature (Elastic positive) and MIB-1 positive (increased proliferation). DERM107 patient sample classified as malignant melanoma, high mitotic index, invasive, stage pT2a, MART-1, SOX-10, P16, and HMB45 positive. DERM110 patient sample classified as malignant melanoma; high mitotic index, Clark's level IV, stage pT1b, the tumor appears to be invading the epidermis with a fusion of malignant sheets of cells, strongly positive for SOX-10, MART-1, HMB45 with a moderate increase in proliferative index.

### Statistics

Data expressed as mean±S.E.M. Statistical significance determined by one-way or two-way ANOVA for multiple comparisons and two-tail unpaired Student's t-test for single variable comparison. P values less than 0.05 were considered significant. Numbers of animals are defined in figure legends.

### REFERENCES FOR EXAMPLE 1

1. P. Mauffrey et al., Progenitors from the central nervous system drive neurogenesis in cancer. Nature 569, 672-678 (2019).
2. C. Magnon et al., Autonomic nerve development contributes to prostate cancer progression. Science 341, 1236361 (2013).
3. A. H. Zahalka et al., Adrenergic nerves activate an angio-metabolic switch in prostate cancer. Science 358, 321-326 (2017).
4. A. H. Zahalka, P. S. Frenette, Nerves in cancer. Nat Rev Cancer 20, 143-157 (2020).
5. M. Amit et al., Loss of p53 drives neuron reprogramming in head and neck cancer. Nature 578, 449-454 (2020).
6. M. Dougan, G. Dranoff, Immune therapy for cancer. Annu Rev Immunol 27, 83-117 (2009).
7. C. A. Chambers, M. S. Kuhns, J. G. Egen, J. P. Allison, CTLA-4-mediated inhibition in regulation of T cell responses: mechanisms and manipulation in tumor immunotherapy. Annu Rev Immunol 19, 565-594 (2001).
8. S. L. Topalian et al., Safety, activity, and immune correlates of anti-PD-1 antibody in cancer. N Engl J Med 366, 2443-2454 (2012).
9. M. Das, C. Zhu, V. K. Kuchroo, Tim-3 and its role in regulating anti-tumor immunity. Immunol Rev 276, 97-111 (2017).
10. S. H. Baumeister, G. J. Freeman, G. Dranoff, A. H. Sharpe, Coinhibitory Pathways in Immunotherapy for Cancer. Annu Rev Immunol 34, 539-573 (2016).
11. M. D. Vesely, M. H. Kershaw, R. D. Schreiber, M. J. Smyth, Natural innate and adaptive immunity to cancer. Annu Rev Immunol 29, 235-271 (2011).
12. S. R. Woo, L. Corrales, T. F. Gajewski, Innate immune recognition of cancer. Annual review of immunology 33, 445-474 (2015).
13. P. Anderson, E. Gonzalez-Rey, Vasoactive intestinal peptide induces cell cycle arrest and regulatory functions in human T cells at multiple levels. Mol Cell Biol 30, 2537-2551 (2010).
14. J. M. Li et al., VIPhyb, an antagonist of vasoactive intestinal peptide receptor, enhances cellular antiviral immunity in murine cytomegalovirus infected mice. PLoS One 8, e63381 (2013).
15. J. M. Li et al., Modulation of Immune Checkpoints and Graft-versus-Leukemia in Allogeneic Transplants by Antagonizing Vasoactive Intestinal Peptide Signaling. Cancer Res 76, 6802-6815 (2016).
16. C. T. Petersen, J. M. Li, E. K. Waller, Administration of a vasoactive intestinal peptide antagonist enhances the autologous anti-leukemia T cell response in murine models of acute leukemia. Oncoimmunology 6, e1304336 (2017).
17. D. Pozo, P. Anderson, E. Gonzalez-Rey, Induction of alloantigen-specific human T regulatory cells by vasoactive intestinal peptide. J Immunol 183, 4346-4359 (2009).
18. P. Tebas et al., Reduction of soluble CD163, substance P, programmed death 1 and inflammatory markers: phase 1B trial of aprepitant in HIV-1-infected adults. AIDS 29, 931-939 (2015).
19. G. Chen et al., PD-L1 inhibits acute and chronic pain by suppressing nociceptive neuron activity via PD-1. Nat Neurosci 20, 917-926 (2017).
20. K. Ondicova, B. Mravec, Role of nervous system in cancer aetiopathogenesis. Lancet Oncol 11, 596-601 (2010).
21. J. L. Saloman et al., Ablation of sensory neurons in a genetic model of pancreatic ductal adenocarcinoma slows initiation and progression of cancer. Proc Natl Acad Sci USA 113, 3078-3083 (2016).
22. S. Talbot, S. L. Foster, C. J. Woolf, Neuroimmunity: Physiology and Pathology. Annu Rev Immunol 34, 421-447 (2016).
23. A. Kamiya et al., Genetic manipulation of autonomic nerve fiber innervation and activity and its effect on breast cancer progression. Nat Neurosci 22, 1289-1305 (2019).
24. S. C. Goswami et al., Molecular signatures of mouse TRPV1-lineage neurons revealed by RNA-Seq transcriptome analysis. J Pain 15, 1338-1359 (2014).
25. T. Crosson et al., Profiling of how nociceptor neurons detect danger—new and old foes. J Intern Med 286, 268-289 (2019).
26. J. C. Castle et al., Exploiting the mutanome for tumor vaccination. Cancer Res 72, 1081-1091 (2012).
27. P. Baral et al., Nociceptor sensory neurons suppress neutrophil and gammadelta T cell responses in bacterial lung infections and lethal pneumonia. Nat Med 24, 417-426 (2018).
28. I. M. Chiu et al., Bacteria activate sensory neurons that modulate pain and inflammation. Nature 501, 52-57 (2013).
29. F. A. Pinho-Ribeiro et al., Blocking Neuronal Signaling to Immune Cells Treats Streptococcal Invasive Infection. Cell 173, 1083-1097 e1022 (2018).
30. N. Yissachar et al., An Intestinal Organ Culture System Uncovers a Role for the Nervous System in Microbe-Immune Crosstalk. Cell 168, 1135-1148 e1112 (2017).
31. W. Ding, L. L. Stohl, J. A. Wagner, R. D. Granstein, Calcitonin gene-related peptide biases Langerhans cells toward Th2-type immunity. J Immunol 181, 6020-6026 (2008).
32. B. Boilly, S. Faulkner, P. Jobling, H. Hondermarck, Nerve Dependence: From Regeneration to Cancer. Cancer Cell 31, 342-354 (2017).
33. C. M. Zhao et al., Denervation suppresses gastric tumorigenesis. Sci Transl Med 6, 250ra115 (2014).
34. S. W. Kashem et al., Nociceptive Sensory Fibers Drive Interleukin-23 Production from CD301b+ Dermal Dendritic Cells and Drive Protective Cutaneous Immunity. Immunity 43, 515-526 (2015).
35. J. D. Wolchok et al., Nivolumab plus ipilimumab in advanced melanoma. N Engl J Med 369, 122-133 (2013).
36. G. V. Long et al., Nivolumab for Patients With Advanced Melanoma Treated Beyond Progression: Analysis of 2 Phase 3 Clinical Trials. JAMA Oncol 3, 1511-1519 (2017).
37. S. Talbot et al., Silencing Nociceptor Neurons Reduces Allergic Airway Inflammation. Neuron 87, 341-354 (2015).
38. S. Pellett, W. H. Tepp, R. C. Whitemarsh, M. Bradshaw, E. A. Johnson, In vivo onset and duration of action varies for botulinum neurotoxin A subtypes 1-5. Toxicon 107, 37-42 (2015).
39. A. M. Binshtok, B. P. Bean, C. J. Woolf, Inhibition of nociceptors by TRPV1-mediated entry of impermeant sodium channel blockers. Nature 449, 607-610 (2007).
40. N. Cancer Genome Atlas Research et al., The Cancer Genome Atlas Pan-Cancer analysis project. Nat Genet 45, 1113-1120 (2013).
41. C. Haqq et al., The gene expression signatures of melanoma progression. Proc Natl Acad Sci USA 102, 6092-6097 (2005).
42. H. Harlin et al., Chemokine expression in melanoma metastases associated with CD8+ T-cell recruitment. Cancer Res 69, 3077-3085 (2009).
43. A. I. Riker et al., The gene expression profiles of primary and metastatic melanoma yields a transition point of tumor progression and metastasis. BMC Med Genomics 1, 13 (2008).
44. D. Talantov et al., Novel genes associated with malignant melanoma but not benign melanocytic lesions. Clin Cancer Res 11, 7234-7242 (2005).
45. L. Jerby-Arnon et al., A Cancer Cell Program Promotes T Cell Exclusion and Resistance to Checkpoint Blockade. Cell 175, 984-997 e924 (2018).
46. I. Tirosh et al., Dissecting the multicellular ecosystem of metastatic melanoma by single-cell RNA-seq. Science 352, 189-196 (2016).
47. T. S. Heng, M. W. Painter, C. Immunological Genome Project, The Immunological Genome Project: networks of gene expression in immune cells. Nat Immunol 9, 1091-1094 (2008).
48. M. Uhlen et al., Proteomics. Tissue-based map of the human proteome. Science 347, 1260419 (2015).
49. G. Monaco et al., RNA-Seq Signatures Normalized by mRNA Abundance Allow Absolute Deconvolution of Human Immune Cell Types. Cell Rep 26, 1627-1640 e1627 (2019).
50. K. A. Hogquist et al., T cell receptor antagonist peptides induce positive selection. Cell 76, 17-27 (1994).
51. D. J. Cavanaugh et al., Trpv1 reporter mice reveal highly restricted brain distribution and functional expression in arteriolar smooth muscle cells. J Neurosci 31, 5067-5077 (2011).
52. L. Madisen et al., A toolbox of Cre-dependent optogenetic transgenic mice for light-induced activation and silencing. Nat Neurosci 15, 793-802 (2012).
53. L. Madisen et al., A robust and high-throughput Cre reporting and characterization system for the whole mouse brain. Nat Neurosci 13, 133-140 (2010).
54. D. Voehringer, H. E. Liang, R. M. Locksley, Homeostasis and effector function of lymphopenia-induced “memory-like” T cells in constitutively T cell-depleted mice. J Immunol 180, 4742-4753 (2008).
55. S. Lou et al., Genetically Targeted All-Optical Electrophysiology with a Transgenic Cre-Dependent Optopatch Mouse. J Neurosci 36, 11059-11073 (2016).
56. N. Agarwal, S. Offermanns, R. Kuner, Conditional gene deletion in primary nociceptive neurons of trigeminal ganglia and dorsal root ganglia. Genesis 38, 122-129 (2004).
57. I. J. Fidler, Biological behavior of malignant melanoma cells correlated to their survival in vivo. Cancer Res 35, 218-224 (1975).
58. I. J. Fidler, M. L. Kripke, Metastasis results from preexisting variant cells within a malignant tumor. Science 197, 893-895 (1977).
59. K. Meeth, J. X. Wang, G. Micevic, W. Damsky, M. W. Bosenberg, The YUMM lines: a series of congenic mouse melanoma cell lines with defined genetic alterations. Pigment Cell Melanoma Res 29, 590-597 (2016).
60. M. W. Moore, F. R. Carbone, M. J. Bevan, Introduction of soluble protein into the class I pathway of antigen processing and presentation. Cell 54, 777-785 (1988).
61. M. B. Headley et al., Visualization of immediate immune responses to pioneer metastatic cells in the lung. Nature 531, 513-517 (2016).
62. M. L. Broz et al., Dissecting the tumor myeloid compartment reveals rare activating antigen-presenting cells critical for T cell immunity. Cancer Cell 26, 638-652 (2014).
63. D. W. Vermeer et al., Metastatic model of HPV+ oropharyngeal squamous cell carcinoma demonstrates heterogeneity in tumor metastasis. Oncotarget 7, 24194-24207 (2016).
64. A. M. Binshtok, B. P. Bean, C. J. Woolf, Inhibition of nociceptors by TRPV1-mediated entry of impermeant sodium channel blockers. Nature 449, 607-610 (2007).
65. P. Baral et al., Nociceptor sensory neurons suppress neutrophil and gammadelta T cell responses in bacterial lung infections and lethal pneumonia. Nat Med 24, 417-426 (2018).
66. C. Stathopoulou et al., PD-1 Inhibitory Receptor Downregulates Asparaginyl Endopeptidase and Maintains Foxp3 Transcription Factor Stability in Induced Regulatory T Cells. Immunity 49, 247-263 e247 (2018).
67. P. A. Carpenter et al., Non-Fc receptor-binding humanized anti-CD3 antibodies induce apoptosis of activated human T cells. J Immunol 165, 6205-6213 (2000).
68. S. Talbot et al., Silencing Nociceptor Neurons Reduces Allergic Airway Inflammation. Neuron 87, 341-354 (2015).
69. K. Chung et al., Structural and molecular interrogation of intact biological systems. Nature 497, 332-337 (2013).
70. S. Talbot, H. De Brito Gariepy, J. Saint-Denis, R. Couture, Activation of kinin B1 receptor evokes hyperthermia through a vagal sensory mechanism in the rat. J Neuroinflammation 9, 214 (2012).
71. C. Haqq et al., The gene expression signatures of melanoma progression. Proc Natl Acad Sci USA 102, 6092-6097 (2005).
72. H. Harlin et al., Chemokine expression in melanoma metastases associated with CD8+ T-cell recruitment. Cancer Res 69, 3077-3085 (2009).
73. A. I. Riker et al., The gene expression profiles of primary and metastatic melanoma yields a transition point of tumor progression and metastasis. BMC Med Genomics 1, 13 (2008).
74. D. Talantov et al., Novel genes associated with malignant melanoma but not benign melanocytic lesions. Clin Cancer Res 11, 7234-7242 (2005).

### Example 2

Tumor denervation limits cancer growth, but the mechanisms behind this are unknown. Provided herein is the finding that malignant melanoma cells directly interact with pain-initiating nociceptor neurons by increasing neuron outgrowth, responsiveness to noxious ligands and neuropeptide release. In turn, nociceptor neuropeptides increase exhaustion of cytotoxic T cells (PD1⁺Lag3⁺Tim3⁺INFγ⁻), limiting their capacity to eliminate melanoma cells. Genetic ablation (TRPV1^{cre}::DTA^{fl/wt}; Na_{V}1.8^{cre}::DTA^{fl/wt}), local pharmacological silencing (QX-314) as well as blockade of vesicle release (BoNT/a) from tumor-innervating nociceptors enhance tumor-infiltrating leukocyte (TIL) numbers and survival in mice subject to orthotropic melanoma inoculation, blunting tumor growth and TIL exhaustion. Similar findings were obtained in mice inoculated with a Braf^{V600E}Cdkn2a^{-/-} Pten^{-/-} melanoma as well as in a model of lung metastatic HPV⁺ oropharyngeal squamous cell carcinoma. These effects are absent upon CD3⁺ depletion and are phenocopied by local administration of a calcitonin gene-related peptide (CGRP) receptor antagonist. Silencing or ablating sensory neurons also increases the response to systemic PDL1 blockade. Further support for involvement of nociceptors in cancer comes from the increased tumor growth and greater TIL exhaustion produced by optogenetic activation (Na_{V}1.8^{cre}:ChR2^{fl/wt}) of tumor-innervating nociceptors and Tubb3⁺ innervation of metastatic melanoma patients' biopsies. Provided herein is the conclusion reducing neuropeptide release from tumor-innervating nociceptors, by eliminating their immunomodulatory action on cytotoxic CD8 T cell, may be a useful therapeutic intervention to boost immune surveillance.

Shown herein is nociceptor neurons stimulated by B16F10-melanoma cells release neuropeptides that modulate cytotoxic CD8 T cell activity, including an overexpression of immune checkpoint receptors. Targeted genetic ablation or local temporary pharmacological silencing of tumor-innervating sensory neurons decreases the growth of either triple-negative B16F10 melanoma, Braf^{V600E}Cdkn2a^{-/-} Pten^{-/-} melanoma or lung metastatic HPV⁺ oropharyngeal squamous cell carcinomas, in a CD3-dependant manner. These treatments also prevent TIL exhaustion and increase the response to anti-PD-L1 blockade. Optogenetic activation of tumor-innervating sensory neurons, furthermore, drives increased cancer growth and TIL exhaustion. Therefore, it was concluded that nociceptor neurons dampen CD8 T cell activity by expressing ligands for⁵² and controlling the expression of immune checkpoint receptors. In consequence, nociceptor sensory neurons emerge as a therapeutic driver of immune regulation in a cancer setting.

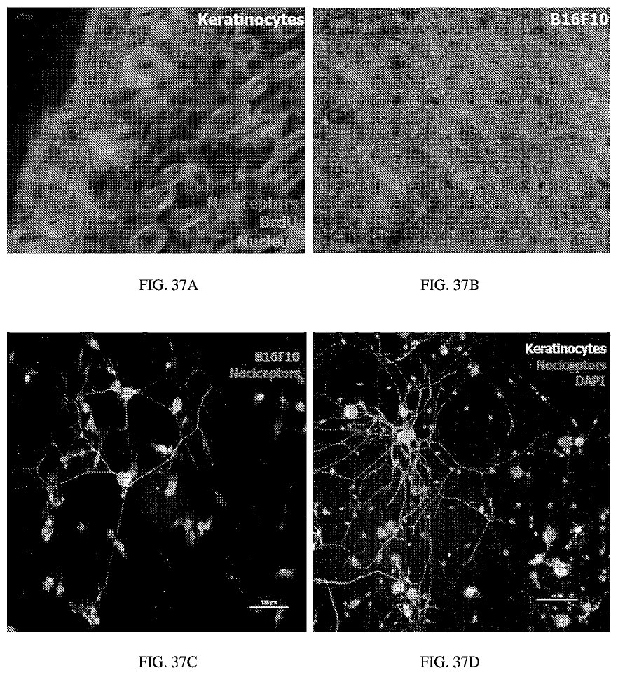

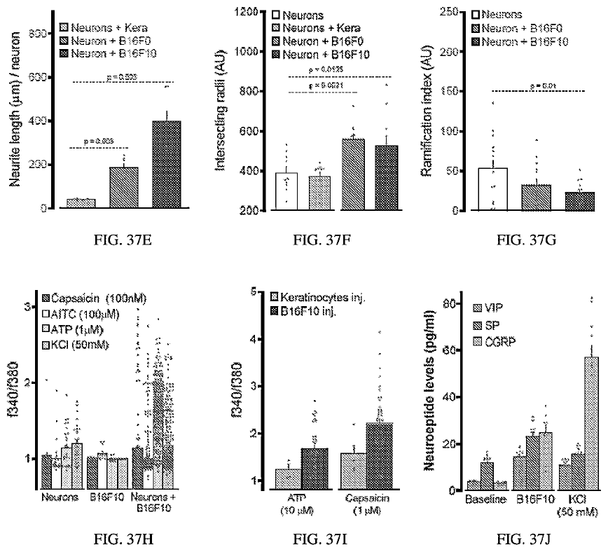

It was first probed whether melanoma modulate the growth and sensitivity of nociceptors. To do this, 8-week-old male and female mice whose sensory neurons were fluorescently labeled (TRPV1^{Cre}::Tdtomato^{fl/wt}; a lineage reporter virtually labelled all nociceptors⁷²) were inoculated intradermally with 10⁵ B16F10 cells or non-cancerous keratinocytes (MPEK-BL6). Fourteen days post-inoculation, the B16F10 tumor showed nociceptor axonal innervation of the BrDu⁺ cells (FIGS. 37A-37B). In support of this, IHC was used and found Tubb3 expressing fibers in three aggressive melanomas biopsies.

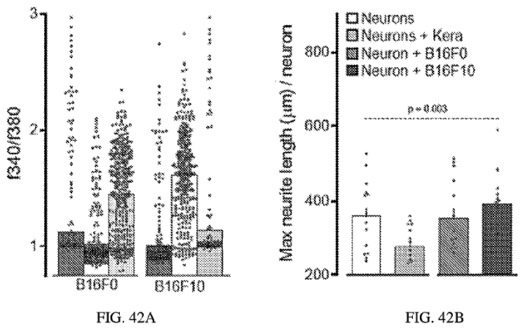

It was then sought to probe the nature of the interplay between melanoma cancer cells and nociceptors. To do this, a double reporter co-culture assay was devised that included fluorescently labeled (TRPV1^{Cre}::Tdtomato^{fl/wt}) DRG nociceptor neurons and eGFP-expressing B16F10 cells, or non-tumorigenic keratinocytes. When co-cultured with B16F10 cells, nociceptor neurites typically extend toward B16F10 cells, forming contacts (FIGS. 37C-37D). In addition, the average neurite length of nociceptor neurons increased (number of intersecting radi; FIGS. 37E, 37F, 42B) while the overall neuronal arborisation decreased (ramification index; FIG. 37G) when co-cultured with B16F10 cells. Ipsilateral L3-L5 DRG nociceptors harvested from tumor-inoculated mice (on d14) put in culture for 72 h extend longer neurites than their counterparts harvested from keratinocyte-injected mice (not shown). These findings indicate that nociceptors grow differently when in proximity to melanoma cells as opposed to 1 keratinocytes.

Given that melanoma promotes axonogenesis, which leads to innervation it was next aimed to test whether this would physical proximity modulates the sensitivity of nociceptor neurons. To test this, the number and the amplitude of calcium flux induced in response to sub-threshold concentration of capsaicin (an exogenous agonist of the heat sensing channel TRPV1 (Transient Receptor Potential Vanilloid-1))-, mustard oil-(an exogenous agonist of the chemical sensing channel TRPA1 (Transient Receptor Potential Anykrin-1)), and ATP-(an exogenous agonist of the proton-sensing channel P2X3R) was measured. It was found that when nociceptors are cultured without melanoma cells the ligands, at the concentration selected activated a few DRG neurons. However, the numbers of responsive neurons increased when co-cultured with B16F10 cells (FIG. 37H; FIG. 42A). Similarly, when tested for the amplitude of calcium flux responses to the ligands, ipsilateral L3-L5 DRG neurons harvested from tumor-inoculated mice (on d14) had heightened sensitivity in comparison to their counterparts harvested from keratinocyte-injected mice (FIG. 37I). In addition, it was found that neurons co-cultured with B16F10 cells (5×10⁴ cells, 24 h) increase release of SP, CGRP, and VIP (FIG. 37J).

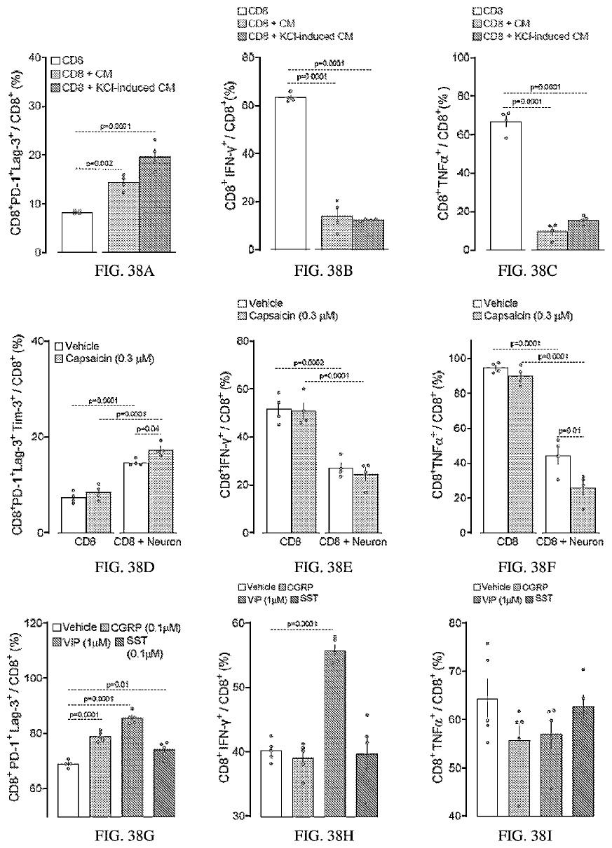

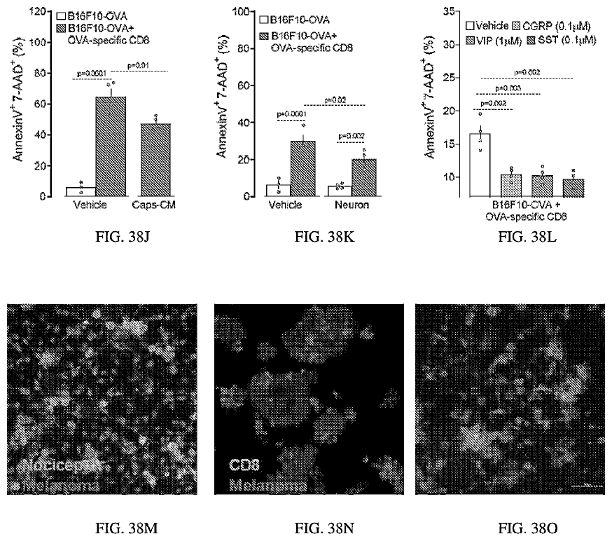

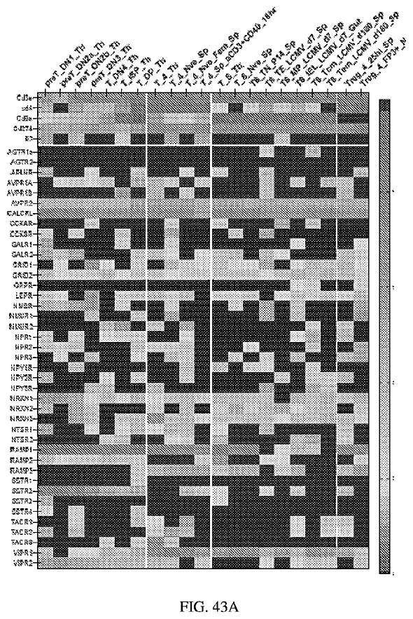

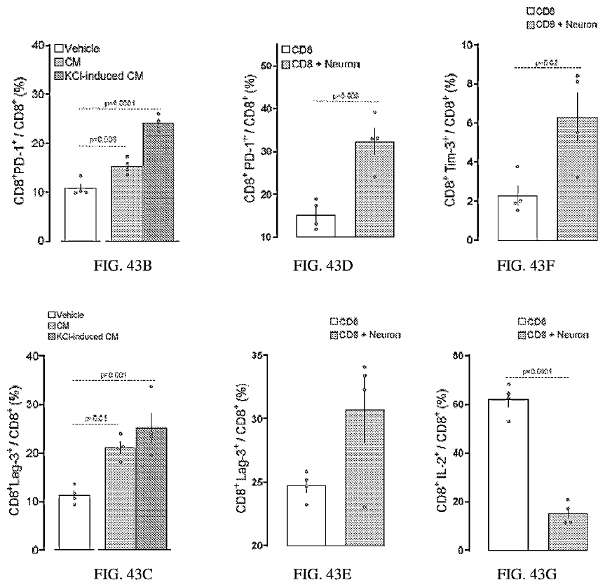

In-silico analysis of the Immgen database (⁷³) revealed that cytotoxic CD8⁺ T cells express a wide variety of (≥10) neuropeptide receptors (FIG. 43A). Given that nociceptors readily interact with CD8 T cells when put in co-culture and that the neuropeptides they release block T_{H}1 immunity⁷⁴⁻⁷⁷, it was tested if these neuropeptides have direct effects on cytotoxic CD8⁺ T cell′ expression of immune checkpoint receptors. It was first sought to test the immunomodulatory effect of KCl-stimulated (30 min) cultured DRG neuron conditioned media (CM), which contains nociceptor-produced neuropeptides. Splenocyte-isolated CD8⁺ T cells were cultured under T_{C1}-stimulating conditions for 48 h and then exposed to CM. In comparison to control media (with KCl), CM increased the proportion of CD8⁺ T cells expressing PD1⁺Lag3⁺ (FIG. 38A), PD1⁺ (FIG. 43B), Lag3⁺ (FIG. 43C), and decreased levels of INFγ⁺ (FIG. 38B), and TNFα⁺ (FIG. 38C) cells. CM from capsaicin-stimulated neuron displayed a similar phenotype (not shown). Next, T_{C1}-stimulated CD8 T cells with neurons were co-cultured to mimic their local interaction. An increased proportion of PD1⁺Lag3⁺Tim3⁺ (FIG. 38D), PD1⁺ (FIG. 43D), Lag3⁺ (FIG. 43E), Tim3⁺ (FIG. 43F), and decreased levels of INFγ⁺ (FIG. 38E), TNFα⁺ (FIG. 38F), and IL2⁺ (FIG. 43G) was noted when CD8⁺ T cells were with DRG neurons.

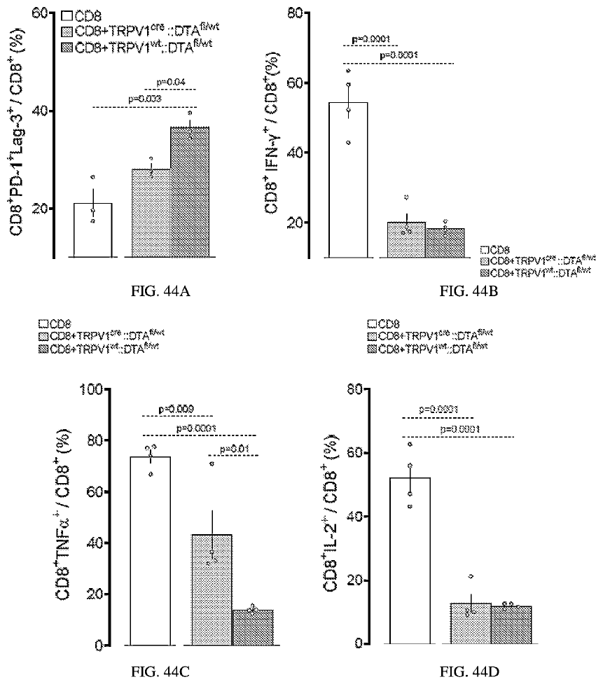

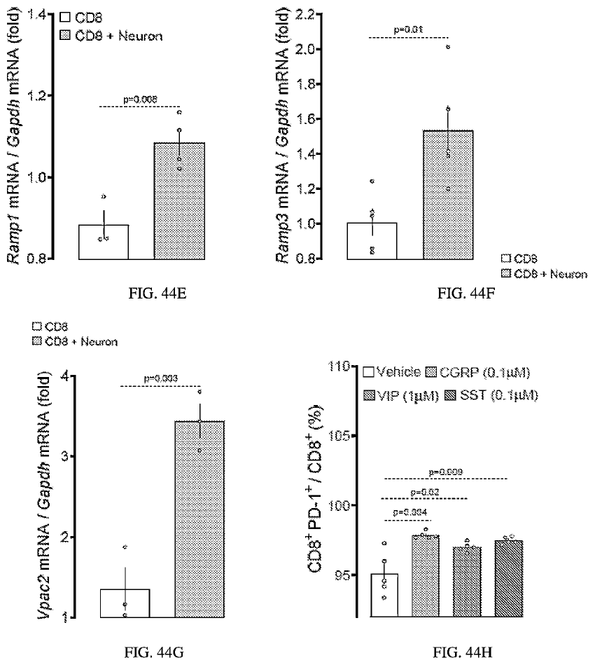

Based on these data, the focus was on investigating the immunomodulatory role of TRPV1⁺ nociceptors, the major NP producers, using pharmacological gain (capsaicin) and loss-of-function (TRPV1^{Cre}::DTA^{fl/wt}) approaches. Lymphocyte′ exhaustion was reduced when CD8 cells were co-cultured with DRG neurons from TRPV1^{Cre}::DTA^{fl/wt} (a mouse genetically-engineered to ablate nociceptors), while capsaicin stimulation of wildtype DRG neurons cultured with CD8 T cells had the opposite effects (FIGS. 44A-44D). Of note, capsaicin had no measurable impact in the absence of neurons. It was also found that CD8 T cells increase the expression of neuropeptide receptors Ramp1, Ramp3, and Vpac1 when co-cultured with sensory neurons (FIGS. 44E-44G), indicating a possible neuropeptide-mediated inhibitory loop between the two populations of cells.

Given the breadth of mediators (SP, CGRP, and VIP) released by nociceptors when exposed to melanoma cells (FIG. 37J), it was sought to define the immunomodulatory effects of the individual peptides. In the presence of a peptidase inhibitor, individual neuropeptides (CGRP, VIP, SST) enhanced the proportion of PD1⁺Lag3⁺Tim3⁺ (FIG. 38G), INFγ⁺ (FIG. 38H), PD1⁺ (FIG. 44H), Lag3⁺ (FIG. 44I), Tim3⁺ (FIG. 44J) and IL2⁺ CD8 T cells (FIG. 44K) while it had no impact on TNFα⁺ expression (FIG. 38I). Overall, the various immunomodulatory effects of the neuron CM, or co-culture, to one NP could not ascribed, it was found that combining neuropeptides prompted a decreased proportion of INFγ⁺ CD8 T cells (FIG. 44L). Taken together, these data support a role for nociceptor-released NP as modulators of CD8 T cell exhaustion.

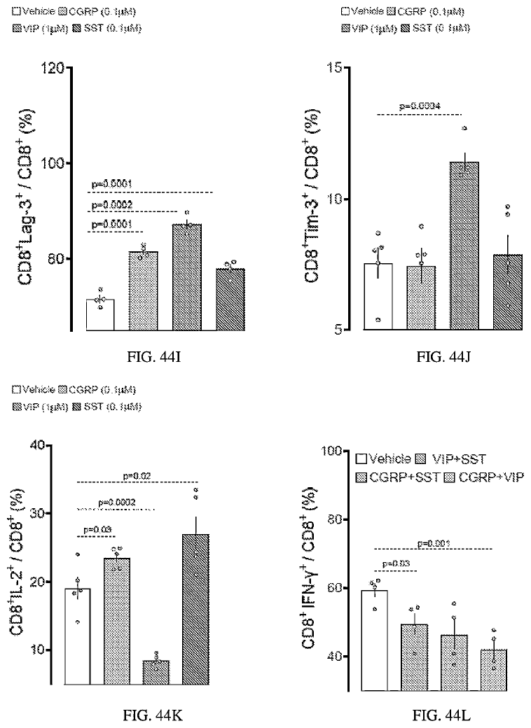

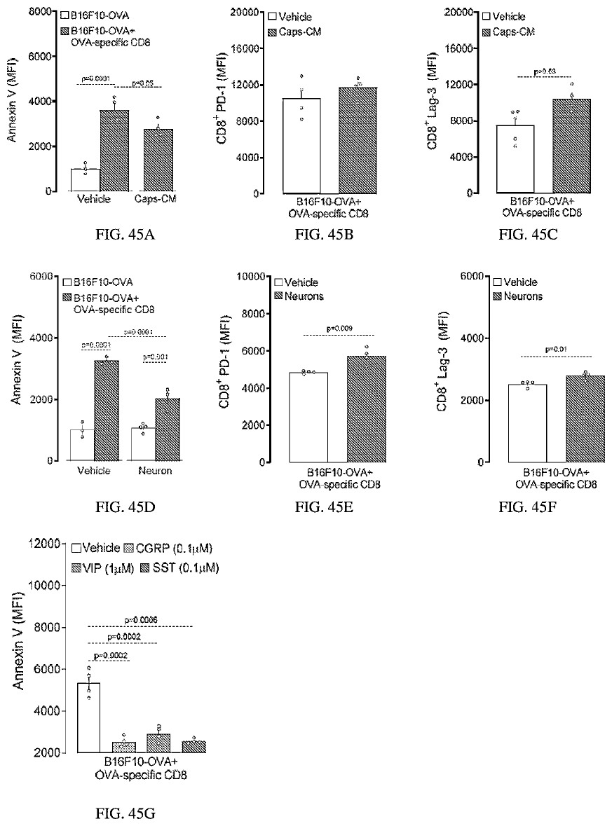

Then in order to test whether neuropeptides released from sensory neuron impact CD8 T cells anti-tumor responses, DRG neurons were co-cultured with B16F10-OVA cancer cells, and OT1 cytotoxic T cells. OT1 cells were harvested from naïve mouse splenocyte CD8 T cells primed under T_{C1}-stimulating conditions for 48 h. As expected, the presence of OT1 cytotoxic T cells drastically increased AnnexinV⁺7AAD⁺ B16F10-OVA cells mediated apoptosis in the cancer cells (FIGS. 38J-38K). It was also observed a decrease in tumor apoptosis when B16F10-OVA and OT1 cytotoxic T cells were stimulated with CM from neurons (induced by capsaicin; FIGS. 38J, 45A). Apoptosis was similarly induced in the cancer cells by co-culture with nociceptors (FIGS. 38K, 45D) or after NP stimulation (FIGS. 38L, 45G). These phenotypes correlate with an enhanced ratio of PD1⁺ and Lag3⁺ cytotoxic T cells (FIGS. 45B, 45C, 45E, 45F). These effects were confirmed using live-cell imaging, which showed that nociceptors (TRPV1^{Cre}::QuASR2-eGFP^{fl/wt}) enhanced B16F10-OVA-mCherry survival by diminishing the activity of OT1 cytotoxic T cells (FIGS. 38M-380).

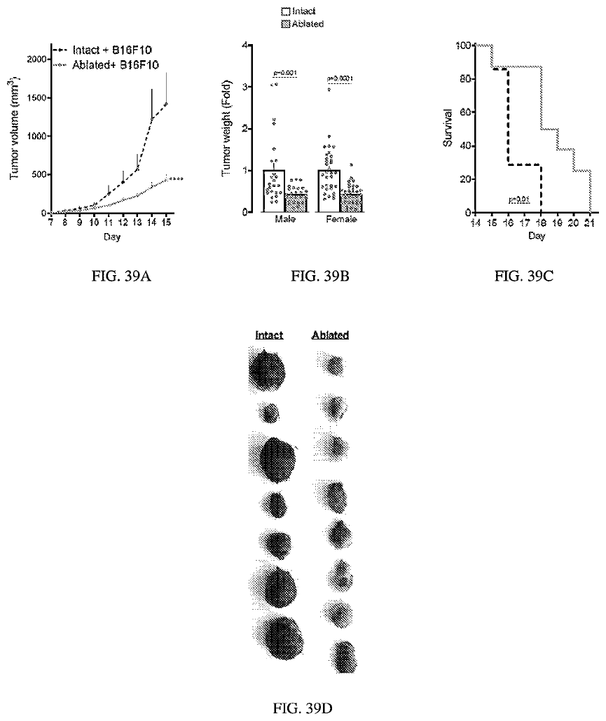

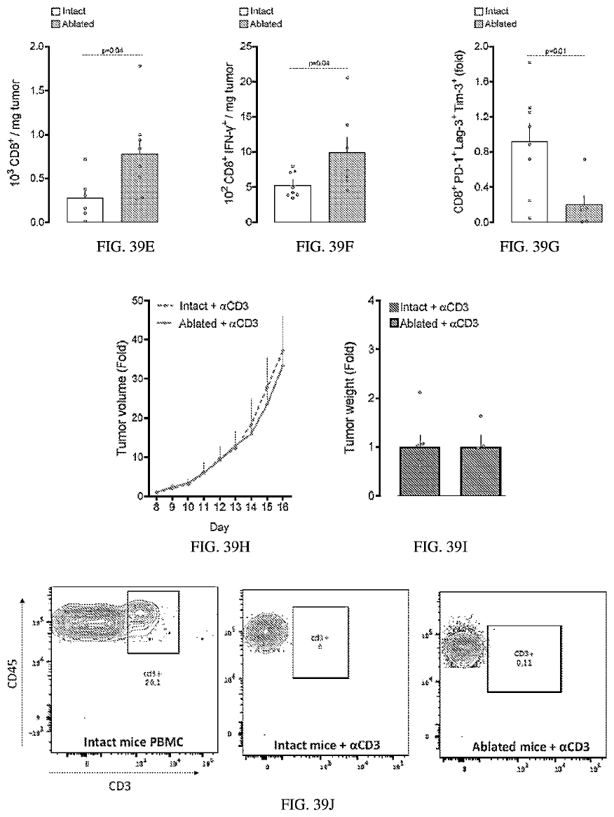

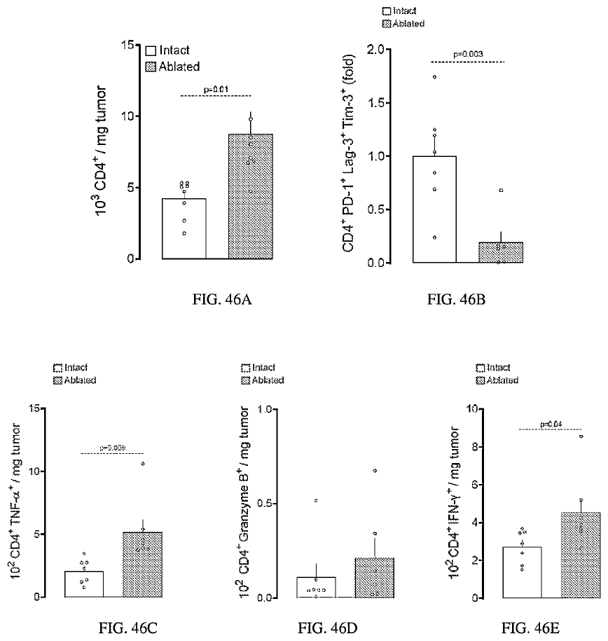

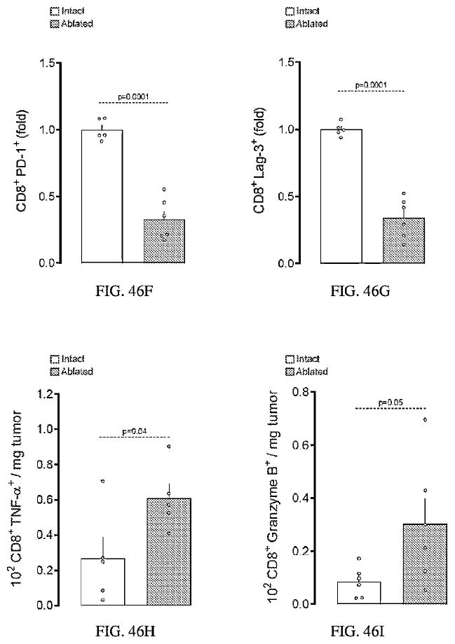

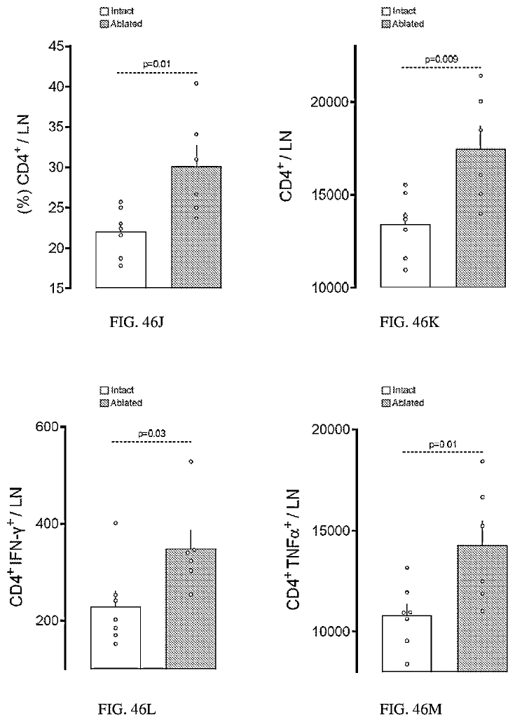

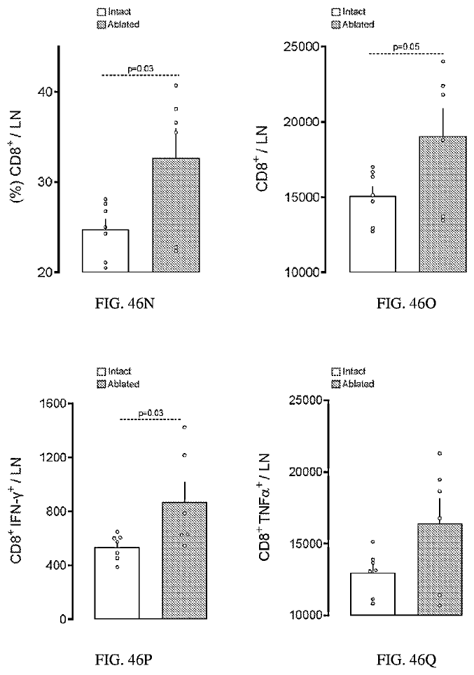

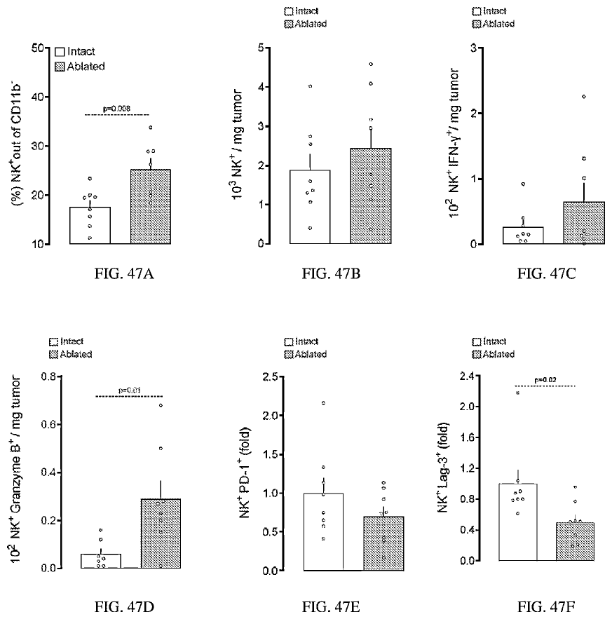

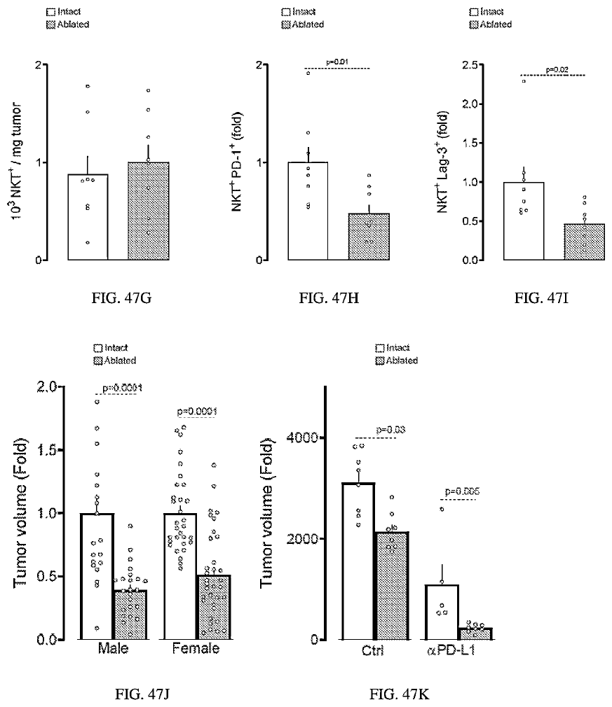

Given that secreted neuropeptides influence the activation of immune cells^{26,74,75,77}, antigen trafficking to skin draining lymph node and drive the expression of immune checkpoint receptors on cytotoxic CD8⁺ T cells (FIGS. 38A-380)⁷⁸⁻⁸³, it was tested whether these neurons modulate the progression of B16F10 tumors in vivo. B16F10 (i.d. 1×10⁵) cells were inoculated in 8-week-old male and female nociceptor ablated (TRPV1^{Cre}::DTA^{fl/wt}) or intact mice. In both male and female, tumor growth, volume, and weight were reduced in mice whose nociceptor were ablated and overall survival was enhanced (FIGS. 39A-39D; 47J). While NKT cells were not impacted (FIG. 47G), the numbers of tumor-infiltrating CD8⁺ (FIG. 39E), CD4⁺ (FIG. 46A) and NK (FIGS. 47A-47B) cells were enhanced in sensory neuron ablated mice, and MDSCs were reduced (not shown).

The genetic ablation of nociceptors preserved the cytotoxic potential of CD8 T cells (increased number of Granzyme B⁺, INFγ⁺, TNFα⁺, INFγ⁺TNFα⁺), while preventing their exhaustion (reduced ratio of PD1⁺Lag3⁺Tim3⁺, PD1⁺Lag3⁺, PD1⁺, Tim3⁺, Lag3⁺) (FIG. 39F-39G, FIG. 46F-46I). TRPV1^{Cre}::DTA^{fl/wt} also preserved CD4+(FIG. 46B-46E), NK (FIGS. 47C-47F) and NKT (FIGS. 47H-47I) cells activation. An absence of sensory neurons had a similar impact on number and activation of lymphocytes within the tumor-draining lymph node (FIG. 46J-46Q). Next, it was probed whether the decrease tumor growth in the TRPV1^{Cre}::DTA^{fl/wt} mice depends on lymphocytes. To test this, a monoclonal antibody was used to systemically deplete CD3⁺ T cells and found after this treatment there was no difference in B16F10 tumor growth and volume between TRPV1^{Cre}::DTA^{fl/wt} and littermate control mice (FIGS. 39H-39J), implying an action of nociceptors on lymphocytes.

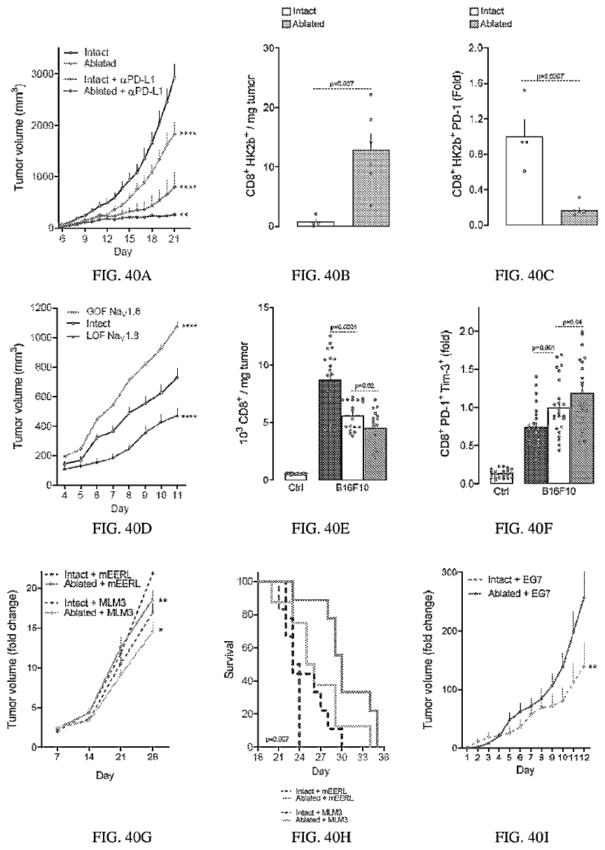

Given that nociceptor neurons drive CD8⁺ T cells exhaustion and that melanoma is highly responsive to immune checkpoint blockers¹⁶, an experiment was devised using a B16F10-OVA cancer cell line to test whether genetic ablation of nociceptor sensory neurons increases tumor elimination upon PDL1 blockade. After tumor-establishment (≥1 cm³ tumor), TRPV1^{Cre}::DTA^{fl/wt} and littermate control mice were treated (on day 7, 10 and 13) with a rat anti-mouse PDL1 (6 mg/kg; clone 29F.1A12; i.p.), and effects assessed on day 14. PDL1 blockade in naïve mice reduced tumor growth and size and increased infiltration of tumor-specific cytotoxic T cells, effects that were enhanced in TRPV1^{Cre}::DTA^{fl/wt} mice (FIGS. 40A-40C). Sensory neuron ablated mice have reduced tumor growth (FIG. 40A), and volume (FIG. 47K) which is coupled with increased infiltration of total (FIG. 40B) and H2 KB-OVA⁺ (FIG. 40C) CD8 T cells.

While not supported by the Immgen database⁸⁴, immune cells have been reported to express TRPV1⁸⁵. As such, it was decided to confirm the findings in Na_{V}1.8^{Cre} mice, since this sodium channel is expressed by mechano- and thermos-sensitive neurons and represents 80% of nociceptors^{86,87} but not in immune cells. Sensory neuron ablation using the Na_{V}1.8^{Cre}::DTA^{fl/wt} line also slowed B16F10 tumor growth and reduced the proportion of PD1⁺Lag3⁺ CD8⁺ T cells within the tumor, while it increases infiltration of total CD8 T cells (FIGS. 40D-40F). Next, these data were confirmed using a nociceptor gain-of-function approach by stimulating (transdermal illumination; 2 mW/mm², 1 Hz, 20 min, BID for 14 d) optogenetically-controlled tumor-innervating neurons. In comparison to light-insensitive littermate control mice, blue light-stimulated Na_{V}1.8^{Cre}::ChR2^{fl/wt} enhanced B16F10 tumor growth, proportion of PD1⁺Lag3⁺ CD8⁺ T cells while it increased the infiltration of total CD8 T cells (FIGS. 40D-40F).

Next, the effect of nociceptors on Braf^{V600E}Cdkn2a^{-/-}Pten^{-/-} melanoma was probed and found reduced tumor growth in the absence of TRPV1⁺ lineage neurons. To determine whether the contribution of nociceptors to tumor growth is a general phenomenon (rather than limited to melanoma), their contribution to tumor growth was tested in an aggressive and innervated model of metastatic head and neck squamous cell carcinoma (MLM3) as well as the parental line from which it was derived (mEERL)⁸⁸. When intradermally implanted in control or TRPV1 nerve ablated mice, slower mEERL and MLM3 growth in absence of sensory neurons was found (FIG. 40G), while their overall survival increased (FIG. 40H). Finally, the impact of nociceptor ablation in a lymphoma model was tested. As opposed to the solid tumor models, ectopic EG7 lymphoma growth (FIG. 401) and spreading (not shown) were enhanced in TRPV1^{Cre}::DTA^{fl/wt} mice.

Neonatal ablation of neuronal subsets using diphtheria toxin may lead to compensatory mechanisms. To confirm the pro-tumorigenic effects of nociceptors in adult, pharmacological approaches were used to block vesicle releases from, or silence, tumor-innervating neurons.

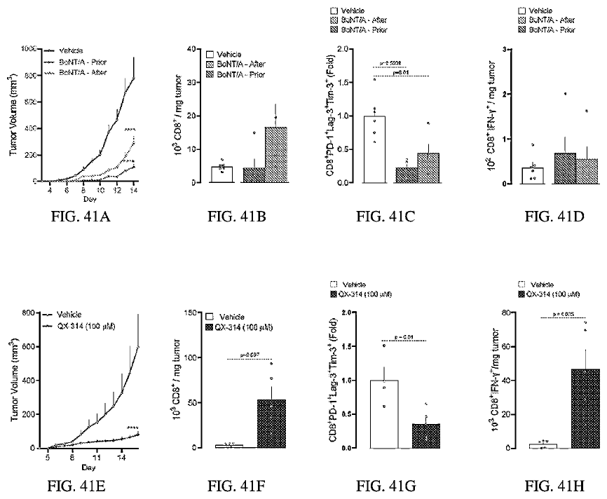

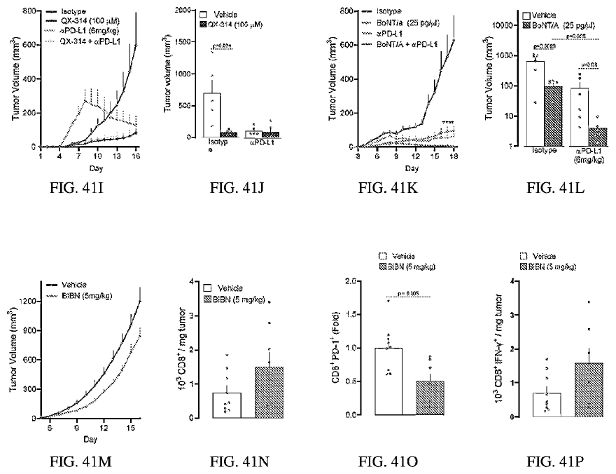

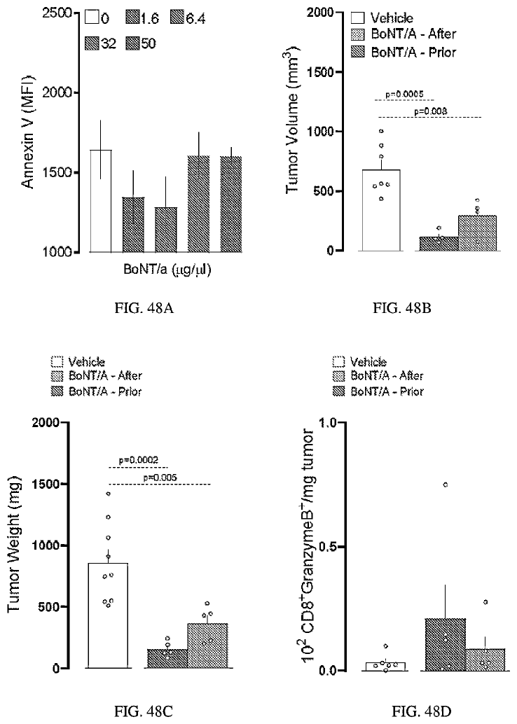

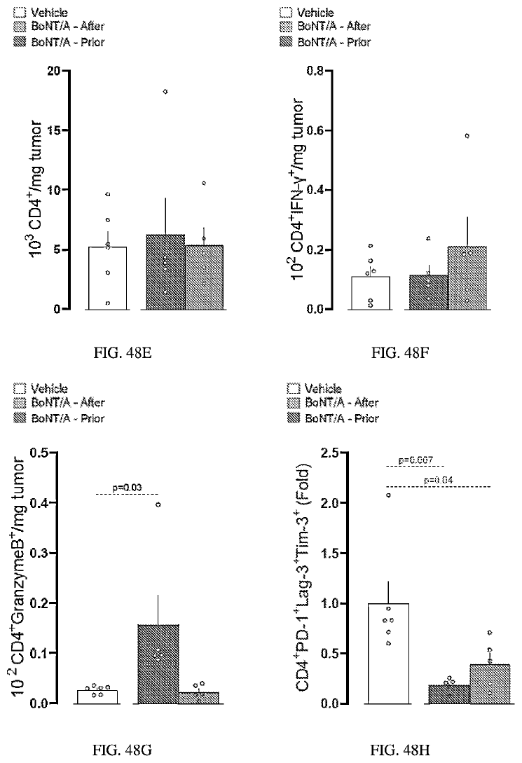

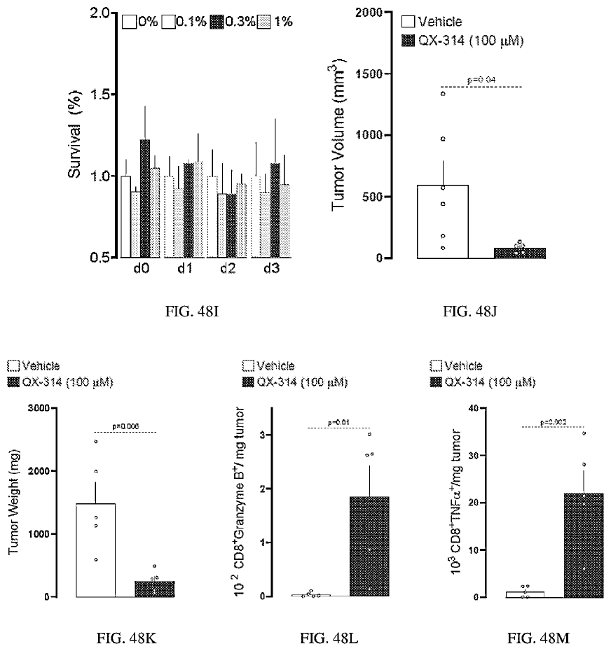

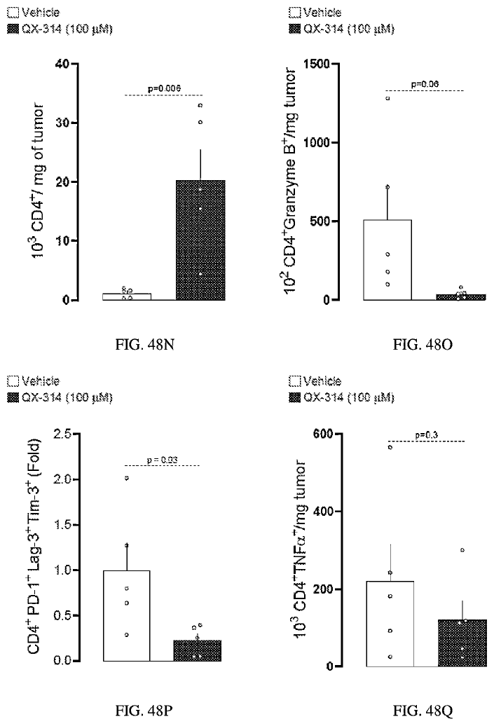

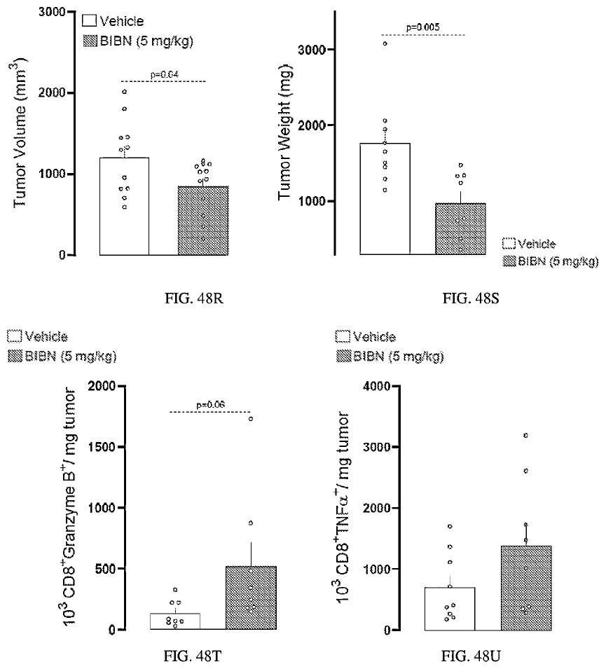

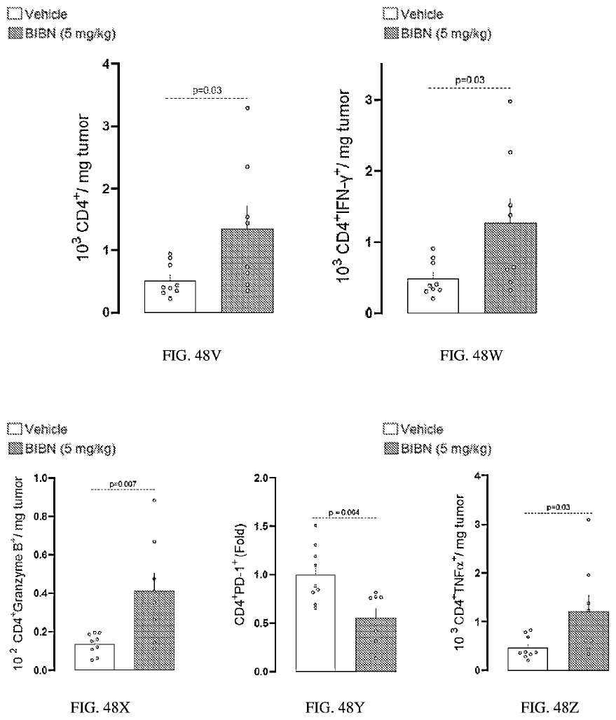

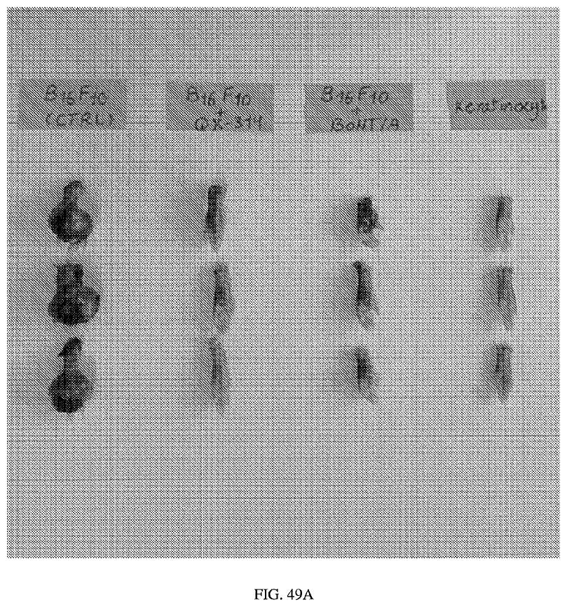

First, the effect of BoNT/a, a neurotoxic protein produced by Clostridium Botulinum was tested. BoNT/a acts by cleaving SNAP-25⁸⁹, a component of the neuronal SNARE complex, triggering a long-lasting (20 days) blockade of neurotransmitter′ release by preventing the vesicle docking with the membrane⁸⁹. This strategy targets all neurons¹², which included skin-innervating autonomic fibers (acetylcholine, noradrenaline) as well as nociceptors (CGRP, SP), and was successfully used to block neuro-immune interplay in skin infection⁷⁶. Thus, BoNT/a was injected (25 pg/μl; 50 ul; 5 injection point) 1 and 3 day prior, or 1 and 3 days after, B16F10 cells inoculation. In both case, BoNT/a reduced tumor growth (FIG. 41A), volume (FIG. 48B), and weight (FIG. 48C) when compared to vehicle-injected skin. BoNT/a treatment after tumor implantation increased CD8 infiltration (FIG. 41B;FIG. 48E), and preserved their cytotoxic potential (increased number of Granzyme B⁺; FIGS. 48D, 48G), and prevented their exhaustion (reduced ratio of PD1⁺Lag3⁺Tim3⁺; FIG. 41C, 48H).

Next, an efficient pain^{26,90,91} and itch⁹² nociceptor-blocking strategy was modified to silence tumor-innervating nociceptors. The protocol uses non-selective large pore ion channels (TRPA1 and TRPV1) as cell-specific drug-entry ports that deliver a charged and membrane-impermeable form of lidocaine (QX-314) into sensory fibers to block sodium currents. During inflammation, as found in tumor micro-environments, these ion channels on the surface of nociceptors open, allowing the small-sized (263 Da) QX-314 to permeate into these neurons resulting in a long-lasting electrical blockade (21457220). Here, it was tested whether skin surrounding tumor-injected with QX-314 amplifies the host anti-tumor activity. It was found that QX-314 mediated sensory neuron silencing (100 μM) reduces tumor growth (FIG. 41E), volume (FIG. 48J) and weight (FIG. 48K) as compared to vehicle-injection. As opposed to BoNT/a, QX-314 enhances the infiltration of CD8+(FIG. 41F) and CD4+(FIG. 48N) T cells, preserved their cytotoxic potential (increased number of INFγ⁺, TNFα⁺, Granzyme B⁺; FIG. 41H, FIGS. 48L, 48M, 480, 48Q), and prevented their exhaustion (decrease ratio of PD1⁺Lag3⁺Tim3⁺; FIG. 41G, FIG. 48P).

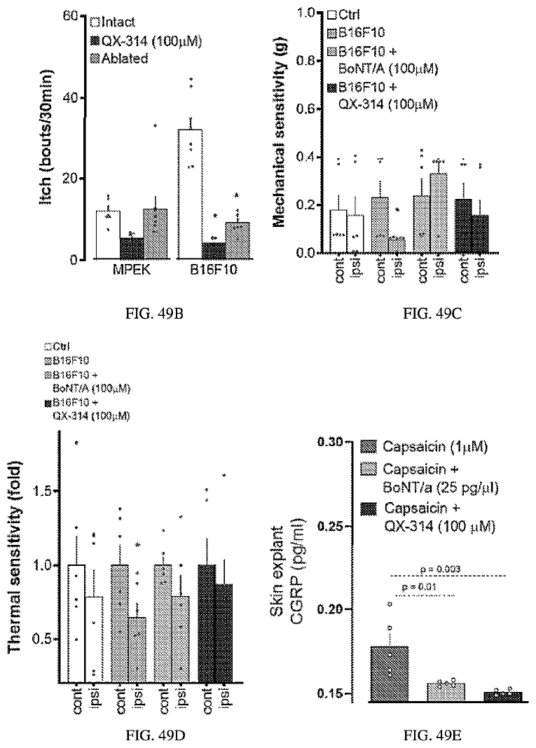

QX-314 (0.1-1%) and BoNT/a (1.6-50 pg/ml) failed to impact B16F10 survival (FIGS. 48A, 48I) or lymphocyte′ function (not shown) when applied directly in cell culture. Intradermal inoculation of B16F10 to the mouse hindpaw led to tumor growth (FIG. 49A), occasional (˜30%) itch (FIG. 49B), mechanical (FIG. 49C) and thermal pain-related hypersensitivity (FIG. 49D). These effects were absent in mice whose sensory neuron are genetically ablated (FIG. 49D) or pharmacologically silenced with QX-314 (100 μM, qd, i.d., FIG. 49A-49D) or treated with BoNT/a (25 pg/μl, FIGS. 49A-49C). In a bath-organ culture of tumor-inoculated skin, it was found that capsaicin-induced CGRP release was absent in samples pre-treated with QX-314 or BoNT/a (FIG. 49E). These data support the capacity of QX-314 or BoNT/a to block neuropeptide release from tumor-innervating neurons, preventing their subsequent immunomodulation of TILs. Finally, it was tested whether silencing tumor-innervating neurons would modulate the response to PDL1 blockade and found that QX-314 or BoNT/a enhanced αPDL1-mediated tumor shrinkage (FIGS. 41I-41L).

CGRP administration increased CD8 T cell exhaustion and reduced their production of cytotoxic granules when co-cultured with melanoma while TILs expressed higher levels of CGRP receptor RAMP1 and RAMP3 when co-cultured with sensory neurons (FIGS. 38A-380, FIGS. 43A-43G, FIGS. 44A-44L, FIGS. 45A-45G). Given that the selective CGRP receptor antagonist BIBN blocks deleterious neuro-immune interplay during microbe infection and rescues host anti-bacterial activity⁷⁴, studies were carried out to test its effect within the tumor microenvironment. Similar to QX-314, CGRP blockade with BIBN reduced tumor growth (FIG. 41M), volume (FIG. 48R), and weight (FIG. 48S). While it did not impact CD8⁺ infiltration (FIG. 41N), BIBN preserved these cells cytotoxic potential (increasing relative infiltration of INFγ⁺, TNFα⁺, Granzyme B⁺; FIG. 41P, FIGS. 48T-48U), and prevented their exhaustion (decrease ratio of PD1⁺; FIG. 41O). BIBN also enhanced the influx of total and cytotoxic CD4 T cells, and stopped their exhaustion (FIGS. 48V-48Z).

In contrast to tumor neo-angiogenesis, the impact of cancer neo-innervation on clinical prognosis remains unclear. While recent data highlight the importance of parasympathetic and sympathetic neurons on tumor growth^{12,14,15,57,58,93-97}, the role for nociceptor neurons in skin and other cancers remain unclear, even though the skin is densely innervated with nociceptor^{11,15,94}.

Given that nociceptor-produced neuropeptides alter immune responses⁹⁸, shutting down T_{H}1 immunity against bacteria 75 and fungi 34 the nature of cancer-nociceptor-CD8⁺ interplay was probed using a xenograft mouse model of triple-negative melanoma skin cancer, a robust and established model of immune surveillance^{16,21,23} It was found that nociceptor neurons are activated by cancer cells, responding with local release of neuropeptides from their peripheral terminal, which, then through an action on cytotoxic T lymphocytes leads to a failure of immune surveillance and tumor growth. Overall, by expressing ligand for¹⁰, and controlling the expression of, immune checkpoint receptors, sensory neurons emerge as a driver of regulatory immunity. Since silencing tumor-innervating neurons rescues the host anti-tumor immunity and improves response to checkpoint blockade, this may constitute an adjuvant therapy for the treatment of skin cancers.

The discovery of axonal guidance molecules demonstrated that nerves are directed to their targets by attractive and repulsive cues^{99,100} Similar signals help blood vessels reach their targets¹⁰¹⁻¹⁰³ Such parallelism emerged between the actions of growth factors that direct angiogenic sprouting and those that regulate axon terminal arborisation^{104,105}. Here, it was found that melanoma, both in vitro and in vivo, drives axonogenesis in nociceptor neurons, which might be the driver for the innervation of these tumors. It likely involves melanoma-released growth factors¹⁰⁶, exosome or other modulators that act on the neurons. Thus, given the high exosome cargo produced by melanoma¹⁰⁷ and its negative clinical impact¹⁰⁸ as well as response to checkpoint blockers¹⁰⁹, may also contribute to melanoma hyper-innervation.

Clinically, vagal denervation increases the risk of some cancer^{6,124,125} while, higher densities of adrenergic and cholinergic nerve fibers correlated with poor clinical outcome¹⁴. Human breast, melanoma, and prostate cancer patients taking β-blockers have lower recurrence and mortality^{122,126,127}. Breast cancer biopsies showed increased sympathetic and decreased parasympathetic nerve density¹¹⁷, while prostate cancers are infiltrated with cholinergic fibers and surrounded by adrenergic fibers¹⁴. Consistent with adrenergic signaling as cancer-promoting, norepinephrine upregulates VEGF, IL-6, and IL-8, in turn, increasing melanoma aggressiveness¹²⁸.

Cancer cells produced mediators may sensitize nociceptors by increasing the expression and lowering the activation threshold of ion channel transducer (Julius, 2013). Here, it was found that hindpaw inoculation of B16F10 cells led to mechanical allodynia and thermal hyperalgesia, while flank injection led to spontaneous itching (in thirty percent of the tested animals). Both the pain hypersensitivity and pruritus were relieved by sensory neuron silencing (QX-314) or by blocking neuropeptide release (BoNT/a) and absent in mice whose nociceptor neurons are genetically ablated (TRPV1^{cre}DTA^{fl/wt}). Sensitizing effects of B16F10 cells was observed in vitro when DRG nociceptor neurons were co-cultured with melanoma, as well as in neurons harvested from tumor-inoculated mice. Such drivers may include melanoma produced TSLP, IL-1β, INFγ, all known nociceptor sensitizers in other contexts²⁵, or melanoma-produced growth factors such as platelet-derived growth factor or transforming growth factor¹⁰⁶ sensitize nociceptors in the context of painful neuropathy¹¹⁰. Finally, the alarmin HMGB1 which is expressed by melanoma¹¹¹ and correlate with disease severity¹¹² can also initiate pain¹¹³.

Lumbar nociceptor neurons express PD-1, whose activation by PD-L1 blocks pain hypersensitivity in melanoma-bearing mice¹⁰. Such data contrast with the clinical neuropathies experienced by melanoma patients and with experimental findings provided herein. The absence of T cells or other PD-L1 bearing cells in this in vitro co-culture system could explain such discrepancy. However, it is unclear why PD-L1 expressing TIL do not readily silence tumor-innervating neurons in vivo. Given that TILs are drawn to fight the cancer cells it may limit their crosstalk with neurons to secreted signals.

Noxious stimuli activate membrane transducers in the peripheral terminals of nociceptors initiate reflexes, sensations, and local release of neuropeptides²⁵. Here, it was found that the B16F10-nociceptor co-culture lead to the release of SP, VIP, and CGRP. Such neuropeptides may have multiple roles; driving i) tumor progression, ii) secretion of tumor-associated cytokines, iii) neo-vascularization, iv) metastasis or v) controlling immune responses^{3,7-9,11,12,114-116} Neuropeptides promote antigen trafficking in the LN and the chemotaxis and polarization of lymphocytes; influencing the localization, extent, and type of inflammation (Cunin et al., 2011; Ganea and Delgado, 2001; Goetzl et al., 2001; Nussbaum et al., 2013; Talbot et al., 2015). Here, it was found that nociceptor-produced neuropeptides induced single (PD1⁺), double (PD1⁺Lag3⁺) and triple (PD1⁺Lag3⁺Tim3⁺) expression of immune checkpoint receptors on cytotoxic CD8 T cells. It was also found that CD8 T cells exposed to sensory neurons upregulate the expression of CGRP receptor RAMP1 and RAMP3 as well as the VIP receptor VPAC1. CGRP decreases the proliferation (IL-2⁺), and cytotoxic capacity (TNFα⁺, INFγ⁺, Granzyme B⁺) of these cytotoxic CD8 T cells, while local treatment with BIBN, a RAMP1 blocker, prevents cytotoxic CD8 T cells exhaustion and rescues anti-tumor immunity.

The elimination of B16F10-OVA cancer cells by OT1 cytotoxic CD8 T cells decreases when they are co-cultured with nociceptors or exposed to nociceptor-produced neuropeptides. Such as in the case of pancreatic ductal cancer¹¹, B16F10 cancer cell growth slowed down in nociceptor ablated mice. Furthermore, these tumors now have a higher content of cytotoxic CD8 T cells, which bear less immune checkpoint receptor expression and have a lower content of cytotoxic granules. This decrease in tumor growth was prevented by systemic αCD3 depletion, confirming a nociceptor-T cell-mediated effect. These are the first to show that nociceptor neuropeptide (CGRP) drives immune checkpoint receptor expression on TILs.

Tumor-specific sympathetic denervation downregulates the expression of PDL1, PD1 as well as FOXP3 while parasympathetic innervation decreased TIL expression of PD-1 and PD-L1¹¹⁷. The authors also discovered that exhaustion of TILs correlates with their distance from sympathetic nerves¹¹⁷. It is unclear how sympathetic nerves drive these effects, and whether there is a contribution of sensory neurons, but the data suggests that nociceptors help control the expression of immune checkpoint receptors, and promote regulatory immunity within the tumor microenvironment.

At neuro-neoplastic contact, cancer cells produce mediators (e.g. alarmin, TSLP or IFNγ, growth factors, or sEVs) that may prompt nociceptor axonogenesis increasing innervation of the tumor, sensitization which will produce pain or itch and neuropeptide release which alters the immune system. Does this constitute a host warning response to danger or more plausibly a maladaptive maladaptive neuro-immune crosstalk similar to that occurring in diseased contexts²⁵, but which here contributes to tumor growth? Ablation of skin neurons enhanced the local immune infiltration, in a manner similar to microbes which hijack the nociceptor interplay with the immune systems to modify regulatory-mediated immunity^{74,77} and facilitates the survival of the cancer cells. Supporting this, it was found that while DRG neurons do not upregulate the proliferation of melanoma cells in vitro, their presence increased tumor growth by enhancing the migration of myeloid-derived suppressor cells supporting the onset of an immunosuppressive and pro-tumorigenic microenvironment¹¹⁵.

The neuropeptides expressed by and released from nociceptors also depend on the type of bacteria activating them, indicating that different microbes tweak nociceptor activity in a way that promotes their survival⁷⁷. It is conceivable that different cancer types may act similarly, either blocking or increasing neuropeptide release from different sensory neurons. This could explain why tumor denervation increases breast cancer growth¹¹⁷ while it suppresses prostate, melanoma or ovarian cancer development^{14,52,58,94}.

Despite tremendous strides, the success of immune therapy in cancer is limited by its variability amongst patients and cancer types, and the adverse effects it produces¹⁶. Here, it was found that targeting nociceptors with QX-314 or BoNT/A increased the response to immune checkpoint ligands, and may, therefore, be a potential adjuvant therapy to PD-L1 and other blockers.

QX-314 and BoNT/A abolished the local neuro-immune interplay^{26,76}, and by decreasing immune checkpoint receptor expression, safeguard host anti-tumor immune activity resulting in a decrease in tumor growth and increased survival. Silencing tumor-innervating sensory neurons is, therefore, an innovative strategy to attenuate the immunomodulatory power of the nervous system to promote anti-tumor activity. Local administration of a CGRP receptor blocker⁷⁶ also prevents TIL exhaustion and may be worth exploring as adjuvant therapy for tumor types responsive to checkpoint blockade and where tumor innervation by nociceptors is a feature.

QX-314 also reversed melanoma-induced pain and itch and its activity support the presence of a sufficient levels of inflammation in the tumor micro-environment to allow its uptake into tumor-innervating nociceptors through large pore ion channels. This symptom suppression and immune surveillance enhancing strategy offers three potential advantages: (1) high specificity (the effect is limited to sensory neurons that express activated large pore channels), (2) long-lasting activity, and (3) limited side-effects, the charge on QX-314 would limit diffusion through lipid membranes and redistribution²⁶.

In summary, the data supports a regulatory role for nociceptor over immune response to tumor growth through the regulation of the expression of immune checkpoint receptors on cytotoxic CD8⁺ T cells. Silencing cancer-innervating nociceptors or blocking the release or action of neuropeptides prevents cytotoxic T cell exhaustion and represents an innovative strategy to safeguard host anti-tumor immune responses.

### Methods for Example 2

### Experimental Procedures

The Institutional Animal Care and Use Committees of Boston Children's Hospital, and the Université de Montréal (CDEA #19027; #19028) approved animal procedures. Either of MPEK-BL6 (non-tumorigenic keratinocytes), B16F10¹³³ Braf^{V600E}Cdkn2a^{-/-} Pten^{-/- 134}, mEERL¹³⁵ or MLM3¹³⁵ cancer cells were implanted (10⁵ cells; i.d., left flank) to 8-weeks old male and female wildtype, littermate control or to genetically-engineered mice whose sensory neurons are fluorescent, ablated or controlled by light. Tumor growth and survival was monitored daily, and animals were euthanized when the tumor reached 1 cm³. Tumors and sdLN were harvested, and TILs number and innervation were respectively analyzed by flow cytometry or immunofluorescence. QX-314 (100 μM, 100 μL, i.d)⁹² and BIBN (5 mg/kg, 100 μL, i.d)⁷⁶ were administered once daily after tumors become visible (˜day 5), while BoNT/a was administered (25 pg/μl, 100 μL, i.d)⁷⁶ prior to or right after tumor inoculation.

### Statistics

Data expressed as mean±S.E.M. Statistical significance determined by one-way or two-way ANOVA for multiple comparisons and two-tail unpaired Student's t-test for single variable comparison. P values less than 0.05 were considered significant. Numbers of animals are defined in figure legends.

### Animals

Mice were housed in standard environmental conditions (12 h light/dark cycle; 23° C.; food and water ad libitum) at facilities accredited by the Association for Assessment and Accreditation of Laboratory Animal Care. 8-week old C57BL6 (Jax, #000664); BALB/c (Jax, #001026), OT1 (Jax, #003831), TRPV1^{cre} (Jax, #017769), ChR2^{fl/fl}(Jax, #012567), td-tomato^{fl/fl} (Jax, #007908), DTA^{fl/fl} (Jax, #009669),^{136 136 13 135 136 138 138 142 142 142 142 142 142 142 142 142 141 140 141 141} (Voehringer et al., 2008) GCaMP6^{fl/fl} (Jax, #024105) mice were purchased from Jackson Laboratory. Na_{V}1.8^{cre} mice were generously supplied by Professor Rohini Kuner (Heidelberg University). The cre/lox toolbox was used to genetically-engineered the various mice lines used (TRPV1^{cre}::DTA^{fl/wt}, TRPV1^{cre}::GCaMP6^{fl/wt}, TRPV1^{cre}::Tdtomato^{fl/wt}, NaV1.8^{cre}::DTA^{fl/wt}, NaV1.8^{cre}::ChR2^{fl/wt} and littermate control) by crossing male heterozygote Cre mice to female homozygous loxP mice. All Cre driver lines used are viable and fertile and abnormal phenotypes were not detected. Offspring were tail clipped; tissue was used to assess the presence of transgene by standard PCR, as described by Jackson Laboratory. Offspring were used at 8 weeks of age.

### Cell Line

B16F0 (ATCC), B16-F10 (ATCC), B16F10-OVA (ATCC), B16F10-mCherry2 (ATCC), mEERL (ATCC), MLM3 (ATCC), EG7 (ATCC) were cultured in complete Dulbecco's Modified Eagle's Medium high glucose (DMEM, Gibco) supplemented with 10% fetal bovine serum (Biochrom) and 1% penicillin/steptomycin (Gibco), and maintained at 37° C. in a humidified incubator with 5% CO₂.

### Cancer Inoculation

Cancer cells (1×10⁵) were resuspended in PBS and injected (i.d., 100 l) to the mice right flank. Growth was daily assessed using a handheld caliper. Mice were euthanized when cancer reach 1000-1500 mm³, tumors and tumor draining lymph node (tdLN) harvested and immunophenotype by flow cytometry.

### Tumor Immonophenotyping

Tumor were enzymatically digested (DMEM+2 mg/ml collagenase D (Sigma)+0.03 mg/ml DNAse I (Sigma) under constant shaking (30 min, 37° C.). The cell suspension was then strained (70 um), washed and RBC lysed (Life Technologies, ACK lysis buffer, 2 min). Single cells were then resuspended in FACS buffer (PBS, 2% FCS, EDTA), Fc blocked (0.5 mg/ml, 10 min; BD Biosciences) and stained (45 min, 4° C.) with monoclonal antibodies (anti-CD45-BV421 (1:100, Biolegend), anti-CD11b-APC-cy7 (1:100, Biolegend), anti-CD8-PercP (1:100, Biolegend), anti-CD4-FITC (1:100, Biolegend), anti-PD-1-PE-cy7 (1:100, Biolegend), anti-Lag3-PE (1:100, Biolegend), anti-Tim-3-APC (1:100, Biolegend).

### Intracellular Cytokine Staining

Cells were stimulated (4 h) with Brefeldin A (Biolegend, #423304), washed, fixed/permeabilized (BD Biosciences; #554714) and stained (anti-IFN-γ FITC (Biolegend), anti-TNF-α PE (Biolegend), anti-Granzyme B APC (Biolegend, #504118).

### Drugs

QX-314 (Tocris, #2313; 100 μM) was injected (i.d.) daily in 5 points around the tumor (treatment began once tumor was visible). BIBN4096 (Tocris, #4561; 5 mg/kg) was injected (i.d.) on day 6, 8, 10, 12 and 14. Botulinum neurotoxin A (List biological labs, #130B; 25 pg/μl) was injected (i.d.) three and one day prior to, or one and three days after, tumor inoculation. In either of vehicle, QX-314, BoNT/a, littermate control or TRPV1^{cre}::DTA^{fl/wt} mice, αPD-L1 (Bioxcell, 6 mg/kg) was injected (i.p.) on day 5, 8 and 11.

### Skin Explant

1 h post exposure to vehicle (100 μL), QX-314 (100 μM, 100 μL) or BoNT/a (25 pg/μL, 50 L), tumor-surrounding skin was harvested using 10 mm punch biopsies and transferred to a 24-well plates (DMEM+protease inhibitor (1 ul/ml). After 30 min incubation (37° C.), the supernatant was collected and CGRP analyzed by commercial ELISA (Cayman Chemical).

### Neuron Culture

Mice were sacrificed and dorsal root ganglia (DRG) were dissected out into DMEM medium (Corning, #10-013-CV), completed with 50 U/mL penicillin and 50 μg/mL streptomycin (Fisher, #MT-3001-C1), and 10% FBS (Seradigm, #3100). Cells were then dissociated in HEPES buffered saline (Sigma) completed with 1 mg/mL collagenase IV (Sigma, #C0130)+2.4 U/mL dispase II (Sigma, #04942078001) and incubated for 80 minutes at 37° C. Ganglia were triturated with glass Pasteur pipettes of decreasing size in supplemented DMEM medium, then centrifuged over a 10% BSA gradient, plated on Laminin (Sigma, #L2020) coated cell culture dishes. Cells were cultured with Neurobasal-A medium (Gibco, #21103-049) completed with 0.05 ng/μL NGF (Life Technologies, #13257-019), 0.002 ng/μL GDNF (Peprotech, #450-51-10), 0.01 mM AraC (Sigma, #C6645) and 200 nM L-Glutamin (VWR, #02-0131).

### CD8 Isolation

Adult mice (8-10 weeks-old) were euthanized, spleen harvested (ice-cold PBS, 5% FBS), and mechanically dissociated. The cells were strained (70-μm), RBC lysed (ACK lysing buffer) and counted using a hemocytometer. Naïve CD8⁺ T cells were magnet sorted (Stem cell, #19853A) or FACS sorted and cultured (DMEM+FBS 10%, Pen/Stp, non-essential amino acid, vitamin, β-mercaptoethanol, L-glutamine, and sodium pyruvate). To generate cytotoxic T lymphocytes, 1×106 naïve CD8⁺ T cells will be seeded and stimulated for 48 h under TC1 inflammatory condition (2 μg/ml αCD3/αCD28 (Bioxcell)+10 ng/ml rIL-12 (Biolegend)+10 μg/ml of anti-IL-4 (Bioxcell).

### Co-Culture

Naïve DRG neurons (1×10⁴) were seeded in T cell media (supplemented with 0.05 ng/μL NGF (Life Technologies, #13257-019), 0.002 ng/μL GDNF (Peprotech, #450-51-10), IL-2 and co-cultured with Tc1 CD8 T cells (1×10⁵). After 48 h co-culture, Tc1 cells were collected by centrifugation (5 min at 1300 rpm), stained and immunophenotyped by flow cytometry.

### Apoptosis

Naïve TRPV1^{Cre}::Tdtomato^{fl/wt} DRG neurons (1×10⁴) were co-cultured (24-72 h) with OT1 T_{C1} CD8 T cells (1×10⁵) and B16F10-mCherry2-OVA (1×10⁵). in T cell media (supplemented with 0.05 ng/μL NGF (Life Technologies, #13257-019), 0.002 ng/μL GDNF (Peprotech, #450-51-10), IL-2 After 48 h co-culture, Tc1 cells were collected by centrifugation (5 min at 1300 rpm), stained and immunophenotyped by flow cytometry. Anti-annexin V APC (Biolegend), 7-AAD

### Immunofluorescence

2×10³ DRG neurons were co-cultured with 2×10⁴ B16F10-mCherry2 for 24-48 h. The cells were then fixed (4% paraformaldehyde; 30 min), permeabilized (0.1% Triton X-100, 20 min), and blocked (PBS, 0.1% Triton X-100, 5% BSA, 30 min). The cells were rinsed (PBS), and stained with mounted with Vectashield (Vector Laboratories, #H-1000) containing DAPI (Tocris, #5748). Images were acquired using a Ti2 Nikon fluorescent microscope.

### References for Example 2

1 Albo, D. et al. Neurogenesis in colorectal cancer is a marker of aggressive tumor behavior and poor outcomes. Cancer 117, 4834-4845, doi:10.1002/cncr.26117 (2011).
2 Entschladen, F., Palm, D., Niggemann, B. & Zaenker, K. S. The cancer's nervous tooth: Considering the neuronal crosstalk within tumors. Semin Cancer Biol 18, 171-175, doi:10.1016/j.semcancer.2007.12.004 (2008).
3 Li, S., Sun, Y. & Gao, D. Role of the nervous system in cancer metastasis. Oncol Lett 5, 1101-1111, doi:10.3892/ol.2013.1168 (2013).
4 Palm, D. & Entschladen, F. Neoneurogenesis and the neuro-neoplastic synapse. Prog Exp Tumor Res 39, 91-98, doi:10.1159/000100049 (2007).
5 Zanker, K. S. The neuro-neoplastic synapse: does it exist? Prog Exp Tumor Res 39, 154-161, doi:10.1159/000100075 (2007).
6 Boilly, B., Faulkner, S., Jobling, P. & Hondermarck, H. Nerve Dependence: From Regeneration to Cancer. Cancer Cell 31, 342-354, doi:10.1016/j.ccell.2017.02.005 (2017).
7 Khasraw, M. & Posner, J. B. Neurological complications of systemic cancer. Lancet Neurol 9, 1214-1227, doi:10.1016/S1474-4422(10)70220-9 (2010).
8 Gidron, Y., Perry, H. & Glennie, M. Does the vagus nerve inform the brain about preclinical tumours and modulate them? Lancet Oncol 6, 245-248, doi:10.1016/S1470-2045(05)70096-6 (2005).
9 Ondicova, K. & Mravec, B. Role of nervous system in cancer aetiopathogenesis. Lancet Oncol 11, 596-601, doi:10.1016/S1470-2045(09)70337-7 (2010).
10 Chen, G. et al. PD-L1 inhibits acute and chronic pain by suppressing nociceptive neuron activity via PD-1. Nature neuroscience 20, 917-926, doi:10.1038/nn.4571 (2017).
11 Saloman, J. L. et al. Ablation of sensory neurons in a genetic model of pancreatic ductal adenocarcinoma slows initiation and progression of cancer. Proceedings of the National Academy of Sciences of the United States of America 113, 3078-3083, doi:10.1073/pnas.1512603113 (2016).
12 Zhao, C. M. et al. Denervation suppresses gastric tumorigenesis. Sci Transl Med 6, 250ra115, doi:10.1126/scitranslmed.3009569 (2014).
13 Isaacs, J. T. Cancer. Prostate cancer takes nerve. Science 341, 134-135, doi:10.1126/science.1241776 (2013).
14 Magnon, C. et al. Autonomic nerve development contributes to prostate cancer progression. Science 341, 1236361, doi:10.1126/science.1236361 (2013).
15 Saloman, J. L., Albers, K. M., Rhim, A. D. & Davis, B. M. Can Stopping Nerves, Stop Cancer? Trends Neurosci 39, 880-889, doi:10.1016/j.tins.2016.10.002 (2016).
16 Dougan, M. & Dranoff, G. Immune therapy for cancer. Annu Rev Immunol 27, 83-117, doi:10.1146/annurev.immunol.021908.132544 (2009).
17 Chambers, C. A., Kuhns, M. S., Egen, J. G. & Allison, J. P. CTLA-4-mediated inhibition in regulation of T cell responses: mechanisms and manipulation in tumor immunotherapy. Annual review of immunology 19, 565-594, doi:10.1146/annurev.immunol.19.1.565 (2001).
18 Topalian, S. L. et al. Safety, activity, and immune correlates of anti-PD-1 antibody in cancer. N Engl J Med 366, 2443-2454, doi:10.1056/NEJMoa1200690 (2012).
19 Das, M., Zhu, C. & Kuchroo, V. K. Tim-3 and its role in regulating anti-tumor immunity. Immunol Rev 276, 97-111, doi:10.1111/imr.12520 (2017).
20 Baumeister, S. H., Freeman, G. J., Dranoff, G. & Sharpe, A. H. Coinhibitory Pathways in Immunotherapy for Cancer. Annu Rev Immunol 34, 539-573, doi:10.1146/annurev-immunol-032414-112049 (2016).
21 Vesely, M. D., Kershaw, M. H., Schreiber, R. D. & Smyth, M. J. Natural innate and adaptive immunity to cancer. Annu Rev Immunol 29, 235-271, doi:10.1146/annurev-immunol-031210-101324 (2011).
22 Woo, S. R., Corrales, L. & Gajewski, T. F. Innate immune recognition of cancer. Annual review of immunology 33, 445-474, doi:10.1146/annurev-immunol-032414-112043 (2015).
23 Wolchok, J. D. et al. Nivolumab plus ipilimumab in advanced melanoma. N Engl J Med 369, 122-133, doi:10.1056/NEJMoa1302369 (2013).
24 Long, G. V. et al. Nivolumab for Patients With Advanced Melanoma Treated Beyond Progression: Analysis of 2 Phase 3 Clinical Trials. JAMA Oncol 3, 1511-1519, doi:10.1001/jamaoncol.2017.1588 (2017).
25 Talbot, S., Foster, S. L. & Woolf, C. J. Neuroimmunity: Physiology and Pathology. Annu Rev Immunol 34, 421-447, doi:10.1146/annurev-immunol-041015-055340 (2016).
26 Talbot, S. et al. Silencing Nociceptor Neurons Reduces Allergic Airway Inflammation. Neuron 87, 341-354, doi:10.1016/j.neuron.2015.06.007 (2015).
27 Rosas-Ballina, M. et al. Acetylcholine-synthesizing T cells relay neural signals in a vagus nerve circuit. Science 334, 98-101, doi:10.1126/science.1209985 (2011).
28 Downing, J. E. & Miyan, J. A. Neural immunoregulation: emerging roles for nerves in immune homeostasis and disease. Immunol Today 21, 281-289 (2000).
29 Veiga-Fernandes, H. & Mucida, D. Neuro-Immune Interactions at Barrier Surfaces. Cell 165, 801-811, doi:10.1016/j.cell.2016.04.041 (2016).
30 McMahon, S. B., La Russa, F. & Bennett, D. L. Crosstalk between the nociceptive and immune systems in host defence and disease. Nat Rev Neurosci 16, 389-402, doi:10.1038/nrn3946 (2015).
31 Foster, S. L., Talbot, S. & Woolf, C. J. CNS injury: IL-33 sounds the alarm. Immunity 42, 403-405, doi:10.1016/j.immuni.2015.02.019 (2015).
32 Talbot, S. et al. Cellular localization of kinin B1 receptor in the spinal cord of streptozotocin-diabetic rats with a fluorescent [Nalpha-Bodipy]-des-Arg9-bradykinin. Journal of neuroinflammation 6, 11, doi:10.1186/1742-2094-6-11 (2009).
33 Kashem, S. W. & Kaplan, D. H. Skin Immunity to Candida albicans. Trends Immunol 37, 440-450, doi:10.1016/j.it.2016.04.007 (2016).
34 Kashem, S. W. et al. Nociceptive Sensory Fibers Drive Interleukin-23 Production from CD301b+ Dermal Dendritic Cells and Drive Protective Cutaneous Immunity. Immunity 43, 515-526, doi:10.1016/j.immuni.2015.08.016 (2015).
35 Foster, S. L., Seehus, C. R., Woolf, C. J. & Talbot, S. Sense and Immunity: Context-Dependent Neuro-Immune Interplay. Front Immunol 8, 1463, doi:10.3389/fimmu.2017.01463 (2017).
36 Ding, W., Stohl, L. L., Wagner, J. A. & Granstein, R. D. Calcitonin gene-related peptide biases Langerhans cells toward Th2-type immunity. J Immunol 181, 6020-6026, doi:181/9/6020 [pii] (2008).
37 Jimeno, R. et al. Effect of VIP on the balance between cytokines and master regulators of activated helper T cells. Immunol Cell Biol 90, 178-186, doi:10.1038/icb.2011.23 (2012).
38 Mikami, N. et al. Calcitonin gene-related peptide is an important regulator of cutaneous immunity: effect on dendritic cell and T cell functions. Journal of immunology 186, 6886-6893, doi:10.4049/jimmunol.1100028 (2011).
39 Goetzl, E. J. et al. Enhanced delayed-type hypersensitivity and diminished immediate-type hypersensitivity in mice lacking the inducible VPAC(2) receptor for vasoactive intestinal peptide. Proc Natl Acad Sci USA 98, 13854-13859, doi:10.1073/pnas.241503798 (2001).
40 Nussbaum, J. C. et al. Type 2 innate lymphoid cells control eosinophil homeostasis. Nature 502, 245-248, doi:10.1038/nature12526 (2013).
41 Cunin, P. et al. The tachykinins substance P and hemokinin-1 favor the generation of human memory Th17 cells by inducing IL-1beta, IL-23, and TNF-like 1A expression by monocytes. Journal of immunology 186, 4175-4182, doi:10.4049/jimmunol.1002535 (2011).
42 Ganea, D. & Delgado, M. Neuropeptides as modulators of macrophage functions. Regulation of cytokine production and antigen presentation by VIP and PACAP. Arch Immunol Ther Exp (Warsz) 49, 101-110 (2001).
43 Ayala, G. E. et al. In vitro dorsal root ganglia and human prostate cell line interaction: redefining perineural invasion in prostate cancer. Prostate 49, 213-223 (2001).
44 Dubeykovskaya, Z. et al. Neural innervation stimulates splenic TFF2 to arrest myeloid cell expansion and cancer. Nat Commun 7, 10517, doi:10.1038/ncomms10517 (2016).
45 Erin, N., Akdas Barkan, G., Harms, J. F. & Clawson, G. A. Vagotomy enhances experimental metastases of 4THMpc breast cancer cells and alters substance P level. Regul Pept 151, 35-42, doi:10.1016/j.regpep.2008.03.012 (2008).
46 Erin, N., Zhao, W., Bylander, J., Chase, G. & Clawson, G. Capsaicin-induced inactivation of sensory neurons promotes a more aggressive gene expression phenotype in breast cancer cells. Breast Cancer Res Treat 99, 351-364, doi:10.1007/s10549-006-9219-7 (2006).
47 Liu, V. et al. Extrinsic intestinal denervation modulates tumor development in the small intestine of Apc(Min/+) mice. J Exp Clin Cancer Res 34, 39, doi:10.1186/s13046-015-0159-0 (2015).
48 Lu, S. H., Zhou, Y., Que, H. P. & Liu, S. J. Peptidergic innervation of human esophageal and cardiac carcinoma. World J Gastroenterol 9, 399-403 (2003).
49 Mitchell, B. S., Schumacher, U. & Kaiserling, E. Are tumours innervated?Immunohistological investigations using antibodies against the neuronal marker protein gene product 9.5 (PGP 9.5) in benign, malignant and experimental tumours. Tumour Biol 15, 269-274 (1994).
50 Mitchell, B. S., Schumacher, U., Stauber, V. V. & Kaiserling, E. Are breast tumours innervated? Immunohistological investigations using antibodies against the neuronal marker protein gene product 9.5 (PGP 9.5) in benign and malignant breast lesions. Eur J Cancer 30A, 1100-1103 (1994).
51 Papageorgiou, A. et al. Slowing growth and histology changes in Lewis lung carcinoma implanted in a partly denervated muscle. Cancer Invest 21, 869-872 (2003).
52 Romeo, H. E. et al. Slower growth of tumours in sympathetically denervated murine skin. J Auton Nerv Syst 32, 159-164, doi:10.1016/0165-1838(91)90066-c (1991).
53 Seifert, P. & Spitznas, M. Axons in human choroidal melanoma suggest the participation of nerves in the control of these tumors. Am J Ophthalmol 133, 711-713 (2002).
54 Walter, F. M., Humphrys, E., Tso, S., Johnson, M. & Cohn, S. Patient understanding of moles and skin cancer, and factors influencing presentation in primary care: a qualitative study. BMC Fam Pract 11, 62, doi:10.1186/1471-2296-11-62 (2010).
55 Yosipovitch, G. Chronic pruritus: a paraneoplastic sign. Dermatol Ther 23, 590-596, doi:10.1111/j.1529-8019.2010.01366.x (2010).
56 Yosipovitch, G. et al. Association of pain and itch with depth of invasion and inflammatory cell constitution in skin cancer: results of a large clinicopathologic study. JAMA Dermatol 150, 1160-1166, doi:10.1001/jamadermatol.2014.895 (2014).
57 Zahalka, A. H. et al. Adrenergic nerves activate an angio-metabolic switch in prostate cancer. Science 358, 321-326, doi:10.1126/science.aah5072 (2017).
58 Mauffrey, P. et al. Progenitors from the central nervous system drive neurogenesis in cancer. Nature 569, 672-678, doi:10.1038/s41586-019-1219-y (2019).
59 Mancino, M., Ametller, E., Gascon, P. & Almendro, V. The neuronal influence on tumor progression. Biochim Biophys Acta 1816, 105-118, doi:10.1016/j.bbcan.2011.04.005 (2011).
60 Jobling, P. et al. Nerve-Cancer Cell Cross-talk: A Novel Promoter of Tumor Progression. Cancer Res 75, 1777-1781, doi:10.1158/0008-5472.CAN-14-3180 (2015).
61 McDonnell, T. J. et al. Biomarker expression patterns that correlate with high grade features in treatment naive, organ-confined prostate cancer. BMC Med Genomics 1, 1, doi:10.1186/1755-8794-1-1 (2008).
62 Haqq, C. et al. The gene expression signatures of melanoma progression. Proc Natl Acad Sci USA 102, 6092-6097, doi:10.1073/pnas.0501564102 (2005).
63 Cancer Genome Atlas Research, N. et al. The Cancer Genome Atlas Pan-Cancer analysis project. Nat Genet 45, 1113-1120, doi:10.1038/ng.2764 (2013).
64 Ponten, F., Schwenk, J. M., Asplund, A. & Edqvist, P. H. The Human Protein Atlas as a proteomic resource for biomarker discovery. J Intern Med 270, 428-446, doi:10.1111/j.1365-2796.2011.02427.x (2011).
65 Cerami, E. et al. The cBio cancer genomics portal: an open platform for exploring multidimensional cancer genomics data. Cancer Discov 2, 401-404, doi:10.1158/2159-8290.CD-12-0095 (2012).
66 Brown, I. S. Pathology of Perineural Spread. J Neurol Surg B Skull Base 77, 124-130, doi:10.1055/s-0036-1571837 (2016).
67 Bakst, R. L. et al. Perineural Invasion and Perineural Tumor Spread in Head and Neck Cancer. Int J Radiat Oncol Biol Phys 103, 1109-1124, doi:10.1016/j.ijrobp.2018.12.009 (2019).
68 Croker, J., Burmeister, B. & Foote, M. Neurotropic melanoma: the management of localised disease. J Skin Cancer 2012, 706452, doi:10.1155/2012/706452 (2012).
69 Lin, D., Kashani-Sabet, M., McCalmont, T. & Singer, M. I. Neurotropic melanoma invading the inferior alveolar nerve. J Am Acad Dermatol 53, S120-122, doi:10.1016/j.jaad.2004.11.057 (2005).
70 Chang, P. C. et al. Perineural spread of malignant melanoma of the head and neck: clinical and imaging features. AJNR Am J Neuroradiol 25, 5-11 (2004).
71 Walters, H. et al. Neurotropic melanoma of the trigeminal nerve: a case of atypical facial pain. J Oral Maxillofac Surg 66, 547-550, doi:10.1016/j.joms.2006.11.033 (2008).
72 Goswami, S. C. et al. Molecular signatures of mouse TRPV1-lineage neurons revealed by RNA-Seq transcriptome analysis. J Pain 15, 1338-1359, doi:10.1016/j.jpain.2014.09.010 (2014).
73 Shay, T. & Kang, J. Immunological Genome Project and systems immunology. Trends Immunol 34, 602-609, doi:10.1016/j.it.2013.03.004 (2013).
74 Baral, P. et al. Nociceptor sensory neurons suppress neutrophil and gammadelta T cell responses in bacterial lung infections and lethal pneumonia. Nat Med 24, 417-426, doi:10.1038/nm.4501 (2018).
75 Chiu, I. M. et al. Bacteria activate sensory neurons that modulate pain and inflammation. Nature 501, 52-57, doi:10.1038/nature12479 (2013).
76 Pinho-Ribeiro, F. A. et al. Blocking Neuronal Signaling to Immune Cells Treats Streptococcal Invasive Infection. Cell 173, 1083-1097 e1022, doi:10.1016/j.cell.2018.04.006 (2018).
77 Yissachar, N. et al. An Intestinal Organ Culture System Uncovers a Role for the Nervous System in Microbe-Immune Crosstalk. Cell 168, 1135-1148 e1112, doi:10.1016/j.cell.2017.02.009 (2017).
78 Anderson, P. & Gonzalez-Rey, E. Vasoactive intestinal peptide induces cell cycle arrest and regulatory functions in human T cells at multiple levels. Mol Cell Biol 30, 2537-2551, doi:10.1128/MCB.01282-09 (2010).
79 Li, J. M. et al. VIPhyb, an antagonist of vasoactive intestinal peptide receptor, enhances cellular antiviral immunity in murine cytomegalovirus infected mice. PLoS One 8, e63381, doi:10.1371/journal.pone.0063381 (2013).
80 Li, J. M. et al. Modulation of Immune Checkpoints and Graft-versus-Leukemia in Allogeneic Transplants by Antagonizing Vasoactive Intestinal Peptide Signaling. Cancer Res 76, 6802-6815, doi:10.1158/0008-5472.CAN-16-0427 (2016).
81 Petersen, C. T., Li, J. M. & Waller, E. K. Administration of a vasoactive intestinal peptide antagonist enhances the autologous anti-leukemia T cell response in murine models of acute leukemia. Oncoimmunology 6, e1304336, doi:10.1080/2162402X.2017.1304336 (2017).
82 Pozo, D., Anderson, P. & Gonzalez-Rey, E. Induction of alloantigen-specific human T regulatory cells by vasoactive intestinal peptide. J Immunol 183, 4346-4359, doi:10.4049/jimmunol.0900400 (2009).
83 Tebas, P. et al. Reduction of soluble CD163, substance P, programmed death 1 and inflammatory markers: phase 1B trial of aprepitant in HIV-1-infected adults. AIDS 29, 931-939, doi:10.1097/QAD.0000000000000638 (2015).
84 Heng, T. S., Painter, M. W. & Immunological Genome Project, C. The Immunological Genome Project: networks of gene expression in immune cells. Nature immunology 9, 1091-1094, doi:10.1038/ni1008-1091 (2008).
85 Bertin, S. et al. The ion channel TRPV1 regulates the activation and proinflammatory properties of CD4(+) T cells. Nature immunology 15, 1055-1063, doi:10.1038/ni.3009 (2014).
86 Luiz, A. P. et al. Cold sensing by NaV1.8-positive and NaV1.8-negative sensory neurons. Proc Natl Acad Sci USA 116, 3811-3816, doi:10.1073/pnas.1814545116 (2019).
87 Caterina, M. J., Rosen, T. A., Tominaga, M., Brake, A. J. & Julius, D. A capsaicin-receptor homologue with a high threshold for noxious heat. Nature 398, 436-441, doi:10.1038/18906 (1999).
88 Vermeer, D. W. et al. Metastatic model of HPV+ oropharyngeal squamous cell carcinoma demonstrates heterogeneity in tumor metastasis. Oncotarget 7, 24194-24207, doi:10.18632/oncotarget.8254 (2016).
89 Pellett, S., Tepp, W. H., Whitemarsh, R. C., Bradshaw, M. & Johnson, E. A. In vivo onset and duration of action varies for botulinum neurotoxin A subtypes 1-5. Toxicon 107, 37-42, doi:10.1016/j.toxicon.2015.06.021 (2015).
90 Binshtok, A. M., Bean, B. P. & Woolf, C. J. Inhibition of nociceptors by TRPV1-mediated entry of impermeant sodium channel blockers. Nature 449, 607-610, doi:10.1038/nature06191 (2007).
91 Blake, K. J. et al. Staphylococcus aureus produces pain through pore-forming toxins and neuronal TRPV1 that is silenced by QX-314. Nat Commun 9, 37, doi:10.1038/s41467-017-02448-6 (2018).
92 Roberson, D. P., Binshtok, A. M., Blasl, F., Bean, B. P. & Woolf, C. J. Targeting of sodium channel blockers into nociceptors to produce long-duration analgesia: a systematic study and review. Br J Pharmacol 164, 48-58, doi:10.1111/j.1476-5381.2011.01391.x (2011).
93 Ayala, G. E. et al. Cancer-related axonogenesis and neurogenesis in prostate cancer. Clin Cancer Res 14, 7593-7603, doi:10.1158/1078-0432.CCR-08-1164 (2008).
94 Amit, M. et al. Loss of p53 drives neuron reprogramming in head and neck cancer. Nature, doi:10.1038/s41586-020-1996-3 (2020).
95 Gasparini, G. et al. Nerves and Pancreatic Cancer: New Insights into a Dangerous Relationship. Cancers (Basel) 11, doi:10.3390/cancers11070893 (2019).
96 March, B. et al. Tumour innervation and neurosignalling in prostate cancer. Nat Rev Urol 17, 119-130, doi:10.1038/s41585-019-0274-3 (2020).
97 Zahalka, A. H. & Frenette, P. S. Nerves in cancer. Nat Rev Cancer, doi:10.1038/s41568-019-0237-2 (2020).
98 Cohen, J. A. et al. Cutaneous TRPV1(+) Neurons Trigger Protective Innate Type 17 Anticipatory Immunity. Cell 178, 919-932 e914, doi:10.1016/j.cell.2019.06.022 (2019).
99 Klagsbrun, M. & Eichmann, A. A role for axon guidance receptors and ligands in blood vessel development and tumor angiogenesis. Cytokine Growth Factor Rev 16, 535-548, doi:10.1016/j.cytogfr.2005.05.002 (2005).
100 Larrivee, B. et al. Activation of the UNC5B receptor by Netrin-1 inhibits sprouting angiogenesis. Genes Dev 21, 2433-2447, doi:10.1101/gad.437807 (2007).
101 Adams, R. H. & Eichmann, A. Axon guidance molecules in vascular patterning. Cold Spring Harb Perspect Biol 2, a001875, doi:10.1101/cshperspect.a001875 (2010).
102 Lejmi, E. et al. Netrin-4 inhibits angiogenesis via binding to neogenin and recruitment of Unc5B. Proc Natl Acad Sci USA 105, 12491-12496, doi:10.1073/pnas.0804008105 (2008).
103 Walchli, T. et al. Wiring the Vascular Network with Neural Cues: A CNS Perspective. Neuron 87, 271-296, doi:10.1016/j.neuron.2015.06.038 (2015).
104 Lu, X. et al. The netrin receptor UNC5B mediates guidance events controlling morphogenesis of the vascular system. Nature 432, 179-186, doi:10.1038/nature03080 (2004).
105 Carmeliet, P. & Tessier-Lavigne, M. Common mechanisms of nerve and blood vessel wiring. Nature 436, 193-200, doi:10.1038/nature03875 (2005).
106 Rodeck, U. & Herlyn, M. Growth factors in melanoma. Cancer Metastasis Rev 10, 89-101, doi:10.1007/bf00049407 (1991).
107 Surman, M., Stepien, E. & Przybylo, M. Melanoma-Derived Extracellular Vesicles: Focus on Their Proteome. Proteomes 7, doi:10.3390/proteomes7020021 (2019).
108 Peinado, H. et al. Melanoma exosomes educate bone marrow progenitor cells toward a pro-metastatic phenotype through MET. Nat Med 18, 883-891, doi:10.1038/nm.2753 (2012).
109 Chen, G. et al. Exosomal PD-L1 contributes to immunosuppression and is associated with anti-PD-1 response. Nature 560, 382-386, doi:10.1038/s41586-018-0392-8 (2018).
110 Zhu, Y. et al. Transforming growth factor beta induces sensory neuronal hyperexcitability, and contributes to pancreatic pain and hyperalgesia in rats with chronic pancreatitis. Mol Pain 8, 65, doi:10.1186/1744-8069-8-65 (2012).
111 Huber, R. et al. Tumour hypoxia promotes melanoma growth and metastasis via High Mobility Group Box-1 and M2-like macrophages. Sci Rep 6, 29914, doi:10.1038/srep29914 (2016).
112 Li, Q. et al. Overexpression of HMGB1 in melanoma predicts patient survival and suppression of HMGB1 induces cell cycle arrest and senescence in association with p21 (Waf1/Cip1) up-regulation via a p53-independent, Sp1-dependent pathway. Oncotarget 5, 6387-6403, doi:10.18632/oncotarget.2201 (2014).
113 Agalave, N. M. & Svensson, C. I. Extracellular high-mobility group box 1 protein (HMGB1) as a mediator of persistent pain. Mol Med 20, 569-578, doi:10.2119/molmed.2014.00176 (2015).
114 Gidron, Y. et al. The Relationship between a New Biomarker of Vagal Neuroimmunomodulation and Survival in Two Fatal Cancers. J Immunol Res 2018, 4874193, doi:10.1155/2018/4874193 (2018).
115 Keskinov, A. A. et al. Impact of the Sensory Neurons on Melanoma Growth In Vivo. PLoS One 11, e0156095, doi:10.1371/journal.pone.0156095 (2016).
116 Mohammadpour, H. et al. beta2 adrenergic receptor-mediated signaling regulates the immunosuppressive potential of myeloid-derived suppressor cells. J Clin Invest 129, 5537-5552, doi:10.1172/JC1129502 (2019).
117 Kamiya, A. et al. Genetic manipulation of autonomic nerve fiber innervation and activity and its effect on breast cancer progression. Nat Neurosci 22, 1289-1305, doi:10.1038/s41593-019-0430-3 (2019).
118 Harlin, H. et al. Chemokine expression in melanoma metastases associated with CD8+ T-cell recruitment. Cancer Res 69, 3077-3085, doi:10.1158/0008-5472.CAN-08-2281 (2009).
119 Riker, A. I. et al. The gene expression profiles of primary and metastatic melanoma yields a transition point of tumor progression and metastasis. BMC Med Genomics 1, 13, doi:10.1186/1755-8794-1-13 (2008).
120 Talantov, D. et al. Novel genes associated with malignant melanoma but not benign melanocytic lesions. Clin Cancer Res 11, 7234-7242, doi:10.1158/1078-0432.CCR-05-0683 (2005).
121 Laurent, C. et al. High PTP4A3 phosphatase expression correlates with metastatic risk in uveal melanoma patients. Cancer Res 71, 666-674, doi:10.1158/0008-5472.CAN-10-0605 (2011).
122 Lemeshow, S. et al. beta-Blockers and survival among Danish patients with malignant melanoma: a population-based cohort study. Cancer Epidemiol Biomarkers Prev 20, 2273-2279, doi:10.1158/1055-9965.EPI-11-0249 (2011).
123 McCourt, C. et al. Beta-blocker usage after malignant melanoma diagnosis and survival: a population-based nested case-control study. Br J Dermatol 170, 930-938, doi:10.1111/bjd.12894 (2014).
124 De Couck, M., Caers, R., Spiegel, D. & Gidron, Y. The Role of the Vagus Nerve in Cancer Prognosis: A Systematic and a Comprehensive Review. J Oncol 2018, 1236787, doi:10.1155/2018/1236787 (2018).
125 Rabben, H. L., Zhao, C. M., Hayakawa, Y., Wang, T. C. & Chen, D. Vagotomy and Gastric Tumorigenesis. Curr Neuropharmacol 14, 967-972, doi:10.2174/1570159x14666160121114854 (2016).
126 Hicks, B. M., Murray, L. J., Powe, D. G., Hughes, C. M. & Cardwell, C. R. beta-Blocker usage and colorectal cancer mortality: a nested case-control study in the UK Clinical Practice Research Datalink cohort. Ann Oncol 24, 3100-3106, doi:10.1093/annonc/mdt381 (2013).
127 Melhem-Bertrandt, A. et al. Beta-blocker use is associated with improved relapse-free survival in patients with triple-negative breast cancer. J Clin Oncol 29, 2645-2652, doi:10.1200/JCO.2010.33.4441 (2011).
128 Yang, E. V. et al. Norepinephrine upregulates VEGF, IL-8, and IL-6 expression in human melanoma tumor cell lines: implications for stress-related enhancement of tumor progression. Brain Behav Immun 23, 267-275, doi:10.1016/j.bbi.2008.10.005 (2009).
129 Ross, A. S. et al. Diameter of involved nerves predicts outcomes in cutaneous squamous cell carcinoma with perineural invasion: an investigator-blinded retrospective cohort study. Dermatol Surg 35, 1859-1866, doi:10.1111/j.1524-4725.2009.01354.x (2009).
130 Magro, C. M., Crowson, A. N. & Mihm, M. C. Unusual variants of malignant melanoma. Mod Pathol 19 Suppl 2, S41-70, doi:10.1038/modpathol.3800516 (2006).
131 Law, W. P., Pereira, N. & Vaska, K. Perineural spread of recurrent cutaneous melanoma along cervical nerves into the spinal cord. BJR Case Rep 3, 20160122, doi:10.1259/bjrcr.20160122 (2017).
132 Feasel, A. M., Brown, T. J., Bogle, M. A., Tschen, J. A. & Nelson, B. R. Perineural invasion of cutaneous malignancies. Dermatol Surg 27, 531-542, doi:10.1046/j.1524-4725.2001.00330.x (2001).
133 Fidler, I. J. Biological behavior of malignant melanoma cells correlated to their survival in vivo. Cancer Res 35, 218-224 (1975).
134 Meeth, K., Wang, J. X., Micevic, G., Damsky, W. & Bosenberg, M. W. The YUMM lines: a series of congenic mouse melanoma cell lines with defined genetic alterations. Pigment Cell Melanoma Res 29, 590-597, doi:10.1111/pcmr.12498 (2016).
135 Coppock, J. D. et al. mTOR inhibition as an adjuvant therapy in a metastatic model of HPV+ HNSCC. Oncotarget 7, 24228-24241, doi:10.18632/oncotarget.8286 (2016).
136 Voehringer, D., Liang, H. E. & Locksley, R. M. Homeostasis and effector function of lymphopenia-induced “memory-like” T cells in constitutively T cell-depleted mice. J Immunol 180, 4742-4753, doi:10.4049/jimmunol.180.7.4742 (2008).

### INCORPORATION BY REFERENCE

The present application refers to various issued patents, published patent applications, scientific journal articles, and other publications, all of which are incorporated herein by reference. The details of one or more embodiments of the invention are set forth herein. Other features, objects, and advantages of the invention will be apparent from the Detailed Description, the Figures, the Examples, and the Claims.

### EQUIVALENTS AND SCOPE

In the claims, articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

Furthermore, the invention encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the invention, or aspects of the invention, is/are referred to as comprising particular elements and/or features, certain embodiments of the invention or aspects of the invention consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the invention, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present invention that falls within the prior art may be explicitly excluded from any one or more of the claims. Because such embodiments are deemed to be known to one of ordinary skill in the art, they may be excluded even if the exclusion is not set forth explicitly herein. Any particular embodiment of the invention can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present invention, as defined in the following claims.

What is claimed is:

1. A method of treating cancer in a subject, the method comprising silencing tumor-innervating sensory neurons.

2. A method of treating cancer in a subject, the method comprising administering to the subject a therapeutically effective amount of a neuropeptide modulating agent.

3. A method of treating cancer in a subject, the method comprising administering to the subject a therapeutically effective amount of an agent that blocks the release or action of a neuropeptide from tumor-innervating neurons.

4. A method of treating cancer in a subject, the method comprising administering to the subject a therapeutically effective amount of a nociceptor modulating agent.

5. The method of claim 4, wherein the nociceptor modulating agent is a nociceptor antagonist.

6. The method of claim 5, wherein the nociceptor antagonist is a sodium channel blocker, calcium channel blocker, or sodium and calcium channel blocker.

7. The method of claim 6, wherein the sodium channel is Na_{V}1.8.

8. The method of any one of claims 2-7, wherein the neuropeptide modulating agent, agent that blocks the release or action of a neuropeptide, nociceptor modulating agent, or nociceptor antagonist is a compound comprising a quaternary amine.

9. The method of any one of claims 2-8, wherein the neuropeptide modulating agent, agent that blocks the release or action of a neuropeptide, nociceptor modulating agent, or nociceptor antagonist is QX-314:

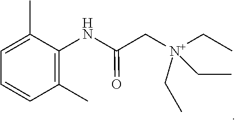 [SMILES: CC[N+](CC)(CC)CC(=O)Nc1c(C)cccc1C]

10. The method of any one of claims 2-8, wherein the neuropeptide modulating agent, agent that blocks the release or action of a neuropeptide, nociceptor modulating agent, or nociceptor antagonist is a compound selected from the group consisting of:

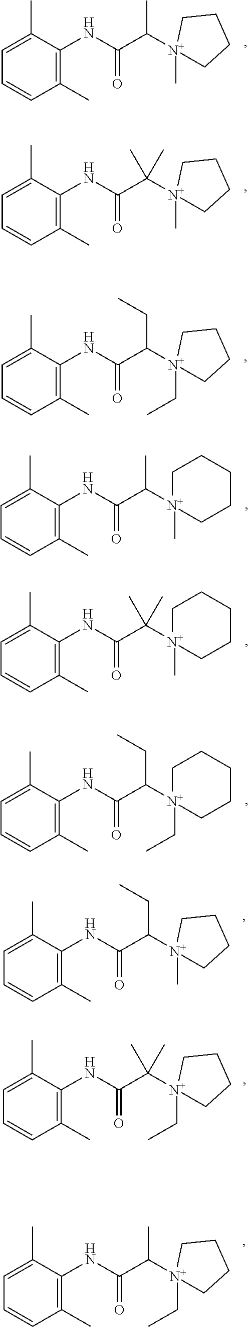

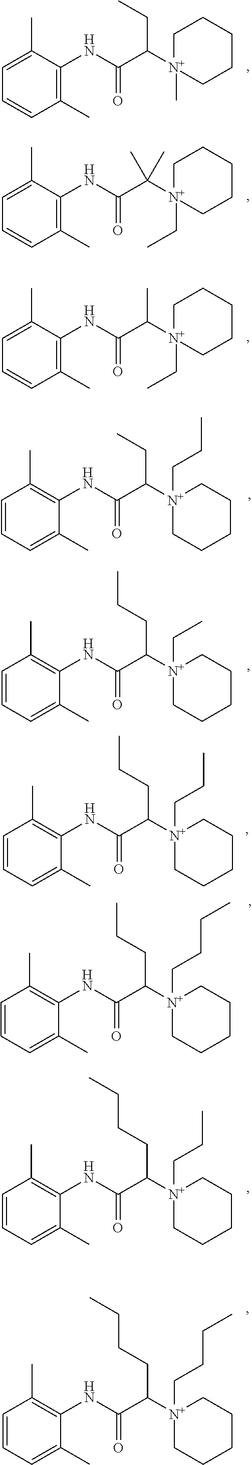

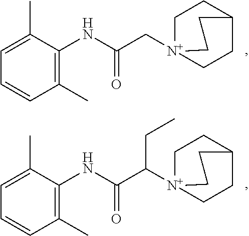 [SMILES: Cc1cccc(C)c1NC(=O)C[N+]12CCC(CC1)CC2 ; CCC(C(=O)Nc1c(C)cccc1C)[N+]12CCC(CC1)CC2]

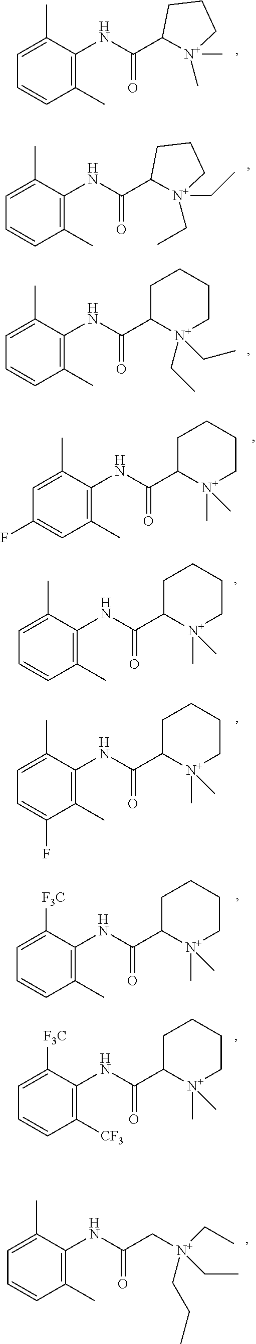

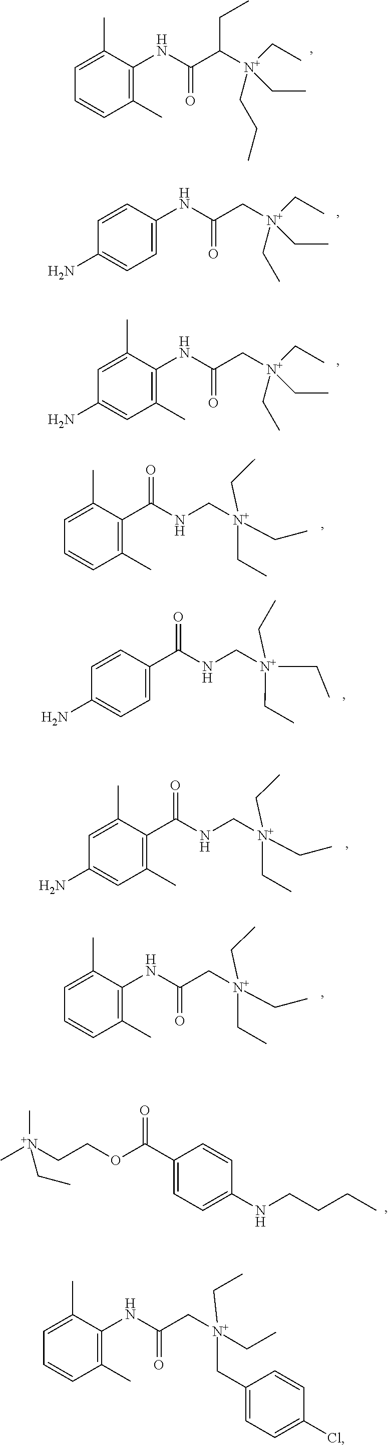

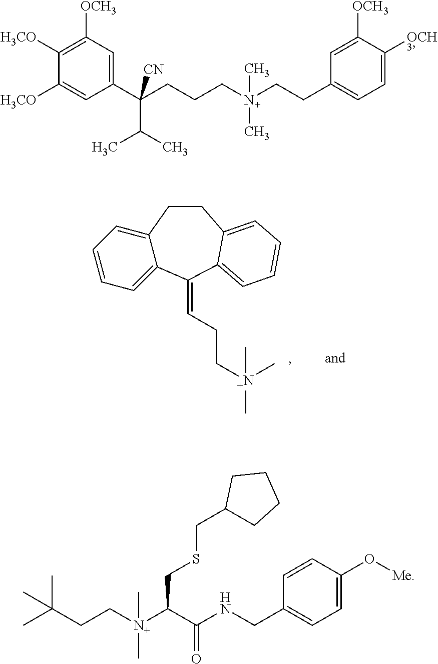 [SMILES: COc1ccc(CC[N+](C)(C)CCC[C@@](C#N)(c2cc(OC)c(OC)c(OC)c2)C(C)C)cc1OC ; C[N+](C)(C)CCC=C1c2ccccc2CCc2ccccc21 ; COc1ccc(CNC(=O)[C@H](CSCC2CCCC2)[N+](C)(C)CCC(C)(C)C)cc1]

11. The method of any one of claims 2-8, wherein the neuropeptide modulating agent, agent that blocks the release or action of a neuropeptide, nociceptor modulating agent, or nociceptor antagonist is a quaternary amine derivative or other permanently charged derivative of a compound selected from riluzole, mexilitine, phenytoin, carbamazepine, procaine, articaine, bupivicaine, mepivicaine, tocainide, prilocaine, diisopyramide, bencyclane, quinidine, bretylium, lifarizine, lamotrigine, flunarizine, and fluspirilene.

12. A method of treating cancer in a subject, the method comprising administrating to the subject a therapeutically effective amount of an agent that blocks vesicle release from tumor-innervating nociceptors.

13. The method of any one of claims 2-7 and 12, wherein the neuropeptide modulating agent, agent that blocks the release or action of a neuropeptide, an agent that blocks vesicle release, nociceptor modulating agent, or nociceptor antagonist is a neurotoxic protein.

14. The method of claim 13, wherein the neurotoxic protein is a botulinum neurotoxin or tetanus toxin (TeNT).

15. The method of claim 14, wherein the botulinum neurotoxin is BoNT/a.

16. A method of treating cancer in a subject, the method comprising administering to a subject a therapeutically effective amount of a calcitonin gene-related peptide (CGRP) modulating agent.

17. The method of any one of the preceding claims, wherein the CGRP modulating agent is a CGRP receptor antagonist.

18. The method of claim 17, wherein the CGRP receptor antagonist is a RAMP1, RAMP3, or Vpac1 blocker.

19. The method of claim 17 or 18, wherein the CGRP receptor antagonist is a RAMP1 blocker.

20. The method of any one of claims 17-19, wherein the CGRP receptor antagonist is erenumab, fremanezumab, fremanezumab, eptinezumab, ubrogepant, or rimegepant.

21. The method of any one of claims 17-19, wherein the CGRP receptor antagonist is BIBN 4096.

22. A method of treating cancer in a subject, the method comprising ablating an ion channel in a subject, wherein the ion channel is a sodium ion channel or TRPV ion channel.

23. The method of claim 22, wherein the sodium ion channel is Na_{V}1.8.

24. The method of claim 22, wherein the TRPV ion channel is TRPV1.

25. The method of any one of claims 22-24, wherein the ion channel is genetically ablated.

26. The method of any one of claims 1-25, wherein the cancer is skin cancer, breast cancer, prostate cancer, ovarian cancer, pancreatic cancer, gastric cancer, or a tumor.

27. The method of any one of claims 1-26, wherein the cancer is melanoma.

28. The method of any one of claims 1-27, wherein the cancer is a tumor.

29. The method of any one of claims 1-28, wherein the method decreases tumor growth, volume, and/or size.

30. The method of any one of claims 1-29, wherein the method inhibits or decreases cancer cell proliferation.

31. The method of any one of claims 1-30, wherein the method increases subject survival.

32. The method of any one of claims 1-31, wherein the method promotes anti-tumor activity.

33. The method of any one of claims 1-32, wherein the method increases lymphocyte numbers.

34. The method of any one of claims 1-33, the method improves response to chemotherapeutics.

35. The method of any one of claims 1-34, wherein the method decreases tumor comorbidities.

36. The method of claim 35, wherein the comorbidity is pain or itch.

37. The method of any one of claims 1-36 further comprising administering to the subject an additional therapy.

38. The method of claim 37, wherein the additional therapy is chemotherapy, radioimmunotherapy, surgical therapy, immunotherapy, radiation therapy, or targeted therapy, or any combination thereof.

39. The method of claim 38, wherein the method increases efficacy of the immunotherapy.

40. The method of any one of claims 1-39, where the method leads to exhaustion of tumor-infiltrating lymphocytes.

41. A method of treating cancer in a subject, the method comprising administering to a subject a therapeutically effective amount of (i) an anti-cancer agent and (ii) QX-314, BoNT/a, or BIBN 4096.

42. The method of claim 41, wherein the anti-cancer agent is a biotherapeutic anti-cancer agent or a chemotherapeutic agent.

43. The method of claim 41 or 42, wherein the anti-cancer agent or chemotherapeutic agent is dacarbazine or cisplatin.

44. A composition comprising (i) an anti-cancer agent, (ii) a nociceptor modulating agent, nociceptor antagonist, neuropeptide modulating agent, an agent that blocks vesicle release, or agent that blocks the release or action of a neuropeptide from tumor-innervating nociceptor described herein, and (iii) optionally a pharmaceutically acceptable excipient.

45. The composition of claim 44, wherein the composition comprises (i) dacarbazine or cisplatin, (ii) QX-314, BoNT/a, or BIBN 4096, and (iii) optionally a pharmaceutically acceptable excipient.
